SEQUENCE LISTING (153 sequences)

SEQ ID NO: 1 - RNA, 9678 nt, Hepatitis C virus
  misc_feature (24)..(56): /note="target region of the 5'UTR"
  misc_feature (323)..(346): /note="target region of the 5'UTR"
  misc_feature (372)..(399): /note="target region of the CDS"
  misc_feature (459)..(474): /note="target region of the CDS"
  misc_feature (651)..(668): /note="target region of the CDS"
  misc_feature (9567)..(9600): /note="target region of the 3'UTR"
accugccccuaauaggggcgacacuccgccaugaaucacuccccugugaggaacuacugucuucacgcagaaagcgccuagccauggcguuaguaugagugucguacagccuccaggccccccccucccgggagagccauaguggucugcggaaccggugaguacaccggaauugccgggaagacuggguccuuucuuggauaaacccacucuaugcccggccauuugggcgugcccccgcaagacugcuagccgaguagcguuggguugcgaaaggccuugugguacugccugauagggcgcuugcgagugccccgggaggucucguagaccgugcaccaugagcacaaauccuaaaccucaaagaaaaaccaaaagaaacaccaaccgucgcccagaagacguuaaguucccgggcggcggccagaucguuggcggaguauacuuguugccgcgcaggggccccagguugggugugcgcacgacaaggaaaacuucggagcggucccagccacgugggagacgccagcccauccccaaagaucggcgcuccacuggcaaggccuggggaaaaccaggucgccccuggccccuauaugggaaugagggacucggcugggcaggauggcuccuguccccccgaggcucucgccccuccuggggccccacugacccccggcauaggucgcgcaacguggguaaagucaucgacacccuaacguguggcuuugccgaccucaugggguacauccccgucguaggcgccccgcuuaguggcgccgccagagcugucgcgcacggcgugagaguccuggaggacgggguuaauuaugcaacagggaaccuacccgguuuccccuuuucuaucuucuugcuggcccuguuguccugcaucaccguuccggucucugcugcccaggugaagaauaccaguagcagcuacauggugaccaaugacugcuccaaugacagcaucacuuggcagcucgaggcugcgguucuccacguccccgggugcgucccgugcgagagaguggggaauacgucacgguguugggugccagucucgccaaacauggcugugcggcagcccggugcccucacgcagggucugcggacgcacaucgauaugguugugauguccgccaccuucugcucugcucucuacgugggggaccucuguggcggggugaugcucgcggcccagguguucaucgucucgccgcaguaccacugguuugugcaagaaugcaauugcuccaucuacccuggcaccaucacuggacaccgcauggcaugggacaugaugaugaacuggucgcccacggccaccaugauccuggcguacgugaugcgcguccccgaggucaucauagacaucguuagcggggcucacuggggcgucauguucggcuuggccuacuucucuaugcagggagcgugggcgaaggucauugucauccuucugcuggccgcugggguggacgcgggcaccaccaccguuggaggcgcuguugcacguuccaccaacgugauugccggcguguucagccauggcccucagcagaacauucagcucauuaacaccaacggcaguuggcacaucaaccguacugccuugaauugcaaugacuccuugaacaccggcuuucucgcggccuuguucuacaccaaccgcuuuaacucgucaggguguccagggcgccuguccgccugccgcaacaucgaggcuuuccggauaggguggggcacccuacaguacgaggauaaugucaccaauccagaggauaugaggccguacugcuggcacuaccccccaaagccguguggcguaguccccgcgaggucuguguguggcccaguguacuguuucacccccagcccgguaguagugggcacgaccgacagacguggagugcccaccuacacauggggagagaaugagacagaugucuuccuacugaacagcacccgaccgccgcagggcucaugguucggcugcacguggaugaacuccacugguuucaccaagacuuguggcgcgccaccuugccgcaccagagcugacuucaacgccagcacggacuuguugugcccuacggauuguuuuaggaagcauccugaugccacuuauauuaagugugguucugggcccuggcucacaccaaagugccugguccacuacccuuacagacucuggcauuaccccugcacagucaauuuuaccaucuucaagauaagaauguauguaggggggguugagcacaggcucacggccgcaugcaacuucacucguggggaucgcugcgacuuggaggacagggacaggagucagcugucuccucuguugcacucuaccacggaaugggccauccugcccugcaccuacucagacuuacccgcuuugucaacuggucuucuccaccuucaccagaacaucguggacguacaauacauguauggccucucaccugcuaucacaaaauacgucguucgaugggagugggugguacucuuauuccugcucuuagcggacgccagagucugcgccugcuuguggaugcucaucuuguugggccaggccgaagcagcauuggagaaguuggucgucuugcacgcugcgagugcggcuaacugccauggccuccuauauuuugccaucuucuucguggcagcuuggcacaucaggggucgggugguccccuugaccaccuauugccucacuggccuauggcccuucugccuacugcucauggcacugccccggcaggcuuaugccuaugacgcaccugugcacggacagauaggcguggguuuguugauauugaucacccucuucacacucaccccgggguauaagacccuccucggccagugucuguggugguugugcuaucuccugacccugggggaagccaugauucaggaguggguaccacccaugcaggugcgcggcggccgcgauggcaucgcgugggccgucacuauauucugcccgggugugguguuugacauuaccaaauggcuuuuggcguugcuugggccugcuuaccucuuaagggccgcuuugacacaugugccguacuucgucagagcucacgcucugauaaggguaugcgcuuuggugaagcagcucgcgggggguagguauguucagguggcgcuauuggcccuuggcagguggacuggcaccuacaucuaugaccaccucacaccuaugucggacugggccgcuagcggccugcgcgacuuagcggucgccguggaacccaucaucuucaguccgauggagaagaaggucaucgucuggggagcggagacggcugcauguggggacauucuacauggacuucccguguccgcccgacucggccaggagauccuccucggcccagcugauggcuacaccuccaagggguggaagcuccuugcucccaucacugcuuaugcccagcaaacacgaggccuccugggcgccauaguggugaguaugacggggcgugacaggacagaacaggccggggaaguccaaauccuguccacagucucucaguccuuccucggaacaaccaucucggggguuuuguggacuguuuaccacggagcuggcaacaagacucuagccggcuuacgggguccggucacgcagauguacucgagugcugagggggacuugguaggcuggcccagccccccugggaccaagucuuuggagccgugcaaguguggagccgucgaccuauaucuggucacgcggaacgcugaugucaucccggcucggagacgcggggacaagcggggagcauugcucuccccgagacccauuucgaccuugaagggguccucgggggggccggugcucugcccuaggggccacgucguugggcucuuccgagcagcugugugcucucggggcguggccaaauccaucgauuucauccccguugagacacucgacguuguuacaaggucucccacuuucagugacaacagcacgccaccggcugugccccagaccuaucaggucggguacuugcaugcuccaacuggcaguggaaagagcaccaaggucccugucgcguaugccgcccagggguacaaaguacuagugcuuaaccccucgguagcugccacccugggguuuggggcguaccuauccaaggcacauggcaucaaucccaacauuaggacuggagucaggaccgugaugaccggggaggccaucacguacuccacauauggcaaauuucucgccgaugggggcugcgcuagcggcgccuaugacaucaucauaugcgaugaaugccacgcuguggaugcuaccuccauucucggcaucggaacgguccuugaucaagcagagacagccggggucagacuaacugugcuggcuacggccacaccccccgggucagugacaaccccccaucccgauauagaagagguaggccucgggcgggagggugagauccccuucuaugggagggcgauuccccuauccugcaucaagggagggagacaccugauuuucugccacucaaagaaaaagugugacgagcucgcggcggcccuucggggcaugggcuugaaugccguggcauacuauagaggguuggacgucuccauaauaccagcucagggagaugugguggucgucgccaccgacgcccucaugacgggguacacuggagacuuugacuccgugaucgacugcaauguagcggucacccaagcugucgacuucagccuggaccccaccuucacuauaaccacacagacugucccacaagacgcugucucacgcagucagcgccgcgggcgcacagguagaggaagacagggcacuuauagguauguuuccacuggugaacgagccucaggaauguuugacaguguagugcuuugugagugcuacgacgcaggggcugcgugguacgaucucacaccagcggagaccaccgucaggcuuagagcguauuucaacacgcccggccuacccgugugucaagaccaucuugaauuuugggaggcaguuuucaccggccucacacacauagacgcccacuuccucucccaaacaaagcaagcgggggagaacuucgcguaccuaguagccuaccaagcuacggugugcgccagagccaaggccccucccccguccugggacgccauguggaagugccuggcccgacucaagccuacgcuugcgggccccacaccucuccuguaccguuugggcccuauuaccaaugaggucacccucacacacccugggacgaaguacaucgccacaugcaugcaagcugaccuugaggucaugaccagcacguggguccuagcuggaggaguccuggcagccgucgccgcauauugccuggcgacuggaugcguuuccaucaucggccgcuugcacgucaaccagcgagucgucguugcgccggauaaggagguccuguaugaggcuuuugaugagauggaggaaugcgccucuagggcggcucucaucgaagaggggcagcggauagccgagauguugaaguccaagauccaaggcuugcugcagcaggccucuaagcaggcccaggacauacaacccgcuaugcaggcuucauggcccaaaguggaacaauuuugggccagacacauguggaacuucauuagcggcauccaauaccucgcaggauugucaacacugccagggaaccccgcgguggcuuccaugauggcauucagugccgcccucaccaguccguugucgaccaguaccaccauccuucucaacaucaugggaggcugguuagcgucccagaucgcaccacccgcgggggccaccggcuuugucgucaguggccuggugggggcugccgugggcagcauaggccuggguaaggugcugguggacauccuggcaggauauggugcgggcauuucgggggcccucgucgcauucaagaucaugucuggcgagaagcccucuauggaagaugucaucaaucuacugccugggauccugucuccgggagcccugguggugggggucaucugcgcggccauucugcgccgccacgugggaccgggggagggcgcgguccaauggaugaacaggcuuauugccuuugcuuccagaggaaaccacgucgccccuacucacuacgugacggagucggaugcgucgcagcgugugacccaacuacuuggcucucuuacuauaaccagccuacucagaagacuccacaauuggauaacugaggacugccccaucccaugcuccggauccuggcuccgcgacgugugggacuggguuugcaccaucuugacagacuucaaaaauuggcugaccucuaaauuguuccccaagcugcccggccuccccuucaucucuugucaaaagggguacaagggugugugggccggcacuggcaucaugaccacgcgcugcccuugcggcgccaacaucucuggcaauguccgccugggcucuaugaggaucacagggccuaaaaccugcaugaacaccuggcaggggaccuuuccuaucaauugcuacacggagggccagugcgcgccgaaaccccccacgaacuacaagaccgccaucuggaggguggcggccucggaguacgcggaggugacgcagcaugggucguacuccuauguaacaggacugaccacugacaaucugaaaauuccuugccaacuaccuucuccagaguuuuucuccuggguggacggugugcagauccauagguuugcacccacaccaaagccguuuuuccgggaugaggucucguucugcguugggcuuaauuccuaugcugucgggucccagcuucccugugaaccugagcccgacgcagacguauugagguccaugcuaacagauccgccccacaucacggcggagacugcggcgcggcgcuuggcacggggaucaccuccaucugaggcgagcuccucagugagccagcuaucagcaccgucgcugcgggccaccugcaccacccacagcaacaccuaugacguggacauggucgaugccaaccugcucauggagggcgguguggcucagacagagccugaguccagggugcccguucuggacuuucucgagccaauggccgaggaagagagcgaccuugagcccucaauaccaucggagugcaugcuccccaggagcggguuuccacgggccuuaccggcuugggcacggccugacuacaacccgccgcucguggaaucguggaggaggccagauuaccaaccgcccaccguugcugguugugcucuccccccccccaagaaggccccgacgccucccccaaggagacgccggacagugggucugagcgagagcaccauaucagaagcccuccagcaacuggccaucaagaccuuuggccagccccccucgagcggugaugcaggcucguccacgggggcgggcgccgccgaauccggcgguccgacguccccuggugagccggcccccucagagacagguuccgccuccucuaugcccccccucgagggggagccuggagauccggaccuggagucugaucagguagagcuucaaccucccccccaggggggggggguagcucccgguucgggcucggggucuuggucuacuugcuccgaggaggacgauaccaccgugugcugcuccaugucauacuccuggaccggggcucuaauaacucccuguagccccgaagaggaaaaguugccaaucaacccuuugaguaacucgcuguugcgauaccauaacaagguguacuguacaacaucaaagagcgccucacagagggcuaaaaagguaacuuuugacaggacgcaagugcucgacgcccauuaugacucagucuuaaaggacaucaagcuagcggcuuccaaggucagcgcaaggcuccucaccuuggaggaggcgugccaguugacuccaccccauucugcaagauccaaguauggauucggggccaaggagguccgcagcuuguccgggagggccguuaaccacaucaaguccguguggaaggaccuccuggaagacccacaaacaccaauucccacaaccaucauggccaaaaaugagguguucugcguggaccccgccaagggggguaagaaaccagcucgccucaucguuuacccugaccucggcguccgggucugcgagaaaauggcccucuaugacauuacacaaaagcuuccucaggcgguaaugggagcuuccuauggcuuccaguacuccccugcccaacggguggaguaucucuugaaagcaugggcggaaaagaaggaccccauggguuuuucguaugauacccgaugcuucgacucaaccgucacugagagagacaucaggaccgaggaguccauauaccaggccugcucccugcccgaggaggcccgcacugccauacacucgcugacugagagacuuuacguaggagggcccauguucaacagcaagggucaaaccugcgguuacagacguugccgcgccagcggggugcuaaccacuagcauggguaacaccaucacaugcuaugugaaagcccuagcggccugcaaggcugcggggauaguugcgcccacaaugcugguaugcggcgaugaccuaguagucaucucagaaagccaggggacugaggaggacgagcggaaccugagagccuucacggaggccaugaccagguacucugccccuccuggugauccccccagaccggaauaugaccuggagcuaauaacauccuguuccucaaaugugucuguggcguugggcccgcggggccgccgcagauacuaccugaccagagacccaaccacuccacucgcccgggcugccugggaaacaguuagacacuccccuaucaauucauggcugggaaacaucauccaguaugcuccaaccauauggguucgcaugguccuaaugacacacuucuucuccauucucaugguccaagacacccuggaccagaaccucaacuuugagauguauggaucaguauacuccgugaauccuuuggaccuuccagccauaauugagagguuacacgggcuugacgccuuuucuaugcacacauacucucaccacgaacugacgcggguggcuucagcccucagaaaacuuggggcgccaccccucaggguguggaagagucgggcucgcgcagucagggcgucccucaucucccguggagggaaagcggccguuugcggccgauaucucuucaauugggcggugaagaccaagcucaaacucacuccauugccggaggcgcgccuacuggacuuauccaguugguucaccgucggcgccggcgggggcgacauuuuucacagcgugucgcgcgcccgaccccgcucauuacucuucggccuacuccuacuuuucguagggguaggccucuuccuacuccccgcucgguagagcggcacacacuagguacacuccauagcuaacuguuccuuuuuuuuuuuuuuuuuuuuuuuuuuuuuuuuuuuuuuuuuuucuuuuuuuuuuuuuucccucuuucuucccuucucaucuuauucuacuuucuuucuugguggcuccaucuuagcccuagucacggcuagcugugaaagguccgugagccgcaugacugcagagagugccguaacuggucucucugcagaucaugu

SEQ ID NO: 2 - RNA, 294 nt, Artificial sequence
  Circular Artificial sequence to IRES1 (hepatitis C virus) (circ_hcv_ires1)
  misc_feature (1)..(33): /note="hybridization site to IRES (hepatitis c virus)"
  misc_feature (1)..(33): /note="hybridization site to IRES (hepatitis c virus)"
  misc_feature (43)..(75): /note="hybridization site to IRES (hepatitis c virus)"
  misc_feature (85)..(117): /note="hybridization site to IRES (hepatitis c virus)"
  misc_feature (127)..(159): /note="hybridization site to IRES (hepatitis c virus)"
  misc_feature (169)..(201): /note="hybridization site to IRES (hepatitis c virus)"
  misc_feature (169)..(201): /note="hybridization site to IRES (hepatitis c virus)"
  misc_feature (211)..(243): /note="hybridization site to IRES (hepatitis c virus)"
  misc_feature (253)..(285): /note="hybridization site to IRES (hepatitis c virus)"
uaguuccucacgggggagugguucgugguggagcggcgccaaugguuuuuugcgggggagugguucguggcggggguaaccccuuaguucuucaugggggggugguucaugguggggaaacuaccguaguuuuucacaggggggugguucauggcggagagaagcgcuuaguuucuuaugggggagugauucauggcggaguguggggcaugguuuuucgcaggggagugauucgugguggggagaguuuugugguucuuuguaggggggugauucgugguggggacaucauug

SEQ ID NO: 3 - RNA, 373 nt, Artificial sequence
  Circular Artificial sequence to IRES2 (hepatitis C virus)
  misc_feature (11)..(34): /note="hybridization site to IRES (hepatitis c virus)"
  misc_feature (44)..(67): /note="hybridization site to IRES (hepatitis c virus)"
  misc_feature (77)..(100): /note="hybridization site to IRES (hepatitis c virus)"
  misc_feature (110)..(123): /note="hybridization site to IRES (hepatitis c virus)"
  misc_feature (133)..(166): /note="hybridization site to IRES (hepatitis c virus)"
  misc_feature (176)..(199): /note="hybridization site to IRES (hepatitis c virus)"
  misc_feature (209)..(232): /note="hybridization site to IRES (hepatitis c virus)"
  misc_feature (242)..(265): /note="hybridization site to IRES (hepatitis c virus)"
  misc_feature (275)..(298): /note="hybridization site to IRES (hepatitis c virus)"
  misc_feature (308)..(331): /note="hybridization site to IRES (hepatitis c virus)"
  misc_feature (341)..(373): /note="hybridization site to IRES (hepatitis c virus)"
ugccgaaaaugcuuauggugcacgguuugcgagagcaagaagagcuuguggugcaugguuugcggggacaguaaaggcucauggugcauggucuacgaggugagauagcguuuaugguguacggucugugggaaauuccgguguucguggugugcggucugugggauauuagaucguucaugguguacgguuuguggggauaauagcggcuuauggugcacggucuaugggacgcgagaaggcucauggugugugguuugugaggcagugugcgguucauggugcacggucugcgagacaccaggcggcucguggugcacggucuauggggugucgucgugcuuguggugugcggucuaugagggcggcugaa

SEQ ID NO: 4 - RNA, 346 nt, Artificial sequence
  Circular Artificial sequence to CDS1 (hepatitis C virus) (circ_hcv_cds1)
  misc_feature (11)..(38): /note="hybridization site to CDS1 (hepatitis C virus)"
  misc_feature (53)..(80): /note="hybridization site to CDS1 (hepatitis C virus)"
  misc_feature (95)..(122): /note="hybridization site to CDS1 (hepatitis C virus)"
  misc_feature (137)..(164): /note="hybridization site to CDS1 (hepatitis C virus)"
  misc_feature (179)..(206): /note="hybridization site to CDS1 (hepatitis C virus)"
  misc_feature (221)..(248): /note="hybridization site to CDS1 (hepatitis C virus)"
  misc_feature (263)..(290): /note="hybridization site to CDS1 (hepatitis C virus)"
  misc_feature (305)..(332): /note="hybridization site to CDS1 (hepatitis C virus)"
cugcacuggguuugggcgacgguugguguuuuuuuuggacgagauuucuugcucugggcgacgguugguguuucuuuuggguguuagugcguacucugggcgaugguugguguuucuuuugguaauuauuacuuucuuugggcgaugguugguguuuuuuuugguggggugaaaagaguuugggcggugguugguguuucuuuuggcguggugaguaacauuuggguggcgguugguguuuuuuuuggcugcaacgcaccgguuugggcgacgguugguguuucuuuugggggucaaccgggaaucuggguggcgguugguguuuuuuuuggugauggccgaggua

SEQ ID NO: 5 - RNA, 355 nt, Artificial sequence
  Circular Artificial sequence to IRES1, IRES2 and CDS1 (hepatitis C virus) (circ_hcv_combo1)
  misc_feature (11)..(43): /note="hybridization site to IRES1 (hepatitis C virus)"
  misc_feature (54)..(77): /note="hybridization site to IRES2 (hepatitis C virus)"
  misc_feature (88)..(115): /note="hybridization site to CDS1 (hepatitis C virus)"
  misc_feature (126)..(158): /note="hybridization site to IRES1 (hepatitis C virus)"
  misc_feature (169)..(192): /note="hybridization site to IRES2 (hepatitis C virus)"
  misc_feature (203)..(230): /note="hybridization site to CDS1 (hepatitis C virus)"
  misc_feature (241)..(273): /note="hybridization site to IRES1 (hepatitis C virus)"
  misc_feature (284)..(307): /note="hybridization site to IRES2 (hepatitis C virus)"
  misc_feature (318)..(345): /note="hybridization site to IRES2 (hepatitis C virus)"
gcgccaaguauaguuccucacaggggagugauuuaugguggagaccccuaaacgcuuguggugcacggucuacgggguaccgagaaguuugggcggugguugguguuuuuuuuggcgcuugugggugguuuuuugcaggggagugauuuaugguggaggcaagaguuugcuuaugguguacggucugugagaugacaucagguuugggcggcgguugguguuuuuuuuggaugaggaaccuaguucuucauaggggagugauuugugguggagacagcgaguuguuuguggugcacgguuuacgagaagaaugaagaucuggguggcgguugguguuucuuuugguagcacaugu

SEQ ID NO: 6 - RNA, 288 nt, Artificial sequence
  Circular Artificial sequence to CDS2 (hepatitis C virus) (circ_hcv_cds2)
  misc_feature (4)..(14): /note="hybridization site to CDS2 (hepatitis C virus)"
  misc_feature (28)..(38): /note="hybridization site to CDS2 (hepatitis C virus)"
  misc_feature (52)..(62): /note="hybridization site to CDS2 (hepatitis C virus)"
  misc_feature (76)..(86): /note="hybridization site to CDS2 (hepatitis C virus)"
  misc_feature (100)..(110): /note="hybridization site to CDS2 (hepatitis C virus)"
  misc_feature (124)..(134): /note="hybridization site to CDS2 (hepatitis C virus)"
  misc_feature (148)..(158): /note="hybridization site to CDS2 (hepatitis C virus)"
  misc_feature (172)..(182): /note="hybridization site to CDS2 (hepatitis C virus)"
  misc_feature (196)..(206): /note="hybridization site to CDS2 (hepatitis C virus)"
  misc_feature (220)..(230): /note="hybridization site to CDS2 (hepatitis C virus)"
  misc_feature (244)..(254): /note="hybridization site to CDS2 (hepatitis C virus)"
  misc_feature (266)..(278): /note="hybridization site to CDS2 (hepatitis C virus)"
cccccuggggcucugaugaggaaccucucugggguccccacagcgagucuccuuggggccccuagaaugaaguucuuugggguuucauagcacgguucuccugggguuuucauacgauugucucuuggggucuugcguguuuacccuuuugggguccugcuaagggggcuuucuggggccuuucgaauaagucuuuuuggggccucguuuuaucacucuucugggguucccagccuuucccuucuuggggcuccuccgaccaugccccuugggguucuacccuguaug

SEQ ID NO: 7 - RNA, 10722 nt, Dengue virus
  misc_feature (108)..(140): /note="target region of cHP"
  misc_feature (10615)..(10646): /note="target region of 3UTR"
aguuguuagucuacguggaccgacaaagacagauucuuugagggagcuaagcucaacguaguucuaacaguuuuuuaauuagagagcagaucucugaugaauaaccaacggaaaaaggcgaaaaacacgccuuucaauaugcugaaacgcgagagaaaccgcgugucgacugugcaacagcugacaaagagauucucacuuggaaugcugcagggacgaggaccauuaaaacuguucauggcccugguggcguuccuucguuuccuaacaaucccaccaacagcagggauauugaagagauggggaacaauuaaaaaaucaaaagcuauuaauguuuugagaggguucaggaaagagauuggaaggaugcugaacaucuugaauaggagacgcagaucugcaggcaugaucauuaugcugauuccaacagugauggcguuccauuuaaccacacguaacggagaaccacacaugaucgucagcagacaagagaaagggaaaagucuucuguuuaaaacagaggauggcgugaacauguguacccucauggccauggaccuuggugaauugugugaagacacaaucacguacaaguguccccuucucaggcagaaugagccagaagacauagacuguuggugcaacucuacguccacguggguaacuuaugggacguguaccaccaugggagaacauagaagagaaaaaagaucaguggcacucguuccacaugugggaaugggacuggagacacgaacugaaacauggaugucaucagaaggggccuggaaacauguccagagaauugaaacuuggaucuugagacauccaggcuucaccaugauggcagcaauccuggcauacaccauaggaacgacacauuuccaaagagcccugauuuucaucuuacugacagcugucacuccuucaaugacaaugcguugcauaggaaugucaaauagagacuuuguggaagggguuucaggaggaagcuggguugacauagucuuagaacauggaagcugugugacgacgauggcaaaaaacaaaccaacauuggauuuugaacugauaaaaacagaagccaaacagccugccacccuaaggaaguacuguauagaggcaaagcuaaccaacacaacaacagaaucucgcugcccaacacaaggggaacccagccuaaaugaagagcaggacaaaagguucgucugcaaacacuccaugguagacagaggauggggaaauggauguggacuauuuggaaagggaggcauugugaccugugcuauguucagaugcaaaaagaacauggaaggaaaaguugugcaaccagaaaacuuggaauacaccauugugauaacaccucacucaggggaagagcaugcagucggaaaugacacaggaaaacauggcaaggaaaucaaaauaacaccacagaguuccaucacagaagcagaauugacagguuauggcacugucacaauggagugcucuccaagaacgggccucgacuucaaugagaugguguugcugcagauggaaaauaaagcuuggcuggugcacaggcaaugguuccuagaccugccguuaccaugguugcccggagcggacacacaagggucaaauuggauacagaaagagacauuggucacuuucaaaaauccccaugcgaagaaacaggauguuguuguuuuaggaucccaagaaggggccaugcacacagcacuuacaggggccacagaaauccaaaugucaucaggaaacuuacucuucacaggacaucucaagugcaggcugagaauggacaagcuacagcucaaaggaaugucauacucuaugugcacaggaaaguuuaaaguugugaaggaaauagcagaaacacaacauggaacaauaguuaucagagugcaauaugaaggggacggcucuccaugcaagaucccuuuugagauaauggauuuggaaaaaagacaugucuuaggucgccugauuacagucaacccaauugugacagaaaaagauagcccagucaacauagaagcagaaccuccauucggagacagcuacaucaucauaggaguagagccgggacaacugaagcucaacugguuuaagaaaggaaguucuaucggccaaauguuugagacaacaaugaggggggcgaagagaauggccauuuuaggugacacagccugggauuuuggauccuugggaggaguguuuacaucuauaggaaaggcucuccaccaagucuuuggagcaaucuauggagcugccuucagugggguuucauggacuaugaaaauccucauaggagucauuaucacauggauaggaaugaauucacgcagcaccucacugucugugacacuaguauuggugggaauugugacacuguauuugggagucauggugcaggccgauagugguugcguugugagcuggaaaaacaaagaacugaaauguggcagugggauuuucaucacagacaacgugcacacauggacagaacaauacaaguuccaaccagaauccccuucaaaacuagcuucagcuauccagaaagcccaugaagagggcauuuguggaauccgcucaguaacaagacuggagaaucugauguggaaacaaauaacaccagaauugaaucacauucuaucagaaaaugaggugaaguuaacuauuaugacaggagacaucaaaggaaucaugcaggcaggaaaacgaucucugcggccucagcccacugagcugaaguauucauggaaaacauggggcaaagcaaaaaugcucucuacagagucucauaaccagaccuuucucauugauggccccgaaacagcagaaugccccaacacaaauagagcuuggaauucguuggaaguugaagacuauggcuuuggaguauucaccaccaauauauggcuaaaauugaaagaaaaacaggauguauucugcgacucaaaacucaugucagcggccauaaaagacaacagagccguccaugccgauauggguuauuggauagaaagugcacucaaugacacauggaagauagagaaagccucuuucauugaaguuaaaaacugccacuggccaaaaucacacacccucuggagcaauggagugcuagaaagugagaugauaauuccaaagaaucucgcuggaccagugucucaacacaacuauagaccaggcuaccauacacaaauaacaggaccauggcaucuagguaagcuugagauggacuuugauuucugugauggaacaacagugguagugacugaggacugcggaaauagaggacccucuuugagaacaaccacugccucuggaaaacucauaacagaauggugcugccgaucuugcacauuaccaccgcuaagauacagaggugaggaugggugcugguacgggauggaaaucagaccauugaaggagaaagaagagaauuuggucaacuccuuggucacagcuggacaugggcaggucgacaacuuuucacuaggagucuugggaauggcauuguuccuggaggaaaugcuuaggacccgaguaggaacgaaacaugcaauacuacuaguugcaguuucuuuugugacauugaucacagggaacauguccuuuagagaccugggaagagugaugguuaugguaggcgccacuaugacggaugacauagguaugggcgugacuuaucuugcccuacuagcagccuucaaagucagaccaacuuuugcagcuggacuacucuugagaaagcugaccuccaaggaauugaugaugacuacuauaggaauuguacuccucucccagagcaccauaccagagaccauucuugaguugacugaugcguuagccuuaggcaugaugguccucaaaauggugagaaauauggaaaaguaucaauuggcagugacuaucauggcuaucuugugcgucccaaacgcagugauauuacaaaacgcauggaaagugaguugcacaauauuggcagugguguccguuuccccacugcucuuaacauccucacagcaaaaaacagauuggauaccauuagcauugacgaucaaaggucucaauccaacagcuauuuuucuaacaacccucucaagaaccagcaagaaaaggagcuggccauuaaaugaggcuaucauggcagucgggauggugagcauuuuagccaguucucuccuaaaaaaugauauucccaugacaggaccauuaguggcuggagggcuccucacugugugcuacgugcucacuggacgaucggccgauuuggaacuggagagagcagccgaugucaaaugggaagaccaggcagagauaucaggaagcaguccaauccugucaauaacaauaucagaagaugguagcaugucgauaaaaaaugaagaggaagaacaaacacugaccauacucauuagaacaggauugcuggugaucucaggacuuuuuccuguaucaauaccaaucacggcagcagcaugguaccugugggaagugaagaaacaacgggccggaguauugugggauguuccuucacccccacccaugggaaaggcugaacuggaagauggagccuauagaauuaagcaaaaagggauucuuggauauucccagaucggagccggaguuuacaaagaaggaacauuccauacaauguggcaugucacacguggcgcuguucuaaugcauaaaggaaagaggauugaaccaucaugggcggacgucaagaaagaccuaauaucauauggaggaggcuggaaguuagaaggagaauggaaggaaggagaagaaguccagguauuggcacuggagccuggaaaaaauccaagagccguccaaacgaaaccuggucuuuucaaaaccaacgccggaacaauaggugcuguaucucuggacuuuucuccuggaacgucaggaucuccaauuaucgacaaaaaaggaaaaguugugggucuuuaugguaaugguguuguuacaaggaguggagcauaugugagugcuauagcccagacugaaaaaagcauugaagacaacccagagaucgaagaugacauuuuccgaaagagaagacugaccaucauggaccuccacccaggagcgggaaagacgaagagauaccuuccggccauagucagagaagcuauaaaacgggguuugagaacauuaaucuuggcccccacuagaguuguggcagcugaaauggaggaagcccuuagaggacuuccaauaagauaccagaccccagccaucagagcugagcacaccgggcgggagauuguggaccuaaugugucaugccacauuuaccaugaggcugcuaucaccaguuagagugccaaacuacaaccugauuaucauggacgaagcccauuucacagacccagcaaguauagcagcuagaggauacaucucaacucgaguggagaugggugaggcagcugggauuuuuaugacagccacucccccgggaagcagagacccauuuccucagagcaaugcaccaaucauagaugaagaaagagaaaucccugaacguucguggaauuccggacaugaaugggucacggauuuuaaagggaagacuguuugguucguuccaaguauaaaagcaggaaaugauauagcagcuugccugaggaaaaauggaaagaaagugauacaacucaguaggaagaccuuugauucugaguaugucaagacuagaaccaaugauugggacuucgugguuacaacugacauuucagaaaugggugccaauuucaaggcugagaggguuauagaccccagacgcugcaugaaaccagucauacuaacagauggugaagagcgggugauucuggcaggaccuaugccagugacccacucuagugcagcacaaagaagagggagaauaggaagaaauccaaaaaaugagaaugaccaguacauauacaugggggaaccucuggaaaaugaugaagacugugcacacuggaaagaagcuaaaaugcuccuagauaacaucaacacgccagaaggaaucauuccuagcauguucgaaccagagcgugaaaagguggaugccauugauggcgaauaccgcuugagaggagaagcaaggaaaaccuuuguagacuuaaugagaagaggagaccuaccagucugguuggccuacagaguggcagcugaaggcaucaacuacgcagacagaaggugguguuuugauggagucaagaacaaccaaauccuagaagaaaacguggaaguugaaaucuggacaaaagaaggggaaaggaagaaauugaaacccagaugguuggaugcuaggaucuauucugacccacuggcgcuaaaagaauuuaaggaauuugcagccggaagaaagucucugacccugaaccuaaucacagaaauggguaggcucccaaccuucaugacucagaaggcaagagacgcacuggacaacuuagcagugcugcacacggcugaggcagguggaagggcguacaaccaugcucucagugaacugccggagacccuggagacauugcuuuuacugacacuucuggcuacagucacgggagggaucuuuuuauucuugaugagcggaaggggcauagggaagaugacccugggaaugugcugcauaaucacggcuagcauccuccuaugguacgcacaaauacagccacacuggauagcagcuucaauaauacuggaguuuuuucucuaguuuugcuuauuccagaaccugaaaaacagagaacaccccaagacaaccaacugaccuacguugucauagccauccucacagugguggccgcaaccauggcaaacgagauggguuuccuagaaaaaacgaagaaagaucucggauugggaagcauugcaacccagcaacccgagagcaacauccuggacauagaucuacguccugcaucagcauggacgcuguaugccguggccacaacauuuguuacaccaauguugagacauagcauugaaaauuccucagugaaugugucccuaacagcuauagccaaccaagccacaguguuaaugggucucgggaaaggauggccauugucaaagauggacaucggaguuccccuucucgccauuggaugcuacucacaagucaaccccauaacucucacagcagcucuuuucuuauugguagcacauuaugccaucauagggccaggacuccaagcaaaagcaaccagagaagcucagaaaagagcagcggcgggcaucaugaaaaacccaacugucgauggaauaacagugauugaccuagauccaauaccuuaugauccaaaguuugaaaagcaguugggacaaguaaugcuccuaguccucugcgugacucaaguauugaugaugaggacuacaugggcucugugugaggcuuuaaccuuagcuaccgggcccaucuccacauugugggaaggaaauccagggagguuuuggaacacuaccauugcggugucaauggcuaacauuuuuagagggaguuacuuggccggagcuggacuucucuuuucuauuaugaagaacacaaccaacacaagaaggggaacuggcaacauaggagagacgcuuggagagaaauggaaaagccgauugaacgcauugggaaaaagugaauuccagaucuacaagaaaaguggaauccaggaaguggauagaaccuuagcaaaagaaggcauuaaaagaggagaaacggaccaucacgcugugucgcgaggcucagcaaaacugagaugguucguugagagaaacauggucacaccagaagggaaaguaguggaccucgguuguggcagaggaggcuggucauacuauuguggaggacuaaagaauguaagagaagucaaaggccuaacaaaaggaggaccaggacacgaagaacccauccccaugucaacauauggguggaaucuagugcgucuucaaaguggaguugacguuuucuucaucccgccagaaaagugugacacauuauugugugacauaggggagucaucaccaaaucccacaguggaagcaggacgaacacucagaguccuuaacuuaguagaaaauugguugaacaacaacacucaauuuugcauaaagguucucaacccauauaugcccucagucauagaaaaaauggaagcacuacaaaggaaauauggaggagccuuagugaggaauccacucucacgaaacuccacacaugagauguacuggguauccaaugcuuccgggaacauagugucaucagugaacaugauuucaaggauguugaucaacagauuuacaaugagauacaagaaagccacuuacgagccggauguugaccucggaagcggaacccguaacaucgggauugaaagugagauaccaaaccuagauauaauugggaaaagaauagaaaaaauaaagcaagagcaugaaacaucauggcacuaugaccaagaccacccauacaaaacgugggcauaccaugguagcuaugaaacaaaacagacuggaucagcaucauccauggucaacggaguggucaggcugcugacaaaaccuugggacgucguccccauggugacacagauggcaaugacagacacgacuccauuuggacaacagcgcguuuuuaaagagaaaguggacacgagaacccaagaaccgaaagaaggcacgaagaaacuaaugaaaauaacagcagaguggcuuuggaaagaauuagggaagaaaaagacacccaggaugugcaccagagaagaauucacaagaaaggugagaagcaaugcagccuugggggccauauucacugaugagaacaaguggaagucggcacgugaggcuguugaagauaguagguuuugggagcugguugacaaggaaaggaaucuccaucuugaaggaaagugugaaacauguguguacaacaugaugggaaaaagagagaagaagcuaggggaauucggcaaggcaaaaggcagcagagccauaugguacauguggcuuggagcacgcuucuuagaguuugaagcccuaggauucuuaaaugaagaucacugguucuccagagagaacucccugaguggaguggaaggagaagggcugcacaagcuagguuacauucuaagagacgugagcaagaaagagggaggagcaauguaugccgaugacaccgcaggaugggauacaagaaucacacuagaagaccuaaaaaaugaagaaaugguaacaaaccacauggaaggagaacacaagaaacuagccgaggccauuuucaaacuaacguaccaaaacaagguggugcgugugcaaagaccaacaccaagaggcacaguaauggacaucauaucgagaagagaccaaagagguaguggacaaguuggcaccuauggacucaauacuuucaccaauauggaagcccaacuaaucagacagauggagggagaaggagucuuuaaaagcauucagcaccuaacaaucacagaagaaaucgcugugcaaaacugguuagcaagaguggggcgcgaaagguuaucaagaauggccaucaguggagaugauuguguugugaaaccuuuagaugacagguucgcaagcgcuuuaacagcucuaaaugacaugggaaagauuaggaaagacauacaacaaugggaaccuucaagaggauggaaugauuggacacaagugcccuucuguucacaccauuuccaugaguuaaucaugaaagacggucgcguacucguuguuccauguagaaaccaagaugaacugauuggcagagcccgaaucucccaaggagcaggguggucuuugcgggagacggccuguuuggggaagucuuacgcccaaauguggagcuugauguacuuccacagacgcgaccucaggcuggcggcaaaugcuauuugcucggcaguaccaucacauuggguuccaacaagucgaacaaccugguccauacaugcuaaacaugaauggaugacaacggaagacaugcugacagucuggaacaggguguggauucaagaaaacccauggauggaagacaaaacuccaguggaaucaugggaggaaaucccauacuuggggaaaagagaagaccaauggugcggcucauugauuggguuaacaagcagggccaccugggcaaagaacauccaagcagcaauaaaucaaguuagaucccuuauaggcaaugaagaauacacagauuacaugccauccaugaaaagauucagaagagaagaggaagaagcaggaguucugugguagaaagcaaaacuaacaugaaacaaggcuagaagucaggucggauuaagccauaguacggaaaaaacuaugcuaccugugagccccguccaaggacguuaaaagaagucaggccaucauaaaugccauagcuugaguaaacuaugcagccuguagcuccaccugagaagguguaaaaaauccgggaggccacaaaccauggaagcuguacgcauggcguaguggacuagcgguuagaggagaccccucccuuacaaaucgcagcaacaaugggggcccaaggcgagaugaagcuguagucucgcuggaaggacuagagguuagaggagacccccccgaaacaaaaaacagcauauugacgcugggaaagaccagagauccugcugucuccucagcaucauuccaggcacagaacgccagaaaauggaauggugcuguugaaucaacagguucu

SEQ ID NO: 8 - RNA, 293 nt, Artificial sequence
  Circular Artificial sequence to 3UTR (Dengue virus) (circ_dv_3utr)
  misc_feature (7)..(38): /note="hybridization site to 3UTR (Dengue virus)"
  misc_feature (48)..(79): /note="hybridization site to 3UTR (Dengue virus)"
  misc_feature (89)..(120): /note="hybridization site to 3UTR (Dengue virus)"
  misc_feature (130)..(161): /note="hybridization site to 3UTR (Dengue virus)"
  misc_feature (171)..(202): /note="hybridization site to 3UTR (Dengue virus)"
  misc_feature (212)..(243): /note="hybridization site to 3UTR (Dengue virus)"
  misc_feature (253)..(284): /note="hybridization site to 3UTR (Dengue virus)"
gcgccgucuuuggucuuuuuuggcgucaguauguuguuuuuuaaccaucuuugguuuuuccuggcgucagugugcuguuauaguauggucucugguuuuuuuuagcguuagugugcuguuaaagcuuucucucuggucuuuuccagugucaauaugcuguuuagugugcuucuuugguuuuucuugguguuggugugcuguuucagcaaguucucuggucuuuucuggcguugauguguuguucgcuggcgaucuuuggucuuucucggugucgauauguuguuaguggccca

SEQ ID NO: 9 - RNA, 300 nt, Artificial sequence
  Circular Artificial sequence to ChP (Dengue virus) (circ_dv_cHP_v1)
  misc_feature (7)..(39): /note="hybridization site to ChP (Dengue virus)"
  misc_feature (49)..(81): /note="hybridization site to ChP (Dengue virus)"
  misc_feature (91)..(123): /note="hybridization site to ChP (Dengue virus)"
  misc_feature (133)..(165): /note="hybridization site to ChP (Dengue virus)"
  misc_feature (175)..(207): /note="hybridization site to ChP (Dengue virus)"
  misc_feature (217)..(249): /note="hybridization site to ChP (Dengue virus)"
  misc_feature (259)..(291): /note="hybridization site to ChP (Dengue virus)"
gcgccgauguugggggguguguuuuuugucuuuuuucguugggguguaauguugagaggcguguuuuuugcuuuuuuuugugcaccuuauauauugggagguguguuuuucgcuuuuuuccgucuggcuggcauauuggggggcguguuuuucguuuuuuuucguuacaagcuaauauugaaggguguguuuuucgccuuuuuccguguuaaagauauguugaaaggcguguuuuucguuuuuuuccguaaguuuuaaauauuggagggcguguuuuucgcuuuuuucuguagaccggaa

SEQ ID NO: 10 - RNA, 294 nt, Artificial sequence
  Circular Artificial sequence to ChP2 (Dengue virus) (circ_dv_cHP_v2)
  misc_feature (7)..(39): /note="hybridization site to ChP2 (Dengue virus)"
  misc_feature (55)..(87): /note="hybridization site to ChP2 (Dengue virus)"
  misc_feature (103)..(135): /note="hybridization site to ChP2 (Dengue virus)"
  misc_feature (151)..(183): /note="hybridization site to ChP2 (Dengue virus)"
  misc_feature (199)..(231): /note="hybridization site to ChP2 (Dengue virus)"
  misc_feature (247)..(279): /note="hybridization site to ChP2 (Dengue virus)"
gcgccgauguugagagguguguuuuuugcuuuuuuucguuuauuacuccaacagauauugagaggcguguuuuucgcuuuuuuucguagaagguaauagaaaauauuggggggcguguuuuucgucuuuuuucgugacucuuacgaguuuauguugaaagguguguuuuucgcuuuuuuuuguagggaacagucgcaaauauugggggguguguuuuucgccuuuuuucguggcaccguaauccgcauguugaaagguguguuuuucguuuuuuuccgucaacucguucuuaua

SEQ ID NO: 11 - RNA, 11840 nt, Chikungunya virus
  misc_feature (14)..(48): /note="target region of 5UTR"
  misc_feature (11367)..(11399): /note="target region of RSE"
auggcugcgugagacacacguagccuaccaguuucuuacugcucuacucugcaaagcaagagauuaauaacccaucauggauccuguguacguggacauagacgcugacagcgccuuuuugaaggcccugcaacgugcguaccccauguuugagguggaaccaaggcaggucacaccgaaugaccaugcuaaugcuagagcguucucgcaucuagcuauaaaacuaauagagcaggaaauugaccccgacucaaccauccuggauaucggcagugcgccagcaaggaggaugaugucggacaggaaguaccacugcgucugcccgaugcgcagugcggaagaucccgagagacucgccaauuaugcgagaaagcuagcaucugccgcaggaaaaguccuggacagaaacaucucuggaaagaucggggacuuacaagcaguaauggccgugccagacacggagacgccaacauucugcuuacacacagacgucucauguagacagagagcagacgucgcuauauaccaagacgucuaugcuguacacgcacccacgucgcuauaccaccaggcgauuaaagggguccgaguggcguacuggguuggguucgacacaaccccguucauguacaaugccauggcgggugccuaccccucauacucgacaaacugggcagaugagcagguacugaaggcuaagaacauaggauuauguucaacagaccugacggaagguagacgaggcaaguugucuauuaugagagggaaaaagcuaaaaccgugcgaccgugugcuguucucaguagggucaacgcucuacccggaaagccgcaagcuacuuaagagcuggcaccugccaucgguguuccauuuaaagggcaaacucagcuucacaugccgcugugauacagugguuucgugugagggcuacgucguuaagagaauaacgaugagcccaggccuuuauggaaaaaccacaggguaugcgguaacccaccacgcagacggauuccugaugugcaagacuaccgacacgguugacggcgaaagarugucauucucggugugcacauacgugccggcgaccauuugugaucaaaugaccggcauccuugcuacagaagucacgccggaggaugcacagaagcuguugguggggcugaaccagagaauagugguuaacggcagaacgcaacggaauacgaacaccaugaaaaauuaucugcuucccguggucgcccaagccuucaguaagugggcaaaggagugccggaaagacauggaagaugaaaaacuccugggggucagagaaagaacacugaccugcugcugucuaugggcauucaagaagcagaaaacacacacggucuacaagaggccugauacccagucaauucagaagguucaggccgaguuugacagcuuugugguaccgagucuguggucguccggguugucaaucccuuugaggacuagaaucaaaugguuguuaagcaaggugccaaaaaccgaccugaucccauacagcggagacgcccgagaagcccgggacgcagaaaaagaagcagaggaagaacgagaagcagaacugacucgcgaagcccuaccaccucuacaggcagcacaggaagauguucaggucgaaaucgacguggaacagcuugaggacagagcgggcgcaggaauaauagagacuccgagaggagcuaucaaaguuacugcccaaccaacagaccacgucgugggagaguaccugguacucuccccgcagaccguacuacguagccagaagcucagucugauucacgcuuuggcggagcaagugaagacgugcacgcacaacggacgagcagggagguaugcggucgaagcguacgacggccgaguccuagugcccucaggcuaugcaaucucgccugaagacuuccagagucuaagcgaaagcgcaacgaugguguauaacgaaagagaguucguaaacagaaagcuacaccauauugcgaugcacggaccagcccugaacaccgacgaagagucguaugagcuggugagggcagagaggacagaacacgaguacgucuacgacguggaucagagaagaugcuguaagaaggaagaagccgcaggacugguacuggugggcgacuugacuaauccgcccuaccacgaauucgcauaugaagggcuaaaaauccgcccugccugcccauacaaaauugcagucauaggagucuucggaguaccgggaucuggcaagucagcuauuaucaagaaccuaguuaccaggcaggaccuggugacuagcggaaagaaagaaaacugccaagaaaucaccaccgacgugaugagacagagaggucuagagauaucugcacguacgguugacucgcugcucuugaauggaugcaacagaccagucgacguguuguacguagacgaggcguuugcgugccacucuggaacgcuacuugcuuugaucgccuuggugagaccaaggcagaaaguuguacuuuguggugacccgaagcagugcggcuucuucaauaugaugcagaugaaagucaacuauaaucacaacaucugcacccaaguguaccacaaaaguaucuccaggcgguguacacugccugugaccgccauugugucaucguugcauuacgaaggcaaaaugcgcacuacgaaugaguacaacaagccgauuguaguggacacuacaggcucaacaaaaccugacccuggagaccucguguuaacgugcuucagaggguggguuaaacaacugcaaauugacuaucguggauacgaggucaugacagcagccgcaucccaaggguuaaccagaaaaggaguuuacgcaguuagacaaaaaguuaaugaaaacccgcucuaugcaucaacgucagagcacgucaacguacuccuaacgcguacggaagguaaacugguauggaagacacuuuccggcgacccguggauaaagacgcugcagaacccaccgaaaggaaacuucaaagcaacuauuaaggagugggagguggagcaugcaucaauaauggcgggcaucugcagucaccaaaugaccuucgauacauuccaaaauaaagccaacguuuguugggcuaagagcuuggucccuauccucgaaacagcggggauaaaacuaaaugauaggcaguggucucagauaauucaagccuucaaagaagacaaagcauacucaccugaaguagcccugaaugaaauauguacgcgcauguaugggguggaucuagacagcgggcuauuuucuaaaccguuggugucuguguauuacgcggauaaccacugggauaauaggccuggagggaaaauguucggauuuaaccccgaggcagcauccauucuagaaagaaaguauccauucacaaaagggaaguggaacaucaacaagcagaucugcgugacuaccaggaggauagaagacuuuaacccuaccaccaacaucauaccggccaacaggagacuaccacacucauuaguggccgaacaccgcccaguaaaaggggaaagaauggaauggcugguuaacaagauaaacggccaccacgugcuccuggucaguggcuauaaccuugcacugccuacuaagagagucacuuggguagcgccguuagguguccgcggagcggacuacacauacaaccuagaguugggucugccagcaacgcuugguagguaugaccuaguggucauaaacauccacacaccuuuucgcauacaccauuaccaacagugcgucgaccacgcaaugaaacugcaaaugcucgggggugacucauugagacugcucaaaccgggcggcucucuauugaucagagcauaugguuacgcagauagaaccagugaacgagucaucugcguauugggacgcaaguuuagaucgucuagagcguugaaaccaccaugugucaccagcaacacugagauguuuuuccuauucagcaacuuugacaauggcagaaggaauuucacaacucaugucaugaacaaucaacugaaugcagccuucguaggacaggucacccgagcaggaugugcaccgucguaccggguaaaacgcauggacaucgcgaagaacgaugaagagugcguagucaacgccgcuaacccucgcggguuaccgggugrcgguguuugcaaggcaguauacaaaaaauggccggaguccuuuaagaacagugcaacaccagugggaaccgcaaaaacaguuaugugcgguacguauccaguaauccacgcuguuggaccaaacuucucuaauuauucggagucugaaggggaccgggaauuggcagcugccuaucgagaagucgcaaaggaaguaacuaggcugggaguaaauaguguagcuauaccucuccucuccacagguguauacucaggagggaaagacaggcugacccagucacugaaccaccucuuuacagccauggacucgacggaugcagacguggucaucuacugccgcgacaaagaaugggagaagaaaauaucugaggccauacagaugcggacccaaguagagcugcuggaugagcacaucuccauagacugcgauauuguucgcgugcacccugacagcagcuuggcaggcagaaaaggauacagcaccacggaaggcgcacuguacucauaucuagaagggacccguuuucaucagacggcuguggauauggcggagauacauacuauguggccaaagcaaacagaggccaaugagcaagucugccuauaugcccugggggaaaguauugaaucgaucaggcagaaaugcccgguggaugaugcagacgcaucaucuccccccaaaacugucccgugccuuugccguuacgcuaugacuccagaacgcgucacccggcuucgcaugaaccacgucacaagcauaauuguguguucuucguuuccccucccaaaguacaaaauagaaggagugcaaaaagucaaaugcucuaagguaaugcuauuugaccacaacgugccaucgcgcguaaguccaagggaauauakaucuucccaggagucugcacaggaggcgaguacaaucacgucacugacgcauagucaauucgaccuaagcguugauggcgagauacugcccgucccgucagaccuggaugcugacgccccagcccuagaaccagcacuagacgacggggcgacacacacgcugccauccacaaccggaaaccuugcggccgugucugauuggguaaugagcaccguaccugucgcgccgcccagaagaaggcgagggagaaaccugacugugacaugugacgagagagaagggaauauaacacccauggcuagcguccgauucuuuagggcagagcuguguccggucguacaagaaacagcggagacgcgugacacagcaaugucucuucaggcaccaccgaguaccgccacggaaccgaaucauccgccgaucuccuucggagcaucaagcgagacguuccccauuacauuuggggacuucaacgaaggagaaaucgaaagcuugucuucugagcuacuaacuuucggagacuucuuaccaggagaaguggaugacuugacagacagcgacugguccacgugcucagacacggacgacgaguuaugacuagacagggcagguggguauauauucucgucggacaccgguccaggucauuuacaacagaagucaguacgccagucagugcugccggugaacacccuggaggaaguccacgaggagaaguguuacccaccuaagcuggaugaagcaaaggagcaacuauuacuuaagaaacuccaggagagugcauccauggccaacagaagcagguaucagucgcgcaaaguagaaaacaugaaagcagcaaucauccagagacuaaagagaggcuguagacuauacuuaaugucagagaccccaaaagucccuacuuaccggacuacauauccggcgccuguguacucgccuccgaucaacguccgauuguccaaucccgaguccgcaguggcagcaugcaaugaguucuuagcuagaaacuauccaacugucucaucauaccaaauuaccgacgaguaugaugcauaucuagacaugguggacgggucggagaguugccuggaccgagcgacauucaauccgucaaaacucaggagcuacccgaaacagcacgcuuaccacgcgcccuccaucagaagcgcuguaccguccccauuccagaacacacuacagaauguacuggcagcagccacgaaaagaaacugcaacgucacacagaugagggaauuacccacuuuggacucagcaguauucaacguggaguguuucaaaaaauucgcaugcaaccaagaauacugggaagaauuugcugccagcccuauuaggauaacaacugagaauuuagcaaccuauguuacuaaacuaaaagggccaaaagcagcagcgcuauucgcaaaaacccauaaucuacugccacuacaggaaguaccaauggauagguucacaguagauaugaaaagggacguaaaggugacuccugguacaaagcauacagaggaaagaccuaaggugcagguuauacaggcggcugaacccuuggcgacagcauaccuaugugggauucacagagagcugguuaggaggcugaacgccguccuccuacccaauguacauacacuauuugacaugucugccgaggauuucgaugccaucauagccgcacacuuuaagccaggagacacuguuuuggaaacggacauagccuccuuugauaagagccaagaugauucacuugcgcuuacugcuuugaugcuguuagaggauuuagggguggaucacucccugcuggacuugauagaggcugcuuucggagagauuuccagcugucaccuaccgacagguacgcgcuucaaguucggcgccaugaugaaaucagguauguuccuaacucuguucgucaacacauuguuaaacaucaccaucgccagccgagugcuggaagaucgucugacaaaauccgcgugcgcggccuucaucggcgacgacaacauaauacauggagucgucuccgaugaauugauggcagccagaugugccacuuggaugaacauggaagugaagaucauagaugcaguuguauccuugaaagccccuuacuuuuguggaggguuuauacugcacgauacugugacaggaacagcuugcagaguggcagacccgcuaaaaaggcuuuuuaaacugggcaaaccgcuagcggcaggugacgaacaagaugaagauagaagacgagcgcuggcugacgaagugaucagauggcaacgaacagggcuaauugaugagcuggagaaagcgguauacucuagguacgaagugcaggguauaucaguugugguaauguccauggccaccuuugcaagcuccagauccaacuucgagaagcucagaggacccgucauaacuuuguacggcgguccuaaauagguacgcacuacagcuaccuauuuugcagaagccgacagcaaguaucuaaacacuaaucagcuacaauggaguucaucccaacccaaacuuuuuacaauaggagguaccagccucgacccuggacuccgcgcccuacuauccaagucaucaggcccagaccgcgcccucagaggcaagcugggcaacuugcccagcugaucucagcaguuaauaaacugacaaugcgcgcgguaccccaacagaagccacgcaggaaucggaagaauaagaagcaaaagcaaaaacaacaggcgccacaaaacaacacaaaucaaaagaagcagccaccuaaaaagaaaccggcucaaaagaaaaagaagccgggccgcagagagaggaugugcaugaaaaucgaaaaugauuguauuuucgaagucaagcacgaagguaagguaacagguuacgcgugccuggugggggacaaaguaaugaaaccagcacacguaaaggggaccaucgauaacgcggaccuggccaaacuggccuuuaagcggucaucuaaguaugaccuugaaugcgcgcagauacccgugcacaugaaguccgacgcuucgaaguucacccaugagaaaccggagggguacuacaacuggcaccacggagcaguacaguacucaggaggccgguucaccaucccuacaggugcuggcaaaccaggggacagcggcagaccgaucuucgacaacaagggacgcgugguggccauagucuuaggaggagcuaaugaaggagcccguacagcccucucgguggugaccuggaauaaagacauugucacuaaaaucacccccgagggggccgaagaguggagucuugccaucccaguuaugugccuguuggcaaacaccacguuccccugcucccagcccccuugcacgcccugcugcuacgaaaaggaaccggaggaaacccuacgcaugcuugaggacaacgucaugagaccuggguacuaucagcugcuacaagcauccuuaacauguucuccccaccgccagcgacgcagcaccaaggacaacuucaaugucuauaaagccacaagaccauacuuagcucacugucccgacuguggagaagggcacucgugccauagucccguagcacuagaacgcaucagaaaugaagcgacagacgggacgcugaaaauccaggucuccuugcaaaucggaauaaagacggaugacagccacgauuggaccaagcugcguuauauggacaaccacaugccagcagacgcagagagggcggggcuauuuguaagaacaucagcaccguguacgauuacuggaacaaugggacacuucauccuggcccgauguccaaaaggggaaacucugacggugggauucacugacaguaggaagauuagucacucauguacgcacccauuucaccacgacccuccugugauaggucgggaaaaauuccauucccgaccgcagcacgguaaagagcuaccuugcagcacguacgugcagagcaccgccgcaacuaccgaggagauagagguacacaugcccccagacaccccugaucgcacauuaaugucacaacaguccggcaacguaaagaucacagucaauggccagacggugcgguacaaguguaauugcgguggcucaaaugaaggacuaacaacuacagacaaagugauuaauaacugcaagguugaucaaugucaugccgcggucaccaaucacaaaaaguggcaguauaacuccccucuggucccgcguaaugcugaacuuggggaccgaaaaggaaaaauucacaucccguuuccgcuggcaaauguaacaugcagggugccuaaagcaaggaaccccaccgugacguacgggaaaaaccaagucaucaugcuacuguauccugaccacccaacacuccuguccuaccggaauaugggagaagaaccaaacuaucaagaagagugggugaugcauaagaaggaagucgugcuaaccgugccgacugaagggcucgaggucacguggggcaacaacgagccguauaaguauuggccgcaguuaucuacaaacgguacagcccauggccacccgcaugagauaauucuguauuauuaugagcuguaccccacuaugacuguaguaguugugucaguggccacguucauacuccugucgaugguggguauggcagcggggaugugcaugugugcacgacgcagaugcaucacaccguaugaacugacaccaggagcuaccgucccuuuccugcuuagccuaauaugcugcaucagaacagcuaaagcggccacauaccaagaggcugcgauauaccuguggaacgagcagcaaccuuuguuuuggcuacaagcccuuauuccgcuggcagcccugauuguucuaugcaacugucugagacucuuaccaugcugcuguaaaacguuggcuuuuuuagccguaaugagcgucggugcccacacugugagcgcguacgaacacguaacagugaucccgaacacggugggaguaccguauaagacucuagucaauagaccuggcuacagccccaugguauuggagauggaacuacugucagucacuuuggagccaacacuaucgcuugauuacaucacgugcgaguacaaaaccgucaucccgucuccguacgugaagugcugcgguacagcagagugcaaggacaaaaaccuaccugacuacagcuguaaggucuucaccggcgucuacccauuuauguggggcggcgccuacugcuucugcgacgcugaaaacacgcaguugagcgaagcacacguggagaaguccgaaucaugcaaaacagaauuugcaucagcauacagggcucauaccgcaucugcaucagcuaagcuccgcguccuuuaccaaggaaauaacaucacuguaacugccuaugcaaacggcgaccaugccgucacaguuaaggacgccaaauucauuguggggccaaugucuucagccuggacaccuuucgacaacaaaauugugguguacaaaggugacgucuauaacauggacuacccgcccuuuggcgcaggaagaccaggacaauuuggcgauauccaaagucgcacaccugagaguaaagacgucuaugcuaauacacaacugguacugcagagaccggcuguggguacgguacacgugccauacucucaggcaccaucuggcuuuaaguauuggcuaaaagaacgcggggcgucgcugcagcacacagcaccauuuggcugccaaauagcaacaaacccgguaagagcggugaacugcgccguagggaacaugcccaucuccaucgacauaccggaagcggccuucacuagggucgucgacgcgcccucuuuaacggacaugucgugcgagguaccagccugcacccauuccucagacuuugggggcgucgccauuauuaaauaugcagccagcaagaaaggcaagugugcggugcauucgaugacuaacgccgucacuauucgggaagcugagauagaaguugaagggaauucucagcugcaaaucucuuucucgacggccuuagccagcgccgaauuccgcguacaagucuguucuacacaaguacacugugcagccgagugccaccccccgaaggaccacauagucaacuacccggcgucacauaccacccucgggguccaggacaucuccgcuacggcgaugucaugggugcagaagaucacgggaggugugggacugguuguugcuguugccgcacugauucuaaucguggugcuaugcgugucguucagcaggcacuaacuugacaauuaaguaugaagguauauguguccccuaagagacacacuguacauagcaaauaaucuauagaucaaagggcuacgcaaccccugaauaguaacaaaauacaaaaucacuaaaaauuauaaaaacagaaaaauacauaaauagguauacguguccccuaagagacacauuguauguaggugauaaguauagaucaaagggccgaauaaccccugaauaguaacaaaauaugaaaaucaauaaaaaucauaaaauagaaaaaccauaaacagaaguaguucaaagggcuauaaaaccccugaauaguaacaaaacauaaaauuaauaaaaaucaaaugaauaccauaauuggcaaacggaagagauguagguacuuaagcuuccuaaaagcagccgaacucacuuugagaaguaggcauagcauaccgaacucuuccacgauucuccgaacccacagggacguaggagauguuauuuuguuuuuaauauuucaaaaaaaaaaaaaaaaaaaaaaaaaaaaa

SEQ ID NO: 12 - RNA, 294 nt, Artificial sequence
  Circular Artificial sequence to 5UTR (Chikungunya virus) (chikv_5utr1)
  misc_feature (7)..(41): /note="hybridization site to 5UTR (Chikungunya virus)"
  misc_feature (55)..(89): /note="hybridization site to 5UTR (Chikungunya virus)"
  misc_feature (103)..(137): /note="hybridization site to 5UTR (Chikungunya virus)"
  misc_feature (151)..(185): /note="hybridization site to 5UTR (Chikungunya virus)"
  misc_feature (199)..(233): /note="hybridization site to 5UTR (Chikungunya virus)"
  misc_feature (247)..(281): /note="hybridization site to 5UTR (Chikungunya virus)"
gcgccgaguagagcagugaggaacugguggguugcguguguuuugucacuacguaguagagugguaggaaauuggugggcugcguguguggcuuccagugccaguagagcggugagagauugguagguuauguguguguucucgcccgaaaguagaguggugagggguugguagguuacguguguucaaauaugacggaguagaguaguggggggcugguggguuguguguguucggagaccagacaguagaguaguaagagguuggugggcuacgugugugccacaccacccg

SEQ ID NO: 13 - RNA, 314 nt, Artificial sequence
  Circular Artificial sequence to 5UTR (Chikungunya virus) (chikv_5utr2)
  misc_feature (7)..(41): /note="hybridization site to 5UTR (Chikungunya virus)"
  misc_feature (51)..(85): /note="hybridization site to 5UTR (Chikungunya virus)"
  misc_feature (95)..(129): /note="hybridization site to 5UTR (Chikungunya virus)"
  misc_feature (139)..(173): /note="hybridization site to 5UTR (Chikungunya virus)"
  misc_feature (183)..(217): /note="hybridization site to 5UTR (Chikungunya virus)"
  misc_feature (227)..(261): /note="hybridization site to 5UTR (Chikungunya virus)"
  misc_feature (271)..(305): /note="hybridization site to 5UTR (Chikungunya virus)"
gcgccgaguagaguggugggggguugguagguugcgugugucuuagcgccaguagagcaguaagggacugguaggcugcgugugugguacuuacaguagagcggugagagacuggugggcuaugugugugccauccuuaguagagugguagggaguuggugggcuguguguguggggccuauaguagaguagugggagauugguagguuacguguguagcugcacgaguagagcagugggaaguugguggguugugugugucuccgacgaaguagaguaguaagaaauugguggguuauguguguaccugaguc

SEQ ID NO: 14 - RNA, 288 nt, Artificial sequence
  Circular Artificial sequence to RSE (Chikungunya virus) (chikv_RSE1)
  misc_feature (7)..(39): /note="hybridization site to RSE (Chikungunya virus)"
  misc_feature (54)..(86): /note="hybridization site to RSE (Chikungunya virus)"
  misc_feature (101)..(133): /note="hybridization site to RSE (Chikungunya virus)"
  misc_feature (148)..(180): /note="hybridization site to RSE (Chikungunya virus)"
  misc_feature (195)..(227): /note="hybridization site to RSE (Chikungunya virus)"
  misc_feature (242)..(274): /note="hybridization site to RSE (Chikungunya virus)"
gcgccguugcguggcucuuuggucuauagauuauuuguuugcggguugcccucuugcgugguucuuuggucuguggauuguuugcuugcccgcgagcagguuguguaguccuuugaucuguagguuguuuguuguucguaacacuucuugcguagcucuuugaucuauagguuauuugcucuagguucgaaccguugcguggucuuuugaucuauggguuguuuguucaucaaaaccuuucuuguguggccuuuuggucuguagauuguuuguuuccaacgacguacu

SEQ ID NO: 15 - RNA, 288 nt, Artificial Sequence
  Circular Artificial sequence to RSE2 (Chikungunya virus) (chikvRSE)
  misc_feature (7)..(39): /note="hybridization site to RSE2 (Chikungunya virus)"
  misc_feature (54)..(86): /note="hybridization site to RSE2 (Chikungunya virus)"
  misc_feature (101)..(133): /note="hybridization site to RSE2 (Chikungunya virus)"
  misc_feature (148)..(180): /note="hybridization site to RSE2 (Chikungunya virus)"
  misc_feature (195)..(227): /note="hybridization site to RSE2 (Chikungunya virus)"
  misc_feature (242)..(274): /note="hybridization site to RSE2 (Chikungunya virus)"
gcgccguugcguggcucuuuggucuauggguuguuugcuugcggguugcccucuuguguagcuuuuugaucuguggguuauuugcuugcccgcgagcagguugcguaguucuuugauuuguagguuguuugcuguucguaacacuucuugcguaguuuuuugaucuauggauuguuugcucuagguucgaaccguugcguagcucuuugguuuauagauuauuugcucaucaaaaccuuucuugugugguccuuugaucuauagguuauuugcuuccaacgacguacu

SEQ ID NO: 16 - RNA, 620 nt, Artificial sequence
  Broadspectrum circular RNA to hepatitis C virus and Dengue virus (DENV1 cHP_ HCV CDS2_T)
  misc_feature (16)..(48): /note="hybridization site to chp (Dengue virus)"
  misc_feature (58)..(90): /note="hybridization site to chp (Dengue virus)"
  misc_feature (100)..(132): /note="hybridization site to chp (Dengue virus)"
  misc_feature (142)..(174): /note="hybridization site to chp (Dengue virus)"
  misc_feature (184)..(216): /note="hybridization site to chp (Dengue virus)"
  misc_feature (226)..(258): /note="hybridization site to chp (Dengue virus)"
  misc_feature (268)..(300): /note="hybridization site to chp (Dengue virus)"
  misc_feature (330)..(340): /note="hybridization site to CDS2 (hepatitis C virus)"
  misc_feature (354)..(364): /note="hybridization site to CDS2 (hepatitis C virus)"
  misc_feature (378)..(388): /note="hybridization site to CDS2 (hepatitis C virus)"
  misc_feature (402)..(412): /note="hybridization site to CDS2 (hepatitis C virus)"
  misc_feature (426)..(436): /note="hybridization site to CDS2 (hepatitis C virus)"
  misc_feature (450)..(460): /note="hybridization site to CDS2 (hepatitis C virus)"
  misc_feature (474)..(484): /note="hybridization site to CDS2 (hepatitis C virus)"
  misc_feature (498)..(508): /note="hybridization site to CDS2 (hepatitis C virus)"
  misc_feature (522)..(532): /note="hybridization site to CDS2 (hepatitis C virus)"
  misc_feature (546)..(556): /note="hybridization site to CDS2 (hepatitis C virus)"
  misc_feature (570)..(580): /note="hybridization site to CDS2 (hepatitis C virus)"
  misc_feature (594)..(604): /note="hybridization site to CDS2 (hepatitis C virus)"
ggauccgcggcgccgauguugggggguguguuuuuugucuuuuuucguugggguguaauguugagaggcguguuuuuugcuuuuuuuugugcaccuuauauauugggagguguguuuuucgcuuuuuuccgucuggcuggcauauuggggggcguguuuuucguuuuuuuucguuacaagcuaauauugaaggguguguuuuucgccuuuuuccguguuaaagauauguugaaaggcguguuuuucguuuuuuuccguaaguuuuaaauauuggagggcguguuuuucgcuuuuuucuguagaccggaaccgggaauucacucgagcccccuggggcucugaugaggaaccucucugggguccccacagcgagucuccuuggggccccuagaaugaaguucuuugggguuucauagcacgguucuccugggguuuucauacgauugucucuuggggucuugcguguuuacccuuuugggguccugcuaagggggcuuucuggggccuuucgaauaagucuuuuuggggccucguuuuaucacucuucugggguucccagccuuucccuucuuggggcuccuccgaccaugccccuugggguucuacccuguaugggaucc

SEQ ID NO: 17 - RNA, 298 nt, Artificial sequence
  Broadspectrum circular RNA to hepatitis C virus and Dengue virus (DENV1 cHP_ HCV CDS2_1)
  misc_feature (7)..(22): /note="hybridization site to CDS2(2) (hepatitis C virus)"
  misc_feature (35)..(67): /note="hybridization site to chp (Dengue virus)"
  misc_feature (80)..(95): /note="hybridization site to CDS2(2) (hepatitis C virus)"
  misc_feature (108)..(140): /note="hybridization site to chp (Dengue virus)"
  misc_feature (153)..(168): /note="hybridization site to CDS2(2) (hepatitis C virus)"
  misc_feature (181)..(213): /note="hybridization site to chp (Dengue virus)"
  misc_feature (226)..(241): /note="hybridization site to CDS2(2) (hepatitis C virus)"
  misc_feature (254)..(286): /note="hybridization site to chp (Dengue virus)"
gcgccgccuggcuugggguuuucccaggacucuaauauuggaaggcguguuuuuugucuuuuuucguguccugccuccucuuagccugggguuuuacgaccgcaccuauguugagagguguguuuuuugccuuuuuucguuugauacccucauuuaaucuggggcucuaggugcagcaaaauguuggagggcguguuuuucguuuuuuucuguccugcacgaucucccgauuugggguucucguuggugacguauauugagggguguguuuuucgcuuuuuuucguauuggacgucgu

SEQ ID NO: 18 - RNA, 41 nt, Artificial Sequence
  Artificial sequence for showing an squematic example of the mode of action according to figure 4 wherein the sequence has no particular value
aacgccaugcaagcgcgcagaucgagcugugcgcuuuuuuu

SEQ ID NO: 19 - RNA, 299 nt, Artificial sequence
  circ_wnv_sIII_2
ugcaguuagauaaacuuuccgguuugauuuucucuucaaaagacaguucuucgaacuucccggcuugguuuucuuuucaaaaauggggccccucaacuuccuggucugguuuucuccuuaaaaggcuauacgaucaacuuucuggccugacuuucuucuuaaaaagcuaucaucgcaacuucucggcuugacuuucucuuuaaaacccucgguaaccaacuuuucggccugauuuucuucucaaaaguuuuggcgacuaacuucuugguuuggcuuucuccucaaaaaauauaaacagc

SEQ ID NO: 20 - RNA, 11029 nt, West Nile virus
  misc_feature (1)..(11029): Genome West Nile virus
aguaguucgccugugugagcugacaaacuuaguaguguuugugaggauuaacaacaauuaacacagugcgagcuguuucuuagcacgaagaucucgaugucuaagaaaccaggagggcccggcaagagccgggcugucaauaugcuaaaacgcggaaugccccgcguguuguccuugauuggacugaagagggcuauguugagccugaucgacggcaaggggccaauacgauuuguguuggcucucuuggcguucuucagguucacagcaauugcuccgacccgagcagugcuggaucgauggagaggugugaacaaacaaacagcgaugaaacaccuucugaguuuuaagaaggaacuagggaccuugaccagugcuaucaaucggcggagcucaaaacaaaagaaaagaggaggaaagaccggaauugcagucaugauuggccugauugccagcguaggagcaguuacccucucuaacuuccaagggaaggugaugaugacgguaaaugcuacugacgucacagaugucaucacgauuccaacagcugcuggaaagaaccuaugcauugucagagcaauggaugugggauacaugugcgaugauacuaucacuuaugaaugcccagugcugucggcugguaaugauccagaagacaucgacuguuggugcacaaagucagcagucuacgucagguauggaagaugcaccaagacacgccacucaagacgcagucggaggucacugacagugcagacacacggagaaagcacucuagcgaacaagaagggggcuuggauggacagcaccaaggccacaagguauuugguaaaaacagaaucauggaucuugaggaacccuggauaugcccugguggcagccgucauugguuggaugcuugggagcaacaccaugcagagaguuguguuugucgugcuauugcuuuugguggccccagcuuacagcuucaacugccuuggaaugagcaacagagacuucuuggaaggagugucuggagcaacauggguggauuugguucucgaaggcgacagcugcgugacuaucaugucuaaggacaagccuaccaucgaugugaagaugaugaauauggaggcggccaaccuggcagagguccgcaguuauugcuauuuggcuaccgucagcgaucucuccaccaaagcugcgugcccgaccaugggagaagcucacaaugacaaacgugcugacccagcuuuugugugcagacaaggagugguggacaggggcuggggcaacggcugcggacuauuuggcaaaggaagcauugacacaugcgccaaauuugccugcucuaccaaggcaauaggaagaaccaucuugaaagagaauaucaaguacgaaguggccauuuuuguccauggaccaacuacuguggagucgcacggaaacuacuccacacagguuggagccacucaggcagggagauucagcaucacuccugcggcgccuucauacacacuaaagcuuggagaauauggagaggugacaguggacugugaaccacggucagggauugacaccaaugcauacuacgugaugacuguuggaacaaagacguucuugguccaucgugagugguucauggaccucaaccucccuuggagcagugcuggaaguacuguguggaggaacagagagacguuaauggaguuugaggaaccacacgccacgaagcagucugugauagcauugggcucacaagagggagcucugcaucaagcuuuggcuggagccauuccuguggaauuuucaagcaacacugucaaguugacgucgggucauuugaaguguagagugaagauggaaaaauugcaguugaagggaacaaccuauggcgucuguucaaaggcuuucaaguuucuugggacucccgcagacacaggucacggcacugugguguuggaauugcaguacacuggcacggauggaccuugcaaaguuccuaucucgucaguggcuucauugaacgaccuaacgccagugggcagauuggucacugucaacccuuuuguuucaguggccacggccaacgcuaagguccugauugaauuggaaccacccuuuggagacucauacauaguggugggcagaggagaacaacagaucaaucaccauuggcacaagucuggaagcagcauuggcaaagccuuuacaaccacccucaaaggagcgcagagacuagccgcucuaggagacacagcuugggacuuuggaucaguuggagggguguucaccucaguugggaaggcuguccaucaaguguucggaggagcauuccgcucacuguucggaggcauguccuggauaacgcaaggauugcugggggcucuccuguuguggaugggcaucaaugcucgugauagguccauagcucucacguuucucgcaguuggaggaguucugcucuuccucuccgugaacgugcacgcugacacugggugugccauagacaucagccggcaagagcugagauguggaaguggaguguucauacacaaugauguggaggcuuggauggaccgguacaaguauuacccugaaacgccacaaggccuagccaagaucauucagaaagcucauaaggaaggagugugcggucuacgaucaguuuccagacuggagcaucaaaugugggaagcagugaaggacgagcugaacacucuuuugaaggagaaugguguggaccuuagugucgugguugagaaacaggagggaauguacaagucagcaccuaaacgccucaccgccaccacggaaaaauuggaaauuggcuggaaggccuggggaaagaguauuuuauuugcaccagaacucgccaacaacaccuuugugguugaugguccggagaccaaggaauguccgacucagaaucgcgcuuggaauagcuuagaaguggaggauuuuggauuuggucucaccagcacucggauguuccugaaggucagagagagcaacacaacugaaugugacucgaagaucauuggaacggcugucaagaacaacuuggcgauccacagugaccuguccuauuggauugaaagcaggcucaaugauacguggaagcuugaaagggcaguucugggugaagucaaaucauguacguggccugagacgcauaccuuguggggcgauggaauccuugagagugacuugauaauaccagucacacuggcgggaccacgaagcaaucacaaucggagaccuggguacaagacacaaaaccagggcccaugggacgaaggccggguagagauugacuucgauuacugcccaggaacuacggucacccugagugagagcugcggacaccguggaccugccacucgcaccaccacagagagcggaaaguugauaacagauuggugcugcaggagcugcaccuuaccaccacugcgcuaccaaacugacagcggcuguugguaugguauggagaucagaccacagagacaugaugaaaagacccucgugcagucacaagugaaugcuuauaaugcugauaugauugacccuuuucaguugggccuucuggucguguucuuggccacccaggagguccuucgcaagagguggacagccaagaucagcaugccagcuauacugauugcucugcuaguccugguguuugggggcauuacuuacacugauguguuacgcuaugucaucuuggugggggcagcuuucgcagaaucuaauucgggaggagacgugguacacuuggcgcucauggcgaccuucaagauacaaccaguguuuaugguggcaucguuucucaaagcgagauggaccaaccaggagaacauuuuguugauguuggcggcuguuuucuuucaaauggcuuaucacgaugcccgccaaauucugcucugggagaucccugauguguugaauucacuggcgguagcuuggaugauacugagagccauaacauucacaacgacaucaaacgugguuguuccgcugcuagcccugcuaacacccgggcugagacgcuugaaucuggauguguacaggauacugcuguugauggucggaauaggcagcuugaucagggagaagaggagugcagcugcaaaaaagaaaggagcaagucugcuaugcuuggcucuagccucaacaggacuuuucaaccccaugauccuugcugcuggacugauugcaugugaucccaaccguaaacgcggauggcccgcaacugaagugaugacagcugucggccuaauguuugccaucgucggagggcuggcagagcuugacauugacuccauggccauuccaaugacuaucgcggggcucauguuugcugcuuucgugauuucugggaaaucaacagauauguggauugagagaacggcggacauuuccugggaaagugaugcagaaauuacaggcucgagcgaaagaguugaugugcggcuugaugaugauggaaacuuccagcucaugaaugauccaggagcaccuuggaagauauggaugcucagaauggucugucucgcgauuagugcguacacccccugggcaaucuugcccucaguaguuggauuuuggauaacucuccaauacacaaagagaggaggcguguugugggacacucccucaccaaaggaguacaaaaagggggacacgaccaccggcgucuacaggaucaugacucgugggcugcucggcaguuaucaagcaggagcgggcgugaugguugaagguguuuuccacacccuuuggcauacaacaaaaggagccgcuuugaugagcggagagggccgccuggacccauacuggggcagugucaaggaggaucgacuuuguuacggaggacccuggaaauugcagcacaaguggaacgggcaggaugaggugcagaugauugugguggaaccuggcaagaacguuaagaacguccggacgaaaccagggguguucaaaacaccugaaggagaaaucggggccgugacuuuggacuuccccacuggaacaucaggcucaccaauaguggacaaaaacggugaugugauugggcuuuauggcaauggagucauaaugcccaacggcucauacauaagcgcgauagugcagggugaaaggauggaugagccaaucccagccggauucgaaccugagaugcugaggaaaaaacagaucacuguacuggaucuccaucccggcgccgguaaaacaaggaggauucugccacagaucaucaaagaggccauaaacagaagacugagaacagccgugcuagcaccaaccaggguuguggcugcugagauggcugaagcacugagaggacugcccauccgguaccagacauccgcagugcccagagaacauaauggaaaugagauuguugaugucaugugucaugcuacccucacccacaggcugaugucuccucacagggugccgaacuacaaccuguucgugauggaugaggcucauuucaccgacccagcuagcauugcagcaagagguuacauuuccacaaaggucgagcuaggggaggcggcggcaauauucaugacagccaccccaccaggcacuucagauccauucccagaguccaauucaccaauuuccgacuuacagacugagaucccggaucgagcuuggaacucuggauacgaauggaucacagaauacaccgggaagacgguuugguuugugccuagugucaagauggggaaugagauugcccuuugccuacaacgugcuggaaagaaaguaguccaauugaacagaaagucguacgagacggaguacccaaaauguaagaacgaugauugggacuuuguuaucacaacagacauaucugaaaugggggcuaacuucaaggcgagcagggugauugacagccggaagagugugaaaccaaccaucauaacagaaggagaagggagagugauccugggagaaccaucugcagugacagcagcuagugccgcccagagacguggacguaucgguagaaauccgucgcaaguuggugaugaguacuguuauggggggcacacgaaugaagacgacucgaacuucgcccauuggacugaggcacgaaucaugcuggacaacaucaacaugccaaacggacugaucgcucaauucuaccaaccagagcgugagaagguauauaccauggauggggaauaccggcucagaggagaagagagaaaaaacuuucuggaacuguugaggacugcagaucugccaguuuggcuggcuuacaagguugcagcggcuggagugucauaccacgaccggagguggugcuuugaugguccuaggacaaacacaauuuuagaagacaacaacgaaguggaagucaucacgaagcuuggugaaaggaagauucugaggccgcgcuggauugacgccaggguguacucggaucaccaggcacuaaaggcguucaaggacuucgccucgggaaaacguucucagauagggcucauugagguucugggaaagaugccugagcacuucauggggaagacaugggaagcacuugacaccauguacguuguggccacugcagagaaaggaggaagagcucacagaauggcccuggaggaacugccagaugcucuucagacaauugccuugauugccuuauugagugugaugaccaugggaguauucuuccuccucaugcagcggaagggcauuggaaagauagguuugggaggcgcugucuugggagucgcgaccuuuuucuguuggauggcugaaguuccaggaacgaagaucgccggaauguugcugcucucccuucucuugaugauugugcuaauuccugagccagagaagcaacguucgcagacagacaaccagcuagccguguuccugauuugugucaugacccuugugagcgcaguggcagccaacgagauggguuggcuagauaagaccaagagugacauaagcaguuuguuugggcaaagaauugaggucaaggagaauuucagcaugggagaguuccuucuggacuugaggccggcaacagccuggucacuguacgcugugacaacagcgguccucacuccacugcuaaagcauuugaucacgucagauuacaucaacaccucauugaccucaauaaacguucaggcaagugcacuauucacacucgcgcgaggcuuccccuucgucgauguuggagugucggcucuccugcuagcagccggaugcuggggacaagucacccucaccguuacgguaacagcggcaacacuccuuuuuugccacuaugccuacaugguucccgguuggcaagcugaggcaaugcgcucagcccagcggcggacagcggccggaaucaugaagaacgcuguaguggauggcaucguggccacggacgucccagaauuagagcgcaccacacccaucaugcagaagaaaguuggacagaucaugcugaucuuggugucucuagcugcaguaguagugaacccgucugugaagacaguacgagaagccggaauuuugaucacggccgcagcggugacgcuuugggagaauggagcaagcucuguuuggaacgcaacaacugccaucggacucugccacaucaugcguggggguugguugucaugucuauccauaacauggacacucauaaagaacauggaaaaaccaggacuaaaaagagguggggcaaaaggacgcaccuugggagagguuuggaaagaaagacucaaccagaugacaaaagaagaguucacuagguaccgcaaagaggccaucaucgaagucgaucgcucagcggcaaaacacgccaggaaagaaggcaaugucacuggagggcauccagucucuaggggcacagcaaaacugagauggcuggucgaacggagguuucucgaaccggucggaaaagugauugaccuuggauguggaagaggcgguugguguuacuauauggcaacccaaaaaagaguccaagaagucagaggguacacaaagggcggucccggacaugaagagccccaacuagugcaaaguuauggauggaacauugucaccaugaagaguggaguggauguguucuacagaccuucugaguguugugacacccuccuuugugacaucggagaguccucgucaagugcugagguugaagagcauaggacgauucggguccuugaaaugguugaggacuggcugcaccgagggccaagggaauuuugcgugaaggugcucugccccuacaugccgaaagucauagagaagauggagcugcuccaacgccgguauggggggggacuggucagaaacccacucucacggaauuccacgcacgagauguauugggugagucgagcuucaggcaaugugguacauucagugaauaugaccagccaggugcuccuaggaagaauggaaaaaaggaccuggaagggaccccaauacgaggaagauguaaacuugggaaguggaaccagggcggugggaaaaccccugcucaacucagacaccaguaaaaucaagaacaggauugaacgacucaggcgugaguacaguucgacguggcaccacgaugagaaccacccauauagaaccuggaacuaucacggcaguuaugaugugaagcccacaggcuccgccaguucgcuggucaauggaguggucaggcuccucucaaaaccaugggacaccaucacgaauguuaccaccauggccaugacugacacuacucccuucgggcagcagcgaguguucaaagagaagguggacacgaaagcuccugaaccgccagaaggagugaaguacgugcucaacgagaccaccaacugguugugggcguuuuuggccagagaaaaacgucccagaaugugcucucgagaggaauucauaagaaaggucaacagcaaugcagcuuugggugccauguuugaagagcagaaucaauggaggagcgccagagaagcaguugaagauccaaaauuuugggagaugguggaugaggagcgcgaggcacaucugcggggggaaugucacacuugcauuuacaacaugaugggaaagagagagaaaaaacccggagaguucggaaaggccaagggaagcagagccauuugguucauguggcucggagcucgcuuucuggaguucgaggcucuggguuuucucaaugaagaccacuggcuuggaagaaagaacucaggaggaggugucgagggcuugggccuccaaaaacuggguuacauccugcgugaaguuggcacccggccugggggcaagaucuaugcugaugacacagcuggcugggacacccgcaucacgagagcugacuuggaaaaugaagcuaaggugcuugagcugcuugauggggaacaucggcgucuugccagggccaucauugagcucaccuaucgucacaaaguugugaaagugaugcgcccggcugcugauggaagaaccgucauggauguuaucuccagagaagaucagagggggaguggacaaguugucaccuacgcccuaaacacuuucaccaaccuggccguccagcuggugaggaugauggaaggggaaggagugauuggcccagaugauguggagaaacucacaaaagggaaaggacccaaagucaggaccuggcuguuugagaauggggaagaaagacucagccgcauggcugucaguggagaugacugugugguaaagccccuggacgaucgcuuugccaccucgcuccacuuccucaaugcuaugucaaagguucgcaaagacauccaagaguggaaaccgucaacuggaugguaugauuggcagcagguuccauuuugcucaaaccauuucacugaauugaucaugaaagauggaagaacacuggugguuccaugccgaggacaggaugaauugguaggcagagcucgcauaucuccaggggccggauggaacguccgcgacacugcuugucuggcuaagucuuaugcccagauguggcugcuucuguacuuccacagaagagaccugcggcucauggccaacgccauuugcuccgcugucccugugaauugggucccuaccggaagaaccacgugguccauccaugcaggaggagaguggaugacaacagaggacauguuggaggucuggaaccguguuuggauagaggagaaugaauggauggaagacaaaaccccaguggagaaauggagugacgucccauauucaggaaaacgagaggacaucugguguggcagccugauuggcacaagagcccgagccacgugggcagaaaacauccagguggcuaucaaccaagucagagcaaucaucggagaugagaaguauguggauuacaugaguucacuaaagagauaugaagacacaacuuugguugaggacacaguacuguagauauuuaaucaauuguaaauagacaauauaaguaugcauaaaaguguaguuuuauaguaguauuuagugguguuaguguaaauaguuaagaaaauuuugaggagaaagucaggccgggaaguucccgccaccggaaguugaguagacggugcugccugcgacucaaccccaggaggacugggugaacaaagccgcgaagugauccauguaagcccucagaaccguuucggaaggaggaccccacauguuguaacuucaaagcccaaugucagaccacgcuacggcgugcuacucugcggagagugcagucugcgauagugccccaggaggacuggguuaacaaaggcaaaccaacgccccacgcggcccuagccccgguaaugguguuaaccagggcgaaaggacuagagguuagaggagaccccgcgguuuaaagugcacggcccagccuggcugaagcuguaggucaggggaaggacuagagguuaguggagaccccgugccacaaaacaccacaacaaaacagcauauugacaccugggauagacuaggagaucuucugcucugcacaaccagccacacggcacagugcgccgacaaugguggcugguggugcgagaacacaggaucu

SEQ ID NO: 21 - RNA, 270 nt, Artificial Sequence
  dchp_wsII_A
ugcaguuagauaauauugggagguguguuuuuugccuuuuuccguagauccucgcgaaacuucuuggccugguuuucuuuucaaaagaguccuuacguauauuggaggguguguuuuucgcuuuuuuccgucucggaaacgauaacuuccuggcuugauuuuuucuucaaaagcugcguuaucuauauugagaggcguguuuuucgucuuuuuccguuccaaccuggagaacuuuuuggccugauuuucucuucaaaaccaggccguacc

SEQ ID NO: 22 - RNA, 270 nt, Artificial Sequence
  dchp_wsII_B
ugcaguuagauaauauuggaaggcguguuuuuugcuuuuuuccguagauccuaacggaacuuccuggccugauuuucuucuuaaaagaguccuuacguauauugaaagguguguuuuucgccuuuuuccgucucggaaacgauaacuuuccggccugguuuuuuccuuaaaagcugcguuaucuauauugagggguguguuuuuugucuuuuuccguuccaaccuggagaacuuccugguuuggcuuuuuucuugaaaccaggccguacc

SEQ ID NO: 23 - RNA, 270 nt, Artificial Sequence
  dchp_wsII_C
ugcaguuagauaauauugggagguguguuuuuugccuuuuuccguagauccucgcgaaacuucuuggccugguuuucuuuucaaaagaguccuuacguauauuggaggguguguuuuucgcuuuuuuccgucucggaaacgauaacuuccuggcuugauuuuuucuucaaaagcugcguuaucuauauugagaggcguguuuuucgucuuuuuccguuccaaccuggagaacuucccggccugacuuuuuccuuaaaaccaggccguacc

SEQ ID NO: 24 - RNA, 299 nt, Artificial Sequence
  circ_wnv_sIII_1
ugcaguuagauaaacuuucugguuugguuuucuccucaaaagacaguucuucgaacuucccgguuugauuuucucuuuaaaaauggggccccucaacuucuuggucugacuuucucuucaaaaggcuauacgaucaacuucucggccugauuuucuuuucaaaaagcuaucaucgcaacuuccuggucugguuuucuucucaaaacccucgguaaccaacuuuccggcuugacuuucuucuuaaaacguuuugcgacaaacuuuucggcuuggcuuucuccuuaaaauauguaaacagc

SEQ ID NO: 25 - RNA, 33 nt, Artificial Sequence
  Target sequence IRES Circ HCV1
cuccgccaugaaucacuccccugugaggaacua

SEQ ID NO: 26 - RNA, 24 nt, Artificial Sequence
  Target sequence IRES Circ HCV2
ucucguagaccgugcaccaugagc

SEQ ID NO: 27 - RNA, 28 nt, Artificial Sequence
  Target sequence cHP (CDS1) circ_hcv_cds1
ccaaaagaaacaccaaccgucgcccaga

SEQ ID NO: 28 - RNA, 11 nt, Artificial Sequence
  Target sequence CDS2 circ_hcv_cds2
ggggccccagg

SEQ ID NO: 29 - RNA, 32 nt, Artificial Sequence
  Target sequence sHP (3' UTR) circ_dv_3utr
aacagcauauugacgcugggaaagaccagaga

SEQ ID NO: 30 - RNA, 33 nt, Artificial Sequence
  Target sequence cHP1/2 circ_dv_cHP_v1/2
acggaaaaaggcgaaaaacacgccuuucaauau

SEQ ID NO: 31 - RNA, 34 nt, Artificial Sequence
  Target sequence Chikv_re
gcugcuguaaaacguuggcuuuuuuagccguaau

SEQ ID NO: 32 - RNA, 294 nt, Artificial Sequence
  Broad Spectrum Circular Artificial sequence to DENV cHP and HCV CDS (circ_dv_cHP_v1_circ_hcv_cds2_2)
gcgccgauguugaaagguguguuuuucguuuuuuucugucaaguguaggaucuucuggggucucgggccccacgaccaauguuggggggcguguuuuucgcuuuuuucugucaaaauaauguugaccugggguucuuuacugaacucauuauguugggaggcguguuuuuugucuuuuuuuguuuuaucuacacccgucugggguuuugucggcgccacgacauguugggggguguguuuuuugccuuuuuccgucucagugcggcgcccuugggguucccuuucgcaucacgu

SEQ ID NO: 33 - RNA, 35 nt, Artificial Sequence
  Target sequence 5 UTR chikv5utr
acacacguagccuaccaguuucuuacugcucuacu

SEQ ID NO: 34 - RNA, 29903 nt, Coronaviridae
  misc_feature (1)..(29903): NC_045512.2 Severe acute respiratory syndrome coronavirus 2 (SARS-CoV-2) isolate Wuhan-Hu-1, complete genome
auuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuugaacagcccuauguguucaucaaacguucggaugcucgaacugcaccucauggucauguuaugguugagcugguagcagaacucgaaggcauucaguacggucguaguggugagacacuugguguccuugucccucaugugggcgaaauaccaguggcuuaccgcaagguucuucuucguaagaacgguaauaaaggagcugguggccauaguuacggcgccgaucuaaagucauuugacuuaggcgacgagcuuggcacugauccuuaugaagauuuucaagaaaacuggaacacuaaacauagcagugguguuacccgugaacucaugcgugagcuuaacggaggggcauacacucgcuaugucgauaacaacuucuguggcccugauggcuacccucuugagugcauuaaagaccuucuagcacgugcugguaaagcuucaugcacuuuguccgaacaacuggacuuuauugacacuaagagggguguauacugcugccgugaacaugagcaugaaauugcuugguacacggaacguucugaaaagagcuaugaauugcagacaccuuuugaaauuaaauuggcaaagaaauuugacaccuucaauggggaauguccaaauuuuguauuucccuuaaauuccauaaucaagacuauucaaccaaggguugaaaagaaaaagcuugauggcuuuauggguagaauucgaucugucuauccaguugcgucaccaaaugaaugcaaccaaaugugccuuucaacucucaugaagugugaucauuguggugaaacuucauggcagacgggcgauuuuguuaaagccacuugcgaauuuuguggcacugagaauuugacuaaagaaggugccacuacuugugguuacuuaccccaaaaugcuguuguuaaaauuuauuguccagcaugucacaauucagaaguaggaccugagcauagucuugccgaauaccauaaugaaucuggcuugaaaaccauucuucguaaggguggucgcacuauugccuuuggaggcuguguguucucuuauguugguugccauaacaagugugccuauuggguuccacgugcuagcgcuaacauagguuguaaccauacagguguuguuggagaagguuccgaaggucuuaaugacaaccuucuugaaauacuccaaaaagagaaagucaacaucaauauuguuggugacuuuaaacuuaaugaagagaucgccauuauuuuggcaucuuuuucugcuuccacaagugcuuuuguggaaacugugaaagguuuggauuauaaagcauucaaacaaauuguugaauccugugguaauuuuaaaguuacaaaaggaaaagcuaaaaaaggugccuggaauauuggugaacagaaaucaauacugaguccucuuuaugcauuugcaucagaggcugcucguguuguacgaucaauuuucucccgcacucuugaaacugcucaaaauucugugcguguuuuacagaaggccgcuauaacaauacuagauggaauuucacaguauucacugagacucauugaugcuaugauguucacaucugauuuggcuacuaacaaucuaguuguaauggccuacauuacaggugguguuguucaguugacuucgcaguggcuaacuaacaucuuuggcacuguuuaugaaaaacucaaacccguccuugauuggcuugaagagaaguuuaaggaagguguagaguuucuuagagacgguugggaaauuguuaaauuuaucucaaccugugcuugugaaauugucgguggacaaauugucaccugugcaaaggaaauuaaggagaguguucagacauucuuuaagcuuguaaauaaauuuuuggcuuugugugcugacucuaucauuauugguggagcuaaacuuaaagccuugaauuuaggugaaacauuugucacgcacucaaagggauuguacagaaaguguguuaaauccagagaagaaacuggccuacucaugccucuaaaagccccaaaagaaauuaucuucuuagagggagaaacacuucccacagaaguguuaacagaggaaguugucuugaaaacuggugauuuacaaccauuagaacaaccuacuagugaagcuguugaagcuccauugguugguacaccaguuuguauuaacgggcuuauguugcucgaaaucaaagacacagaaaaguacugugcccuugcaccuaauaugaugguaacaaacaauaccuucacacucaaaggcggugcaccaacaaagguuacuuuuggugaugacacugugauagaagugcaagguuacaagagugugaauaucacuuuugaacuugaugaaaggauugauaaaguacuuaaugagaagugcucugccuauacaguugaacucgguacagaaguaaaugaguucgccuguguuguggcagaugcugucauaaaaacuuugcaaccaguaucugaauuacuuacaccacugggcauugauuuagaugaguggaguauggcuacauacuacuuauuugaugagucuggugaguuuaaauuggcuucacauauguauuguucuuucuacccuccagaugaggaugaagaagaaggugauugugaagaagaagaguuugagccaucaacucaauaugaguaugguacugaagaugauuaccaagguaaaccuuuggaauuuggugccacuucugcugcucuucaaccugaagaagagcaagaagaagauugguuagaugaugauagucaacaaacuguuggucaacaagacggcagugaggacaaucagacaacuacuauucaaacaauuguugagguucaaccucaauuagagauggaacuuacaccaguuguucagacuauugaagugaauaguuuuagugguuauuuaaaacuuacugacaauguauacauuaaaaaugcagacauuguggaagaagcuaaaaagguaaaaccaacagugguuguuaaugcagccaauguuuaccuuaaacauggaggagguguugcaggagccuuaaauaaggcuacuaacaaugccaugcaaguugaaucugaugauuacauagcuacuaauggaccacuuaaagugggugguaguuguguuuuaagcggacacaaucuugcuaaacacugucuucauguugucggcccaaauguuaacaaaggugaagacauucaacuucuuaagagugcuuaugaaaauuuuaaucagcacgaaguucuacuugcaccauuauuaucagcugguauuuuuggugcugacccuauacauucuuuaagaguuuguguagauacuguucgcacaaaugucuacuuagcugucuuugauaaaaaucucuaugacaaacuuguuucaagcuuuuuggaaaugaagagugaaaagcaaguugaacaaaagaucgcugagauuccuaaagaggaaguuaagccauuuauaacugaaaguaaaccuucaguugaacagagaaaacaagaugauaagaaaaucaaagcuuguguugaagaaguuacaacaacucuggaagaaacuaaguuccucacagaaaacuuguuacuuuauauugacauuaauggcaaucuucauccagauucugccacucuuguuagugacauugacaucacuuucuuaaagaaagaugcuccauauauagugggugauguuguucaagaggguguuuuaacugcugugguuauaccuacuaaaaaggcugguggcacuacugaaaugcuagcgaaagcuuugagaaaagugccaacagacaauuauauaaccacuuacccgggucaggguuuaaaugguuacacuguagaggaggcaaagacagugcuuaaaaaguguaaaagugccuuuuacauucuaccaucuauuaucucuaaugagaagcaagaaauucuuggaacuguuucuuggaauuugcgagaaaugcuugcacaugcagaagaaacacgcaaauuaaugccugucuguguggaaacuaaagccauaguuucaacuauacagcguaaauauaaggguauuaaaauacaagagggugugguugauuauggugcuagauuuuacuuuuacaccaguaaaacaacuguagcgucacuuaucaacacacuuaacgaucuaaaugaaacucuuguuacaaugccacuuggcuauguaacacauggcuuaaauuuggaagaagcugcucgguauaugagaucucucaaagugccagcuacaguuucuguuucuucaccugaugcuguuacagcguauaaugguuaucuuacuucuucuucuaaaacaccugaagaacauuuuauugaaaccaucucacuugcugguuccuauaaagauugguccuauucuggacaaucuacacaacuagguauagaauuucuuaagagaggugauaaaaguguauauuacacuaguaauccuaccacauuccaccuagauggugaaguuaucaccuuugacaaucuuaagacacuucuuucuuugagagaagugaggacuauuaagguguuuacaacaguagacaacauuaaccuccacacgcaaguuguggacaugucaaugacauauggacaacaguuugguccaacuuauuuggauggagcugauguuacuaaaauaaaaccucauaauucacaugaagguaaaacauuuuauguuuuaccuaaugaugacacucuacguguugaggcuuuugaguacuaccacacaacugauccuaguuuucuggguagguacaugucagcauuaaaucacacuaaaaaguggaaauacccacaaguuaaugguuuaacuucuauuaaaugggcagauaacaacuguuaucuugccacugcauuguuaacacuccaacaaauagaguugaaguuuaauccaccugcucuacaagaugcuuauuacagagcaagggcuggugaagcugcuaacuuuugugcacuuaucuuagccuacuguaauaagacaguaggugaguuaggugauguuagagaaacaaugaguuacuuguuucaacaugccaauuuagauucuugcaaaagagucuugaacgugguguguaaaacuuguggacaacagcagacaacccuuaaggguguagaagcuguuauguacaugggcacacuuucuuaugaacaauuuaagaaagguguucagauaccuuguacgugugguaaacaagcuacaaaauaucuaguacaacaggagucaccuuuuguuaugaugucagcaccaccugcucaguaugaacuuaagcaugguacauuuacuugugcuagugaguacacugguaauuaccaguguggucacuauaaacauauaacuucuaaagaaacuuuguauugcauagacggugcuuuacuuacaaaguccucagaauacaaagguccuauuacggauguuuucuacaaagaaaacaguuacacaacaaccauaaaaccaguuacuuauaaauuggaugguguuguuuguacagaaauugacccuaaguuggacaauuauuauaagaaagacaauucuuauuucacagagcaaccaauugaucuuguaccaaaccaaccauauccaaacgcaagcuucgauaauuuuaaguuuguaugugauaauaucaaauuugcugaugauuuaaaccaguuaacugguuauaagaaaccugcuucaagagagcuuaaaguuacauuuuucccugacuuaaauggugaugugguggcuauugauuauaaacacuacacacccucuuuuaagaaaggagcuaaauuguuacauaaaccuauuguuuggcauguuaacaaugcaacuaauaaagccacguauaaaccaaauaccugguguauacguugucuuuggagcacaaaaccaguugaaacaucaaauucguuugauguacugaagucagaggacgcgcagggaauggauaaucuugccugcgaagaucuaaaaccagucucugaagaaguaguggaaaauccuaccauacagaaagacguucuugaguguaaugugaaaacuaccgaaguuguaggagacauuauacuuaaaccagcaaauaauaguuuaaaaauuacagaagagguuggccacacagaucuaauggcugcuuauguagacaauucuagucuuacuauuaagaaaccuaaugaauuaucuagaguauuagguuugaaaacccuugcuacucaugguuuagcugcuguuaauagugucccuugggauacuauagcuaauuaugcuaagccuuuucuuaacaaaguuguuaguacaacuacuaacauaguuacacgguguuuaaaccguguuuguacuaauuauaugccuuauuucuuuacuuuauugcuacaauuguguacuuuuacuagaaguacaaauucuagaauuaaagcaucuaugccgacuacuauagcaaagaauacuguuaagagugucgguaaauuuugucuagaggcuucauuuaauuauuugaagucaccuaauuuuucuaaacugauaaauauuauaauuugguuuuuacuauuaaguguuugccuagguucuuuaaucuacucaaccgcugcuuuagguguuuuaaugucuaauuuaggcaugccuucuuacuguacugguuacagagaaggcuauuugaacucuacuaaugucacuauugcaaccuacuguacugguucuauaccuuguaguguuugucuuagugguuuagauucuuuagacaccuauccuucuuuagaaacuauacaaauuaccauuucaucuuuuaaaugggauuuaacugcuuuuggcuuaguugcagagugguuuuuggcauauauucuuuucacuagguuuuucuauguacuuggauuggcugcaaucaugcaauuguuuuucagcuauuuugcaguacauuuuauuaguaauucuuggcuuaugugguuaauaauuaaucuuguacaaauggccccgauuucagcuaugguuagaauguacaucuucuuugcaucauuuuauuauguauggaaaaguuaugugcauguuguagacgguuguaauucaucaacuuguaugauguguuacaaacguaauagagcaacaagagucgaauguacaacuauuguuaaugguguuagaagguccuuuuaugucuaugcuaauggagguaaaggcuuuugcaaacuacacaauuggaauuguguuaauugugauacauucugugcugguaguacauuuauuagugaugaaguugcgagagacuugucacuacaguuuaaaagaccaauaaauccuacugaccagucuucuuacaucguugauaguguuacagugaagaaugguuccauccaucuuuacuuugauaaagcuggucaaaagacuuaugaaagacauucucucucucauuuuguuaacuuagacaaccugagagcuaauaacacuaaagguucauugccuauuaauguuauaguuuuugaugguaaaucaaaaugugaagaaucaucugcaaaaucagcgucuguuuacuacagucagcuuaugugucaaccuauacuguuacuagaucaggcauuagugucugauguuggugauagugcggaaguugcaguuaaaauguuugaugcuuacguuaauacguuuucaucaacuuuuaacguaccaauggaaaaacucaaaacacuaguugcaacugcagaagcugaacuugcaaagaauguguccuuagacaaugucuuaucuacuuuuauuucagcagcucggcaaggguuuguugauucagauguagaaacuaaagauguuguugaaugucuuaaauugucacaucaaucugacauagaaguuacuggcgauaguuguaauaacuauaugcucaccuauaacaaaguugaaaacaugacaccccgugaccuuggugcuuguauugacuguagugcgcgucauauuaaugcgcagguagcaaaaagucacaacauugcuuugauauggaacguuaaagauuucaugucauugucugaacaacuacgaaaacaaauacguagugcugcuaaaaagaauaacuuaccuuuuaaguugacaugugcaacuacuagacaaguuguuaauguuguaacaacaaagauagcacuuaagggugguaaaauuguuaauaauugguugaagcaguuaauuaaaguuacacuuguguuccuuuuuguugcugcuauuuucuauuuaauaacaccuguucaugucaugucuaaacauacugacuuuucaagugaaaucauaggauacaaggcuauugaugguggugucacucgugacauagcaucuacagauacuuguuuugcuaacaaacaugcugauuuugacacaugguuuagccagcguggugguaguuauacuaaugacaaagcuugcccauugauugcugcagucauaacaagagaaguggguuuugucgugccugguuugccuggcacgauauuacgcacaacuaauggugacuuuuugcauuucuuaccuagaguuuuuagugcaguugguaacaucuguuacacaccaucaaaacuuauagaguacacugacuuugcaacaucagcuuguguuuuggcugcugaauguacaauuuuuaaagaugcuucugguaagccaguaccauauuguuaugauaccaauguacuagaagguucuguugcuuaugaaaguuuacgcccugacacacguuaugugcucauggauggcucuauuauucaauuuccuaacaccuaccuugaagguucuguuagagugguaacaacuuuugauucugaguacuguaggcacggcacuugugaaagaucagaagcugguguuuguguaucuacuagugguagauggguacuuaacaaugauuauuacagaucuuuaccaggaguuuucugugguguagaugcuguaaauuuacuuacuaauauguuuacaccacuaauucaaccuauuggugcuuuggacauaucagcaucuauaguagcuggugguauuguagcuaucguaguaacaugccuugccuacuauuuuaugagguuuagaagagcuuuuggugaauacagucauguaguugccuuuaauacuuuacuauuccuuaugucauucacuguacucuguuuaacaccaguuuacucauucuuaccugguguuuauucuguuauuuacuuguacuugacauuuuaucuuacuaaugauguuucuuuuuuagcacauauucaguggaugguuauguucacaccuuuaguaccuuucuggauaacaauugcuuauaucauuuguauuuccacaaagcauuucuauugguucuuuaguaauuaccuaaagagacguguagucuuuaaugguguuuccuuuaguacuuuugaagaagcugcgcugugcaccuuuuuguuaaauaaagaaauguaucuaaaguugcguagugaugugcuauuaccucuuacgcaauauaauagauacuuagcucuuuauaauaaguacaaguauuuuaguggagcaauggauacaacuagcuacagagaagcugcuuguugucaucucgcaaaggcucucaaugacuucaguaacucagguucugauguucuuuaccaaccaccacaaaccucuaucaccucagcuguuuugcagagugguuuuagaaaaauggcauucccaucugguaaaguugaggguuguaugguacaaguaacuugugguacaacuacacuuaacggucuuuggcuugaugacguaguuuacuguccaagacaugugaucugcaccucugaagacaugcuuaacccuaauuaugaagauuuacucauucguaagucuaaucauaauuucuugguacaggcugguaauguucaacucaggguuauuggacauucuaugcaaaauuguguacuuaagcuuaagguugauacagccaauccuaagacaccuaaguauaaguuuguucgcauucaaccaggacagacuuuuucaguguuagcuuguuacaaugguucaccaucugguguuuaccaaugugcuaugaggcccaauuucacuauuaaggguucauuccuuaaugguucaugugguaguguugguuuuaacauagauuaugacugugucucuuuuuguuacaugcaccauauggaauuaccaacuggaguucaugcuggcacagacuuagaagguaacuuuuauggaccuuuuguugacaggcaaacagcacaagcagcugguacggacacaacuauuacaguuaauguuuuagcuugguuguacgcugcuguuauaaauggagacaggugguuucucaaucgauuuaccacaacucuuaaugacuuuaaccuuguggcuaugaaguacaauuaugaaccucuaacacaagaccauguugacauacuaggaccucuuucugcucaaacuggaauugccguuuuagauaugugugcuucauuaaaagaauuacugcaaaaugguaugaauggacguaccauauuggguagugcuuuauuagaagaugaauuuacaccuuuugauguuguuagacaaugcucagguguuacuuuccaaagugcagugaaaagaacaaucaaggguacacaccacugguuguuacucacaauuuugacuucacuuuuaguuuuaguccagaguacucaauggucuuuguucuuuuuuuuguaugaaaaugccuuuuuaccuuuugcuauggguauuauugcuaugucugcuuuugcaaugauguuugucaaacauaagcaugcauuucucuguuuguuuuuguuaccuucucuugccacuguagcuuauuuuaauauggucuauaugccugcuaguugggugaugcguauuaugacaugguuggauaugguugauacuaguuugucugguuuuaagcuaaaagacuguguuauguaugcaucagcuguaguguuacuaauccuuaugacagcaagaacuguguaugaugauggugcuaggagaguguggacacuuaugaaugucuugacacucguuuauaaaguuuauuaugguaaugcuuuagaucaagccauuuccaugugggcucuuauaaucucuguuacuucuaacuacucagguguaguuacaacugucauguuuuuggccagagguauuguuuuuauguguguugaguauugcccuauuuucuucauaacugguaauacacuucaguguauaaugcuaguuuauuguuucuuaggcuauuuuuguacuuguuacuuuggccucuuuuguuuacucaaccgcuacuuuagacugacucuugguguuuaugauuacuuaguuucuacacaggaguuuagauauaugaauucacagggacuacucccacccaagaauagcauagaugccuucaaacucaacauuaaauuguuggguguugguggcaaaccuuguaucaaaguagccacuguacagucuaaaaugucagauguaaagugcacaucaguagucuuacucucaguuuugcaacaacucagaguagaaucaucaucuaaauugugggcucaauguguccaguuacacaaugacauucucuuagcuaaagauacuacugaagccuuugaaaaaaugguuucacuacuuucuguuuugcuuuccaugcagggugcuguagacauaaacaagcuuugugaagaaaugcuggacaacagggcaaccuuacaagcuauagccucagaguuuaguucccuuccaucauaugcagcuuuugcuacugcucaagaagcuuaugagcaggcuguugcuaauggugauucugaaguuguucuuaaaaaguugaagaagucuuugaauguggcuaaaucugaauuugaccgugaugcagccaugcaacguaaguuggaaaagauggcugaucaagcuaugacccaaauguauaaacaggcuagaucugaggacaagagggcaaaaguuacuagugcuaugcagacaaugcuuuucacuaugcuuagaaaguuggauaaugaugcacucaacaacauuaucaacaaugcaagagaugguuguguucccuugaacauaauaccucuuacaacagcagccaaacuaaugguugucauaccagacuauaacacauauaaaaauacgugugaugguacaacauuuacuuaugcaucagcauugugggaaauccaacagguuguagaugcagauaguaaaauuguucaacuuagugaaauuaguauggacaauucaccuaauuuagcauggccucuuauuguaacagcuuuaagggccaauucugcugucaaauuacagaauaaugagcuuaguccuguugcacuacgacagaugucuugugcugccgguacuacacaaacugcuugcacugaugacaaugcguuagcuuacuacaacacaacaaagggagguagguuuguacuugcacuguuauccgauuuacaggauuugaaaugggcuagauucccuaagagugauggaacugguacuaucuauacagaacuggaaccaccuuguagguuuguuacagacacaccuaaagguccuaaagugaaguauuuauacuuuauuaaaggauuaaacaaccuaaauagagguaugguacuugguaguuuagcugccacaguacgucuacaagcugguaaugcaacagaagugccugccaauucaacuguauuaucuuucugugcuuuugcuguagaugcugcuaaagcuuacaaagauuaucuagcuagugggggacaaccaaucacuaauuguguuaagauguuguguacacacacugguacuggucaggcaauaacaguuacaccggaagccaauauggaucaagaauccuuugguggugcaucguguugucuguacugccguugccacauagaucauccaaauccuaaaggauuuugugacuuaaaagguaaguauguacaaauaccuacaacuugugcuaaugacccuguggguuuuacacuuaaaaacacagucuguaccgucugcgguauguggaaagguuauggcuguaguugugaucaacuccgcgaacccaugcuucagucagcugaugcacaaucguuuuuaaacggguuugcgguguaagugcagcccgucuuacaccgugcggcacaggcacuaguacugaugucguauacagggcuuuugacaucuacaaugauaaaguagcugguuuugcuaaauuccuaaaaacuaauuguugucgcuuccaagaaaaggacgaagaugacaauuuaauugauucuuacuuuguaguuaagagacacacuuucucuaacuaccaacaugaagaaacaauuuauaauuuacuuaaggauuguccagcuguugcuaaacaugacuucuuuaaguuuagaauagacggugacaugguaccacauauaucacgucaacgucuuacuaaauacacaauggcagaccucgucuaugcuuuaaggcauuuugaugaagguaauugugacacauuaaaagaaauacuugucacauacaauuguugugaugaugauuauuucaauaaaaaggacugguaugauuuuguagaaaacccagauauauuacgcguauacgccaacuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccagguaguggaguuccuguuguagauucuuauuauucauuguuaaugccuauauuaaccuugaccagggcuuuaacugcagagucacauguugacacugacuuaacaaagccuuacauuaagugggauuuguuaaaauaugacuucacggaagagagguuaaaacucuuugaccguuauuuuaaauauugggaucagacauaccacccaaauuguguuaacuguuuggaugacagaugcauucugcauugugcaaacuuuaauguuuuauucucuacaguguucccaccuacaaguuuuggaccacuagugagaaaaauauuuguugaugguguuccauuuguaguuucaacuggauaccacuucagagagcuagguguuguacauaaucaggauguaaacuuacauagcucuagacuuaguuuuaaggaauuacuuguguaugcugcugacccugcuaugcacgcugcuucugguaaucuauuacuagauaaacgcacuacgugcuuuucaguagcugcacuuacuaacaauguugcuuuucaaacugucaaacccgguaauuuuaacaaagacuucuaugacuuugcugugucuaaggguuucuuuaaggaaggaaguucuguugaauuaaaacacuucuucuuugcucaggaugguaaugcugcuaucagcgauuaugacuacuaucguuauaaucuaccaacaaugugugauaucagacaacuacuauuuguaguugaaguuguugauaaguacuuugauuguuacgaugguggcuguauuaaugcuaaccaagucaucgucaacaaccuagacaaaucagcugguuuuccauuuaauaaaugggguaaggcuagacuuuauuaugauucaaugaguuaugaggaucaagaugcacuuuucgcauauacaaaacguaaugucaucccuacuauaacucaaaugaaucuuaaguaugccauuagugcaaagaauagagcucgcaccguagcuggugucucuaucuguaguacuaugaccaauagacaguuucaucaaaaauuauugaaaucaauagccgccacuagaggagcuacuguaguaauuggaacaagcaaauucuauggugguuggcacaacauguuaaaaacuguuuauagugauguagaaaacccucaccuuauggguugggauuauccuaaaugugauagagccaugccuaacaugcuuagaauuauggccucacuuguucuugcucgcaaacauacaacguguuguagcuugucacaccguuucuauagauuagcuaaugagugugcucaaguauugagugaaauggucauguguggcgguucacuauauguuaaaccagguggaaccucaucaggagaugccacaacugcuuaugcuaauaguguuuuuaacauuugucaagcugucacggccaauguuaaugcacuuuuaucuacugaugguaacaaaauugccgauaaguauguccgcaauuuacaacacagacuuuaugagugucucuauagaaauagagauguugacacagacuuugugaaugaguuuuacgcauauuugcguaaacauuucucaaugaugauacucucugacgaugcuguuguguguuucaauagcacuuaugcaucucaaggucuaguggcuagcauaaagaacuuuaagucaguucuuuauuaucaaaacaauguuuuuaugucugaagcaaaauguuggacugagacugaccuuacuaaaggaccucaugaauuuugcucucaacauacaaugcuaguuaaacagggugaugauuauguguaccuuccuuacccagauccaucaagaauccuaggggccggcuguuuuguagaugauaucguaaaaacagaugguacacuuaugauugaacgguucgugucuuuagcuauagaugcuuacccacuuacuaaacauccuaaucaggaguaugcugaugucuuucauuuguacuuacaauacauaagaaagcuacaugaugaguuaacaggacacauguuagacauguauucuguuaugcuuacuaaugauaacacuucaagguauugggaaccugaguuuuaugaggcuauguacacaccgcauacagucuuacaggcuguuggggcuuguguucuuugcaauucacagacuucauuaagauguggugcuugcauacguagaccauucuuauguuguaaaugcuguuacgaccaugucauaucaacaucacauaaauuagucuugucuguuaauccguauguuugcaaugcuccagguugugaugucacagaugugacucaacuuuacuuaggagguaugagcuauuauuguaaaucacauaaaccacccauuaguuuuccauugugugcuaauggacaaguuuuugguuuauauaaaaauacauguguugguagcgauaauguuacugacuuuaaugcaauugcaacaugugacuggacaaaugcuggugauuacauuuuagcuaacaccuguacugaaagacucaagcuuuuugcagcagaaacgcucaaagcuacugaggagacauuuaaacugucuuaugguauugcuacuguacgugaagugcugucugacagagaauuacaucuuucaugggaaguugguaaaccuagaccaccacuuaaccgaaauuaugucuuuacugguuaucguguaacuaaaaacaguaaaguacaaauaggagaguacaccuuugaaaaaggugacuauggugaugcuguuguuuaccgagguacaacaacuuacaaauuaaauguuggugauuauuuugugcugacaucacauacaguaaugccauuaagugcaccuacacuagugccacaagagcacuauguuagaauuacuggcuuauacccaacacucaauaucucagaugaguuuucuagcaauguugcaaauuaucaaaagguugguaugcaaaaguauucuacacuccagggaccaccugguacugguaagagucauuuugcuauuggccuagcucucuacuacccuucugcucgcauaguguauacagcuugcucucaugccgcuguugaugcacuaugugagaaggcauuaaaauauuugccuauagauaaauguaguagaauuauaccugcacgugcucguguagaguguuuugauaaauucaaagugaauucaacauuagaacaguaugucuuuuguacuguaaaugcauugccugagacgacagcagauauaguugucuuugaugaaauuucaauggccacaaauuaugauuugaguguugucaaugccagauuacgugcuaagcacuauguguacauuggcgacccugcucaauuaccugcaccacgcacauugcuaacuaagggcacacuagaaccagaauauuucaauucaguguguagacuuaugaaaacuauagguccagacauguuccucggaacuugucggcguuguccugcugaaauuguugacacugugagugcuuugguuuaugauaauaagcuuaaagcacauaaagacaaaucagcucaaugcuuuaaaauguuuuauaaggguguuaucacgcaugauguuucaucugcaauuaacaggccacaaauaggcgugguaagagaauuccuuacacguaacccugcuuggagaaaagcugucuuuauuucaccuuauaauucacagaaugcuguagccucaaagauuuugggacuaccaacucaaacuguugauucaucacagggcucagaauaugacuaugucauauucacucaaaccacugaaacagcucacucuuguaauguaaacagauuuaauguugcuauuaccagagcaaaaguaggcauacuuugcauaaugucugauagagaccuuuaugacaaguugcaauuuacaagucuugaaauuccacguaggaauguggcaacuuuacaagcugaaaauguaacaggacucuuuaaagauuguaguaagguaaucacuggguuacauccuacacaggcaccuacacaccucaguguugacacuaaauucaaaacugaagguuuauguguugacauaccuggcauaccuaaggacaugaccuauagaagacucaucucuaugauggguuuuaaaaugaauuaucaaguuaaugguuacccuaacauguuuaucacccgcgaagaagcuauaagacauguacgugcauggauuggcuucgaugucgaggggugucaugcuacuagagaagcuguugguaccaauuuaccuuuacagcuagguuuuucuacagguguuaaccuaguugcuguaccuacagguuauguugauacaccuaauaauacagauuuuuccagaguuagugcuaaaccaccgccuggagaucaauuuaaacaccucauaccacuuauguacaaaggacuuccuuggaauguagugcguauaaagauuguacaaauguuaagugacacacuuaaaaaucucucugacagagucguauuugucuuaugggcacauggcuuugaguugacaucuaugaaguauuuugugaaaauaggaccugagcgcaccuguugucuaugugauagacgugccacaugcuuuuccacugcuucagacacuuaugccuguuggcaucauucuauuggauuugauuacgucuauaauccguuuaugauugauguucaacaaugggguuuuacagguaaccuacaaagcaaccaugaucuguauugucaaguccaugguaaugcacauguagcuaguugugaugcaaucaugacuaggugucuagcuguccacgagugcuuuguuaagcguguugacuggacuauugaauauccuauaauuggugaugaacugaagauuaaugcggcuuguagaaagguucaacacaugguuguuaaagcugcauuauuagcagacaaauucccaguucuucacgacauugguaacccuaaagcuauuaaguguguaccucaagcugauguagaauggaaguucuaugaugcacagccuuguagugacaaagcuuauaaaauagaagaauuauucuauucuuaugccacacauucugacaaauucacagaugguguaugccuauuuuggaauugcaaugucgauagauauccugcuaauuccauuguuuguagauuugacacuagagugcuaucuaaccuuaacuugccugguugugaugguggcaguuuguauguaaauaaacaugcauuccacacaccagcuuuugauaaaagugcuuuuguuaauuuaaaacaauuaccauuuuucuauuacucugacaguccaugugagucucauggaaaacaaguagugucagauauagauuauguaccacuaaagucugcuacguguauaacacguugcaauuuagguggugcugucuguagacaucaugcuaaugaguacagauuguaucucgaugcuuauaacaugaugaucucagcuggcuuuagcuuguggguuuacaaacaauuugauacuuauaaccucuggaacacuuuuacaagacuucagaguuuagaaaauguggcuuuuaauguuguaaauaagggacacuuugauggacaacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuuagucauagucaguuaggugguuuacaucuacugauuggacuagcuaaacguuuuaaggaaucaccuuuugaauuagaagauuuuauuccuauggacaguacaguuaaaaacuauuucauaacagaugcgcaaacagguucaucuaaguguguguguucuguuauugauuuauuacuugaugauuuuguugaaauaauaaaaucccaagauuuaucuguaguuucuaagguugucaaagugacuauugacuauacagaaauuucauuuaugcuuugguguaaagauggccauguagaaacauuuuacccaaaauuacaaucuagucaagcguggcaaccggguguugcuaugccuaaucuuuacaaaaugcaaagaaugcuauuagaaaagugugaccuucaaaauuauggugauagugcaacauuaccuaaaggcauaaugaugaaugucgcaaaauauacucaacugugucaauauuuaaacacauuaacauuagcuguacccuauaauaugagaguuauacauuuuggugcugguucugauaaaggaguugcaccagguacagcuguuuuaagacagugguugccuacggguacgcugcuugucgauucagaucuuaaugacuuugucucugaugcagauucaacuuugauuggugauugugcaacuguacauacagcuaauaaaugggaucucauuauuagugauauguacgacccuaagacuaaaaauguuacaaaagaaaaugacucuaaagaggguuuuuucacuuacauuuguggguuuauacaacaaaagcuagcucuuggagguuccguggcuauaaagauaacagaacauucuuggaaugcugaucuuuauaagcucaugggacacuucgcaugguggacagccuuuguuacuaaugugaaugcgucaucaucugaagcauuuuuaauuggauguaauuaucuuggcaaaccacgcgaacaaauagaugguuaugucaugcaugcaaauuacauauuuuggaggaauacaaauccaauucaguugucuuccuauucuuuauuugacaugaguaaauuuccccuuaaauuaagggguacugcuguuaugucuuuaaaagaaggucaaaucaaugauaugauuuuaucucuucuuaguaaagguagacuuauaauuagagaaaacaacagaguuguuauuucuagugauguucuuguuaacaacuaaacgaacaauguuuguuuuucuuguuuuauugccacuagucucuagucaguguguuaaucuuacaaccagaacucaauuacccccugcauacacuaauucuuucacacgugguguuuauuacccugacaaaguuuucagauccucaguuuuacauucaacucaggacuuguucuuaccuuucuuuuccaauguuacuugguuccaugcuauacaugucucugggaccaaugguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuucaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggauguuaacugcacagaagucccuguugcuauucaugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauuuuggugguuuuaauuuuucacaaauauuaccagauccaucaaaaccaagcaagaggucauuuauugaagaucuacuuuucaacaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacauaaacgaacuuauggauuuguuuaugagaaucuucacaauuggaacuguaacuuugaagcaaggugaaaucaaggaugcuacuccuucagauuuuguucgcgcuacugcaacgauaccgauacaagccucacucccuuucggauggcuuauuguuggcguugcacuucuugcuguuuuucagagcgcuuccaaaaucauaacccucaaaaagagauggcaacuagcacucuccaaggguguucacuuuguuugcaacuugcuguuguuguuuguaacaguuuacucacaccuuuugcucguugcugcuggccuugaagccccuuuucucuaucuuuaugcuuuagucuacuucuugcagaguauaaacuuuguaagaauaauaaugaggcuuuggcuuugcuggaaaugccguuccaaaaacccauuacuuuaugaugccaacuauuuucuuugcuggcauacuaauuguuacgacuauuguauaccuuacaauaguguaacuucuucaauugucauuacuucaggugauggcacaacaaguccuauuucugaacaugacuaccagauuggugguuauacugaaaaaugggaaucuggaguaaaagacuguguuguauuacacaguuacuucacuucagacuauuaccagcuguacucaacucaauugaguacagacacugguguugaacauguuaccuucuucaucuacaauaaaauuguugaugagccugaagaacauguccaaauucacacaaucgacgguucauccggaguuguuaauccaguaauggaaccaauuuaugaugaaccgacgacgacuacuagcgugccuuuguaagcacaagcugaugaguacgaacuuauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucuaaacgaacuaaauauuauauuaguuuuucuguuuggaacuuuaauuuuagccauggcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcuguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacaguaagugacaacagauguuucaucucguugacuuucagguuacuauagcagagauauuacuaauuauuaugaggacuuuuaaaguuuccauuuggaaucuugauuacaucauaaaccucauaauuaaaaauuuaucuaagucacuaacugagaauaaauauucucaauuagaugaagagcaaccaauggagauugauuaaacgaacaugaaaauuauucuuuucuuggcacugauaacacucgcuacuugugagcuuuaucacuaccaagaguguguuagagguacaacaguacuuuuaaaagaaccuugcucuucuggaacauacgagggcaauucaccauuucauccucuagcugauaacaaauuugcacugacuugcuuuagcacucaauuugcuuuugcuuguccugacggcguaaaacacgucuaucaguuacgugccagaucaguuucaccuaaacuguucaucagacaagaggaaguucaagaacuuuacucuccaauuuuucuuauuguugcggcaauaguguuuauaacacuuugcuucacacucaaaagaaagacagaaugauugaacuuucauuaauugacuucuauuugugcuuuuuagccuuucugcuauuccuuguuuuaauuaugcuuauuaucuuuugguucucacuugaacugcaagaucauaaugaaacuugucacgccuaaacgaacaugaaauuucuuguuuucuuaggaaucaucacaacuguagcugcauuucaccaagaauguaguuuacagucauguacucaacaucaaccauauguaguugaugacccguguccuauucacuucuauucuaaaugguauauuagaguaggagcuagaaaaucagcaccuuuaauugaauugugcguggaugaggcugguucuaaaucacccauucaguacaucgauaucgguaauuauacaguuuccuguuuaccuuuuacaauuaauugccaggaaccuaaauuggguagucuuguagugcguuguucguucuaugaagacuuuuuagaguaucaugacguucguguuguuuuagauuucaucuaaacgaacaaacuaaaaugucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaaacucaugcagaccacacaaggcagaugggcuauauaaacguuuucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 35 - RNA, 33 nt, Artificial Sequence
  Target sequence RSE Circ chikvRSE
agcaaauaaucuauagaucaaagggcuacgcaa

SEQ ID NO: 36 - RNA, 294 nt, Artificial Sequence
  Artificial circular ARN 1; target disruption structure comprised in SARS-CoV-2 3'UTR replication site
ugcaguuagauacugcuuguguuaugugguugugaggguuuauuuugagauccugcugguuacuugugcuguguaguuaugagaguuuguuuuggauaccuuuagccugcuuguguugugugguugcgaggauuuauucugcucggaaacgauuuguuuguguuauguaguuguggggauucauucuggcugcuuccucuuuauuuguguuguguaguuacgggaguucguuuugucccacauagaguugcuugugcuaugugguuacggggauuuauuuugccagggccaaac

SEQ ID NO: 37 - RNA, 29 nt, Artificial Sequence
  Target sequence SL III 3' UTR circ_wnv_slII_1/2
uuuugaggagaaagucaggccgggaaguu

SEQ ID NO: 38 - RNA, 294 nt, Artificial Sequence
  Artificial circular ARN 2; target disruption structure comprised in SARS-CoV-2 3'UTR replication site
ugcaguuagauacugcuuguguuaugugguugugaggguuuauuuugagauccugcugguuacuugugcuguguaguuaugagaguuuguuuuggauaccuuuagccugcuuguguugugugguugcgaggauuuauucugcucggaaacgauuuguuuguguuauguaguuguggggauucauucuggcugcuuccucuuuguuuguguuguguaguuacggggguucguuuugucccacauagaguuacuugugcuauguaguuaugaggauucauuuugccagggccaaac

SEQ ID NO: 39 - RNA, 294 nt, Artificial Sequence
  Circ Chikv_re
gcgccgauuauggcuaaaagagucaacguuuuguaguagcugcggguugcccucauuacgguuaagaggguuaauguuuuauagcagcugcccgcgagcaggauuaugguuggaaagguuaacguuuugcagcagcguucguaacacuucauuauggcuaaggaagccaacguuuugugguagccuagguucgaaccgauuauggcuagaggggucagcguuuuaugguagccaucaaaaccuuucauuacggcugagagagccaauguuuuacaguagcuccaacgacguacu

SEQ ID NO: 40 - RNA, 294 nt, Artificial Sequence
  Artificial circular RNA 3; target disruption structure comprised in SARS-CoV-2 3'UTR replication site
ugcaguuagauacugcuuguguuaugugguugugaggguuuauuuugagauccugcugguuguuugugcuauguaguuacgaggguucauuuuggauaccuuuagccugcuuguguugugugguugcgaggauuuauucugcucggaaacgauuuguuuguguuauguaguuguggggauucauucuggcugcuuccucucugcuugugcuguguaguuaugagaguuuguuuugucccacauagaguuauuuguguuguguaguugugggaguuuguuuugccagggccaaac

SEQ ID NO: 41 - RNA, 294 nt, Artificial Sequence
  Artificial circular RNA 4; target disruption structure comprised in SARS-CoV-2 3'UTR replication site
ugcaguuagauauuguuugugcuaugugguuguggggauucauucugaugucguauuguuugcuuguguugugugguuacgggaauuuguuuugaacucuuaguuguuguuugugcuguguaguuguggggguuuguucugauguucugaucuuuguuuguguuauguaguuacgaggauuuauucuggugcaacaaguucuauuuguguuaugugguugcggggguucguuuugcccggccuucgcuuacuuguguuguguaguugugaggauucguuuuggaugcaucgaac

SEQ ID NO: 42 - RNA, 294 nt, Artificial Sequence
  Artificial circular RNA 5; target disruption structure comprised in SARS-CoV-2 3'UTR replication site
ugcaguuagauacuauuugugcuauguaguugcggggguuuguuuuguggcuuucuuaccuguuuguguuaugugguugugaggauuuguuuuggccgacucguuccugcuugugcuguguaguugugggaguucauuuuguucagaguuagccuacuuguguuauguaguuaugagaguucguuuuggagagacgcacacuguuuguguugugugguuauggggauucguuuuguggguccggcuccuguuugugcugugugguuaugagaauuuguucugaagauuuauucc

SEQ ID NO: 43 - RNA, 292 nt, Artificial Sequence
  Artificial circular RNA 1; target disruption structure comprised in SARS-CoV-2 5'UTR (SlII)
ugcaguuagauaagaucugcaggggguugaggguugguugauccaggcugcuagaucuauaagggguugggaguugguucuuuagcuaucccagauuugugagggguuggggguugguuaggcuuacaguuaagaucuaugggagguuggagguugguuaccgccgaguuauagauuuauaggggaucggaaguugguuaaagcuagggcgaagaucugcgggggguuggaaguugguuauacaacgaauuaagauuuauggggggucggggguugguuuaguaguuggaug

SEQ ID NO: 44 - RNA, 292 nt, Artificial Sequence
  Artificial circular RNA 2; target disruption structure comprised in SARS-CoV-2 5'UTR (SlII)
ugcaguuagauaggaucuauaagggguuggaaguugguuauguggucgcgcgaggucuguaagagauugaaaguugguugucacugacguaaagauuuacgagagaucgaggguugguugaaugucggucugggguuugugggggguuggagguugguuauugguauaacggaggucuaugagagguugaggguugguucgcgauagacaccgggucugcaggaggucggaaguugguuaguugaacagacuggguuuauggggggucgagaguugguuauuuguuccgagg

SEQ ID NO: 45 - RNA, 292 nt, Artificial Sequence
  Artificial circular RNA 3; target disruption structure comprised in SARS-CoV-2 5'UTR (SlII)
ugcaguuagauaagguuugcgagggauuggaaguugguugaaauguggaaggggauuugcgagagguugaaaguugguuuuuauuaaagguaggaucuacaagggauugaagguugguuacccaguagcucaggguuuacaggaggucggagguugguuuuucuggauagaaaggucuauaagagguugaggguugguuuggagauuguaacggauuuaugagagaucgagaguugguuaaaaacaaggaacggauuuacaagggguuggggguugguuauacuuagcggug

SEQ ID NO: 46 - RNA, 282 nt, Artificial Sequence
  Artificial circular RNA 1; target hybridization region: SARS-CoV-2 Target A
ugcaguuagauauggugcugugucgucgucuauucuaaguuugggagauccugcuggugguauuauguuacugucuguucuaaauuugaggauaccuuuagcuggugucguguuauugucuguuuuggacuuaggcucggaaacgauugguacugugucacugucuguuuuaggcuugaagcugcuuccucuugguguuaugucaucgucuauuuugaacuugggucccacauagagugguaccaugucguugucuguucuggauuuaaaccagggccaaac

SEQ ID NO: 47 - RNA, 282 nt, Artificial Sequence
  Artificial circular RNA 2; target hybridization region: SARS-CoV-2 Target A
ugcaguuagauaugguacugugucacugucuguucugaacuugagcguucacgucucuggugcuauguugucgucuauucuaaacuuaagaacuagguauaguggugucguguuaucgucuauuuugggcuugggggcuccccuuggugguaccaugucaccgucuauucugaauuuaaaucucccauagugugguaucguguugccgucuauuuuagacuuggaccaccgucuguuugguacuauguugcugucuguuuugaacuugagaucagauuaagg

SEQ ID NO: 48 - RNA, 282 nt, Artificial Sequence
  Artificial circular RNA 3; target hybridization region: SARS-CoV-2 Target A
ugcaguuagauaugguaccgugucgucgucuauuuuggauuuaaaagauccugcuggugguauuaugucguuguuuguucugaguuuaaagauaccuuuagcugguaccauguuaccgucuguucuaaguuuaaacucggaaacgauugguauuguguugucguuuguucuagauuuaaagcugcuuccucuugguaucgugucgccgucuauucugagcuuaaaucccacauagagugguacuauguuguugucuauuuugaguuuaaaccagggccaaac

SEQ ID NO: 49 - RNA, 282 nt, Artificial Sequence
  Artificial circular RNA 4; target hybridization region: SARS-CoV-2 Target A
ugcaguuagauaugguaccgugucgccguuuguucugagcuuaaaguucuugcgcucugguaucauguugcuguuuauucuaggcuuaaaucggccgucuggugguauugugucguugucuguucugaguuuaaaucucuuucccggugguacuguguugucgucuauucuggguuuaaaacucgaccccggugguaccaugucaccgucuguuuuagacuuaaaguuacaagucgcugguauuauguugcugucuauucuagauuuaaaauucccccagcc

SEQ ID NO: 50 - RNA, 299 nt, Artificial Sequence
  Artificial circular RNA 1; target hybridization region: SARS-CoV-2 Target C
ugcaguuagauauagaggccuugguggcagguuucuuggugaagugaguauaguagaggucuuaguagcagguuuuuuagugauguaaccuauguagaagccucagcagcagauuucuugguggggggucaaggguaggggucuuggugguagauuuuuugguggcacuugaggaguaggaguuucggcgguggauuucuuagugggcuaggaaauuugggagcuuuggugguagguuuuuuggugaggugggaggguugggggcuucgguagcggauuuuuuaguggaacugaaagcg

SEQ ID NO: 51 - RNA, 292 nt, Artificial Sequence
  Artificial circular RNA 2; target hybridization region: SARS-CoV-2 Target C
ugcaguuagauauaggaguuucggcggcggguuuuuuggugaaaguugaauuuagaaguuucgguggcggguuucuuagugagaaauucagauagaaguuuuggcaguggauuucuuggugaggaauggguuuggaagccuugguaguggauuuuuuaguggccacccuugguagaagcuuuggugguagauuuuuugguggguaacuuaauugggaguuuugguggcagguuuuuuagugauaggagaccguagggguuucagcaguagauuucuuagugggagacagacu

SEQ ID NO: 52 - RNA, 292 nt, Artificial Sequence
  Artificial circular RNA 3; target hybridization region: SARS-CoV-2 Target C
ugcaguuagauauaggagucucggcagcggauuuuuuaguggacaugacuuuuagaggccuugguagcggauuucuuaguggucuacagcgaugggaguuuuggcgguggguuuuuuggugaccucuaauguuggaggcuucagcagcagguuuuuuagugauaagaguagguaggggccuuaguagcagauuucuuggugggauucgaauuuagagguuuuaguaguagguuucuuagugaagggcuuuauuggggguuuuagcggcagauuuuuugguggauucgaucug

SEQ ID NO: 53 - RNA, 292 nt, Artificial Sequence
  Artificial circular RNA 4; target hybridization region: SARS-CoV-2 Target C
ugcaguuagauauggaaguuuuggcaguggguuucuuaguggugggacuugauagaagucuuagugguagguuucuugguggggcggaguguuggaggcuuugguagcggguuuuuuaguggggcuaucucgugggagucuugguaguagauuucuuggugaaggaugcggguggaagucuuagcggcggauuuuuugguggccaguuaguauggggguuuugguagcagguuuuuuggugauaguagacucuggaggcuucgguagcagauuuuuuagugaguuauaaagg

SEQ ID NO: 54 - RNA, 270 nt, Artificial Sequence
  Artificial circular RNA 1; target hybridization region: SARS-CoV-2 Target D
ugcaguuagauaaaaccguaagcagccuguagaagguagacgaguccagacguacaaaccguaggcagcuugcagaagauagacgaagaacgaguacuaaaccgugggcaguuugcgggggauagacgaagcuuggagacuaaaccguaaguagucugugggggauggacgaauuucuggcaauaaaccguagguaguuugcagaggguagacgagcucguagcuagaaaccgugaguagccuguagaggauggacgacugagaccucaa

SEQ ID NO: 55 - RNA, 270 nt, Artificial Sequence
  Artificial circular RNA 2; target hybridization region: SARS-CoV-2 Target D
ugcaguuagauaaaaccguaggcagccuguaggagguagacgaguuuucucacggaaaccguggguagcuuguaggagauagacgaguugaggaccagaaaccguaagugguuugcaggagauggacgaguuugggcaguuaaaccgugagcagucugcaggggguagacgaauguucggagcaaaaccgugaguaguuuguagaagguggacgagaggagaguaggaaaccguaagcagucuguaggggauggacgauuuauaaucagu

SEQ ID NO: 56 - RNA, 270 nt, Artificial Sequence
  Artificial circular RNA 3; target hybridization region: SARS-CoV-2 Target D
ugcaguuagauaaagccgugagcggcuuguggaagauagaugaaaggauuggaaaagaccguaagcagcuuguagaagaugggugacaaguaauggaaaaauuguaaguggucugcggaagguggguggauaaggaacuugagaucguagguaguuuguagaggguaggugggacuauggggaugaacugugggcggucugcaggagguggacgagagagguacauaagaccguaaguaguuugugggggaugggcgauguauugcacac

SEQ ID NO: 57 - RNA, 270 nt, Artificial Sequence
  Artificial circular RNA 4; target hybridization region: SARS-CoV-2 Target D
ugcaguuagauaaagccgugagcggcuuguggaagauagaugaaaggauuggaaaagaccguaagcagcuuguagaagaugggugacaaguaauggaaaaauuguaaguggucugcggaagguggguggauaaggaacuugagaucguagguaguuuguagaggguaggugggacuauggggaugaacugugggcggucugcaggagguggacgagagagguacauaaagccguaaguagcuuguaggggauagaugguguauugcacac

SEQ ID NO: 58 - RNA, 27 nt, Artificial Sequence
  SARS-CoV-2 target hybridization region - comprised in 5' UTR SLII
aaccaacuuucgaucucuuguagaucu

SEQ ID NO: 59 - RNA, 33 nt, Artificial Sequence
  SARS-CoV-2 target hybridization region - Target A
uuuaaguuuagaauagacggugacaugguacca

SEQ ID NO: 60 - RNA, 30 nt, Artificial Sequence
  SARS-CoV-2 target hybridization region - Target C
ucacuaagaaaucugcugcugaggcuucua

SEQ ID NO: 61 - RNA, 31 nt, Artificial Sequence
  SARS-CoV-2 target hybridization region - Target D
ucgucuaucuucugcaggcugcuuacgguuu

SEQ ID NO: 62 - RNA, 35 nt, Artificial Sequence
  SARS-CoV-2 target hybridization region - comprised in 3UTR Replication Site
cagaaugaauucucguaacuacauagcacaaguag

SEQ ID NO: 63 - RNA, 289 nt, Artificial Sequence
  Artificial circular RNA against WNV and DENV (circWD1-in vitro)
ugcaguuagauaugcaguuagauaauauugggagguguguuuuuugccuuuuuccguagauccucgcgaaacuucuuggccugguuuucuuuucaaaagaguccuuacguauauuggaggguguguuuuucgcuuuuuuccgucucggaaacgauaacuuccuggcuugauuuuuucuucaaaagcugcguuaucuauauugagaggcguguuuuucgucuuuuuccguuccaaccuggagaacuuuuuggccugauuuucucuucaaaaccaggccguaccgacaguc

SEQ ID NO: 64 - RNA, 307 nt, Artificial Sequence
  Artificial circular RNA against HCV (circHCV-in vitro)
ugcaguuagauacccccuggggcucugaugaggaaccucucugggguccccacagcgagucuccuuggggccccuagaaugaaguucuuugggguuucauagcacgguucuccugggguuuucauacgauugucucuuggggucuugcguguuuacccuuuugggguccugcuaagggggcuuucuggggccuuucgaauaagucuuuuuggggccucguuuuaucacucuucugggguucccagccuuucccuucuuggggcuccuccgaccaugccccuugggguucuacccuguauggacaguc

SEQ ID NO: 65 - DNA, 289 bp, Artificial Sequence
  Artificial circular RNA against SARS-CoV-2
cccggttccgcactacttgtgctgtgtagttacgaggattcgttctgaccgtgtcttcgctatttgtgctatgtagttatgggagtttattctgctgctcgtggacctacttgtgttatgtagttgtgggagttcgttctggggcagttcgctctatttgtgctgtgtggttgcgggggttcattttgatcccgataagcctacttgtgttgtgtagttatgagggtttgttttgaccatacgacagctacttgtgctatgtggttgtgaggattcattctggacagtc

SEQ ID NO: 66 - DNA, 286 bp, Artificial Sequence
  Artificial circular RNA against SARS-CoV-2
cccggttccgcaagatttgtaggaggtcgaaagttggtttacaacgctactggggtctacaggaggttgaaggttggttgtcataatgcagtggatctacagggggtcgagagttggttgccgccattcacagggtctgcagggggtcggaagttggttagcagatgtttgaaggtctacgagagattgggggttggttaggctcggaagaaagatttatagggggttggaggttggttccagtcagtttacggatctacaaggggttgggagttggttgacagtc

SEQ ID NO: 67 - DNA, 286 bp, Artificial Sequence
  Artificial circular RNA against SARS-CoV-2
cccggttccgcaagatttgtaggaggtcgaaagttggtttacaacgctactggggtctacaggaggttgaaggttggttgtcataatgcagtggatctacagggggtcgagagttggttgccgccattcacagggtctgcgagaggtcggaagttggttagcagatgtttgaaggtctacgagagattgggggttggttaggctcggaagaaagatttatagggggttggaggttggttccagtcagtttacggatctacaaggggttgggagttggttgacagtc

SEQ ID NO: 68 - DNA, 287 bp, Artificial Sequence
  Artificial circular RNA against SARS-CoV-2
cccggttccgcatggtattgtgttaccgtctattttaagcttaaggcccccaaactagttggtgccgtgttgccgtctgttctaagcttaagctcgatcccgtgtttggtgctatgtcaccgtctattctagacttaggccgtcactatcatttggtgttgtgttattgtctattttggacttgaacatgattacacaactggtatcgtgttgtcgtctgttttagacttaggtcgcttttctttcctggtaccatgtcgccgtctgttttgggcttgaggacagtc

SEQ ID NO: 69 - DNA, 287 bp, Artificial Sequence
  Artificial circular RNA against SARS-CoV-2
cccggttccgcatggtattatgttgccgtctattctggacttaaacattccgtcatagctggtgtcgtgttatcgtctattctaaacttgaggtcacagtcgaacctggtgctatgtcgttgtctgttctgggtttaagctactttttcatgatggtactgtgttactgtctgttctagacttgggcgagcagagatctttggtactatgtcaccgtctattttaagcttgggattaaagcacctggtggtaccgtgtcgctgtctattttgggcttagagacagtc

SEQ ID NO: 70 - DNA, 287 bp, Artificial Sequence
  Artificial circular RNA against SARS-CoV-2
cccggttccgcatggtattatgttattgtttgttctgggcttaaagaagtaccattaggtggtatcgtgttgttgtttattctagatttaaagacctgggggcatctggtactgtgttgtcgtctattttagacttaaagtgggcgatcgagttggtaccatgtcatcgtttattttaggcttaaacacagattcgcctctggtactatgtcgccgtttattttgggtttaaatatgcgtggagcaatggtatcatgttaccgtctattttgaacttaaagacagtc

SEQ ID NO: 71 - DNA, 289 bp, Artificial Sequence
  Artificial circular RNA SARS-CoV-2
cccggttccgcataggggccttggcagtaggttttttagtggagtagccatttaggggtttcagtggtagatttcttagtggaaccatattgtaggagtctcggtagtggatttcttggtgaatcgcttggctggaagccttagtagcaggttttttggtggcagctaagcatagaggcctcggtagtagattttttagtgatcgtagaggataggggctttggcagcagattttttggtgggagattgccttaggagcctcggcagcaggtttcttggtgagacagtc

SEQ ID NO: 72 - DNA, 289 bp, Artificial Sequence
  Artificial circular RNA SARS-CoV-
cccggttccgcataggagccttagcggtaggtttcttagtgaactattattttagaagctttagtagcggattttttggtggtttttataactggaggtttcagtggcaggttttttagtggaggagtgagctggaagtctcggtagcaggttttttggtgatggcattgactggaagtcttggcggtagatttcttggtgagtctcaccgatggaagtttcagcggcgggtttcttggtggtgaatgaacttaggggcttcagcggcggatttcttagtgggacagtc

SEQ ID NO: 73 - DNA, 24 bp, Artificial Sequence
  probe 2019-nCoV_N1-P
accccgcattacgtttggtggacc

SEQ ID NO: 74 - DNA, 20 bp, Artificial Sequence
  primer 2019-nCoV_N1-F
gaccccaaaatcagcgaaat

SEQ ID NO: 75 - DNA, 24 bp, Artificial Sequence
  primer 2019-nCoV_N1-R
tctggttactgccagttgaatctg

SEQ ID NO: 76 - RNA, 97 nt, Artificial Sequence
  Target 1: IRES 1
cuccgccaugaaucacuccccugugaggaacuacugucuucacgcagaaagcgccuagccauggcguuaguaugagugucguacagccuccaggccc

SEQ ID NO: 77 - RNA, 33 nt, Artificial Sequence
  Target 2: IRES 2 (Domain IV)
ucucguagaccgugcaccaugagcacaaauccu

SEQ ID NO: 78 - RNA, 52 nt, Artificial Sequence
  Target 3: CDS1 (cHP)
ccaaaagaaacaccaaccgucgcccagaagacguuaaguucccgggcggcgg

SEQ ID NO: 79 - RNA, 81 nt, Artificial Sequence
  Target 4: CDS2 (SL427)
gaucguuggcggaguauacuuguugccgcgcaggggccccagguugggugugcgcacgacaaggaaaacuucggagcgguc

SEQ ID NO: 80 - RNA, 70 nt, Artificial Sequence
  Target 1: 5UTR
auggcugcgugagacacacguagccuaccaguuucuuacugcucuacucugcaaagcaagagauuaauaa

SEQ ID NO: 81 - RNA, 41 nt, Artificial Sequence
  Target 2: Repetitive Sequence Element:
agcaaauaaucuauagaucaaagggcuacgcaaccccugaa

SEQ ID NO: 82 - RNA, 69 nt, Artificial Sequence
  Target 3: Recoding Element:
ugucugagacucuuaccaugcugcuguaaaacguuggcuuuuuuagccguaaugagcgucggugcccac

SEQ ID NO: 83 - RNA, 67 nt, Artificial Sequence
  Target 1: SLII:
uuuugaggagaaagucaggccgggaaguucccgccaccggaaguugaguagacggugcugccugcga

SEQ ID NO: 84 - RNA, 48 nt, Artificial Sequence
  Target 1: Replication site Fully conserved in pangolin and RatG13
cagaaugaauucucguaacuacauagcacaaguagauguaguuaacuu

SEQ ID NO: 85 - RNA, 56 nt, Artificial Sequence
  Target 3: Target A
cuuuaaguuuagaauagacggugacaugguaccacauauaucacgucaacgucuua

SEQ ID NO: 86 - RNA, 46 nt, Artificial Sequence
  Target 4: Target C
ucacuaagaaaucugcugcugaggcuucuaagaagcccucggcaaa

SEQ ID NO: 87 - RNA, 49 nt, Artificial Sequence
  Target 5: Target D
ucgucuaucuucugcaggcugcuuacgguuucguccguguugcagcgau

SEQ ID NO: 88 - DNA, 289 bp, Artificial Sequence
  Artificial circular RNA against SARS-CoV-2
cccggttccgcactacttgtgttatgtagttacgaggattcgttttgctcttttctcccttatttgtgttgtgtagttatgggagtttgttctggcgtggtacgtcttacttgtgttgtgtggttatgagagttcattctgtatcaccactagctgcttgtgctgtgtagttatgaggatttattctggaataccgttccctgtttgtgctgtgtggttatgggagttcgttctgtgaggtgttccactgcttgtgctatgtggttacgagaatttgttttggacagtc

SEQ ID NO: 89 - DNA, 289 bp, Artificial Sequence
  Artificial circular RNA against SARS-CoV-2
cccggttccgcactacttgtgctgtgtagttacgaggattcgttctgaccgtgtcttcgctatttgtgctatgtagttatgggagtttattctgcaaccagtggacctacttgtgttatgtagttgtgggagttcgttctggggcagttcgctctatttgtgctgtgtggttgcgggggttcattttgatcccgataagcctacttgtgttgtgtagttatgagggtttgttttgaccatacgacagctacttgtgctatgtggttgtgaggattcattctggacagtc

SEQ ID NO: 90 - RNA, 24 nt, Artificial Sequence
  Fig. 28
aauagaguccugcccauuggcggg

SEQ ID NO: 91 - RNA, 10 nt, Artificial Sequence
  target hybridization region of Fig. 28
gaguccugcc

SEQ ID NO: 92 - RNA, 35 nt, Artificial Sequence
  #RNAseqcon
  misc_feature (1)..(10): /note="n= A, C, G, U or T"
  misc_feature (11)..(11): /note="n=&, sequence separator"
nnnnnnnnnnnaauagaguccugcccauuggcggg

SEQ ID NO: 93 - RNA, 35 nt, Artificial Sequence
  1st sample set of the 10 solutions returned by RNA inverse folding tool
  misc_feature (11)..(11): /note="n = &"
ggcggggcucnaauagaguccugcccauuggcggg

SEQ ID NO: 94 - RNA, 35 nt, Artificial Sequence
  2nd sample set of the 10 solutions returned by RNA inverse folding tool
  misc_feature (11)..(11): /note="n = &"
gguggggcucnaauagaguccugcccauuggcggg

SEQ ID NO: 95 - RNA, 35 nt, Artificial Sequence
  3rd sample set of the 10 solutions returned by RNA inverse folding tool
  misc_feature (11)..(11): /note="n=&, sequence separator"
ggcagggcucnaauagaguccugcccauuggcggg

SEQ ID NO: 96 - RNA, 35 nt, Artificial Sequence
  4th sample set of the 10 solutions returned by RNA inverse folding tool
  misc_feature (11)..(11): /note="n=&, sequence separator"
gguagggcucnaauagaguccugcccauuggcggg

SEQ ID NO: 97 - RNA, 35 nt, Artificial Sequence
  5th sample set of the 10 solutions returned by RNA inverse folding tool
  misc_feature (11)..(11): /note="n=&, sequence separator"
ggugggacucnaauagaguccugcccauuggcggg

SEQ ID NO: 98 - RNA, 35 nt, Artificial Sequence
  6th sample set of the 10 solutions returned by RNA inverse folding tool
  misc_feature (11)..(11): /note="n=&, sequence separator"
ggcgggacucnaauagaguccugcccauuggcggg

SEQ ID NO: 99 - RNA, 35 nt, Artificial Sequence
  7th sample set of the 10 solutions returned by RNA inverse folding tool
  misc_feature (11)..(11): /note="n=&, sequence separator"
ggcaggacucnaauagaguccugcccauuggcggg

SEQ ID NO: 100 - RNA, 35 nt, Artificial Sequence
  8th sample set of the 10 solutions returned by RNA inverse folding tool
  misc_feature (11)..(11): /note="n=&, sequence separator"
gguaggacucnaauagaguccugcccauuggcggg

SEQ ID NO: 101 - RNA, 35 nt, Artificial Sequence
  9th sample set of the 10 solutions returned by RNA inverse folding tool
  misc_feature (11)..(11): /note="n=&, sequence separator"
ggcgggauucnaauagaguccugcccauuggcggg

SEQ ID NO: 102 - RNA, 35 nt, Artificial Sequence
  10th sample set of the 10 solutions returned by RNA inverse folding tool
  misc_feature (11)..(11): /note="n=&, sequence separator"
ggugggauucnaauagaguccugcccauuggcggg

SEQ ID NO: 103 - RNA, 81 nt, Artificial Sequence
  Example 14 HCV RNAseqcon sequence
  misc_feature (1)..(28): /note="n= U, T, G, C, or U"
  misc_feature (29)..(29): /note="n=&, sequence separator"
nnnnnnnnnnnnnnnnnnnnnnnnnnnnnccaaaagaaacaccaaccgucgcccagaagacguuaaguucccgggcggcgg

SEQ ID NO: 104 - RNA, 81 nt, Artificial Sequence
  Example 14 HCV output sequence 1/10
  misc_feature (29)..(29): /note="n=&, sequence separator"
ucugggcgacgguugguguuucuuuuggnccaaaagaaacaccaaccgucgcccagaagacguuaaguucccgggcggcgg

SEQ ID NO: 105 - RNA, 81 nt, Artificial Sequence
  Example 14 HCV output sequence 2/10
  misc_feature (29)..(29): /note="n=&, sequence separator"
uuugggcgacgguugguguuucuuuuggnccaaaagaaacaccaaccgucgcccagaagacguuaaguucccgggcggcgg

SEQ ID NO: 106 - RNA, 81 nt, Artificial Sequence
  Example 14 HCV output sequence 3/10
  misc_feature (29)..(29): /note="n=&, sequence separator"
ucugggugacgguugguguuucuuuuggnccaaaagaaacaccaaccgucgcccagaagacguuaaguucccgggcggcgg

SEQ ID NO: 107 - RNA, 81 nt, Artificial Sequence
  Example 14 HCV output sequence 4/10
  misc_feature (29)..(29): /note="n=&, sequence separator"
uuugggugacgguugguguuucuuuuggnccaaaagaaacaccaaccgucgcccagaagacguuaaguucccgggcggcgg

SEQ ID NO: 108 - RNA, 81 nt, Artificial Sequence
  Example 14 HCV output sequence 5/10
  misc_feature (29)..(29): /note="n=&, sequence separator"
ucugggcggcgguugguguuucuuuuggnccaaaagaaacaccaaccgucgcccagaagacguuaaguucccgggcggcgg

SEQ ID NO: 109 - RNA, 81 nt, Artificial Sequence
  Example 14 HCV output sequence 6/10
  misc_feature (29)..(29): /note="n=&, sequence separator"
uuugggcggcgguugguguuucuuuuggnccaaaagaaacaccaaccgucgcccagaagacguuaaguucccgggcggcgg

SEQ ID NO: 110 - RNA, 81 nt, Artificial Sequence
  Example 14 HCV output sequence 7/10
  misc_feature (29)..(29): /note="n=&, sequence separator"
ucuggguggcgguugguguuucuuuuggnccaaaagaaacaccaaccgucgcccagaagacguuaaguucccgggcggcgg

SEQ ID NO: 111 - RNA, 81 nt, Artificial Sequence
  Example 14 HCV output sequence 8/10
  misc_feature (29)..(29): /note="n=&, sequence separator"
uuuggguggcgguugguguuucuuuuggnccaaaagaaacaccaaccgucgcccagaagacguuaaguucccgggcggcgg

SEQ ID NO: 112 - RNA, 81 nt, Artificial Sequence
  Example 14 HCV output sequence 9/10
  misc_feature (29)..(29): /note="n=&, sequence separator"
ucugggcgaugguugguguuucuuuuggnccaaaagaaacaccaaccgucgcccagaagacguuaaguucccgggcggcgg

SEQ ID NO: 113 - RNA, 81 nt, Artificial Sequence
  Example 14 HCV output sequence 10/10
  misc_feature (29)..(29): /note="n=&, sequence separator"
uuugggcgaugguugguguuucuuuuggnccaaaagaaacaccaaccgucgcccagaagacguuaaguucccgggcggcgg

SEQ ID NO: 114 - RNA, 75 nt, Artificial Sequence
  EXAMPLE 14 CHIKV target disruption structure RSE #RNAseqcon
  misc_feature (1)..(33): /note="n=A, G, T, C, or U"
  misc_feature (34)..(34): /note="n=&, sequence separator"
nnnnnnnnnnnnnnnnnnnnnnnnnnnnnnnnnnagcaaauaaucuauagaucaaagggcuacgcaaccccugaa

SEQ ID NO: 115 - RNA, 75 nt, Artificial Sequence
  Example 14 CHICKV output sequence 1/10
  misc_feature (34)..(34): /note="n=&, sequence separator"
uugcguggcccuuuggucuguagguuguuugcunagcaaauaaucuauagaucaaagggcuacgcaaccccugaa

SEQ ID NO: 116 - RNA, 75 nt, Artificial Sequence
  Example 14 CHICKV output sequence 2/10
  misc_feature (34)..(34): /note="n=&, sequence separator"
uuguguggcccuuuggucuguagguuguuugcunagcaaauaaucuauagaucaaagggcuacgcaaccccugaa

SEQ ID NO: 117 - RNA, 75 nt, Artificial Sequence
  Example 14 CHICKV output sequence 3/10
  misc_feature (34)..(34): /note="n=&, sequence separator"
uugcguagcccuuuggucuguagguuguuugcunagcaaauaaucuauagaucaaagggcuacgcaaccccugaa

SEQ ID NO: 118 - RNA, 75 nt, Artificial Sequence
  Example 14 CHICKV output sequence 4/10
  misc_feature (34)..(34): /note="n=&, sequence separator"
uuguguagcccuuuggucuguagguuguuugcunagcaaauaaucuauagaucaaagggcuacgcaaccccugaa

SEQ ID NO: 119 - RNA, 75 nt, Artificial Sequence
  Example 14 CHICKV output sequence 5/10
  misc_feature (34)..(34): /note="n=&, sequence separator"
uugcgugguccuuuggucuguagguuguuugcunagcaaauaaucuauagaucaaagggcuacgcaaccccugaa

SEQ ID NO: 120 - RNA, 75 nt, Artificial Sequence
  Example 14 CHICKV output sequence 6/10
  misc_feature (34)..(34): /note="n=&, sequence separator"
uugugugguccuuuggucuguagguuguuugcunagcaaauaaucuauagaucaaagggcuacgcaaccccugaa

SEQ ID NO: 121 - RNA, 75 nt, Artificial Sequence
  Example 14 CHICKV output sequence 7/10
  misc_feature (34)..(34): /note="n=&, sequence separator"
uugcguaguccuuuggucuguagguuguuugcunagcaaauaaucuauagaucaaagggcuacgcaaccccugaa

SEQ ID NO: 122 - RNA, 75 nt, Artificial Sequence
  Example 14 CHICKV output sequence 8/10
  misc_feature (34)..(34): /note="n=&, sequence separator"
uuguguaguccuuuggucuguagguuguuugcunagcaaauaaucuauagaucaaagggcuacgcaaccccugaa

SEQ ID NO: 123 - RNA, 75 nt, Artificial Sequence
  Example 14 CHICKV output sequence 9/10
  misc_feature (34)..(34): /note="n=&, sequence separator"
uugcguagcucuuuggucuguagguuguuugcunagcaaauaaucuauagaucaaagggcuacgcaaccccugaa

SEQ ID NO: 124 - RNA, 75 nt, Artificial Sequence
  Example 14 CHICKV output sequence 10/10
  misc_feature (34)..(34): /note="n=&, sequence separator"
uuguguagcucuuuggucuguagguuguuugcunagcaaauaaucuauagaucaaagggcuacgcaaccccugaa

SEQ ID NO: 125 - RNA, 97 nt, Artificial Sequence
  Example 14 WNV target disruption structure SLII #RNAseqcon
  misc_feature (1)..(29): /note="n=A, G, C, T, or U"
  misc_feature (30)..(30): /note="n=&, sequence separator"
nnnnnnnnnnnnnnnnnnnnnnnnnnnnnnuuuugaggagaaagucaggccgggaaguucccgccaccggaaguugaguagacggugcugccugcga

SEQ ID NO: 126 - RNA, 97 nt, Artificial Sequence
  Example 14 WNV output sequence 1/10
  misc_feature (30)..(30): /note="n=30"
gauuuucuggccuggcuuuuuccucaaganuuuugaggagaaagucaggccgggaaguucccgccaccggaaguugaguagacggugcugccugcga

SEQ ID NO: 127 - RNA, 97 nt, Artificial Sequence
  Example 14 WNV output sequence 2/10
  misc_feature (30)..(30): /note="n=&, sequence separator"
aauuuucuggccuggcuuuuuccucaaganuuuugaggagaaagucaggccgggaaguucccgccaccggaaguugaguagacggugcugccugcga

SEQ ID NO: 128 - RNA, 97 nt, Artificial Sequence
  Example 14 WNV output sequence 3/10
  misc_feature (30)..(30): /note="n=&, sequence separator"
gauuuuuuggccuggcuuuuuccucaaganuuuugaggagaaagucaggccgggaaguucccgccaccggaaguugaguagacggugcugccugcga

SEQ ID NO: 129 - RNA, 97 nt, Artificial Sequence
  Example 14 WNV output sequence 4/10
  misc_feature (30)..(30): /note="n=&, sequence separator"
aauuuuuuggccuggcuuuuuccucaaganuuuugaggagaaagucaggccgggaaguucccgccaccggaaguugaguagacggugcugccugcga

SEQ ID NO: 130 - RNA, 97 nt, Artificial Sequence
  Example 14 WNV output sequence 5/10
  misc_feature (30)..(30): /note="n=&, sequence separator"
gauuuucuggcuuggcuuuuuccucaaganuuuugaggagaaagucaggccgggaaguucccgccaccggaaguugaguagacggugcugccugcga

SEQ ID NO: 131 - RNA, 97 nt, Artificial Sequence
  Example 14 WNV output sequence 6/10
  misc_feature (30)..(30): /note="n=&, sequence separator"
aauuuucuggcuuggcuuuuuccucaaganuuuugaggagaaagucaggccgggaaguucccgccaccggaaguugaguagacggugcugccugcga

SEQ ID NO: 132 - RNA, 97 nt, Artificial Sequence
  Example 14 WNV output sequence 7/10
  misc_feature (30)..(30): /note="n=&, sequence separator"
gauuuuuuggcuuggcuuuuuccucaaganuuuugaggagaaagucaggccgggaaguucccgccaccggaaguugaguagacggugcugccugcga

SEQ ID NO: 133 - RNA, 97 nt, Artificial Sequence
  Example 14 WNV output sequence 8/10
  misc_feature (30)..(30): /note="n=&, sequence separator"
aauuuuuuggcuuggcuuuuuccucaaganuuuugaggagaaagucaggccgggaaguucccgccaccggaaguugaguagacggugcugccugcga

SEQ ID NO: 134 - RNA, 97 nt, Artificial Sequence
  Example 14 WNV output sequence 9/10
  misc_feature (30)..(30): /note="n=&, sequence separator"
gacuuucuggccuggcuuuuuccucaaganuuuugaggagaaagucaggccgggaaguucccgccaccggaaguugaguagacggugcugccugcga

SEQ ID NO: 135 - RNA, 97 nt, Artificial Sequence
  Example 14 WNV output sequence 10/10
  misc_feature (30)..(30): /note="n=&, sequence separator"
aacuuucuggccuggcuuuuuccucaaganuuuugaggagaaagucaggccgggaaguucccgccaccggaaguugaguagacggugcugccugcga

SEQ ID NO: 136 - RNA, 84 nt, Artificial Sequence
  Example 14 SARS-CoV-2 target disruption structure Replication Site
  misc_feature (1)..(35): /note="n=A, G, C, T, or U"
  misc_feature (36)..(36): /note="n=&, sequence separator"
nnnnnnnnnnnnnnnnnnnnnnnnnnnnnnnnnnnncagaaugaauucucguaacuacauagcacaaguagauguaguuaacuu

SEQ ID NO: 137 - RNA, 84 nt, Artificial Sequence
  Example 14 SARS-CoV-2 output sequence 1/10
  misc_feature (36)..(36): /note="n=&, sequence separator"
cuacuugugcuauguaguuacgggaguucguucugncagaaugaauucucguaacuacauagcacaaguagauguaguuaacuu

SEQ ID NO: 138 - RNA, 84 nt, Artificial Sequence
  Example 14 SARS-CoV-2 output sequence 2/10
  misc_feature (36)..(36): /note="n=&, sequence separator"
uuacuugugcuauguaguuacgggaguucguucugncagaaugaauucucguaacuacauagcacaaguagauguaguuaacuu

SEQ ID NO: 139 - RNA, 84 nt, Artificial Sequence
  Example 14 SARS-CoV-2 output sequence 3/10
  misc_feature (36)..(36): /note="n=&, sequence separator"
cugcuugugcuauguaguuacgggaguucguucugncagaaugaauucucguaacuacauagcacaaguagauguaguuaacuu

SEQ ID NO: 140 - RNA, 84 nt, Artificial Sequence
  Example 14 SARS-CoV-2 output sequence 4/10
  misc_feature (36)..(36): /note="n=&, sequence separator"
uugcuugugcuauguaguuacgggaguucguucugncagaaugaauucucguaacuacauagcacaaguagauguaguuaacuu

SEQ ID NO: 141 - RNA, 84 nt, Artificial Sequence
  Example 14 SARS-CoV-2 output sequence 5/10
  misc_feature (36)..(36): /note="n=&, sequence separator"
cuauuugugcuauguaguuacgggaguucguucugncagaaugaauucucguaacuacauagcacaaguagauguaguuaacuu

SEQ ID NO: 142 - RNA, 84 nt, Artificial Sequence
  Example 14 SARS-CoV-2 output sequence 6/10
  misc_feature (36)..(36): /note="n=&, sequence separator"
uuauuugugcuauguaguuacgggaguucguucugncagaaugaauucucguaacuacauagcacaaguagauguaguuaacuu

SEQ ID NO: 143 - RNA, 84 nt, Artificial Sequence
  Example 14 SARS-CoV-2 output sequence 7/10
  misc_feature (36)..(36): /note="n=&, sequence separator"
cuguuugugcuauguaguuacgggaguucguucugncagaaugaauucucguaacuacauagcacaaguagauguaguuaacuu

SEQ ID NO: 144 - RNA, 84 nt, Artificial Sequence
  Example 14 SARS-CoV-2 output sequence 8/10
  misc_feature (36)..(36): /note="n=&, sequence separator"
uuguuugugcuauguaguuacgggaguucguucugncagaaugaauucucguaacuacauagcacaaguagauguaguuaacuu

SEQ ID NO: 145 - RNA, 84 nt, Artificial Sequence
  Example 14 SARS-CoV-2 output sequence 9/10
  misc_feature (36)..(36): /note="n=&, sequence separator"
cugcuuguguuauguaguuacgggaguucguucugncagaaugaauucucguaacuacauagcacaaguagauguaguuaacuu

SEQ ID NO: 146 - RNA, 84 nt, Artificial Sequence
  Example 14 SARS-CoV-2 output sequence 10/10
  misc_feature (36)..(36): /note="n=&, sequence separator"
uugcuuguguuauguaguuacgggaguucguucugncagaaugaauucucguaacuacauagcacaaguagauguaguuaacuu

SEQ ID NO: 147 - RNA, 41 nt, Artificial Sequence; identical to SEQ ID NO: 18
  FIGURE 4

SEQ ID NO: 148 - RNA, 20 nt, Artificial Sequence
  Figure 4 hybridization region from circRNA
cugcgcgcuugcauggcguu

SEQ ID NO: 149 - RNA, 39 nt, Artificial Sequence
  Fig. 22 cHP
caggagggcccggcaaaagccgggcugucaauaugcuaa

SEQ ID NO: 150 - RNA, 112 nt, Artificial Sequence
  Fig. 22 ShP
aacacagcauauugacaccugggauagacuaggagaucuucugcucugcacaaccagccacacggcacagugcgccgacaaugguggcugguggugcgagaacacaggaucu

SEQ ID NO: 151 - RNA, 91 nt, Artificial Sequence
  Fig. 22 SLII
uuuuagugaggaagucaggccggaaaauucccgccaccggaaguugaguagacggugcugccugcgacucaaccccaggaggacuggguga

SEQ ID NO: 152 - RNA, 24 nt, Artificial Sequence
  Fig. 28 target disruption structure
aauagaguccugcccauuggcggg

SEQ ID NO: 153 - RNA, 18 nt, Artificial Sequence
  Fig. 28 CircRNA
auaucgcaggacuccaau

(19) United States
(12) Patent Application Publication
(10) Pub. No.: US 2023/0323354 A1
(43) Pub. Date: Oct. 12, 2023

(54) ARTIFICIAL RNAS FOR MODULATING RNA FRAGMENTS

(71) Applicant: UNIVERSITAT POMPEU FABRA, Barcelona (ES)

(72) Inventors: Juana María DÍEZ ANTÓN, Barcelona (ES); Ivan Javier DOTU RODRÍGUEZ, Barcelona (ES); Marc TALLÓ PARRA, Barcelona (ES)

(21) Appl. No.: 18/012,867

(22) PCT Filed: Jun. 28, 2021
(86) PCT No.: PCT/EP2021/067756

§ 371 Date: Dec. 23, 2022

(30) Foreign Application Priority Data
Jun. 26, 2020 (EP) 20382569.0

(51) Int. Cl. C12N 15/113

(52) U.S. Cl. CPC C12N 15/1131; C12N 2310/532

(57) ABSTRACT

The present invention relates to an artificial RNA having at least one hybridization region against one or more target disruption structures of one or more RNA fragments, wherein such an artificial RNA suitable for disrupting by hybridization one or more target disruption structures of one or more RNA fragments, thereby modulating the functionality of the one or more RNA fragments.

### FIELD OF THE INVENTION

The present invention relates to the field of biotechnology. In particular, the present invention relates to artificial RNAs as defined in the present invention. More particularly, the present invention relates to artificial RNAs suitable for disrupting by hybridization one or more target disruption structures of one or more RNA fragments, thereby modulating the one or more RNA fragments.

### BACKGROUND OF THE INVENTION

A vast amount of research and development is underway to extend the range of medicines in general, such as antivirals to treat pathogens currently without commercial treatments or to treat cancer. Also, with the tendency of viruses and tumoral cells to mutate and become drug resistant, the need of the hour is to develop and refresh the pipeline of antitumorals and/or antivirals with new therapies. The opportunities available for this market, according to a recent market report (https://www.mordorintelligence.com/industry-reports/global-antiviral-drugs-market-industry), are (i) an increasing need for broad-spectrum antiviral drugs and (ii) access to antiviral drugs in the pharmerging markets.

Current approved and effective antiviral treatments include molecules that specifically interact with viral proteins essential for the viral life cycle. Developing these therapeutic molecules is costly and highly time consuming. Moreover, every molecule functions solely for a specific virus. Multiple efforts have been made to develop RNA-based antiviral treatments to target the viral RNA genome [Brice A Sullenger and Smita Nair. From the RNA world to the clinic. Science 352 (6292), 1417-1420]. These include the design of 19 nucleotide-long micro RNAs (miRNAs) that specifically interact with viral RNA genomes, leading to their subsequent degradation by the cellular RISC system. Although successful in cell culture, a major drawback for their clinical use is the rapid emergence and selection of mutations in the RNA genome that inhibit the interaction with the miRNAs. Another drawback of miRNAs as antiviral molecules is their stability, as they are naturally degraded by the cellular decay machinery.

Because of the cost and time required to develop new therapeutic agents against viral proteins, RNA-based therapeutics has generated a lot of attention in the last years. RNA-based therapies have been able to address targets untreatable with antibody and small-molecule approaches [Ling-Ling Chen. The biogenesis and emerging roles of circular RNAs. Nature Reviews Molecular Cell Biology 17, 205-211 (2016), doi:10.1038/nrm.2015.32]. Consequently, multiple companies have been created to develop RNA-based therapies to treat multiple diseases. For instance, the company Moderna is developing an mRNA-based vaccine against infection caused by SARS-CoV-2 (Covid-19). These companies focus on the use of antisense, siRNAs, aptamers and microRNA mimics/anti-microRNAs. However, these molecules (i) are rapidly degraded and (ii) share with protein- and antibodies-based therapies the emergence of drug resistance by the high tendency of viruses to mutate.

The present inventors have particularly focused their approach on stable artificial RNAs, such as circular RNAs. Circular RNAs (circRNAs) are back-splicing products of precursor mRNAs that appear naturally in the cell (see review [Ling-Ling Chen. The biogenesis and emerging roles of circular RNAs. Nature Reviews Molecular Cell Biology 17, 205-211 (2016), doi:10.1038/nrm.2015.32]). Previously thought to be irrelevant byproducts, are now proven to perform several functions, such as miRNA sponges and RBP (RNA binding proteins) sponges. Circular RNA has no ends. This is very important, since most mRNA degradation pathways in the cell require 5′or 3′ ends for the cellular exonucleases to carry out their degradation function. Consequently, circRNAs are extremely stable molecules. The potential of circRNAs as novel therapeutic platforms have not been fully exploited.

WO2017/222911 discloses the use of circular RNAs generated with exogenous introns to stimulate immune response or circular RNAs generated with endogenous introns to prevent immune recognition of foreign RNA.

WO0061595 discloses a covalently-closed multiple antisense (CMAS)-oligo, which is constructed to form a closed type by ligation using complementary primer, and a ribbon-type antisense (RiAS)-oligo, which is composed of two loops containing multiple antisense sequences and a stem connecting the two loops that is constructed to by ligation using complementary sequences at both 5 prime ends.

WO 2013/162350 A2 relates to the use of circular RNAs composed of 2 purine rich domains that can target viral pyrimidine rich regions in order to form triple helices that can block viral replication.

CN 108165549 A relates to the use of circular RNAs as micro-RNA sponges. It presents an artificial formulation of the well-known function of endogenous circRNAs, where multiple partial complementarity regions target mature micro-RNAs that activate AGO2 pathway upon hybridization.

In addition, there is an increasing need for better understanding molecular mechanisms underlying many pathologies, such as cancer, viral infections, autoimmune diseases, neurological diseases, genetic disorders, etc. In most of these pathologies, RNAs play a role in the underlying mechanisms. Hence, there is a need for tools which would allow the study of the specific functions of the RNAs involved in the molecular mechanisms involving pathologies.

The present invention addresses the above problems, and is directed to tools which allow the modulation of the functionality of RNA fragments, such as, e.g., viral genome, which allow multiple applications. For instance, with the tools of the present invention (artificial RNAs) it is possible to study the structure-function relationship of a certain RNA fragment, or certain regions within a certain RNA fragment. In addition, with the RNAs of the present invention, it is possible to prevent and/or treat diseases where RNA fragments are involved, such as viral infections, cancer, immune diseases, genetic disorders, etc. For instance, with the artificial RNA of the present invention it is possible to study RBPs (ribosome binding proteins) that bind to double stranded RNA motifs.

### SUMMARY OF THE INVENTION

In a first aspect, the present invention provides an artificial circular RNA suitable for disrupting by hybridization one or more target disruption structures of one or more RNA fragments,
(a) wherein the artificial circular RNA comprises between 150 and 800 nucleotides, preferably between 200 and 600 nucleotides;
(b) wherein the artificial circular RNA comprises two or more hybridization regions which:
   (i) completely hybridize with at least one target hybridization region comprised in the one or more target disruption structures of the one or more RNA fragments; and
   (ii) have a total of between 7 and 100 nucleotides, preferably between 10 and 50 nucleotides;
(c) wherein the one or more target disruption structures:
   (i) comprises at least a hairpin loop preceded or followed by a region of unpaired nucleotides; and
   (ii) comprises at least one target hybridization region which comprises a single-stranded region of at least 2 nucleotides, preferably 3 nucleotides or more preceded or followed by a double-stranded region of at least 5 nucleotides, preferably 10 nucleotides or more, wherein the at least one target hybridization region completely hybridizes with each of the two or more hybridization regions of the artificial circular RNA; and
(d) wherein the two or more hybridization regions comprised in the artificial circular RNA are further characterized because, when hybridizing with the target hybridization region, the energy of hybridization, as measured by RNAcofold, between the hybridization region and the at least one target hybridization region is more negative than the energy of the target disruption region, thereby disrupting the target disruption structure.

Preferably, the artificial circular RNA according to the first aspect or any of its embodiments comprises between 6 and 20 hybridization regions. Preferably, the least two, and preferably all, of the hybridization regions are capable of completely hybridizing with the same target hybridization region. Preferably, the least two, and preferably all, of the hybridization regions have different nucleotide sequences.

Preferably, in the artificial circular RNA according to the first aspect or any of its embodiments, the 2 or more hybridization regions are:
a) separated by non-hybridization regions of sizes up to 20 nucleotides; or
b) are not separated by non-hybridization regions; or
c) are overlapping.

Preferably, in the artificial circular RNA according to the first aspect or any of its embodiments, the one or more RNA fragments is selected from mRNA, tRNA, rRNA, non-coding RNA and viral genomic RNA.

Preferably, in the artificial circular RNA according to the first aspect or any of its embodiments, the one or more RNA fragments is viral genomic RNA.

Preferably, in the artificial circular RNA according to the first aspect or any of its embodiments, the one or more RNA fragments is positive-sense single-stranded viral genomic RNA.

Preferably, in the artificial circular RNA according to the first aspect or any of its embodiments, the viral genomic RNA is selected from Influenza virus, HAV, Poliovirus, Coxsackie B virus, Coronavirus and Rhinovirus (common cold).

Preferably, in the artificial circular RNA according to the first aspect or any of its embodiments, the viral genomic RNA is selected from Hepatitis C virus, Dengue, Zika, Chikungunya, West Nile and Yellow Fever virus.

Preferably, in the artificial circular RNA according to the first aspect or any of its embodiments, the viral genomic RNA is from Severe acute respiratory syndrome coronavirus 2 (SARS-CoV-2)

Preferably, in the artificial circular RNA according to the first aspect or any of its embodiments, the at least one or more target disruption structures are selected from the group consisting of:
(a) Internal Ribosome Entry (IRES) Domain IV and Domain V, capsid-coding region hairpin element (cHP) or SL427 from Hepatitis C Virus,
(b) short Stem Loop (sHP) or capsid-coding region hairpin element (cHP) from Dengue virus,
(c) 5′ untranslated region (5′UTR), Repetitive Sequence Element (RSE) or Recoding Element from Chikungunya,
(d) Stem loop III (SLIII) from West Nile, and/or
(e) SL-2, Replication site, Target A, Target C, Target D from Coronavirus, preferably from Severe acute respiratory syndrome coronavirus 2 (SARS-CoV-2).

Preferably, in the artificial circular RNA according to the first aspect or any of its embodiments, the at least one or more target disruption structures comprises or consists of the target disruption structures selected from the list consisting of SEQ ID NOs.:76, 77, 78 and/or 79 from Hepatitis C virus, and wherein the target hybridization regions, for each of these target disruption structures, are respectively selected from SEQ ID NOs: 25, 26, 27 and 28.

Preferably, in the artificial circular RNA according to the first aspect or any of its embodiments, the at least one or more target disruption structures comprises or consists of the target disruption structures selected from the list consisting of SEQ ID NO.:29 and/or 30 from Dengue virus, and wherein the target hybridization regions, for each of these target disruption structures, are respectively selected from SEQ ID NOs.: 29 and 30.

Preferably, in the artificial circular RNA according to the first aspect or any of its embodiments, the at least one or more target disruption structures comprises or consists of the target disruption structures selected from the list consisting of SEQ ID NOs.: 80, 81 and/or 82 from Chikungunya, and wherein the target hybridization regions, for each of these target disruption structures, are respectively selected from SEQ ID NOs.: 33, 35, and 31.

Preferably, in the artificial circular RNA according to the first aspect or any of its embodiments, the at least one or more target disruption structures comprise or consist of the target disruption structure of SEQ ID NO.: 83 from West Nile and the target hybridization regions is SEQ ID NO.: 37.

Preferably, in the artificial circular RNA according to the first aspect or any of its embodiments, the at least one or more target disruption structures comprise or consists of the target disruption structures selected from the list consisting of SEQ ID NOs.: 84, 58, 85, 86, and/or 87 from Severe acute respiratory syndrome coronavirus 2 (SARS-CoV-2), and wherein the target hybridization regions, for each of these target disruption structures, are respectively selected from SEQ ID NOs.: 62, 58, 59, 60, and 61.

Preferably, in the artificial circular RNA according to the first aspect or any of its embodiments, the at least one or more target disruption structures comprise or consists of the target disruption structures selected from the list consisting of SEQ ID NOs.: 30 and/or 79 from Dengue Virus and Hepatitis C Virus, and wherein the target hybridization regions, for each of these target disruption structures, are respectively selected from SEQ ID NOs.: 30 and 28.

Preferably, in the artificial circular RNA according to the first aspect or any of its embodiments, the at least one or more target disruption structures comprise or consists of the target disruption structures selected from the list consisting of SEQ ID NOs SEQ ID NOs.: 30 and/or 83 from Dengue Virus and West Nile Virus, and wherein the target hybridization regions, for each of these target disruption structures, are respectively selected from SEQ ID NOs.: 30 and 37.

Preferably, the sequence of the artificial circular RNA according to the first aspect or any of its embodiments comprises, or preferably, consists of, the following nucleotides defined in: SEQ ID NO: 2, 3, 4, 5, 6 (for Hepatitis C virus); 8, 9, 10 (for Dengue virus); 12, 13, 14, 15, 39 (for Chikungunya virus); 16 and 17 (broad spectrum activity for both Hepatitis C virus and Dengue Virus); 24 and 19 (for West Nile Virus); 21, 22 and 23 (broad spectrum activity for both Dengue and West Nile Viruses); 32 (broad spectrum activity for both Hepatitis C virus and Dengue Virus); 36, 38, 40, 41, 42, 43, 44, 45, 46, 47, 48, 49, 50, 51, 52, 53, 54, 55, 56, 57, 65, 66, 67, 68, 69, 70, 71, 72 (for Severe acute respiratory syndrome coronavirus 2).

Preferably, the one or more target hybridization region of the artificial circular RNA which completely hybridize with the two or more hybridization regions is comprised in the artificial RNA defined in SEQ ID NO.: 1, SEQ ID NO.: 7, SEQ ID NO.: 11, SEQ ID NO.: 34 and/or SEQ ID NO.: 20.

In a second aspect, the present invention relates to a composition comprising the artificial RNA of the invention.

In a third aspect, the present invention relates to a kit comprising the artificial RNA and/or the composition of the present invention.

In a fourth aspect, the present invention relates to the artificial RNA and/or the composition of the present invention for use as a medicament, preferably for use in a method of preventing and/or treating a viral infection. Preferably, the viral infection is caused by Hepatitis C virus, Hepatitis A virus, Poliovirus, Influenza virus, Coxsackie B virus, rhinovirus (common cold), Dengue, Zika, Chikungunya, West Nile, Yellow Fever virus or coronavirus, such as SARS and/or MERS, preferably SARS-CoV-2.

In a sixth aspect, the present invention provides a method of screening for artificial circular RNA comprising two or more hybridization regions capable of disrupting by hybridization one or more target disruption structures of one or more RNA fragments, wherein the target disruption structures are defined as:
i. a first region with at least a hairpin loop preceded or followed by a second region of unpaired nucleotides; and
ii. as comprising at least one target hybridization region which comprises a single-stranded region of at least 2 nucleotides, preferably 3 nucleotides or more preceded or followed by a double-stranded region of at least 5 nucleotides, preferably 10 nucleotides or more, and wherein the method comprises the steps of:
   a) identifying the two or more hybridization regions of the artificial circular RNA as those regions that have a total of between 7 and 100 nucleotides in length, preferably between 10 and 50 nucleotides that, when hybridizing with the at least one target hybridization region, the energy of the hybridization between the two or more hybridization regions and the at least one target hybridization region is more negative than the energy of the target disruption structure, thereby disrupting the one or more target disruption structure; wherein the the two or more hybridization regions comprised in the artificial circular RNA, are identified by RNA inverse folding tools, such as NUPACK, RNAifold, or MoiRNAiFold;
   b) designing an artificial circular RNA comprising the two or more hybridization regions capable of disrupting the one or more target disruption structures as identified in step a), wherein said artificial circular RNA is between 150 and 800 nucleotides in length, preferably between 200 and 600 nucleotides; and
   c) optionally selecting the artificial circular RNA capable of disrupting by hybridization the one or more target disruption structures as designed in step b), and optionally packaging it into a product.

### BRIEF DESCRIPTION OF THE DRAWINGS

FIG. 1 represents schematically the intracellular production of antiviral circRNA. This figure describes how a circular RNA can be produced within the target cell. First, the figure shows a plasmid containing a CMV promoter, two complementary and repetitive regions surrounding the candidate circular RNA and flanking splicing acceptor and donor sites. This plasmid is transfected into the target cell. The cell then transcribes the linear RNA as shown, and the area between the splice donor and acceptor sites gets circularized through backsplicing.

FIG. 2. Synthesis and circularization of RNA. Workflow composed of four major steps: (A) production of T7 promoter-flanked templates by PCR. (B) RNA preparation by in vitro transcription. (C) Generation of specific termini for ligation. In vitro transcribed RNAs have to be dephosphorylated prior in vitro circularization. (D) Ligation with T4 RNA Ligase 1 or circRNA Ligase. Circular or linear oligomers can be formed as side products.

FIG. 3 represents schematically the mode of action.This figure shows the mode of action of a circular RNA as it hinders viral cycle. Viruses contain RNA structures in their genome that are vital for their life cycles. The circular RNA targets those structures by binding to them in a way that a conformational change occurs that hinders vital steps of the virus life cycle.

FIG. 4 represents schematically an example of a relevant mode of action. This figure shows an example of how hybridization of the circular RNA can alter viral cycle. First, RNA binding proteins (RBPs) are necessary for many steps in the viral life cycle. These RBPs generally bind specific regions of the viral genome, where both sequence and structure (or structural context) are necessary. Similar to the figure, an example is the binding of PTB in the last domain of the IRES element of many picornaviruses, which binds to a box of single stranded pyrimidines immediately 3′ of a stable haripin. As in the right drawing, when a circular RNA binds a certain structured region of the IRES and the single stranded region preceding this structured region, it changes the RNA structure in such a way that the box of pyrimidines is not in the same estructural context anymore, and therefore the RBP cannot bind to it.

FIG. 5 represents an example of a circRNA that targets 3 different regions of the viral genome (or 3 different viral genomes) with 2 hybridization sequences per target region. Regions with the same color represent that they target the same viral region.

FIG. 6 represents schematically the RNA secondary structure of the beginning and end of the HCV genome. Highlighted are the regions to which our circular RNAs are designed to target to.

FIG. 7 represents schematically the results of 4 of the designed RNAs against HCV genome. The ids of the circRNAs correspond to the target regions shown in the previous figure. As it can be seen, infection is lowered up to 20% with respect to the control.

FIG. 8 represents schematically the RNA secondary structure of the DENV genome. Highlighted are the regions to which our circular RNAs are designed to target to.

FIG. 9 represents schematically the results of 3 of the designed RNAs against DENV genome. The ids of the circRNAs correspond to the target regions shown in the previous figure. As it can be seen, infection is lowered up to 40% with respect to the control.

FIG. 10 represents the results of the 4 designed RNAs against CHIKV genome. As it can be seen, infection is lowered up to 50% with respect to the control.

FIG. 11 represents the results of a broadspectrum circRNA designed against both DENV and HCV genomes when used to treat DENV infection. HCV_CDS2 is one of the circRNAs in example 1 and used here as negative control. DENV1_chp is one of the circRNAs in example 2 and used here as positive control.

FIG. 12 represents the results of a broadspectrum circRNA designed against both DENV and HCV genomes when used to treat HCV infection. HCV_CDS2 is one of the circRNAs in example 1 and used here as positive control. DENV1_chp is one of the circRNAs in example 2 and used here as negative control.

FIG. 13 represents the results of a second broadspectrum circRNA designed against both DENV and HCV genomes when used to treat DENV infection. HCV_CDS2 is one of the circRNAs in example 1 and used here as negative control. DENV1_chp is one of the circRNAs in example 2 and used here as positive control.

FIG. 14 represents the results of a second broadspectrum circRNA designed against both DENV and HCV genomes when used to treat HCV infection. HCV_CDS2 is one of the circRNAs in example 1 and used here as positive control. DENV1_chp is one of the circRNAs in example 2 and used here as negative control.

FIG. 15 represents the results of the two designed RNAs against West Nile virus (WNV) genome, circ wnv_slll 1 and circ wnv_slll 2. As it can be seen, infection is lowered with respect to the control.

FIG. 16 represents the results of three broadspectrum circRNAs designed against both WNV and DENV (dchp_wsll_A, dchp_wsll_B and dchp_wsll_C) when used to treat WNV infection. Positive and negative controls from examples 2 and 5 were used. As it can be seen, all circRNA showed inhibition.

FIG. 17 represents the results of three broadspectrum circRNAs designed against both WNV and DENV (dchp_wsll_A, dchp_wsll_B and dchp_wsll_C) when used to treat DENV infection. Positive and negative controls from examples 2 and 5 were used. As it can be seen, all circRNA showed inhibition.

FIG. 18 represents the capacity of the designed RNA against HCV in inhibiting chronically infected cells with HCV. CircRNAs inhibit infectivity in HCV chronically infected cells. Huh7/Scr cells were infected with HCV and 48hpi transfected with circ_hcv_cds2. Two days later luciferase values were measured. Effects on infectivity are determined by changes in luciferase expression levels.

FIG. 19 represents the result of the designed RNA against a region required for HCV RNA replication and its ability to inhibit HCV replication. Circ_hcv_cds2, which targets a region required for HCV RNA replication, inhibits HCV RNA replication. Circ_hcv_cds2 inhibited luciferase levels 48 hours post infection when HCV RNA is translated and replicated but not at 4 hours post infection when HCV RNA is solely translated. Effects on infectivity are determined by changes in luciferase expression levels. Statistical significance was calculated using a T-test (*represents p-value < 0.05).

FIG. 20 represents the result of the designed RNA against a region required for DENV RNA replication and its ability to inhibit DENV replication. Circ_dv_3utr and circ_dv_cHP_v1, designed to target structures within the DENV RNA genome directing RNA replication, inhibit DENV RNA replication. The circ_dv_3utr and circ_dv_cHP_v1 inhibit luciferase expression levels at 48 hours when the RNA genome is translated and replicated but not at 8 hours when is solely translated. All Results were obtained from at least three biological replicates. Statistical significance was calculated using a T-test (*represents p-value < 0.05).

FIG. 21. Artificial circRNA structure and mode of action. 1) CircRNAs contain several different hybridization (brown regions H) and separation sequences (light grey regions S). Hybridization sequences target the viral RNA genome (dark grey regions) and separation sequences allow for structural flexibility and physical separation among the hybridization ones. Note that all H and S sequences are different among themselves. 2) Hybridization regions are designed to target and disrupt a particular viral RNA genome structure, leading to a decrease in infectivity. The hybridization starts in a single stranded region, an external or hairpin loop or a pseudoknot and finish within a helix, so that the helix is disrupted.

FIG. 22. WNV structures used for the design of circWNV. Representation of the 5′-UTR (above) and 3′-UTR (below) from WNV genome. In red the hybridization regions used for the design of the circWNV. UTR: untranslated region [Adapted from Fernandez-Sanlés et al., 2017, Front. Microbiol. 8, 1-16].

FIG. 23. Schematic CHIKV structures used for the design of circCHIKV. Representation of the predicted 5′-UTR (A), RSE (B) and Recording Element (C). In A and B all the structure is targeted by the circRNA in C, the hybridization region is depicted in red. UTR: untranslated region; RSE: repetitive sequence elements. [Adapted from Kendra et al., 2018, Virology 339, 200-212; and from Khan et al., 2002, J. Gen. Virol. 83, 3075-3084].

FIG. 24. Designed broad-spectrum circRNAs impair both DENV and HCV infectivity. Cells were transfected with the circRNA dv_chp_v1, the circRNA hcv_cds2 or the broad-spectrum circRNA (circ dchp_hcv_cds2_2) containing hybridization sequences from circRNA dv_chp_v1 and circRNA hcv_cds2. Next, cells were infected either with DENV or HCV harbouring the luciferase reporter gene and 48h later the infectivity was measured. All results were obtained from at least three biological replicates. Effects on infectivity are determined by changes in luciferase expression levels. Statistical significance was calculated using a T-test (*represents p-value < 0.05).

FIG. 25. Depicts the protocol to obtain circular RNAs in vitro.

FIG. 26. Shows the results of both a circular RNA against DNV and WNV and a circular RNA against HCV generated in vitro.

FIG. 27. Shows types of target disruption structures (inner line) and target hybridization regions (outer line).

FIG. 28. Exemplifies the relationship between target disruption structure, target hybridization region and hybridization region, and it shows the mechanism of disruption by hybridization.

FIG. 29. Comparison of cleavage efficiency of different Hammerhead ribozymes.

FIG. 30. Gel with different magnesium conditions.

FIG. 31. Gel showing different IVT times.

FIG. 32. Gel showing the RNAse R effect without gel purification.

FIG. 33. Shows the efficacy results of the circRNAs designed against SARS-Cov2.

FIG. 34. Shows the efficacy of an in vitro generated circRNA against SARS-Cov2.

### DETAILED DESCRIPTION OF THE INVENTION

### Definitions

In describing the present disclosure, the following terms will be used and are defined as indicated below.

The forms “a”, “an” and “the” include plural referents unless the context states otherwise.

The term “about” when referred to a given amount or quantity is meant to include deviations of plus or minus five percent.

The term “artificial circular RNAs” or “circular RNAs” or “circRNAs” is used herein to refer to non-coding RNAs forming covalently closed continuous loops which have the 3′ and 5′ ends joint together, thus they are missing the 5′-cap and in consequence the polyadenylated tail. Therefore, they are resistant to exonuclease-mediated degradation and to debranching enzymes, which confers them higher stability with respect to other RNAs.

The term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% (or total) complementary refers to the situation in which each nucleotide unit of one polynucleotide strand or region can hydrogen bond with each nucleotide unit of a second polynucleotide strand or region. Less than perfect (or partial) complementarity refers to the situation in which some, but not all, nucleotide units of two strands or two regions can hydrogen bond with each other and can be expressed as a percentage.

The term “hybridization” is used to refer to the structure formed by 2 independent strands of RNA that form a double stranded structure via base pairings from one strand to the other. These base pairs are considered to be G-C, A-U and G-U. (A - Adenine, C -Cytosin, G-Guanine, U - Uracil). As in the case of the complementarity, the hybridization can be total or partial.

The term “transfection or “transfect” is used to refer to the uptake of circular RNA by a cell. A cell has been “transfected” when the circular RNA has been introduced inside the cell membrane.

“Stem-loop intramolecular base pairing” (“stem-loops”) is a pattern that can occur in single-stranded DNA or, more commonly, in RNA. The structure is also known as a “hairpin” or “hairpin loop”. It occurs when two regions of the same strand, usually complementary in nucleotide sequence when read in opposite directions, base-pair to form a double helix that ends in an unpaired loop. The resulting structure is a key building block of many RNA secondary structures. The base-pairing of the helix need not be complete, there could be stretches of single stranded nucleotides, which are called “bulges” or “internal loops”. As an important secondary structure of RNA, it can direct RNA folding, protect structural stability for messenger RNA (mRNA), provide recognition sites for RNA binding proteins, and serve as a substrate for enzymatic reactions.

“Internal-loops” (also termed “interior loops”), in RNA, are found where the double stranded RNA separates due to no Watson-Crick base pairing between the nucleotides. Internal-loops can be classified as either symmetrical or asymmetrical, with some asymmetrical internal-loops, also known as bulges. Internal-loops differ from stem-loops as they occur in the middle of a stretch of double stranded RNA.

“External-loops” are stretches of single-stranded nucleotides that separate hairpin loops or multi-loops.

“Multi-loops” are branching off of a double-stranded region into several hairpin loops.

In the context of the present invention a “target disruption structure” is a stem loop (or hairpin loop), preferably preceded or followed by a stretch of unpaired nucleotides, (external loop, bulge or internal loop or multiloop, see FIG. 27).

In the context of the present invention, a “target hybridization region” is a region within a target disruption structure composed of a single stranded region preceded or followed by a double-stranded region. The target hybridization region is thus a region that, upon hybridization of another RNA strand, yields the disruption of the structure to which it belongs to.

“Disruption by hybridization”: given a target disruption structure, a target hybridization region and an artificial RNA that completely hybridizes with the target hybridization region, disruption by hybridization occurs when the energy of the hybridization is more favourable than the energy of the target disruption structure (more negative). Upon such hybridization, the structure of the target disruption structure changes, at least, completely for the overlap between the target disruption structure and the target hybridization region. Such energy calculations can be obtained in silico using well established software like RNAcofold (Lorenz, Ronny and Bernhart, Stephan H. and Höner zu Siederdissen, Christian and Tafer, Hakim and Flamm, Christoph and Stadler, Peter F. and Hofacker, Ivo L., ViennaRNA Package 2.0, Algorithms for Molecular Biology, 6:1 26, 2011, doi:10.1186/1748-7188-6-26; Reuter, J. S., & Mathews, D. H. (2010). RNAstructure: software for RNA secondary structure prediction and analysis. BMC Bioinformatics. 11,129; Mathews, D. H., et al., “Predicting oligonucleotide affinity to nucleic acid targets”, RNA, 1999 5: 1458-1469), RNAstructure (https://rna.urmc.rochester.edu/RNAstructure.html), Mfold (http://unafold.rna.albany.edu/?q=mfold, M. Zuker, “Mfold web server for nucleic acid folding and hybridization prediction”, Nucleic Acids Res. 31 (13), 3406-3415, 2003) or Vienna package (http://rna.tbi.univie.ac.at/; Lorenz, Ronny and Bernhart, Stephan H. and Höner zu Siederdissen, Christian and Tafer, Hakim and Flamm, Christoph and Stadler, Peter F. and Hofacker, Ivo L., ViennaRNA Package 2.0, Algorithms for Molecular Biology, 6:1 26, 2011, doi: 10.1186/1748-7188-6-26).

“Viral conserved structure” refers to viral genomic RNA structures which are conserved among members of the same species, genus, family, etc. Those structures are sometimes characterized in the literature and have been experimentally validated. In the absence of such experimental proof, there exists software that can predict such structures in silico. For instance, all viral genomes of the same family can be aligned, using for example ClustalW, and then using RNAz (RNAz 2.0: Improved noncoding RNA detection, Gruber AR, Findeiβ S, Washietl S, Hofacker IL, Stadler PF. Pac Symp Biocomput. 15:69-79, 2010; Nucleic Acids Res. 1994 Nov 11; 22(22): 4673-4680, doi: 10.1093/nar/22.22.4673, PMCID: PMC308517, PMID: 7984417; CLUSTAL W: Improving the sensitivity of progressive multiple sequence alignment through sequence weighting, position-specific gap penalties and weight matrix choice, J D Thompson, D G Higgins, and T J Gibson). RNAz predicts structured regions that are more thermodynamically stable than expected by comparison to random sequences of the same length and sequence composition (z-score), and additionally assesses regions by the support of compensatory and consistent mutations in the sequence alignment. Such conserved structures are believed to be so due to its functional relevance in the life-cycle of the virus. Within example 14, we show how some these viral conserved structures have been found.

The term 5′UTR refers to the region upstream of the coding region, immediately before the start codon.

The term “IRES” is defined as an internal ribosome entry site and is an RNA element that allows for translation initiation in a cap-independent manner, as part of the greater process of protein synthesis. In eukaryotic translation, initiation typically occurs at the 5′ end of mRNA molecules, since 5′ cap recognition is required for the assembly of the initiation complex. The location for IRES elements is often in the 5′UTR, but can also occur elsewhere in mRNAs.

The term CDS refers to the coding region of a messenger RNA or a viral genome. This is the region that codes for the corresponding protein.

The term 3′UTR refers to the region of the viral genome downstream of the CDS, immediately after the stop codon.

The term “cHP” means “capsid-coding region hairpin element” which is a known region of several flavivirus genomes found within the CDS.

The term “RSE” means “conserved repeated sequence element” and it is a known structure found in the 3′UTR of several alphavirus genomes.

“SRVVLC”, structured region vital for the viral life cycle, are structured regions of the RNA viral genome which are vital in the viral replication, encapsidation and/or translation, i.e., if disrupted, the virus is less capable of performing essential functions of its life cycle and thus, the virus is less infective.

“Administering” an artificial RNA to a cell comprises transducing, transfecting, electroporating, translocating, fusing, phagocytosing, shooting or ballistic methods, etc., i.e., any means by which a nucleic acid can be transported across a cell membrane.

“Homology region” refers to regions in different virus strains/serotypes which share common structural and/or functional characteristics. Homologous structures do not imply sequence identity as a necessary condition. The skilled person is able to identify homologous regions in other viral strains/serotypes as the ones defined in the present application by conventional means.

The degree of identity between two sequences can be determined by conventional methods, for example, by means of standard sequence alignment algorithms known in the state of the art, such as, for example BLASTn (Altschul S.F. et al. Basic local alignment search tool. J Mol Biol. 1990 Oct 5; 215(3):403-10).

During the description of the claims, the word “comprising”, and its variants does not intend to exclude other technical characteristics, additives, components or steps. In addition, the term “comprising” may also encompass the term “consisting of”.

### Description of the Invention

In general, RNA fragments such as mRNA, tRNA, rRNA, non-coding RNA, viral RNA genomes, viral mRNAs, etc., contain highly structured regions (regions with secondary structure) which are either essential for their function or which, while not being essential, may play a more or less important role in the functionality of said RNA fragment. These highly structured regions comprise hairpin loops or at least portions of hairpin loops preceded or followed by a region of unpaired nucleotides. If the structure (such as the secondary structure) of these hairpin loops or portions of hairpin loops is altered, conformational changes within the RNA fragment take place. This would lead to changes in the functionality of the RNA fragment.

Biological RNA is single stranded and often forms complex and intricate base-pairing interactions due to its increased ability to form hydrogen bonds stemming from the extra hydroxyl group in the ribose sugar. The secondary structure of RNA consists of a single polynucleotide which is folded within the same molecule. Base pairing in RNA occurs when RNA folds between complementarity regions. Both single- and double-stranded regions are often found in RNA molecules. The antiparallel strands form a helical shape. The four basic elements in the secondary structure of RNA are helices, loops, bulges, and junctions. Stem-loop or hairpin loop is the most common element of RNA secondary structure. Stem-loop is formed when the RNA chains fold back on themselves to form a double helical tract called the stem, the unpaired nucleotides form a single stranded region called the loop.

The secondary structure of RNA can be predicted either computationally or with experimental methods. Computationally, RNA secondary structure can be predicted from one or several nucleic acid sequences using tools publically available to the skilled person, e.g., Vienna package, as described above.

As outlined above, RNA structure such as RNA secondary structure is important in many biological processes, including translation regulation in messenger RNA, replication of single-stranded RNA viruses, and the function of structural RNAs and RNA/protein complexes. In fact, for many RNA molecules, the secondary structure is highly important to the correct function of the RNA — often more so than the actual sequence.

Hence, changes in the secondary structure of the RNA fragments will inevitably lead to changes in the functionality of the RNA fragments.

The artificial RNAs of the present invention, preferably circRNAs, are able to change the secondary structure of the RNA fragments they target. Accordingly, with the artificial RNAs of the present invention it is possible to modulate the functionality of the target RNA fragments. For instance, within viral genomes, there are highly structured regions which are vital for the viral life cycle (SRVVLC). If these regions are altered in their conformation or secondary structure (e.g., disrupted), the virus would be less capable (and, preferably incapable) of performing essential functions of its life cycle. Accordingly, altering the conformation or secondary structure of these regions vital for the viral life cycle would lead the virus less infective, ideally totally ineffective. The same principle can be applied to other RNA fragments. For instance, a change in the conformation of a tRNA molecule can lead to a decrease (or increase) in the efficiency of translation of that specific tRNA. For instance, a change in the secondary structure of an mRNA molecule can lead to a decrease (or to an increase) in the binding affinity of that mRNA molecule and a certain protein or even the ribosome and thus affect its translation rate. Changes in the structure of an IRES element, both viral and cellular, can affect translation initiation. Changes in the structure of the UTRs of an mRNA can obscure miRNA binding sites, thus affecting translation regulation of such mRNA. Changes in the structure of intronic regions can affect splicing, etc.

In particular, the artificial RNAs of the present invention, preferably circRNAs, are able to hybridize with specific regions within the target RNA fragment. In doing so, the artificial RNAs of the present invention, preferably circRNAs, are able to disrupt these specific target regions within the target RNA fragment. These “specific regions within the target RNA fragment” are the so-called “target disruption structures”.

The target disruption structures comprise at least a hairpin loop preceded or followed by a region of unpaired nucleotides, and also comprise the so-called “target hybridization region(s)”. The target hybridization regions are regions within the target disruption structures which comprise a single-stranded region of at least 2 nucleotides, preceded or followed by a double-stranded region.

The artificial RNAs of the present invention, preferably circRNAs, hybridize with the target hybridization regions through the so-called “hybridization regions”. In other words, the artificial RNAs of the present invention, preferably circRNAs, comprise at least one hybridization region, which is a region of the artificial RNA which is able to completely hybridize with one or more target hybridization regions comprised in the target disruption regions of the target RNA fragment.

Hybridization (or hybridisation) is a phenomenon in which two RNA molecules anneal to each other via base pairing interactions. In the context of this invention only canonical base pairings are considered (C-G,A-U and G-U). By “complete hybridization” it is understood that all of the nucleotides of the hybridization region of the artificial RNA anneal with all of the nucleotides of the target hybridization region.

When the hybridization region completely hybridizes with the target hybridization region, the energy of the hybridization between the hybridization region and the target hybridization region is more negative than the energy of the target disruption region. When this happens, the target disruption structure is disrupted, i.e., the secondary structure of the target RNA fragment is altered. Hence, the functionality of the target RNA fragment is also altered: the functionality of the RNA fragment has been modulated by the artificial RNA of the present invention. This can be reproduced, and thus candidates for hybridization region that completely hybridize with the target hybridization region can be easily found, by using RNA design tools such as NUPACK or RNAiFold once the target hybridization region is known as explained below.

In a first aspect, the present invention relates to artificial RNAs, preferably circRNAs, suitable for disrupting by hybridization one or more target disruption structures of one or more RNA fragments.

By “disrupting by hybridization one or more target disruption structures of one or more RNA fragments” it is understood, in the context of the present invention, that the secondary structure of the one or more target disruption structures of one or more RNA fragments is altered when a hybridization region of the artificial RNA of the present invention completely hybridizes with the corresponding target hybridization region of the target disruption structure.

The artificial RNA of the invention, preferably circRNA, can be of any length as long as it comprises at least one hybridization region as described in the present invention and is suitable for disrupting by hybridization one or more target disruption structures of one or more RNA fragments. Preferably, the RNA of the present invention, preferably circRNA, comprises between 100 and 1000 nucleotides, more preferably between 150 and 800 nucleotides, and even more preferably between 200 and 600 nucleotides.

As described above, the artificial RNA of the present invention, which is preferably a circular RNA, comprises at least one hybridization region. The at least one hybridization region is a region of the artificial RNA (i.e., a sequence of nucleotides) which completely hybridizes with at least one target hybridization region comprised in the one or more target disruption structures of the one or more RNA fragments. The number of nucleotides of the hybridization region is not limited as long as it is able to completely hybridize with at least one target hybridization region, although preferably it has the same length as the target hybridization region, comprised in the one or more target disruption structures and, in doing so, disrupts (i.e., alters the secondary structure) of the one or more target disruption structures.

In a preferred embodiment, the at least one hybridization region has a total of between 7 and 100 nucleotides, more preferably between 10 and 50 nucleotides, even more preferably between 15 and 35 nucleotides, such as between 15 and 25 nucleotides.

As described above, the hybridization region completely hybridizes with at least one target hybridization region comprised in the one or more target disruption structures of the one or more RNA fragments. The target disruption structures are regions of the RNA fragments which comprise at least a portion of a hairpin loop preceded or followed by a region of unpaired nucleotides. The target disruption structures comprise at least one target hybridization region. The target hybridization regions are regions (i.e., a sequence of nucleotides) which comprises a single-stranded region of at least 2 nucleotides preceded or followed by a double-stranded region. The length of the target hybridization region is not limited as long as it comprises a single-stranded region of at least 2 nucleotides preceded or followed by a double-stranded region and is able to completely hybridize with the at least one hybridization region of the artificial RNA. Preferably, the single-stranded region of the target hybridization region comprises 3 nucleotides or more, such as 3, 4, 5, 10, 15 nucleotides or more. Preferably the double-stranded region of the target hybridization region comprises 5 nucleotides or more, such as 5, 7, 10, 15, 20, 25 nucleotides or more.

Importantly, the hybridization region of the artificial RNA of the present invention, which is preferably a circular RNA, completely hybridizes with at least one target hybridization region comprised in the one or more target disruption structures of the one or more RNA fragments. Hence, the at least one hybridization region of the artificial RNA of the present invention has exaclty the same number of nucleotides than the at least one target hybridization region with which it hybridizes. Consequently, the at least one hybridization region of the artificial RNA of the present invention has a first region of a certain number of nucleotides which completely hybridizes with the single-stranded region of the target hybridization region, and a second region of a certain number of nucleotides which completely hybridizes with the double-stranded region of the target hybridization region.

In other words, in a first step, the single-stranded region of the target hybridization region anneals with certain nucleotides of the hybridization region of the artificial RNA, which is preferably a circular RNA. In a second step, the double-stranded region of the target hybridization region, which precedes or follows the single-stranded region, is disrupted, and new interactions are formed between one strand of the double-stranded region of the target hybridization region and certain nucleotides of the hybridization region of the artificial RNA, which anneal to each other. The disruption of the double-stranded region of the target hybridization region occurs when the energy of the hybridization between the at least one hybridization region and the at least one target hybridization region is more negative (more favourable) than the energy of the target disruption region. The energy of hybridization and the capacity of disrupting the one or more target disruption structures can be measured, for example, with RNAcofold; the identification of potential candidates of the hybridization regions of the artificial RNA of the present invention characterized by having an energy of the hybridization between the at least one hybridization region and the at least one target hybridization region more negative (more favourable) than the energy of the target disruption region, can be easily identified by RNA inverse folding tools, such as NUPACK, RNAifold, or MoiRNAiFold as illustrated in example 4.

As a result, at least the double-stranded region of the target hybridization region is disrupted and, hence, the target disruption structure is also disrupted. Hence, the structure of the target disruption structure changes, at least, completely for the overlap between the target disruption structure and the target hybridization region. Therefore, the secondary structure of the target RNA fragment is changed and, thus, its functionality is also altered.

The hybridization between the hybridization region of the artificial RNA and the target hybridization region of the target disruption structures of the RNA fragment can be followed in vitro or in vivo. The change in secondary structure of the RNA fragment can also be checked employing techniques well-known in the art such as SHAPE (Poulsen, Line Dahl et al. “SHAPE Selection (SHAPES) enrich for RNA structure signal in SHAPE sequencing-based probing data.” RNA (New York, N.Y.) vol. 21,5 (2015): 1042-52. doi:10.1261/rna.047068.114) or PARIS (Lu Z, Gong J, Zhang QC. PARIS: Psoralen Analysis of RNA Interactions and Structures with High Throughput and Resolution. Methods Mol Biol. 2018;1649:59-84. doi: 10.1007/978-1-4939-7213-5_4).

Consequently, the at least one hybridization region comprised in the artificial RNA of the present invention is further characterized because, when hybridizing with the target hybridization region, the energy of the hybridization between the at least one hybridization region and the at least one target hybridization region is more negative than the energy of the target disruption region, thereby disrupting the target disruption structure. As discussed above, the skilled person is able to predict the energy of the hybridization between the at least one hybridization region and the at least one target hybridization region and the energy of the target disruption region. Available tools for this prediction are, e.g., well established software like RNAcofold (Lorenz, Ronny and Bernhart, Stephan H. and Höner zu Siederdissen, Christian and Tafer, Hakim and Flamm, Christoph and Stadler, Peter F. and Hofacker, Ivo L., ViennaRNA Package 2.0, Algorithms for Molecular Biology, 6:1 26, 2011, doi: 10.1186/1748-7188-6-26; Reuter, J. S., & Mathews, D. H. (2010). RNAstructure: software for RNA secondary structure prediction and analysis. BMC Bioinformatics. 11,129, Mathews, D. H. et al., “Predicting oligonucleotide affinity to nucleic acid targets”, RNA, 1999 5: 1458-1469), RNAstructure (https://rna.urmc.rochester.edu/RNAstructure.html), Mfold (http://unafold.rna.albany.edu/?q=mfold, M. Zuker, “Mfold web server for nucleic acid folding and hybridization prediction”, Nucleic Acids Res. 31 (13), 3406-3415, 2003) or Vienna package (http://rna.tbi.univie.ac.at/), as described in detail above.

Preferably, the energy of the hybridization between the at least one hybridization region and the at least one target hybridization region and the energy of the target disruption region is preferably calculated with RNAcofold software, with the settings as described in Mathews, D. H., et al., “Predicting oligonucleotide affinity to nucleic acid targets”, RNA, 1999 5: 1458-1469. It is however noted that such calculation shall be unnecessary when RNA inverse folding tools, such as NUPACK, RNAifold, or MoiRNAiFold, are used for the identification of potential candidates of the hybridization regions of the artificial RNA of the present invention characterized by having an energy of the hybridization between the at least one hybridization region and the at least one target hybridization region more negative (more favourable) than the energy of the target disruption region.

For a given RNA fragment, the skilled person is able to identify potential target disruption structures, since these are regions of RNA which comprise at least a portion of a hairpin loop preceded or followed by a region of unpaired nucleotides. There are many RNA structure prediction software available to the skilled person and that predict the secondary structure of a given RNA sequence. For instance, we refer to Mathews, D. H., et al. “RNA secondary structure prediction.” Current protocols in nucleic acid chemistry vol. Chapter 11 (2007): Unit 11.2. doi:10.1002/0471142700.nc1102s28.

In addition, the skilled person is able to identify one or more target hybridization regions within the target disruption structure, because the target hybridization region is comprised in the target disruption structure and comprises a single-stranded region of at least 2 nucleotides preceded or followed by a double-stranded region, as defined above.

Once a target hybridization region is identified, the skilled person, as indicated above, is able to design one or more hybridization regions which would completely hybridize with the target hybridization region. Similarly as above, the skilled person is aware of several software for the design of RNA sequences which would completely hybridize with a given RNA sequence, e.g., NUPACK (http://www.nupack.org/design/new, see also B. R. Wolfe, N. J. Porubsky, J. N. Zadeh, R. M. Dirks, and N. A. Pierce, “Constrained multistate sequence design for nucleic acid reaction pathway engineering”, JAm Chem Soc, 139:3134-3144, 2017; B. R. Wolfe and N. A. Pierce, “Sequence design for a test tube of interacting nucleic acid strands”, ACS Synth Biol, 4:1086-1100, 2015; J. N. Zadeh, B. R. Wolfe, and N. A. Pierce, “Nucleic acid sequence design via efficient ensemble defect optimization” J Comput Chem, 32:439-452, 2011; and R. M. Dirks, M. Lin, E. Winfree, and N. A. Pierce, “Paradigms for computational nucleic acid design” Nucl Acids Res, 32:1392-1403, 2004.) or RNAiFold (https://bioinformatics.bc.edu/clotelab/RNAiFold/, see also Juan Antonio Garcia-Martin, Peter Clote, Ivan Dotu, “RNAiFold: A constraint programming algorithm for RNA inverse folding and molecular design, J Bioinform Comput Biol 11(2): 1350001, 2013; and Garcia-Martin JA, Dotu I, Clote P., “RNAiFold 2.0 A web server and software to design custom and Rfam-based RNA molecules”, Nucleic Acids Research Web Server issue, 2015, doi: 10.1093/nar/gkv460, “RNAiFold 2.0 A web server and software to design custom and Rfam-based RNA molecules”). Moreover, it is possible to ascertain whether a certain target disruption structure can be disrupted by hybridization given a particular target hybridization region. This can be achieved using several RNA inverse folding or RNA design tools publicly available, such as NUPACK (http://www.nupack.org/design/new), RNAiFold (http://bioinformatics.bc.edu/clotelab/RNAiFold/) or its recent extension MoiRNAiFold (https://moiraibiodesign.com/design/).

For instance, in RNAiFold (and MoiRNAiFold), given any specific target disruption structure, such as the one shown in FIG. 28 (AAUAGAGUCCUGCCCAUUGGCGGG) and its target hybridization region (GAGUCCUGCC), one can use the following input file:

#RNAscdstr((((((((((&....))))))))))..........#RNAs
eqconNNNNNNNNNN&AAUAGAGUCCUGCCCAUUGGCGGG

Wherein the input of ((((((((((&....)))))))))).......... corresponds to the well established dot bracket notation (see, e.g. RNAlib-2.4.18: RNA Structure Notations (univie.ac.at), and wherein the input NNNNNNN&AAUAGAGUCCUGCCCAUUGGCGGG indicates that every “N” is a nucleotide (any nucleotide A,C,G or U) corresponding with “(” above and that has to hybridize with the corresponding “)” indicated above. That is, the RNAiFold (and MoiRNAiFold) should return a solution (or multiple ones) of potential hybridization regions that can indeed disrupt the target disruption structure AAUAGAGUCCUGCCCAUUGGCGGG upon complete hybridization to the target hybridization region _{GAGUCCUGCC} .

In this case, a sample set of 10 solutions return by the RNA inverse folding tool was the following:

GGCGGGGCUC&AAUAGAGUCCUGCCCAUUGGCGGGGGUGGGGCUC&AAUA
GAGUCCUGCCCAUUGGCGGGGGCAGGGCUC&AAUAGAGUCCUGCCCAUUG
GCGGGGGUAGGGCUC&AAUAGAGUCCUGCCCAUUGGCGGGGGUGGGACUC
&AAUAGAGUCCUGCCCAUUGGCGGGGGCGGGACUC&AAUAGAGUCCUGCC
CAUUGGCGGGGGCAGGACUC&AAUAGAGUCCUGCCCAUUGGCGGGGGUAG GACUC&AAUAGAGUCCUGCCCAUUGGCGGGGGCGGGAUUC&AAUAGAGUC
CUGCCCAUUGGCGGGGGUGGGAUUC&AAUAGAGUCCUGCCCAUUGGCGGG

from which it follows first, that the target disruption structure can be disrupted via hybridization and second, that the hybridization regions (in bold) can be used to construct a circRNA (by separating these regions with random polynucleotide regions) that can disrupt by hybridization the target disruption structure.

Thus provided these input instructions, if the software tool returns a solution (or multiple ones) it means that the target disruption structure can indeed be disrupted by hybridization. From the set of solutions it follows the generation of the circRNA of this invention: by taking any number of these solutions (hybridization regions) and separating them by random nucleotides regions. Example 14 shows additional input files for several of the target disruption structures used as examples through this application.

In the context of this invention, hybridization region is a region within the artificial circular RNA capable of hybridizing with the target hybridization region in the RNA fragment of interest and disrupting by hybridization the target disruption structure (see FIG. 28 for a depiction of how all these concepts relate to each other). In a preferred embodiment, the energy of hybridization and the capacity of disrupting the one or more target disruption structures can be calculated with RNA inverse folding tools, such as NUPACK, RNAifold, MoiRNAiFold.

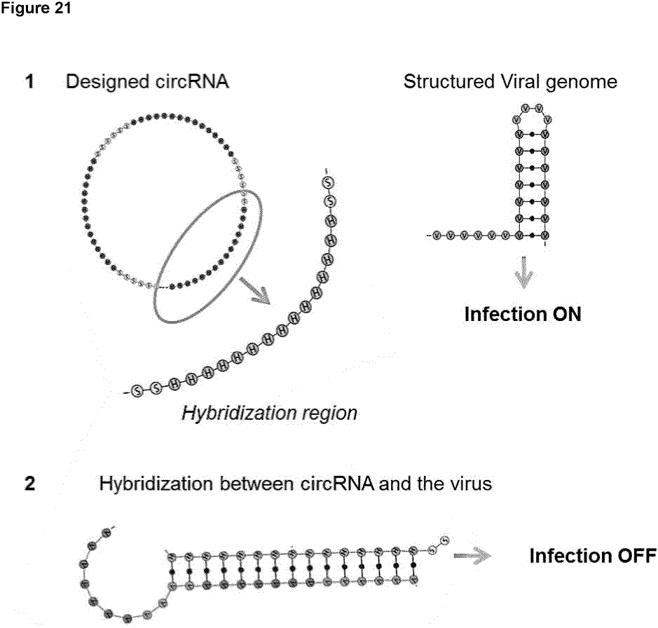

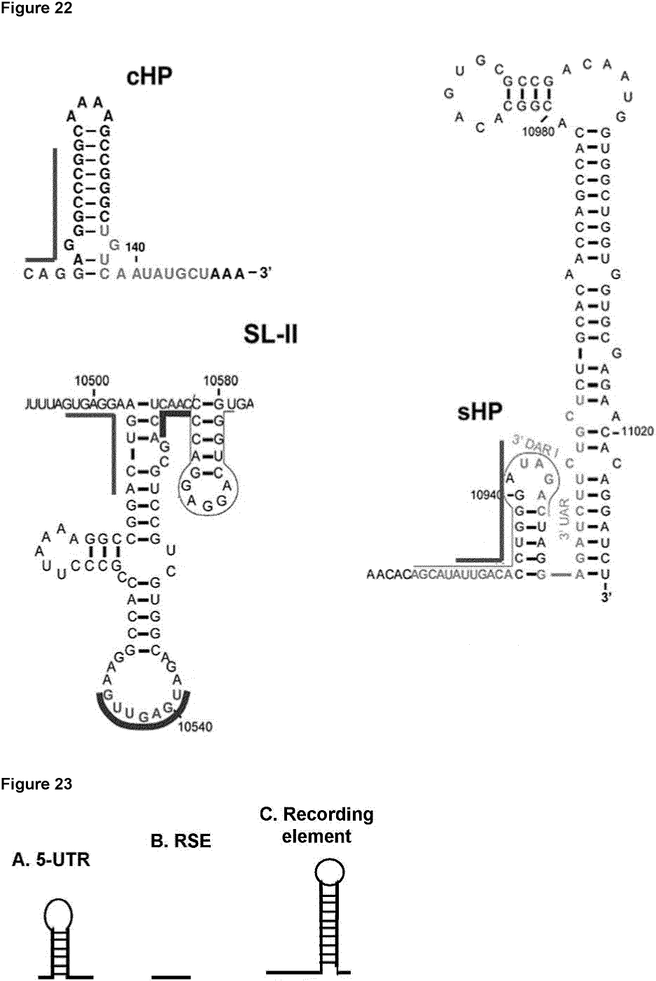

As indicated above, in a much preferred embodiment, the artificial RNA of the invention is a circular RNA. In this sense, the present inventors have found that the use of artificial RNA-based therapies as disclosed herein, preferably circular RNAs, has fundamental advantages over other RNA molecules:
Stability. On one hand, the circRNAs are extremely stable because of their unavailability to the cellular exonucleases. This stability would simplify their use in therapy, in contrast to the other current RNA-based therapies that require chemical modifications that are costly, unnatural, and could lead to toxicity concerns.
Resistance to the emergence and selection of escaping mutants. The designed artificial RNAs, such as circRNAs, preferably contain (i) longer sequences whose hybridization to target hybridization regions will not be affected by single mutations and (ii) multiple hybridization regions capable of completely hybridizing to multiple target hybridization regions in the viral RNA genome, hampering the emergence of resistant mutants by selection of single mutation.
Ability to address multiple targets. All current effective therapies involve molecules that target specific viral proteins and thus can cure only a single infection. By including in the designed artificial RNAs, such as circRNAs, hybridization regions able to hybridize with different target hybridization regions comprised in target disruption structures of different RNA fragments, such as different viral genomes, we will be able to target different RNA fragments, such as different viruses simultaneously. This broad-spectrum therapy is of great value (i) to simplify treatment of co-infections and (ii) to treat acute infections that share geographical locations and initial symptoms such as, for example, those caused by Dengue virus, Zika virus and Chikungynya virus, or those caused by SARS-CoV-2 and influenza. In acute infections early treatment is key to control the disease and epidemics. A broad-spectrum therapy will allow treating even before a final diagnosis is achieved. Moreover, during epidemics the use of such therapies as preventive treatments would be advisable.
Minimize the risk of drug resistance. Moreover, to minimize further the risk of drug resistance, hybridization regions in the designed artificial RNAs, such as circRNAs, (e.g., see FIG. 21) that target the same viral target disruption structure are all different as the artificial RNAs of the present invention, such as circRNAs, may advantageously comprise G-U pairing. In contrast to DNA where complementarity is required, G-U pairing is a valid hybridization pair in RNA.

In a further embodiment, the artificial RNA of the present invention, which is preferably a circular RNA, comprises at least one hybridization region, wherein the at least one hybridization region is capable of hybridizing target hybridization regions from different target disruption structures within the same RNA fragment, or even from different RNA fragments.

In a preferred embodiment, the artificial RNA of the present invention, which is preferably a circular RNA, comprises 2 or more hybridization regions, preferably between 6 and 20 hybridization regions. In a further preferred embodiment, at least two, and preferably all, of the hybridization regions are capable of completely hybridizing with the same target hybridization region. Hence, the artificial RNA according to this preferred embodiment would have more probabilities of disrupting the target disruption structure of the RNA fragment, and would then be more effective in modulating the functionality of the RNA fragment, especially RNA fragments which are prone to mutations (e.g., viruses and/or tumors). Hence, having multiple hybridization regions impacts mainly the mutant escaping capabilities of the virus/tumor.

In another more preferred embodiment, the RNA of the present invention, which is preferably a circular RNA, comprises at least two hybridization regions, wherein at least two, and preferably all, of the hybridization regions have different nucleotide sequences, i.e., are different from each other in terms of nucleotide sequence. Hence, in this preferred embodiment, the at least two hybridization regions, preferably all of the hybridization regions of the artificial RNA, are different from each other, and they all target (are capable of hybridizing) the same target hybridization region (“many-to-one” approach). This way, the artificial RNA according to this preferred embodiment would have more probabilities of disrupting the target disruption structure of the RNA fragment, and would then be more effective in modulating the functionality of the RNA fragment. Alternatively, the at least two hybridization regions, preferably all of the hybridization regions of the artificial RNA, which are different from each other, target (are capable of hybridizing) different target hybridization regions from different target disruption structures, either within the same RNA fragment or even from different RNA fragments.

In a further preferred embodiment, the two or more hybridization regions of the artificial RNA of the present invention are:
a) separated by non-hybridization regions of sizes up to 20 nucleotides; or
b) are not separated by non-hybridization regions; or
c) are overlapping.

Preferably, the one or more RNA fragments is selected from mRNA, tRNA, rRNA, non-coding RNA and viral genomic RNA. More preferably, the RNA fragment is viral genomic RNA. Even more preferably, the one or more RNA fragments is positive-sense single-stranded (ss) viral genomic RNA.

Viral RNA genomes and viral mRNAs contain highly structured regions (“target disruption structures”) comprising at least a portion of a hairpin loop preceded or followed by a region of unpaired nucleotides which are essential for their function. These highly structured regions are preferably structured regions vital for the viral life cycle (SRVVLC). If these regions are disrupted, the virus would be less capable (and, preferably incapable) of performing essential functions of its life cycle. Accordingly, disrupting these regions would render the virus less infective, ideally totally ineffective.

The present inventors have designed artificial RNAs, preferably circular RNAs, comprising at least one (and preferably more than one) hybridization region that hybridizes to at least one target hybridization region (such as one, or two, or more) within at least one target disruption structure (such as one, or two, or more) present in the viral genomic RNA and disrupts it, consequently reducing or even inhibiting viral infection. As described above, in a preferred embodiment, the artificial RNAs of the present invention comprise at least two (and preferably more) hybridization regions, which are different from each other, and which are able to completely hybridize with one target hybridization region within one target disruption structure present in the viral genomic RNA and disrupt it, consequently reducing or even inhibiting viral infection. In this way (“many-to-one” approach) the efficiency of the disruption is increased.

In one embodiment, the one or more target disruption structures is comprised in the IRES element of the viral genome. Preferably, the one or more target disruption structures comprised in the IRES element of the viral genome is a structured region vital for the viral life cycle (SRVVLC).

In another embodiment, the one or more target disruption structures is comprised in the 5′UTR and/or in the 3′UTR of the viral genome. Preferably, the one or more target disruption structures comprised in the 5′UTR and/or in the 3′UTR of the viral genome is a structured region vital for the viral life cycle (SRVVLC).

In a further embodiment, the one or more target disruption structures is comprised in the CDS of the viral genome. Preferably, the one or more target disruption structures comprised in the CDS of the viral genome is a structured region vital for the viral life cycle (SRVVLC).

In a further embodiment, the at least two hybridization region of the artificial RNA are capable of completely hybridizing with at least one target hybridization region comprised in at least one disruption structure present in any combination of two of the following locations: the IRES element, the region of the 5′UTR, the region of the CDS and/or the region of the 3′UTR of the viral genome.

In a further embodiment, the artificial RNA of the present invention, which is preferably a circular RNA, is able to disrupt by hybridization one or more target disruption structures comprised in at least two viral genomic RNAs. Preferably, the artificial RNA of the present invention is able to disrupt by hybridization one or more structured regions vital for the viral life cycle comprised in at least two viral genomic RNAs.

In a preferred embodiment, the disruption of the one or more structured regions vital for the viral life cycle renders the virus less active, more preferably the disruption of the one or more structured regions vital for the viral life cycle renders the virus completely inactive.

The viral genome as target RNA fragment (viral genomic RNA) is not limited. Any viral genome with target disruption structures (i.e., regions comprising at least a hairpin loop preceded or followed by a region of unpaired nucleotides) may be a suitable target RNA fragment for the artificial RNA of the present invention. Examples of viral genomes which may be target RNA fragments according to the present invention are the following: Hepatitis-C-Virus (HCV), Influenza virus, Hepatitis-A-Virus (HAV), Poliovirus, Coxsackie B virus, Coronavirus, Rhinovirus (common cold), Dengue virus, Zika virus, Chikungunya virus, West Nile virus and Yellow Fever virus.

In another preferred embodiment, the target disruption structures that the artificial RNA of the present invention, preferably in the artificial circular RNA of the present invention, is able to disrupt is a target disruption structure comprised in the 5′UTR of the viral genome. In a preferred embodiment, the target disruption structure of the 5′UTR region of the DENV (Dengue virus) genome is cHP (capsid coding hairpin region).

In another preferred embodiment, the target disruption structures that the artificial RNA of the present invention, preferably in the artificial circular RNA of the present invention, is able to disrupt is a target disruption structure comprised in the IRES element of the viral genome. In a preferred embodiment, the target disruption structure of the IRES element of the HCV (Hepatitis-C virus) viral genome is IRES1 and/or IRES2.

In another preferred embodiment, the target disruption structures that the artificial RNA of the present invention, preferably in the artificial circular RNA of the present invention, is able to disrupt is a target disruption structure comprised in the CDS of the viral genome. In a preferred embodiment, the target disruption structure of the CDS of the viral genome is CDS1 and/or CDS2. Preferably, the target disruption structure is selected from regions SL388, SL427, SL588 and/or SL750 of the genome of the HCV. In a preferred embodiment, the target disruption structure is region SL427 and/or region SL588 of the genome of the HCV (SL stands for Stem Loop).

In another preferred embodiment, the target disruption structures that the artificial RNA of the present invention, preferably in the artificial circular RNA of the present invention, is able to disrupt is a target disruption structure comprised in the 3′UTR of the viral genome. In a preferred embodiment, the target disruption structure of the 3′UTR region of the DENV genome is sHP (short Stem Loop or short Hairpin region). In a further preferred embodiment, the target disruption structure of the 3′UTR region of the WNV (West Nile virus) genome is SL_II.

In another preferred embodiment, in the circular RNA according to any of the preceding embodiments, the target disruption structures are found in a combination of two or more of the IRES element, the structured region of the 5′UTR, the structured region of the CDS or the structured region of the 3′UTR of the viral genome.

In another preferred embodiment, the target disruption structure of the CHIKV (Chikungunya virus) genome is the RSE region and/or the Recoding Element (RE).

In a further preferred embodiment, the one or more target hybridization regions to which the at least one hybridization region of the artificial RNA according to the present invention, preferably of the artificial circular RNA of the present invention, completely hybridizes is comprised in SEQ ID NO.: 1. In particular, the target hybridization region comprises the nucleotide sequence as defined in one or more of SEQ ID NO.: 25 to 28, or a nucleotide sequence with at least 70% identity to the nucleotide sequence as defined in SEQ ID NO.: 25 to 28, preferably with at least 80%, more preferably with at least 90%, even more preferably with at least 95% identity to the nucleotide sequence as defined in SEQ ID NO.: 25 to 28, or the homologe regions in another HCV strain/serotype.

In a preferred embodiment, the one or more target hybridization regions to which the at least one hybridization region of the artificial RNA according to the present invention, preferably of the artificial circular RNA of the present invention, completely hybridizes comprises the nucleotide sequence as defined in SEQ ID NO.: 25 to 27 (combination circ_hcv_ires1, circ HCV2 and circ_hcv_cds1, see Table 1 below) or a nucleotide sequence with at least 70% identity to the nucleotide sequence as defined in SEQ ID NO.: 25 to 27, preferably with at least 80%, more preferably with at least 90%, even more preferably with at least 95% identity to the nucleotide sequence as defined in SEQ ID NO.: 25 to 27, or the homologe regions in another HCV strain/serotype.

The degree of identity between two sequences can be determined by conventional methods, for example, by means of standard sequence alignment algorithms known in the state of the art, such as, for example BLASTn (Altschul S.F. et al. Basic local alignment search tool. J Mol Biol. 1990 Oct 5; 215(3):403-10).

“Homology region” refers to regions in different virus strains/serotypes which share common structural and/or functional characteristics. Homologous structures do not imply sequence identity as a necessary condition. The skilled person is able to identify homologe regions in other viral strains/serotypes as the ones defined in the present application by conventional means.

TABLE 1

CircRNAs designed against HCi98V

| Name of the artificial circular RNA | Target disruption structure comprised in | Target hybridization region | SEQ ID NO.: | Hybridization regions of the artificial RNA |
|---|---|---|---|---|
| circ_hcv_ires1 | IRES 1 SEQ ID NO.: 76 | CUCCGCCAUGA AUCACUCCCCU GUGAGGAACUA SEQ ID NO.: 25 | SEQ ID NO.:2 | 7 |
| Circ HCV2 | IRES 2 SEQ ID NO.: 77 | UCUCGUAGACC GUGCACCAUGA GC SEQ ID NO.: 26 | SEQ ID NO.:3 | 11 |
| circ_hcv_cds1 | CDS1 (target disruption structure: cHP) SEQ ID NO.: 78 | CCAAAAGAAAC ACCAACCGUCG CCCAGA SEQ ID NO.: 27 | SEQ ID NO.:4 | 8 |
| circ_hcv_combo1 | IRES- CDS | Combination circ_hcv_ires1, circ HCV2 and circ_hcv_cds1 | SEQ ID NO.:5 | 3xtarget |
| circ_hcv_cds2 | CDS2 SEQ ID NO.: 79 | GGGGCCCCAGG SEQ ID NO.: 28 | SEQ ID NO.:6 SEQ ID NO : 64 | 12 |

IRES: internal ribosomal entry site; CDS: coding sequence; cHP: capsid hairpin.

The different circRNAs designed against HCV are classified in the table with information of the target disruption structures which they disrupt by hybridization, the sequence of the target hybridization region which completely hybridizes with the hybridization region of the artificial RNA, the sequence of the whole circRNA and the number of hybridization regions present in each candidate (each artificial circular RNA).

In a further preferred embodiment, the one or more target hybridization regions to which the at least one hybridization region of the artificial RNA according to the present invention, preferably of the artificial circular RNA of the present invention, completely hybridizes is comprised in SEQ ID NO.: 7. In particular, the target hybridization region comprises the nucleotide sequence as defined in one or more of SEQ ID NO.: 29 to 30 or a nucleotide sequence with at least 70% identity to the nucleotide sequence as defined in SEQ ID NO.: 29 to 30, preferably with at least 80%, more preferably with at least 90%, even more preferably with at least 95% identity to the nucleotide sequence as defined in SEQ ID NO.: 29 to 30, or the homologe regions in another DENV strain/serotype.

TABLE 2

CircRNAs designed against DENV

| Name of the artificial circular RNA | Target disruption structure comprised in | Target hybridization region | SEQ ID NO.: | Hybridization regions of the artificial RNA |
|---|---|---|---|---|
| circ_dv_3utr | 3′ UTR (target disruption structure: sHP) SEQ ID NO.: 29 | AACAGCAUAU UGACGCUGGG AAAGACCAGA GA SEQ ID NO.: 29 | SEQ ID NO.:8 | 7 |
| circ_dv_cHP_v1 | CDS (target disruption structure: cHP ) SEQ ID NO.: 30 | ACGGAAAAAG GCGAAAAACA CGCCUUUCAA UAU SEQ ID NO.: 30 | SEQ ID NO.:9 | 7 |
| circ_dv_cHP_v2 | CDS (target disruption structure: cHP ) SEQ ID NO.: 30 | SEQ ID NO.: 30 | SEQ ID NO.:10 | 7 |

UTR: untranslated region cHP: capsid region hairpin

The different circRNAs designed against DENV are classified in the table with information of the target disruption structures which they disrupt by hybridization, the sequence of the target hybridization region which completely hybridizes with the hybridization region of the artificial RNA, the sequence of the whole circRNA and the number of hybridization regions present in each candidate (each artificial circular RNA).

In a further preferred embodiment, the one or more target hybridization regions to which the at least one hybridization region of the artificial RNA according to the present invention, preferably of the artificial circular RNA of the present invention, completely hybridizes is comprised in one or more of SEQ ID NO.: 1 and SEQ ID NO.: 7. In particular, the target hybridization region comprises the nucleotide sequence as defined in one or more of SEQ ID NO.: 30 and SEQ ID NO.: 28 or a nucleotide sequence with at least 70% identity to the nucleotide sequence as defined in SEQ ID NO.: 30 and SEQ ID NO.: 28, preferably with at least 80%, more preferably with at least 90%, even more preferably with at least 95% identity to the nucleotide sequence as defined in SEQ ID NO.: 30 and SEQ ID NO.: 28, or the homologe regions in another DENV/HCV strain/serotype.

TABLE 3

Broad-spectrum DENV-HCV circRNA

| | | | | |
|---|---|---|---|---|
| Name of the artificial circular RNA | Target disruption structure comprised in | Target hybridization region | SEQ ID NO.: | Hybridization regions of the artificial RNA |
| circ_dv_cHP_v 1_ circ_hcv_cds2_ 2 | DENV: CDS (target disruption structure: cHP) HCV: CDS SEQ ID NOs.: 30 and 79. | ACGGAAAAAGGCGAAA AACACGCCUUUCAAUA U SEQ ID NO.: 30 GGGGCCCCAGG SEQ ID NO.: 28 | SEQ ID NO: 32 | 4 x target |
| DENV cHP_HCV CDS2_T | DENV: CDS (target disruption structure: cHP) HCV: CDS2 SEQ ID NOs.: 30 and 79. | SEQ ID NO.: 30 SEQ ID NO.: 28 | SEQ ID NO: 16 | 19 |
| DENVlcHP_H CV CDS2_1 | DENV: CDS (target disruption structure: cHP) HCV: CDS2 SEQ ID NOs.: 30 and 79. | SEQ ID NO.: 30 SEQ ID NO.: 28 | SEQ ID NO: 17 | 4xtarget |

cHP: capsid region hairpin CDS: coding sequence.

Information for circ_dv_cHP_v1-circ_hcv_cds2 of the two target disruption structures (DENV cHP and HCV CDS), the target hybridization regions, the sequence of the complete circRNA and the number of hybridization regions in each circRNA.

In a further preferred embodiment, the one or more target hybridization regions to which the at least one hybridization region of the artificial RNA according to the present invention, preferably of the artificial circular RNA of the present invention, completely hybridizes is comprised in SEQ ID NO. 11. In particular, the target hybridization region comprises the nucleotide sequence as defined in one or more of SEQ ID NO.: 31, 33 and 35 or a nucleotide sequence with at least 70% identity to the nucleotide sequence as defined in SEQ ID NO.: 31, 33 and 35, preferably with at least 80%, more preferably with at least 90%, even more preferably with at least 95% identity to the nucleotide sequence as defined in SEQ ID NO.: 31, 33 and 35, or the homologe regions in another CHIKV strain/serotype.

TABLE 4

CircRNAs designed against CHIKV

| Name of the artificial circular RNA | Target disruption structure comprised in | Target hybridization region | SEQ ID NO.: | Hybridization regions of the artificial RNA |
|---|---|---|---|---|
| chikv5utr | 5′UTR SEQ ID NO: 80 | ACACACGUAGC CUACCAGUUUC UUACUGCUCUA CU SEQ ID NO.: 33 | SEQ ID NO.:12 | 6 |
| chikv5utr | 5′UTR SEQ ID NO: 80 | SEQ ID NO.: 33 | SEQ ID NO.: 13 | 7 |
| chikvRSE | 3′ UTR (target disruption structure: Repetitive Sequence Element (RSE)) SEQ ID NO: 81 | AGCAAAUAAUC UAUAGAUCAAA GGGCUACGCAA SEQ ID NO.: 35 | SEQ ID NO.: 14 | 6 |
| Chikv_re | Recoding Element (CDS) SEQ ID NO: 82 | GCUGCUGUAAA ACGUUGGCUUU UUUAGCCGUAA U SEQ ID NO.: 31 | SEQ ID NO.:39 | 6 |
| chikvRSE | 3′ UTR (target disruption structure: RSE) SEQ ID NO: 81 | SEQ ID NO.: 35 | SEQ ID NO.: 15 | 6 |

UTR: untranslated region CDS: coding sequence.

The different circRNAs designed against CHIKV are classified in the table with information of the target disruption structure, the sequence of the target hybridization region, the sequence of the whole circRNA and the number of hybridization regions present in each candidate (in each artificial circRNA).

In a further preferred embodiment, the one or more target hybridization regions to which the at least one hybridization region of the artificial RNA according to the present invention, preferably of the artificial circular RNA of the present invention, completely hybridizes is comprised in SEQ ID NO. 20. In particular, the target hybridization region comprises the nucleotide sequence as defined in SEQ ID NO.: 37 or a nucleotide sequence with at least 70% identity to the nucleotide sequence as defined in SEQ ID NO.: 37, preferably with at least 80%, more preferably with at least 90%, even more preferably with at least 95% identity to the nucleotide sequence as defined in SEQ ID NO.: 37, or the homologe regions in another WNV strain/serotype.

TABLE 5

CircRNAs designed against WNV

| Name of the artificial circular RNA | Target disruption structure comprised in | Target hybridization region | SEQ ID NO.: | Hybridization regions of the artificial RNA |
|---|---|---|---|---|
| circ_wnv_s1II_1 | 3′ UTR (target disruption structure SL III) SEQ ID NO.: 83 | UUUUGAGGAGA AAGUCAGGCCG GGAAGUU SEQ ID NO.: 37 | SEQ ID NO.:24 | 7 |
| circ_wnv_s1II_2 | 3′ UTR (target disruption structure SL III) SEQ ID NO.: 83 | UUUUGAGGAGA AAGUCAGGCCG GGAAGUU SEQ ID NO.: 37 | SEQ ID NO.: 19 | 7 |

UTR: untranslated region.

The different circRNAs designed against WNV are classified in the table with information of the target disruption structures, the sequence of the target hybridization region, the sequence of the complete circRNA and the number of hybridization regions present in each candidate (in each artificial circRNA). SLIII: Stem Loop III.

In a further preferred embodiment, the one or more target hybridization regions to which the at least one hybridization region of the artificial RNA according to the present invention, preferably of the artificial circular RNA of the present invention, completely hybridizes is comprised in SEQ ID NO.: 20 and SEQ ID NO.: 7. In particular, the target hybridization region comprises the nucleotide sequence as defined in one or more of SEQ ID NO.: 30 and 37 or a nucleotide sequence with at least 70% identity to the nucleotide sequence as defined in SEQ ID NO.: 30 and 37, preferably with at least 80%, more preferably with at least 90%, even more preferably with at least 95% identity to the nucleotide sequence as defined in SEQ ID NO.: 30 and 37, or the homologe regions in another DENV/WNV strain/serotype.

TABLE 6

CircRNAs designed against WNV and DENV

| Name of the artificial circular RNA | Target disruption structure comprised in | Target hybridization region | SEQ ID NO.: | Hybridization regions of the artificial RNA |
|---|---|---|---|---|
| dchp_ws1I_A | CDS and 3′ UTR (target disruption structures: cHP and SLII ) | SEQ ID NO.: 30 and 37 | SEQ ID NO.:21 SEQ ID NO: 63 | 6 |
| dchp_ws1I_B | CDS and 3′ UTR (target disruption structures: cHP and SLI I) | SEQ ID NO.: 30 and 37 | SEQ ID NO.:22 | 6 |
| dchp_ws1I_C | CDS and 3′ UTR (target disruption structures: cHP and SLI I) | SEQ ID NO.: 30 and 37 | SEQ ID NO.:23 | 6 |

SLI: Stem Loop I. cHP: capsid region hairpin

The different circRNAs designed against WNV and DENV are classified in the table with information of the target disruption structures, the sequence of the target hybridization region, the sequence of the complete circRNA and the number of hybridization regions present in each candidate (in each artificial circRNA).

In a further preferred embodiment, the one or more target hybridization regions to which the at least one hybridization region of the artificial RNA according to the present invention, preferably of the artificial circular RNA of the present invention, completely hybridizes is comprised in one or more of SEQ ID NO.: 1, SEQ ID NO.: 7, SEQ ID NO.: 11 and/or SEQ ID NO. 20. In particular, the target hybridization region comprises the nucleotide sequence as defined in one or more of SEQ ID NO.: 25 to 31, 33 and 35, 37 or a nucleotide sequence with at least 70% identity to the nucleotide sequence as defined in SEQ ID NO.: 25 to 31, 33 and 35, 37, preferably with at least 80%, more preferably with at least 90%, even more preferably with at least 95% identity to the nucleotide sequence as defined in SEQ ID NO.: 25 to 31, 33 and 35, 37.

In another preferred embodiment, the target disruption structures in the HCV viral genome are found in the IRES and/or CDS region and/or a combination of them. More preferably, the target disruption structure of the HCV genome is selected from the list of target disruption structures comprising or consisting of SEQ ID NOs.: 76, 77, 78 or 79 or a nucleotide sequence with at least 60%, 70%, 80%, 90%, 91%, 92%, 93%, 94%, 95%, 96%, 97%, 98%, 99% identity to the nucleotide sequence as defined in SEQ ID NOs.: 76, 77, 78, or 79, or any combination thereof.

In another preferred embodiment, the target disruption structures in the Dengue viral genome are found in the 3′UTR and/or is the cHP and/or a combination of them. More preferably, the target disruption structure of the DENV genome is selected from the list of target disruption structures comprising or consisting of SEQ ID NOs.: 29 or 30, or a nucleotide sequence with at least 60%, 70%, 80%, 90%, 91%, 92%, 93%, 94%, 95%, 96%, 97%, 98%, 99% identity to the nucleotide sequence as defined in SEQ ID NOs.: 29 or 30, or any combination thereof.

In another preferred embodiment, the target disruption structures in the Zika viral genome are found in the 5′UTR and/or are the RSE and/or a combination of them.

In another preferred embodiment, the target disruption structures in the Chikungunya viral genome is found in the 5′UTR and/or is the RSE and/or a combination of them. More preferably, the target disruption structure of the CHIKV genome is selected from the list of target disruption structures comprising or consisting of SEQ ID NOs.: 80, 81, or 82, or a nucleotide sequence with at least 60%, 70%, 80%, 90%, 91%, 92%, 93%, 94%, 95%, 96%, 97%, 98%, 99% identity to the nucleotide sequence as defined in SEQ ID NOs.: 80, 81, or 82, or any combination thereof.

In another preferred embodiment, the target disruption structure in the West Nile viral genome is the stem-loop III (SLIII). More preferably, the target disruption structure of the WNV genome comprises or consists of SEQ ID NO.: 83, or a nucleotide sequence with at least 60%, 70%, 80%, 90%, 91%, 92%, 93%, 94%, 95%, 96%, 97%, 98%, 99% identity to the nucleotide sequence as defined in SEQ ID NO.: 83.

In another preferred embodiment, the hybridization regions of the artificial RNA of the present invention, which is preferably a circular RNA, target (i.e., are able to completely hybridize with target hybridization regions present in) more than one the target disruption structures present in one viral genome.

In a particular embodiment, the artificial RNA of the present invention, preferably the artificial circular RNA of the present invention, has a broad spectrum activity against a RNA viral genome, preferably against HCV, Dengue, Zika, Chikungunya, West Nile and Yellow Fever viral genome. In the present invention “a broad spectrum activity” related to the artificial RNA, preferably the circular RNA of the present invention means that said artificial RNA is effective against a wide range of RNA viruses, preferably against HCV, Dengue, Zika, Chikungunya, West Nile and Yellow Fever.

In a further preferred embodiment, the one or more target hybridization regions to which the at least one hybridization region of the artificial RNA according to the present invention, preferably of the artificial circular RNA of the present invention, completely hybridizes is comprised in SEQ ID NO. 34. In particular, the target hybridization region comprises the nucleotide sequence as defined in one or more of SEQ ID NO.: 58-62 or a nucleotide sequence with at least 70% identity to the nucleotide sequence as defined in SEQ ID NO.: 58-62, preferably with at least 80%, more preferably with at least 90%, even more preferably with at least 95% identity to the nucleotide sequence as defined in SEQ ID NO.: 58-62, or the homologe regions in another SARS-CoV-2 strain/serotype.

In another preferred embodiment, the target disruption structures in the SARS-CoV-2 viral genome is found in the Target A, B, C, D, 3′UTR, 5′UTR, and/or a combination of them. More preferably, the target disruption structure of the SARS-CoV-2 genome is selected from the list of target disruption structures comprising or consisting of SEQ ID NOs.: 84, 58, 85, 86, 87, or a nucleotide sequence with at least 60%, 70%, 80%, 90%, 91%, 92%, 93%, 94%, 95%, 96%, 97%, 98%, 99% identity to the nucleotide sequence as defined in SEQ ID NOs.: 84, 58, 85, 86 or 87, or any combination thereof.

TABLE 7

CircRNAs designed against severe acute respiratory syndrome coronavirus 2 (SARS-CoV-2) isolate Wuhan-Hu-1 (complete genome: SEQ ID NO.: 34)

| Name of the artificial circular RNA | Target disruption structure comprised in | Target hybridization region | SEQ ID NO.: | Hybridization regions of the artificial RNA |
|---|---|---|---|---|
| Artificial circular ARN 1; target disruption structure comprised in SARS-CoV-2 3′UTR replication site | 3′UTR replication site SEQ ID NO.: 84 | CAGAAUGAAUU CUCGUAACUAC AUAGCACAAGU AG SEQ ID NO.: 62 | SEQ ID NO.:36 | 6 |
| Artificial circular ARN 2; target disruption structure comprised in SARS-CoV-2 3′UTR | 3′UTR replication site SEQ ID NO.: 84 | CAGAAUGAAUU CUCGUAACUAC AUAGCACAAGU AG SEQ ID NO.: 62 | SEQ ID NO.:38 | 6 |
| Artificial circular RNA 3; target disruption structure comprised in SARS-CoV-2 3′UTR replication site | 3′UTR replication site SEQ ID NO.: 84 | CAGAAUGAAUU CUCGUAACUAC AUAGCACAAGU AG SEQ ID NO.: 62 | SEQ ID NO.:40 | 6 |
| Artificial circular RNA 4; target disruption structure comprised in SARS-CoV-2 3′UTR replication site | 3′UTR replication site SEQ ID NO.: 84 | CAGAAUGAAUU CUCGUAACUAC AUAGCACAAGU AG SEQ ID NO.: 62 | SEQ ID NO.:41 | 6 |
| Artificial circular RNA 5; target disruption structure comprised in SARS-CoV-2 3′UTR replication site | 3′UTR replication site SEQ ID NO.: 84 | CAGAAUGAAUU CUCGUAACUAC AUAGCACAAGU AG SEQ ID NO.: 62 | SEQ ID NO.:42 | 6 |
| Artificial circular RNA 6; target disruption structure comprised in SARS-CoV-2 3′UTR replication site | 3′UTR replication site SEQ ID NO.: 84 | CAGAAUGAAUU CUCGUAACUAC AUAGCACAAGU AG SEQ ID NO.: 62 | SEQ ID NO.: 88 | 6 |
| Artificial circular RNA 7; target disruption structure comprised in SARS-CoV-2 3′UTR replication site | 3′UTR replication site SEQ ID NO.: 84 | CAGAAUGAAUU CUCGUAACUAC AUAGCACAAGU AG SEQ ID NO.: 62 | SEQ ID NO.: 89 | 6 |
| Artificial circular RNA 8; target disruption structure comprised in SARS-CoV-2 3′UTR replication site | 3′UTR replication site SEQ ID NO.: 84 | CAGAAUGAAUU CUCGUAACUAC AUAGCACAAGU AG SEQ ID NO.: 62 | SEQ ID NO.: 65 | 6 |
| Artificial circular RNA 1; target disruption structure comprised in SARS-CoV-2 5′UTR (S1II) | 5′UTR (target disruption structure: S1II) SEQ ID NO.: 58 | AACCAACUUUC GAUCUCUUGUA GAUCU SEQ ID NO.: 58 | SEQ ID NO.:43 | 7 |
| Artificial circular RNA 2; target disruption structure comprised in SARS-CoV-2 5′UTR (S1II) | 5′UTR (target disruption structure: S1II) SEQ ID NO.: 58 | AACCAACUUUC GAUCUCUUGUA GAUCU SEQ ID NO.: 58 | SEQ ID NO.:44 | 7 |
| Artificial circular RNA 3; target disruption structure comprised in SARS-CoV-2 5′UTR (S1II) | 5′UTR (target disruption structure: S1II) SEQ ID NO.: 58 | AACCAACUUUC GAUCUCUUGUA GAUCU SEQ ID NO.: 58 | SEQ ID NO.:45 | 7 |
| Artificial circular RNA 4; target disruption structure comprised in SARS-CoV-2 5′UTR (S1II) | 5′UTR (target disruption structure: S1II) SEQ ID NO.: 58 | AACCAACUUUC GAUCUCUUGUA GAUCU SEQ ID NO.: 58 | SEQ ID NO.: 66 | 7 |
| Artificial circular RNA 5; target disruption structure comprised in SARS-CoV-2 5′UTR (S1II) | 5′UTR (target disruption structure: S1II) SEQ ID NO.: 58 | AACCAACUUUC GAUCUCUUGUA GAUCU SEQ ID NO.: 58 | SEQ ID NO.: 67 | 7 |
| Artificial circular RNA 1; target hybridization region: SARS-CoV-2 Target A | Target hybridization region: SARS-CoV-2 Target A SEQ ID NO.: 85 | UUUAAGUUUAG AAUAGACGGUG ACAUGGUACCA SEQ ID NO.: 59 | SEQ ID NO.:46 | 6 |
| Artificial circular RNA 2; target hybridization region: SARS-CoV-2 Target A | Target hybridization region: SARS-CoV-2 Target A SEQ ID NO.: 85 | UUUAAGUUUAG AAUAGACGGUG ACAUGGUACCA SEQ ID NO.: 59 | SEQ ID NO.:47 | 6 |
| Artificial circular RNA 3; target hybridization region: SARS-CoV-2 Target A | Target hybridization region: SARS-CoV-2 Target A SEQ ID NO.: 85 | UUUAAGUUUAG AAUAGACGGUG ACAUGGUACCA SEQ ID NO.: 59 | SEQ ID NO.:48 | 6 |
| Artificial circular RNA 4; target hybridization region: SARS-CoV-2 Target A | Target hybridization region: SARS-CoV-2 Target A SEQ ID NO.: 85 | UUUAAGUUUAG AAUAGACGGUG ACAUGGUACCA SEQ ID NO.: 59 | SEQ ID NO.:49 | 6 |
| Artificial circular RNA 5; target hybridization region: SARS-CoV-2 Target A | Target hybridization region: SARS-CoV-2 Target A SEQ ID NO.: 85 | UUUAAGUUUAG AAUAGACGGUG ACAUGGUACCA SEQ ID NO.: 59 | SEQ ID NO.:68 | 6 |
| Artificial circular RNA 6; target hybridization region: SARS-CoV-2 Target A | Target hybridization region: SARS-CoV-2 Target A | UUUAAGUUUAG AAUAGACGGUG ACAUGGUACCA SEQ ID NO.: 59 | SEQ ID NO.:69 | 6 |
| | SEQ ID NO.: 85 | | | |
| Artificial circular RNA 7; target hybridization region: SARS-CoV-2 Target A | Target hybridization region: SARS-CoV-2 Target A SEQ ID NO.: 85 | UUUAAGUUUAG AAUAGACGGUG ACAUGGUACCA SEQ ID NO.: 59 | SEQ ID NO.:70 | 6 |
| Artificial circular RNA 1; target hybridization region: SARS-CoV-2 Target C | Target hybridization region: SARS-CoV-2 Target C SEQ ID NO.: 86 | UCACUAAGAAA UCUGCUGCUGA GGCUUCUA SEQ ID NO.: 60 | SEQ ID NO.:50 | 7 |
| Artificial circular RNA 2; target hybridization region: SARS-CoV-2 Target C | Target hybridization region: SARS-CoV-2 Target C SEQ ID NO.: 86 | UCACUAAGAAA UCUGCUGCUGA GGCUUCUA SEQ ID NO.: 60 | SEQ ID NO.:51 | 7 |
| Artificial circular RNA 3; target hybridization region: SARS-CoV-2 Target C | Target hybridization region: SARS-CoV-2 Target C SEQ ID NO.: 86 | UCACUAAGAAA UCUGCUGCUGA GGCUUCUA SEQ ID NO.: 60 | SEQ ID NO.:52 | 7 |
| Artificial circular RNA 4; target hybridization region: SARS-CoV-2 Target C | Target hybridization region: SARS-CoV-2 Target C SEQ ID NO.: 86 | UCACUAAGAAA UCUGCUGCUGA GGCUUCUA SEQ ID NO.: 60 | SEQ ID NO.:53 | 7 |
| Artificial circular RNA 5; target hybridization region: SARS-CoV-2 Target C | Target hybridization region: SARS-CoV-2 Target C SEQ ID NO.: 86 | UCACUAAGAAA UCUGCUGCUGA GGCUUCUA SEQ ID NO.: 60 | SEQ ID NO.:71 | 7 |
| Artificial circular RNA 6; target hybridization region: SARS-CoV-2 Target C | Target hybridization region: SARS-CoV-2 Target C SEQ ID NO.: 86 | UCACUAAGAAA UCUGCUGCUGA GGCUUCUA SEQ ID NO.: 60 | SEQ ID NO.:72 | 7 |
| Artificial circular RNA 1; target hybridization region: SARS-CoV-2 Target D | Target hybridization region: SARS-CoV-2 Target D SEQ ID NO.: 87 | UCGUCUAUCUU CUGCAGGCUGC UUACGGUUU SEQ ID NO.: 61 | SEQ ID NO.:54 | 6 |
| Artificial circular RNA 2; target hybridization region: SARS-CoV-2 Target D | Target hybridization region: SARS-CoV-2 Target D SEQ ID NO.: 87 | UCGUCUAUCUU CUGCAGGCUGC UUACGGUUU SEQ ID NO.: 61 | SEQ ID NO.:55 | 6 |
| Artificial circular RNA 3; target hybridization region: SARS-CoV-2 Target D | Target hybridization region: SARS-CoV-2 Target D SEQ ID NO.: 87 | UCGUCUAUCUU CUGCAGGCUGC UUACGGUUU SEQ ID NO.: 61 | SEQ ID NO.:56 | 6 |
| Artificial circular RNA 4; target hybridization region: SARS-CoV-2 Target D | Target hybridization region: SARS-CoV-2 Target D SEQ ID NO.: 87 | UCGUCUAUCUU CUGCAGGCUGC UUACGGUUU SEQ ID NO.: 61 | SEQ ID NO.:57 | 6 |

The different circRNAs designed against SARS-CoV-2 are classified in the table with information of the target disruption structures, the sequence of the target hybridization region, the sequence of the complete circRNA and the number of hybridization regions present in each candidate (in each artificial circRNA).

In a further preferred embodiment, the one or more target hybridization regions to which the at least one hybridization region of the artificial RNA according to the present invention, preferably of the artificial circular RNA of the present invention, completely hybridizes is comprised in one or more of SEQ ID NO.: 1, SEQ ID NO.: 7, SEQ ID NO.: 11 and/or SEQ ID NO. 20. In particular, the target hybridization region comprises the nucleotide sequence as defined in one or more of SEQ ID NO.: 25 to 31, 33, 35, 37 and 58-62 or a nucleotide sequence with at least 70% identity to the nucleotide sequence as defined in SEQ ID NO.: 25 to 31, 33 and 35, 37, preferably with at least 80%, more preferably with at least 90%, even more preferably with at least 95% identity to the nucleotide sequence as defined in SEQ ID NO.: 25 to 31, 33, 35, 37, and 58-62.

All the possible combinations of the target disruption structures and/or the target hybridization regions from the same virus or from different viruses disclosed above are included herein as particular embodiments for the design of the circRNAs sequences of the present invention. It is noted that for each target disruption structures disclosed throughtout the present invention (IRES, CDS, etc), different corresponding hybridization regions can be used and/or combined as described herein in order to design the circRNAs.

In a second aspect, the present invention provides a composition comprising the artificial RNA of the present invention, in any of the embodiments, alone or in combination, disclosed herein.

Preferably, the composition is a pharmaceutical composition, and preferably comprises the artificial RNA of the present invention, in any of the embodiments, and one or more pharmaceutically acceptable carriers.

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

Colloidal dispersion systems, such as macromolecule complexes, nanocapsules, microspheres, beads, and lipid-based systems including oil-in-water emulsions, micelles, mixed micelles, and liposomes, may be used as delivery vehicles for artificial RNAs, such as circular RNAs. Commercially available fat emulsions that are suitable for delivering the nucleic acids of the disclosure to tissues include Intralipid, Liposyn, Liposyn II, Liposyn III, Nutrilipid, and other similar lipid emulsions. A colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO 03/093449. In particular, cationic liposome formulations comprising lipofectamine can be used for delivery. Lipofectamine may be formulated with a neutral co-lipid or helper lipid. See e.g., U.S. Pat. No. 7,479,573, Dalby et al. (2004) Science Direct, Methods 33:95-103, Hawley-Nelson et al. (1993) Focus 15 :73-79.

One will generally desire to employ appropriate salts and buffers to render delivery vehicles stable and allow for uptake by target cells. Buffers also will be employed when recombinant cells (e.g., transfected ex vivo with an artificial RNA, such as a circular RNA) are introduced into a patient. Aqueous compositions of the present disclosure comprise an effective amount of the delivery vehicle, dissolved or dispersed in a pharmaceutically acceptable carrier or aqueous medium.

The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art.

Except insofar as any conventional media or agent is incompatible with the active ingredients of the present disclosure, its use in therapeutic compositions is contemplated. Supplementary active ingredients also can be incorporated into the compositions, provided they do not inactivate the nucleic acids of the compositions.

The pharmaceutical forms suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington’s Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologies standards.

The pharmaceutical compositions of the present invention can also be housed in a syringe, an implantation device, or the like, depending upon the intended mode of delivery and use. Preferably, the compositions comprising artificial RNAs, such as artificial circular RNAs, prepared as described herein, are in unit dosage form, meaning an amount of a composition appropriate for a single dose, in a premeasured or pre-packaged form.

As for the administration, at least one therapeutically effective cycle of treatment with an artificial RNA, such as artificial circular RNAs, may be administered to a subject for treatment of a viral infection caused by, e.g., HCV, Dengue virus, Zika virus, Chikungunya virus, West Nile virus, Yellow Fever virus or coronavirus, such as SARS and/or MERS, preferably SARS-CoV-2.

By “therapeutically effective dose or amount” of a composition comprising an artificial RNA, such as artificial circular RNAs, is intended an amount that, when administered as described herein, brings about a positive therapeutic response, such as improved recovery from the treated condition, such as for example the viral infection, the cancer or the genetic disorder.

Multiple therapeutically effective doses of compositions comprising artificial RNAs of the present invention, such as artificial circular RNAs of the present invention, and/or one or more other therapeutic agents, will be administered. The compositions of the present invention are typically, although not necessarily, administered via injection (subcutaneously, intravenously, intra-arterially, or intramuscularly), by infusion, or locally. Additional modes of administration are also contemplated, such as intraperitoneal, intrathecal, intratumor, intralymphatic, intravascular, intralesion, transdermal, and so forth. In some embodiments, the pharmaceutical composition comprising the artificial RNA of the present invention,, such as artificial circular RNAs, is administered locally. The pharmaceutical compositions comprising the artificial RNAs of the present invention, such as artificial circular RNAs, and other agents may be administered using the same or different routes of administration in accordance with any medically acceptable method known in the art.

The pharmaceutical compositions comprising the artificial RNAs of the present invention, such as artificial circular RNAs, may also be administered prophylactically, e.g., to prevent the viral infection and/or cancer and/or genetic disorder.

The pharmaceutical compositions comprising the artificial RNAs of the present invention, such as artificial circular RNAs, and/or other agents are in a sustained-release formulation, or a formulation that is administered using a sustained-release device. Such devices are well known in the art, and include, for example, miniature implantable pumps that can provide for delivery over time in a continuous, steady-state fashion at a variety of doses to achieve a sustained-release effect with a non-sustained-release pharmaceutical composition.

Those of ordinary skill in the art will appreciate which conditions compositions comprising artificial RNAs of the present invention can effectively treat and/or prevent. The actual dose to be administered will vary depending upon the age, weight, and general condition of the subject as well as the severity of the condition being treated, the judgment of the health care professional, and conjugate being administered.

Therapeutically effective amounts can be determined by those skilled in the art, and are adjusted to the particular requirements of each particular case. Compositions comprising the artificial RNAs of the present invention, such as artificial circular RNAs, can be administered alone or in combination with one or more other therapeutic agents. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

Compositions comprising the artificial RNAs of the present invention, such as artificial circular RNAs, can be administered prior to, concurrent with, or subsequent to other agents. If provided at the same time as other agents, the artificial RNAs of the present invention can be provided in the same or in a different composition. Thus, artificial RNAs and one or more other agents can be presented to the individual by way of concurrent therapy.

By “concurrent therapy” is intended administration to a subject such that the therapeutic effect of the combination of the substances is caused in the subject undergoing therapy. For example, concurrent therapy may be achieved by administering a dose of a pharmaceutical composition comprising an artificial RNA according to the present invention, such as artificial circular RNAs, and a dose of a pharmaceutical composition comprising at least one other agent, which in combination comprise a therapeutically effective dose, according to a particular dosing regimen. Similarly, an artificial RNA of the present invention and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

In a third aspect, the present invention provides a kit comprising the artificial RNA of the present invention, in any of its embodiments, or comprising the composition of the present invention, and instructions for using the artificial RNA or composition.

Hence, any of the artificial RNAs and/or compositions described herein may be included in a kit. For example circular RNAs as disclosed herein may be included in a kit. The kit may also include one or more transfection reagents to facilitate delivery of the artificial RNAs to cells. Such kits may also include components that preserve the polynucleotides or that protect against their degradation. Such components may be RNAse-free or protect against RNAses.

Such kits generally will comprise, in suitable means, distinct containers for each individual reagent or solution. The kit may comprise one or more containers holding the artificial RNAs and other agents. Suitable containers for the compositions include, for example, bottles, vials, syringes, and test tubes. Containers can be formed from a variety of materials, including glass or plastic. A container may have a sterile access port (for example, the container may be a vial having a stopper pierceable by a hypodermic injection needle).

The kit can further comprise a container comprising a pharmaceutically acceptable buffer, such as phosphate-buffered saline, Ringer’s solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating the viral infections with the artificiañ RNAs of the present invention. The package insert can be an unapproved draft package insert or can be a package insert approved by the Food and Drug Administration (FDA) or other regulatory body.

As described above, preferably, the artificial RNA of the present invention, in any of its embodiments, which may be comprised in the composition or kit of the present invention, comprises the nucleotide sequence as defined in SEQ ID NO: 2, 3, 4, 5, 6 (for HCV); 8, 9, 10 (for Dengue virus); 12, 13, 14, 15, 39 (for Chikungunya virus); 16 and 17 (Broadspectrum activity for both HCV and Dengue Virus); 24 and 19 (for West Nile Virus); 21, 22 and 23 (Broad spectrum activity for both Dengue and West Nile Viruses); 32 (Broad spectrum activity for both HCV and Dengue Virus), 36, 38, 40, 41, 42, 88, 89, 65, 43, 44, 45, 66, 67, 46, 47, 48, 49, 68, 69, 70, 50, 51, 52, 53, 71, 72, 54, 55, 56, 57 (for SARS-CoV-2 virus). These are the target hybridization regions which completely hybridize with the hybridization regions comprised in the artificial RNAs described in the examples below.

Preferably, the the one or more target hybridization regions which completely hybridize with the at least one hybridization region comprised in the artificial RNA of the present invention, in any of its embodiments, which may also be comprised in the composition and/or kit of the present invention is comprised in SEQ ID NO.: 1, SEQ ID NO.: 7, SEQ ID NO.: 11, SEQ ID NO.: 20 and/or SEQ ID NO.: 34.

In a fourth aspect, the present invention provides the artificial RNA of the present invention, in any of its embodiments, or the composition or the kit of the present invention for use as a medicament. Preferably, the present invention provides the artificial RNA of the present invention, in any of its embodiments, or the composition or the kit of the present invention for use in a method of preventing and/or treating a viral infection. Preferably, the artificial RNA of the present invention has a broad spectrum activity against two or more RNA viruses.

In a further embodiment, the present invention provides the artificial RNA of the present invention, in any of its embodiments, or the composition or the kit of the present invention for use in a method of preventing and/or treating cancer.

In a further embodiment, the present invention provides the artificial RNA of the present invention, in any of its embodiments, or the composition or the kit of the present invention for use in a method of preventing and/or treating genetic disorders.

In the context of the present invention, a genetic disorder refers to a health problem caused by one or more abnormalities in the genome. It can be caused by a mutation in a single gene (monogenic) or multiple genes (polygenic) or by a chromosomal abnormality. Examples of genetic disorders are the following: Familial hypercholesterolemia, Polycystic kidney disease, Neurofibromatosis type I, Hereditary spherocytosis, Marfan syndrome, Huntington’s disease, Sickle cell anaemia, Cystic fibrosis, Tay-Sachs disease, Phenylketonuria, Mucopolysaccharidoses, Lysosomal acid lipase deficiency, Glycogen storage diseases, Galactosemia, Duchenne muscular dystrophy or Hemophilia.

Hence, in the fourth aspect, the present invention provides the use of the artificial RNA of the present invention, in any of its embodiments, or the composition or the kit of the present invention for the manufacture of a medicament for preventing and/or treating a viral infection and/or cancer and/or a genetic disorder.

In a preferred embodiment, the viral infection is caused by HCV, Dengue, Zika, Chikungunya, West Nile, Yellow Fever virus or coronavirus, such as SARS and/or MERS, preferably SARS-CoV-2.

As it is well known in the art (see for example WO 2017/222911), any known method of nucleic acid delivery may be used for administration of the artificial RNAs as disclosed herein (e.g., immunogenic or non-immunogenic) to a subject. For example, an artificial RNA may be administered by transfection in vivo. Alternatively, the artificial RNA may be administered by transfection ex vivo, and subsequent transfer of a cell transfected with the artificial RNA to the subject.

An artificial RNA, such as a circular RNA, can be delivered with a nucleic acid carrier (e.g. cationic carrier) or a nanoparticle (e.g., lipid nanoparticle, polymeric nanoparticle, nanoparticle comprising a combination of polymers and peptides, or an electrostatic complex). The artificial RNA may be delivered to a subject using a recombinant virus, an exosome, liposome, or other lipid vesicle, or a cell engineered to secrete an artificial RNA, such as a circular RNA.

The artificial RNA, such as a circular RNA, can also be conjugated to a targeting ligand (e.g., small molecule, peptide or protein) for localized delivery to a particular site (e.g., cells, tissue, or organ) in the subject. The artificial RNA can be even linked to an internalization sequence, a protein transduction domain, or a cell penetrating peptide to facilitate entry into a cell.

In a fifth aspect, the present invention provides a method for producing the artificial RNA of the invention, preferably the artificial circular RNA of the present invention, wherein in the method comprises the step of:
a) synthesizing the artificial RNA of the invention;
b) screening the artificial RNA of step a) for artificial RNAs which are capable of disrupting the structure one or more target disruption structures of one or more RNA fragments, preferably which are able of disrupting the SRVVLC and/or rendering the virus less capable (ideally incapable) of performing essential functions of its life cycle, i.e., artificial RNAs which are capable of yielding conformational changes in the target RNA fragment, e.g., yielding conformational changes in the RNA of the virus which impede essential functions of the life cycle of the virus.

### Production of CircRNAs

Artificial RNAs, such as circRNAs, are designed using an in-house software tool. This tool allows for several design constraints to be taken into account, for instance GC content range of the sequence. Once the artificial RNAs, such as circRNAs RNAs, are designed, they can be produced intracellularly or by in vitro transcription.

### Intracellular Production of Artificial RNAs, Such as CircRNAs

The corresponding artificial RNAs sequence, such as circRNAs sequence, in DNA form is commercially obtained and cloned to be later transformed into cells using the following steps.

First, the designed artificial RNA sequence is cloned into a plasmid (por example a pcDNA3-CIRS7 plasmid) between two sequences that mediate splicing and circularization, SA (splicing acceptor) and SD (splicing donor) (clone gently provided by Dr. Thomas B. Hansen from the University of Aarhus (Denmark) (Patent application: WO 2014/082644 A1). This plasmid contains a gene conferring resistance to the antibiotic ampicillin. The derived plasmid is then transformed into XL1-Blue competent cells (Sigma). The transformed cells are grown in LB media with ampicillin (LB—Amp) for selection. One of the selected colonies is then grown in LB—Amp liquid medium for 36h for amplification. After this time, the culture is centrifuged to obtain the bacteria cells and the plasmid purified using NucleoSpin® Plasmid kit (Macherey-Nagel).

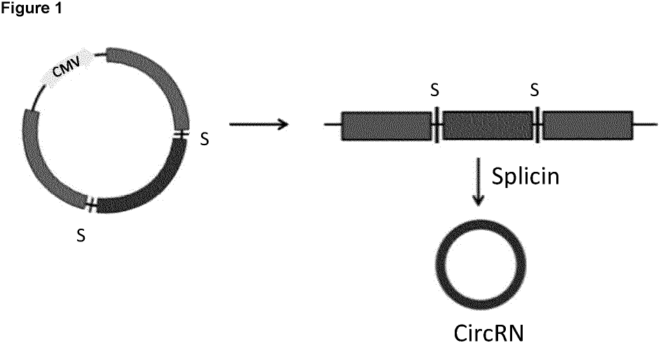

When these derived plasmids are introduced into human cells, the transcription promoter CMV is recognized by the cellular transcription machinery and a mRNA is expressed using the host transcription machinery. For the production of circRNAs, the resultant RNA is circularized by the host splicing machinery generating the designed circRNAs (see FIG. 1).

### In Vitro Production of Artificial CircRNAs

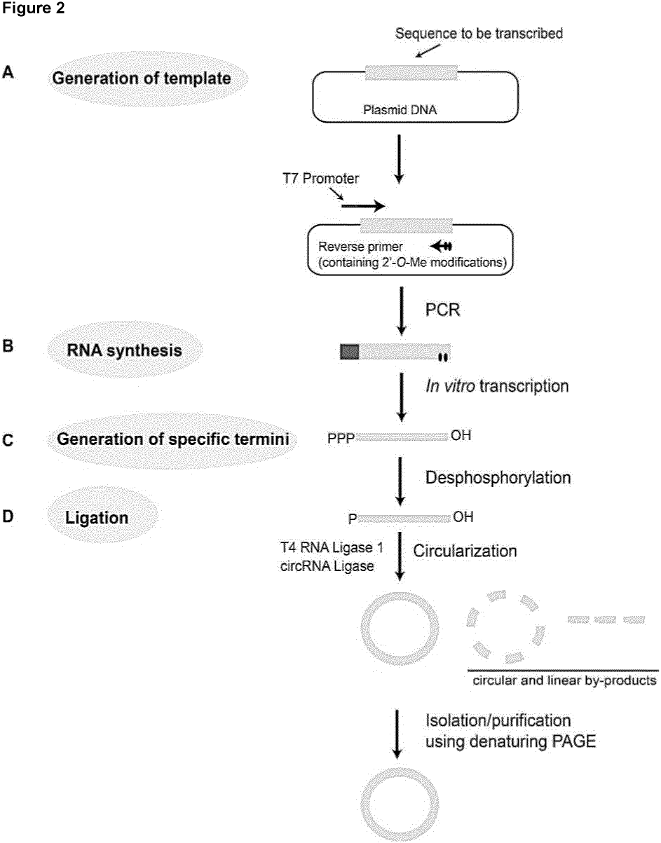

The production of circRNAs can be also achieved in vitro by obtaining the corresponding linear RNA, through chemical synthesis or in vitro transcription, and then circularize it with RNA ligases (see FIG. 2).

A more efficient approach is to produce the circRNAs in vitro from T7 promoter-flanked templates. This plasmid contains self-cleavage ribozymes that after the in vitro transcription will be cleaved generating the ends that will allow the circularization and generation of circRNAs. For each candidate, the T7 promoter plasmid was linearized using Hindlll (FastDigest) during 30 min at 37° C. The purified linearized molecule was in vitro transcribed and purified from a polyacrylamide gel. Afterwards, the RNAs will be circularized using RtcB (NEB) and the circularized molecules were selected after RNAse R (Lucigen) treatment during 30′ at 37° C. (See FIG. 25).

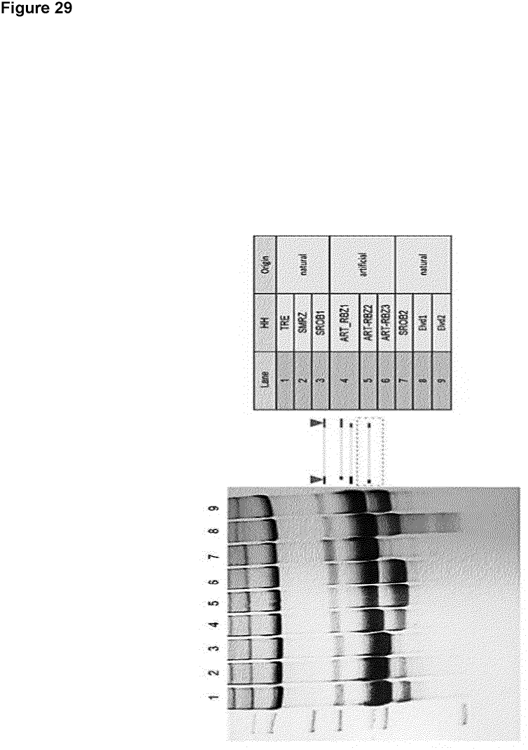

More specifically, circRNAs were produced from a T7-derived plasmid containing the circRNA of interest surrounded in both ends by ribozymes which are RNA molecules with self-cleavage capacity. We used at the 5′ the eggplant latent viroid (ELVd) and at the 3′, the coral one, as the pair of hammerheads. As we saw lower cleavage efficiency in the 5′ hammerhead, we tested several ribozymes both natural and artificial in order to decide which pair of hammerheads were more efficient. As seen in the FIG. 29, all hammerheads tested except number 3 and 7 produced the desired cleaved RNA molecule with different cleavage efficiencies being hammerheads 5-6-9 the best ones. After extracting the bands of the corresponding cleaved RNA molecule from the gel, we saw similar amounts in the three hammerheads, however, we decided to use the artificial ribozyme 3 (ART-RBZ3) as the hammerhead was behaving the same way as in the predictions in silico.

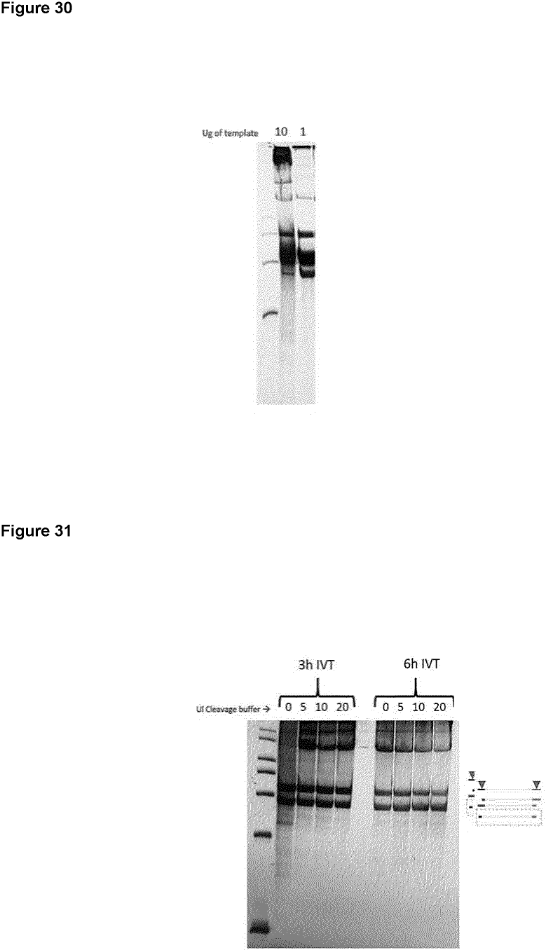

We, then, tested whether we could increase the ratio of partially cleaved/completely cleaved using different concentrations of magnesium in buffers but we did not observe any difference (FIG. 30).

Once the hammerheads were established and to continue with the production of our desired circRNA, we cloned the circRNA sequence into the plasmid and we linearized 10 micrograms using Hind III (Fast Digest) following manufacturer’s instructions. The linearized DNA was phenol-chloroform purified and further in vitro transcribed. Moreover, we tested the optimal amount of linearized plasmid to use as a template and the reaction time. We saw that using either 10 micrograms or 1 microgram of linearized DNA, we obtained the same quantity of cleaved RNA molecule using T7 RNA polymerase (Takara) either incubating 6h (as recommended by the manufacturer’s) or 3h (FIG. 31). Thus, we decided to do the IVT using 1 microgram of template and 3h of reaction.

The IVT was stopped and ran in a urea gel for 4 hours and the desired band (complete cleavage) was cut and RNA was further isolated from there. This last step generates a low yield of the recovered RNA and requires plenty of time to generate sufficient linear molecule to further continue with the protocol. To improve this, we purified the IVT by Chloroform/Isoamyl alcohol extraction instead of running a gel with better results in terms of recovery.

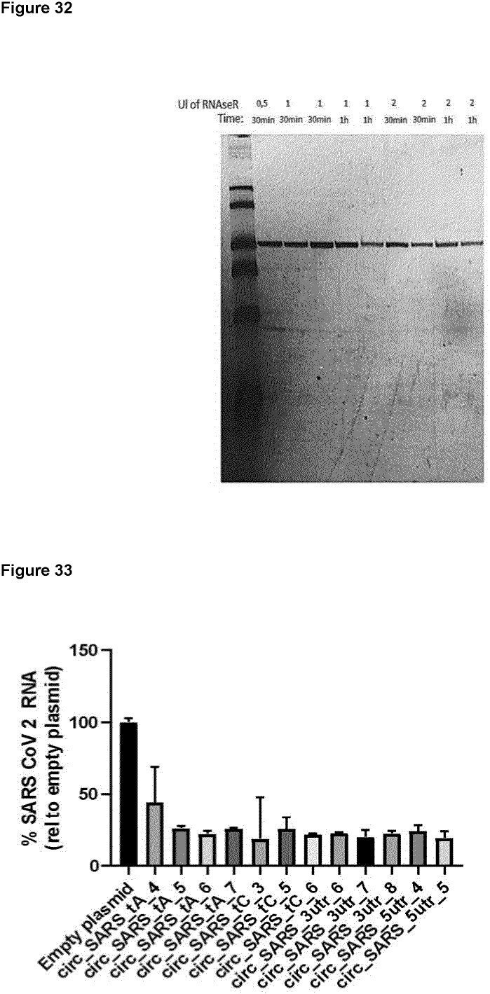

Next, we proceed with the circularization of 10 micrograms of purified linear RNA using RtcB (New England Biolabs) during 1 hour at 37° C. To eliminate non-circular RNA forms, the resulting RNA was RNAse R (Lucigen) treated and purified following manufacturer’s instructions. In order to validate the RNAse R treatment, we ran a urea gel and selected the circRNA band. As said before, the urea gel purification caused a significant loss of RNA material. Therefore, we decided to avoid the gel purification, increasing the RNAse R conditions (FIG. 32) and purifying as done before in the protocol obtaining higher amounts of circRNA. This change in the protocol (see FIG. 25) is key to achieve significant amounts of circRNA at reasonable cost and time. Finally, the presence of circRNA was verified with a TBE-Urea gel only using 1% of the material.

### Mode of Action

The artificial RNAs, such as circRNAs, are designed so that they hybridize with selected regions (target hybridization regions) present in target disruption structures of one or more RNA fragments, such as an RNA viral genome. These target disruption structures are relevant to the functionality of the RNA fragments, as described above. For instance, if the RNA fragment is RNA viral genome, the target disruption structures may be relevant for multiple functions of the viral RNA and infectivity such as translation, replication, localization or/and encapsidation. These signals can be found in the 5′ and 3′untranslated regions and in the coding sequence region (CDS) of the viral RNA genomes. In one embodiment, these target disruption structures can be located within the IRES element (Internal Ribosomal Entry Site), which is responsible for sequestering the host ribosomes to initiate translation of the viral proteins for some viral RNA genomes.

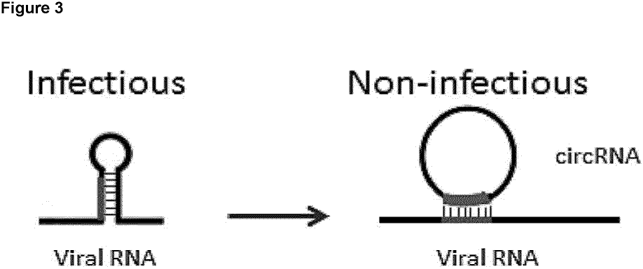

The mechanism of action is NOT just the hybridization between the hybridization regions of the artificial RNA and the target hybridization regions of the target disruption structures, in contrast to the state of the art, but the structural changes triggered upon this hybridization will alter the secondary structure of the RNA fragment, ultimately affecting its function. For instance, viral RNA genomes and viral mRNAs contain highly structured regions essential for their function. Binding of the designed artificial RNAs to target disruption structures in the viral RNA genome will lead to the unfolding of the structure (changes in the secondary structure) and consequently to the reduction or even inhibition of the infectivity (see, e.g., FIG. 3).

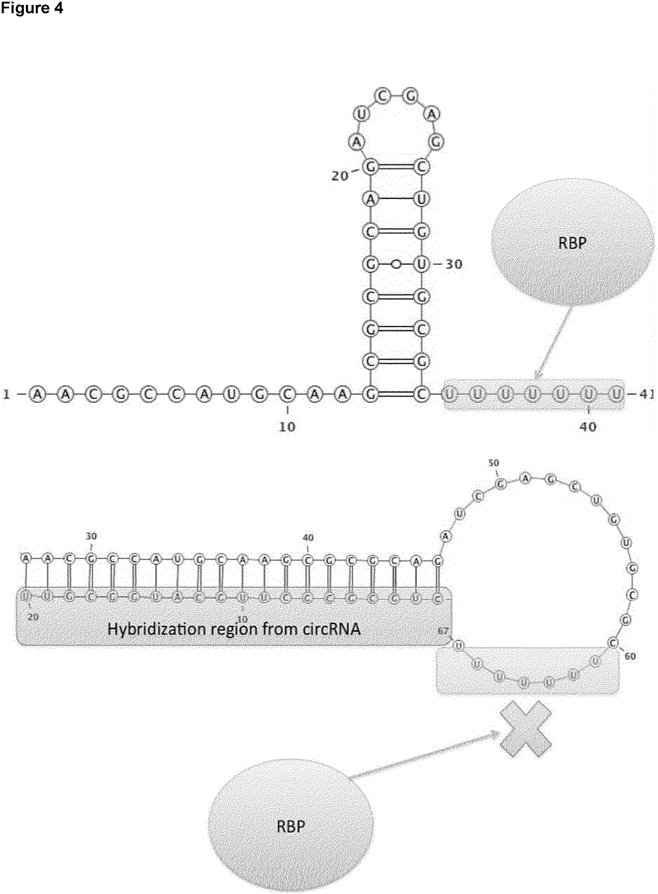

For instance, the disruption of the target disruption structures in the RNA viral genome can disturb the accessibility of RNA binding proteins (RBP) to their target viral RNA sequences. Note that this strategy differs from that whose aim is hybridizing the exact binding site of an RBP (see, e.g., FIG. 4).

Sometimes, RBPs bind single stranded RNA regions that require to be found in a specific structural context, for instance, immediately 3′ of a stem loop (see, e.g., FIG. 4, left side). In our case, hybridizing with a different target hybridization region might disrupt this stem loop (which is part of a target disruption structure) and, although the binding site remains accessible, the structural change renders RBP binding ineffective (see, e.g., FIG. 4, right side).

### Structure

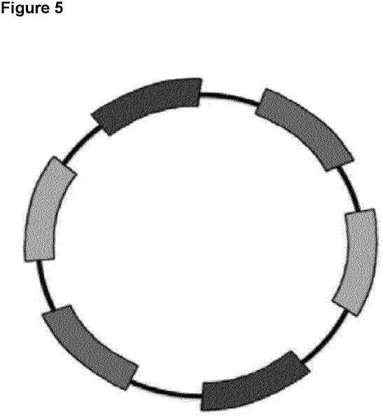

The structure of the designed artificial RNAs, such as circRNAs, is very flexible. Generally, it contains at least one, preferably several hybridization regions that hybridize with at least one, preferably several target hybridization regions present in at least one, preferably several target disruption structures of one or more RNA fragments, preferably in the viral genome. These preferably several hybridization regions in the artificial RNA are preferably separated by sequences that are quasi-random, in the sense that they do not perform any functions other than separate the preferably several hybridization regions, and to allow for the observance of the design constraints (low secondary structure, specific GC content, etc.). For instance, an example of an artificial RNA, which is a circRNA, that targets 3 different target disruption structures of the viral genome with 2 hybridization regions per target disruption structure is depicted in FIG. 5. Regions with the same color represent the fact that they target the same viral target disruption structure.

However, as explained above, these preferably several hybridization regions of the artificial RNA, such as circRNA, that target the same target hybridization region within a certain target disruption structure are preferably designed to be different from each other. This is possible since RNA allows for three different base-pairing types: G-C, A-U and G-U. This fact also entails that, e.g., viral escape (through mutations) from the artificial RNAs is more difficult since it will need to escape all the different hybridization regions at the same time. Note that the hybridization requirement is different from the anti-sense or complementarity requirement.

It is also possible to design the hybridization regions of a single artificial RNA in order to target different regions of different RNA fragments, such as different regions of different RNA viruses to obtain broad-spectrum artificial RNAs.

In a sixth aspect, the present invention provides a method of screening for artificial circular RNA comprising two or more hybridization regions capable of disrupting by hybridization one or more target disruption structures of one or more RNA fragments, wherein the target disruption structures are defined as comprising:
i. a first region with at least a hairpin loop preceded or followed by a second region of unpaired nucleotides; and
ii. at least one target hybridization region which comprises a single-stranded region of at least 2 nucleotides, preferably 3 nucleotides or more preceded or followed by a double-stranded region of at least 5 nucleotides, preferably 10 nucleotides or more, and wherein the method comprises the steps of:
   a) identifying the two or more hybridization regions of the artificial circular RNA as those regions that have a total of between 7 and 100 nucleotides in length, preferably between 10 and 50 nucleotides that, when hybridizing with the at least one target hybridization region, the energy of the hybridization between the two or more hybridization regions and the at least one target hybridization region is more negative than the energy of the target disruption structure, thereby disrupting the one or more target disruption structure, wherein each of the the two or more hybridization regions comprised in the artificial circular RNA, are identified by RNA inverse folding tools, such as NUPACK, RNAifold, or MoiRNAiFold.,
   b) designing an artificial circular RNA comprising the two or more hybridization regions capable of disrupting the one or more target disruption structures as identified in step a), wherein said artificial circular RNA is between 150 and 800 nucleotides in length, preferably between 200 and 600 nucleotides,
   c) optionally selecting the artificial circular RNA capable of disrupting by hybridization the one or more target disruption structures as designed in step b), and optionally packaging it into a product.

As indicated above, in step a) of the method according to the sixth aspect or any of its embodiments, the identification of the two or more hybridization regions according to step a) is performed by RNA inverse folding tools such as NUPACK, RNAifold, MoiRNAiFold, wherein the method preferably comprises the steps of:
a) generating one or more nucleotide sequences of the possible two or more hybridization regions that completely hybridize with the at least one target hybridization region comprised in the one or more target disruption structures of the one or more RNA fragments by providing the RNA inverse folding tools with at least both the target disruption structure/sequence and the target hybridization region,
b) obtaining as output only the one or more nucleotide sequences of those hybridization regions that, when hybridizing with the target hybridization region, the energy of the hybridization between the two or more hybridization region and the at least one target hybridization region is more negative than the energy of the target disruption region, thereby disrupting the target disruption structure.

In a preferred embodiment of the method according to the sixth aspect or any of its embodiments, the method comprises a pre-step that comprises identifying as a target disruption structure those structures of one or more RNA fragments that comprise:
a) a first region with at least a hairpin loop preceded or followed by a second region of unpaired nucleotides; and
b) at least one target hybridization region which comprises a single-stranded region of at least 2 nucleotides, preferably 3 nucleotides or more preceded or followed by a double-stranded region of at least 5 nucleotides, preferably 10 nucleotides or more.

In a preferred embodiment of the method according to the sixth aspect or any of its embodiments, the method comprises a further step d) of confirming in a biological setting the capacity of the designed artificial circular RNA as designed in step b) of disrupting by hybridization one or more target disruption structures of one or more RNA fragments.

In a preferred embodiment, the method according to the sixth aspect or any of its embodiments comprises the use of the target disruption structures and the target hybridization regions defined above in Tables 1 to 7.

The present invention is now further illustrated by reference to the following examples which do not intend to limit the scope of the present invention.

### SEQUENCE LISTING

SEQ ID NO: 1

<211> 9678
<212> RNA
<213> Hepatitis C virus
<220>
<221> misc_ feature
<222> 24..56
<223> /note=“target region of the 5′UTR”
<220>
<221> misc_ feature
<222> 323..346
<223> /note=“target region of the 5′UTR”
<220>
<221> misc_ feature
<222> 372..399
<223> /note=“target region of the CDS”
<220>
<221> misc_ feature
<222> 459..474
<223> /note=“target region of the CDS”
<220>
<221> misc_ feature
<222> 651..668
<223> /note=“target region of the CDS”
<220>
<221> misc_ feature
<222> 9567..9600
<223> /note=“target region of the 3′UTR”
<400> 1

accugccccu aauaggggcg acacuccgcc augaaucacu ccccugugag gaacuacugu 60
cuucacgcag aaagcgccua gccauggcgu uaguaugagu gucguacagc cuccaggccc 120
cccccucccg ggagagccau aguggucugc ggaaccggug aguacaccgg aauugccggg 180
aagacugggu ccuuucuugg auaaacccac ucuaugcccg gccauuuggg cgugcccccg 240
caagacugcu agccgaguag cguuggguug cgaaaggccu ugugguacug ccugauaggg 300
cgcuugcgag ugccccggga ggucucguag accgugcacc augagcacaa auccuaaacc 360
ucaaagaaaa accaaaagaa acaccaaccg ucgcccagaa gacguuaagu ucccgggcgg 420
cggccagauc guuggcggag uauacuuguu gccgcgcagg ggccccaggu ugggugugcg 480
cacgacaagg aaaacuucgg agcgguccca gccacguggg agacgccagc ccauccccaa 540
agaucggcgc uccacuggca aggccugggg aaaaccaggu cgccccuggc cccuauaugg 600
gaaugaggga cucggcuggg caggauggcu ccuguccccc cgaggcucuc gccccuccug 660
gggccccacu gacccccggc auaggucgcg caacgugggu aaagucaucg acacccuaac 720
guguggcuuu gccgaccuca ugggguacau ccccgucgua ggcgccccgc uuaguggcgc 780
cgccagagcu gucgcgcacg gcgugagagu ccuggaggac gggguuaauu augcaacagg 840
gaaccuaccc gguuuccccu uuucuaucuu cuugcuggcc cuguuguccu gcaucaccgu 900
uccggucucu gcugcccagg ugaagaauac caguagcagc uacaugguga ccaaugacug 960
cuccaaugac agcaucacuu ggcagcucga ggcugcgguu cuccacgucc ccgggugcgu 1020
cccgugcgag agagugggga auacgucacg guguugggug ccagucucgc caaacauggc 1080
ugugcggcag cccggugccc ucacgcaggg ucugcggacg cacaucgaua ugguugugau 1140
guccgccacc uucugcucug cucucuacgu gggggaccuc uguggcgggg ugaugcucgc 1200
ggcccaggug uucaucgucu cgccgcagua ccacugguuu gugcaagaau gcaauugcuc 1260
caucuacccu ggcaccauca cuggacaccg cauggcaugg gacaugauga ugaacugguc 1320
gcccacggcc accaugaucc uggcguacgu gaugcgcguc cccgagguca ucauagacau 1380
cguuagcggg gcucacuggg gcgucauguu cggcuuggcc uacuucucua ugcagggagc 1440
gugggcgaag gucauuguca uccuucugcu ggccgcuggg guggacgcgg gcaccaccac 1500
cguuggaggc gcuguugcac guuccaccaa cgugauugcc ggcguguuca gccauggccc 1560
ucagcagaac auucagcuca uuaacaccaa cggcaguugg cacaucaacc guacugccuu 1620
gaauugcaau gacuccuuga acaccggcuu ucucgcggcc uuguucuaca ccaaccgcuu 1680
uaacucguca ggguguccag ggcgccuguc cgccugccgc aacaucgagg cuuuccggau 1740
aggguggggc acccuacagu acgaggauaa ugucaccaau ccagaggaua ugaggccgua 1800
cugcuggcac uaccccccaa agccgugugg cguagucccc gcgaggucug uguguggccc 1860
aguguacugu uucaccccca gcccgguagu agugggcacg accgacagac guggagugcc 1920
caccuacaca uggggagaga augagacaga ugucuuccua cugaacagca cccgaccgcc 1980
gcagggcuca ugguucggcu gcacguggau gaacuccacu gguuucacca agacuugugg 2040
cgcgccaccu ugccgcacca gagcugacuu caacgccagc acggacuugu ugugcccuac 2100
ggauuguuuu aggaagcauc cugaugccac uuauauuaag ugugguucug ggcccuggcu 2160
cacaccaaag ugccuggucc acuacccuua cagacucugg cauuaccccu gcacagucaa 2220
uuuuaccauc uucaagauaa gaauguaugu aggggggguu gagcacaggc ucacggccgc 2280
augcaacuuc acucgugggg aucgcugcga cuuggaggac agggacagga gucagcuguc 2340
uccucuguug cacucuacca cggaaugggc cauccugccc ugcaccuacu cagacuuacc 2400
cgcuuuguca acuggucuuc uccaccuuca ccagaacauc guggacguac aauacaugua 2460
uggccucuca ccugcuauca caaaauacgu cguucgaugg gagugggugg uacucuuauu 2520
ccugcucuua gcggacgcca gagucugcgc cugcuugugg augcucaucu uguugggcca 2580
ggccgaagca gcauuggaga aguuggucgu cuugcacgcu gcgagugcgg cuaacugcca 2640
uggccuccua uauuuugcca ucuucuucgu ggcagcuugg cacaucaggg gucggguggu 2700
ccccuugacc accuauugcc ucacuggccu auggcccuuc ugccuacugc ucauggcacu 2760
gccccggcag gcuuaugccu augacgcacc ugugcacgga cagauaggcg uggguuuguu 2820
gauauugauc acccucuuca cacucacccc gggguauaag acccuccucg gccagugucu 2880
guggugguug ugcuaucucc ugacccuggg ggaagccaug auucaggagu ggguaccacc 2940
caugcaggug cgcggcggcc gcgauggcau cgcgugggcc gucacuauau ucugcccggg 3000
ugugguguuu gacauuacca aauggcuuuu ggcguugcuu gggccugcuu accucuuaag 3060
ggccgcuuug acacaugugc cguacuucgu cagagcucac gcucugauaa ggguaugcgc 3120
uuuggugaag cagcucgcgg gggguaggua uguucaggug gcgcuauugg cccuuggcag 3180
guggacuggc accuacaucu augaccaccu cacaccuaug ucggacuggg ccgcuagcgg 3240
ccugcgcgac uuagcggucg ccguggaacc caucaucuuc aguccgaugg agaagaaggu 3300
caucgucugg ggagcggaga cggcugcaug uggggacauu cuacauggac uucccguguc 3360
cgcccgacuc ggccaggaga uccuccucgg cccagcugau ggcuacaccu ccaaggggug 3420
gaagcuccuu gcucccauca cugcuuaugc ccagcaaaca cgaggccucc ugggcgccau 3480
aguggugagu augacggggc gugacaggac agaacaggcc ggggaagucc aaauccuguc 3540
cacagucucu caguccuucc ucggaacaac caucucgggg guuuugugga cuguuuacca 3600
cggagcuggc aacaagacuc uagccggcuu acgggguccg gucacgcaga uguacucgag 3660
ugcugagggg gacuugguag gcuggcccag ccccccuggg accaagucuu uggagccgug 3720
caagugugga gccgucgacc uauaucuggu cacgcggaac gcugauguca ucccggcucg 3780
gagacgcggg gacaagcggg gagcauugcu cuccccgaga cccauuucga ccuugaaggg 3840
guccucgggg gggccggugc ucugcccuag gggccacguc guugggcucu uccgagcagc 3900
ugugugcucu cggggcgugg ccaaauccau cgauuucauc cccguugaga cacucgacgu 3960
uguuacaagg ucucccacuu ucagugacaa cagcacgcca ccggcugugc cccagaccua 4020
ucaggucggg uacuugcaug cuccaacugg caguggaaag agcaccaagg ucccugucgc 4080
guaugccgcc cagggguaca aaguacuagu gcuuaacccc ucgguagcug ccacccuggg 4140
guuuggggcg uaccuaucca aggcacaugg caucaauccc aacauuagga cuggagucag 4200
gaccgugaug accggggagg ccaucacgua cuccacauau ggcaaauuuc ucgccgaugg 4260
gggcugcgcu agcggcgccu augacaucau cauaugcgau gaaugccacg cuguggaugc 4320
uaccuccauu cucggcaucg gaacgguccu ugaucaagca gagacagccg gggucagacu 4380
aacugugcug gcuacggcca caccccccgg gucagugaca accccccauc ccgauauaga 4440
agagguaggc cucgggcggg agggugagau ccccuucuau gggagggcga uuccccuauc 4500
cugcaucaag ggagggagac accugauuuu cugccacuca aagaaaaagu gugacgagcu 4560
cgcggcggcc cuucggggca ugggcuugaa ugccguggca uacuauagag gguuggacgu 4620
cuccauaaua ccagcucagg gagauguggu ggucgucgcc accgacgccc ucaugacggg 4680
guacacugga gacuuugacu ccgugaucga cugcaaugua gcggucaccc aagcugucga 4740
cuucagccug gaccccaccu ucacuauaac cacacagacu gucccacaag acgcugucuc 4800
acgcagucag cgccgcgggc gcacagguag aggaagacag ggcacuuaua gguauguuuc 4860
cacuggugaa cgagccucag gaauguuuga caguguagug cuuugugagu gcuacgacgc 4920
aggggcugcg ugguacgauc ucacaccagc ggagaccacc gucaggcuua gagcguauuu 4980
caacacgccc ggccuacccg ugugucaaga ccaucuugaa uuuugggagg caguuuucac 5040
cggccucaca cacauagacg cccacuuccu cucccaaaca aagcaagcgg gggagaacuu 5100
cgcguaccua guagccuacc aagcuacggu gugcgccaga gccaaggccc cucccccguc 5160
cugggacgcc auguggaagu gccuggcccg acucaagccu acgcuugcgg gccccacacc 5220
ucuccuguac cguuugggcc cuauuaccaa ugaggucacc cucacacacc cugggacgaa 5280
guacaucgcc acaugcaugc aagcugaccu ugaggucaug accagcacgu ggguccuagc 5340
uggaggaguc cuggcagccg ucgccgcaua uugccuggcg acuggaugcg uuuccaucau 5400
cggccgcuug cacgucaacc agcgagucgu cguugcgccg gauaaggagg uccuguauga 5460
ggcuuuugau gagauggagg aaugcgccuc uagggcggcu cucaucgaag aggggcagcg 5520
gauagccgag auguugaagu ccaagaucca aggcuugcug cagcaggccu cuaagcaggc 5580
ccaggacaua caacccgcua ugcaggcuuc auggcccaaa guggaacaau uuugggccag 5640
acacaugugg aacuucauua gcggcaucca auaccucgca ggauugucaa cacugccagg 5700
gaaccccgcg guggcuucca ugauggcauu cagugccgcc cucaccaguc cguugucgac 5760
caguaccacc auccuucuca acaucauggg aggcugguua gcgucccaga ucgcaccacc 5820
cgcgggggcc accggcuuug ucgucagugg ccuggugggg gcugccgugg gcagcauagg 5880
ccuggguaag gugcuggugg acauccuggc aggauauggu gcgggcauuu cgggggcccu 5940
cgucgcauuc aagaucaugu cuggcgagaa gcccucuaug gaagauguca ucaaucuacu 6000
gccugggauc cugucuccgg gagcccuggu gguggggguc aucugcgcgg ccauucugcg 6060
ccgccacgug ggaccggggg agggcgcggu ccaauggaug aacaggcuua uugccuuugc 6120
uuccagagga aaccacgucg ccccuacuca cuacgugacg gagucggaug cgucgcagcg 6180
ugugacccaa cuacuuggcu cucuuacuau aaccagccua cucagaagac uccacaauug 6240
gauaacugag gacugcccca ucccaugcuc cggauccugg cuccgcgacg ugugggacug 6300
gguuugcacc aucuugacag acuucaaaaa uuggcugacc ucuaaauugu uccccaagcu 6360
gcccggccuc cccuucaucu cuugucaaaa gggguacaag gguguguggg ccggcacugg 6420
caucaugacc acgcgcugcc cuugcggcgc caacaucucu ggcaaugucc gccugggcuc 6480
uaugaggauc acagggccua aaaccugcau gaacaccugg caggggaccu uuccuaucaa 6540
uugcuacacg gagggccagu gcgcgccgaa accccccacg aacuacaaga ccgccaucug 6600
gaggguggcg gccucggagu acgcggaggu gacgcagcau gggucguacu ccuauguaac 6660
aggacugacc acugacaauc ugaaaauucc uugccaacua ccuucuccag aguuuuucuc 6720
cuggguggac ggugugcaga uccauagguu ugcacccaca ccaaagccgu uuuuccggga 6780
ugaggucucg uucugcguug ggcuuaauuc cuaugcuguc gggucccagc uucccuguga 6840
accugagccc gacgcagacg uauugagguc caugcuaaca gauccgcccc acaucacggc 6900
ggagacugcg gcgcggcgcu uggcacgggg aucaccucca ucugaggcga gcuccucagu 6960
gagccagcua ucagcaccgu cgcugcgggc caccugcacc acccacagca acaccuauga 7020
cguggacaug gucgaugcca accugcucau ggagggcggu guggcucaga cagagccuga 7080
guccagggug cccguucugg acuuucucga gccaauggcc gaggaagaga gcgaccuuga 7140
gcccucaaua ccaucggagu gcaugcuccc caggagcggg uuuccacggg ccuuaccggc 7200
uugggcacgg ccugacuaca acccgccgcu cguggaaucg uggaggaggc cagauuacca 7260
accgcccacc guugcugguu gugcucuccc cccccccaag aaggccccga cgccuccccc 7320
aaggagacgc cggacagugg gucugagcga gagcaccaua ucagaagccc uccagcaacu 7380
ggccaucaag accuuuggcc agccccccuc gagcggugau gcaggcucgu ccacgggggc 7440
gggcgccgcc gaauccggcg guccgacguc cccuggugag ccggcccccu cagagacagg 7500
uuccgccucc ucuaugcccc cccucgaggg ggagccugga gauccggacc uggagucuga 7560
ucagguagag cuucaaccuc ccccccaggg ggggggggua gcucccgguu cgggcucggg 7620
gucuuggucu acuugcuccg aggaggacga uaccaccgug ugcugcucca ugucauacuc 7680
cuggaccggg gcucuaauaa cucccuguag ccccgaagag gaaaaguugc caaucaaccc 7740
uuugaguaac ucgcuguugc gauaccauaa caagguguac uguacaacau caaagagcgc 7800
cucacagagg gcuaaaaagg uaacuuuuga caggacgcaa gugcucgacg cccauuauga 7860
cucagucuua aaggacauca agcuagcggc uuccaagguc agcgcaaggc uccucaccuu 7920
ggaggaggcg ugccaguuga cuccacccca uucugcaaga uccaaguaug gauucggggc 7980
caaggagguc cgcagcuugu ccgggagggc cguuaaccac aucaaguccg uguggaagga 8040
ccuccuggaa gacccacaaa caccaauucc cacaaccauc auggccaaaa augagguguu 8100
cugcguggac cccgccaagg gggguaagaa accagcucgc cucaucguuu acccugaccu 8160
cggcguccgg gucugcgaga aaauggcccu cuaugacauu acacaaaagc uuccucaggc 8220
gguaauggga gcuuccuaug gcuuccagua cuccccugcc caacgggugg aguaucucuu 8280
gaaagcaugg gcggaaaaga aggaccccau ggguuuuucg uaugauaccc gaugcuucga 8340
cucaaccguc acugagagag acaucaggac cgaggagucc auauaccagg ccugcucccu 8400
gcccgaggag gcccgcacug ccauacacuc gcugacugag agacuuuacg uaggagggcc 8460
cauguucaac agcaaggguc aaaccugcgg uuacagacgu ugccgcgcca gcggggugcu 8520
aaccacuagc auggguaaca ccaucacaug cuaugugaaa gcccuagcgg ccugcaaggc 8580
ugcggggaua guugcgccca caaugcuggu augcggcgau gaccuaguag ucaucucaga 8640
aagccagggg acugaggagg acgagcggaa ccugagagcc uucacggagg ccaugaccag 8700
guacucugcc ccuccuggug auccccccag accggaauau gaccuggagc uaauaacauc 8760
cuguuccuca aaugugucug uggcguuggg cccgcggggc cgccgcagau acuaccugac 8820
cagagaccca accacuccac ucgcccgggc ugccugggaa acaguuagac acuccccuau 8880
caauucaugg cugggaaaca ucauccagua ugcuccaacc auauggguuc gcaugguccu 8940
aaugacacac uucuucucca uucucauggu ccaagacacc cuggaccaga accucaacuu 9000
ugagauguau ggaucaguau acuccgugaa uccuuuggac cuuccagcca uaauugagag 9060
guuacacggg cuugacgccu uuucuaugca cacauacucu caccacgaac ugacgcgggu 9120
ggcuucagcc cucagaaaac uuggggcgcc accccucagg guguggaaga gucgggcucg 9180
cgcagucagg gcgucccuca ucucccgugg agggaaagcg gccguuugcg gccgauaucu 9240
cuucaauugg gcggugaaga ccaagcucaa acucacucca uugccggagg cgcgccuacu 9300
ggacuuaucc aguugguuca ccgucggcgc cggcgggggc gacauuuuuc acagcguguc 9360
gcgcgcccga ccccgcucau uacucuucgg ccuacuccua cuuuucguag ggguaggccu 9420
cuuccuacuc cccgcucggu agagcggcac acacuaggua cacuccauag cuaacuguuc 9480
cuuuuuuuuu uuuuuuuuuu uuuuuuuuuu uuuuuuuuuu uuuucuuuuu uuuuuuuuuc 9540
ccucuuucuu cccuucucau cuuauucuac uuucuuucuu gguggcucca ucuuagcccu 9600
agucacggcu agcugugaaa gguccgugag ccgcaugacu gcagagagug ccguaacugg 9660
ucucucugca gaucaugu 9678

SEQ ID NO: 2

<211> 294
<212> RNA
<213> Artificial sequence
<220>
<223> Circular Artificial sequence to IRES1 (hepatitis C virus)
(circ_hcv_ires1)
<220>
<221> misc_ feature
<222> 1..33
<223> /note=“hybridization site to IRES (hepatitis c virus)”
<220>
<221> misc_ feature
<222> 1..33
<223> /note=“hybridization site to IRES (hepatitis c virus)”
<220>
<221> misc_ feature
<222> 43..75
<223> /note=“hybridization site to IRES (hepatitis c virus)”
<220>
<221> misc_ feature
<222> 85..117
<223> /note=“hybridization site to IRES (hepatitis c virus)”
<220>
<221> misc_ feature
<222> 127..159
<223> /note=“hybridization site to IRES (hepatitis c virus)”
<220>
<221> misc_ feature
<222> 169..201
<223> /note=“hybridization site to IRES (hepatitis c virus)”
<220>
<221> misc_ feature
<222> 169..201
<223> /note=“hybridization site to IRES (hepatitis c virus)”
<220>
<221> misc_ feature
<222> 211..243
<223> /note=“hybridization site to IRES (hepatitis c virus)”
<220>
<221> misc_ feature
<222> 253..285
<223> /note=“hybridization site to IRES (hepatitis c virus)”
<400> 2

uaguuccuca cgggggagug guucguggug gagcggcgcc aaugguuuuu ugcgggggag 60
ugguucgugg cggggguaac cccuuaguuc uucauggggg ggugguucau gguggggaaa 120
cuaccguagu uuuucacagg ggggugguuc auggcggaga gaagcgcuua guuucuuaug 180
ggggagugau ucauggcgga guguggggca ugguuuuucg caggggagug auucguggug 240
gggagaguuu ugugguucuu uguagggggg ugauucgugg uggggacauc auug 294

SEQ ID NO: 3

<211> 373
<212> RNA
<213> Artificial sequence
<220>
<223> Circular Artificial sequence to IRES2 (hepatitis C virus)
<220>
<221> misc_ feature
<222> 11..34
<223> /note=“hybridization site to IRES (hepatitis c virus)”
<220>
<221> misc_ feature
<222> 44..67
<223> /note=“hybridization site to IRES (hepatitis c virus)”
<220>
<221> misc_ feature
<222> 77..100
<223> /note=“hybridization site to IRES (hepatitis c virus)”
<220>
<221> misc_ feature
<222> 110..123
<223> /note=“hybridization site to IRES (hepatitis c virus)”
<220>
<221> misc_ feature
<222> 133..166
<223> /note=“hybridization site to IRES (hepatitis c virus)”
<220>
<221> misc_ feature
<222> 176..199
<223> /note=“hybridization site to IRES (hepatitis c virus)”
<220>
<221> misc_ feature
<222> 209..232
<223> /note=“hybridization site to IRES (hepatitis c virus)”
<220>
<221> misc_ feature
<222> 242..265
<223> /note=“hybridization site to IRES (hepatitis c virus)”
<220>
<221> misc_ feature
<222> 275..298
<223> /note=“hybridization site to IRES (hepatitis c virus)”
<220>
<221> misc_ feature
<222> 308..331
<223> /note=“hybridization site to IRES (hepatitis c virus)”
<220>
<221> misc_ feature
<222> 341..373
<223> /note=“hybridization site to IRES (hepatitis c virus)”
<400> 3

ugccgaaaau gcuuauggug cacgguuugc gagagcaaga agagcuugug gugcaugguu 60
ugcggggaca guaaaggcuc auggugcaug gucuacgagg ugagauagcg uuuauggugu 120
acggucugug ggaaauuccg guguucgugg ugugcggucu gugggauauu agaucguuca 180
ugguguacgg uuugugggga uaauagcggc uuauggugca cggucuaugg gacgcgagaa 240
ggcucauggu gugugguuug ugaggcagug ugcgguucau ggugcacggu cugcgagaca 300
ccaggcggcu cguggugcac ggucuauggg gugucgucgu gcuuguggug ugcggucuau 360
gagggcggcu gaa 373

SEQ ID NO: 4

<211> 346
<212> RNA
<213> Artificial sequence
<220>
<223> Circular Artificial sequence to CDS1 (hepatitis C virus)
(circ_hcv_cds1 )
<220>
<221> misc_ feature
<222> 11..38
<223> /note=“hybridization site to CDS1 (hepatitis C virus)”
<220>
<221> misc_ feature
<222> 53..80
<223> /note=“hybridization site to CDS1 (hepatitis C virus)”
<220>
<221> misc_ feature
<222> 95..122
<223> /note=“hybridization site to CDS1 (hepatitis C virus)”
<220>
<221> misc_ feature
<222> 137..164
<223> /note=“hybridization site to CDS1 (hepatitis C virus)”
<220>
<221> misc_ feature
<222> 179..206
<223> /note=“hybridization site to CDS1 (hepatitis C virus)”
<220>
<221> misc_ feature
<222> 221..248
<223> /note=“hybridization site to CDS1 (hepatitis C virus)”
<220>
<221> misc_ feature
<222> 263..290
<223> /note=“hybridization site to CDS1 (hepatitis C virus)”
<220>
<221> misc_ feature
<222> 305..332
<223> /note=“hybridization site to CDS1 (hepatitis C virus)”
<400> 4

cugcacuggg uuugggcgac gguugguguu uuuuuuggac gagauuucuu gcucugggcg 60
acgguuggug uuucuuuugg guguuagugc guacucuggg cgaugguugg uguuucuuuu 120
gguaauuauu acuuucuuug ggcgaugguu gguguuuuuu uugguggggu gaaaagaguu 180
ugggcggugg uugguguuuc uuuuggcgug gugaguaaca uuuggguggc gguugguguu 240
uuuuuuggcu gcaacgcacc gguuugggcg acgguuggug uuucuuuugg gggucaaccg 300
ggaaucuggg uggcgguugg uguuuuuuuu ggugauggcc gaggua 346

SEQ ID NO: 5

<211> 355
<212> RNA
<213> Artificial sequence
<220>
<223> Circular Artificial sequence to IRES1, IRES2 and CDS1 (hepatitis
C virus) (circ_hcv_combo1)
<220>
<221> misc_ feature
<222> 11..43
<223> /note=“hybridization site to IRES1 (hepatitis C virus)”
<220>
<221> misc_ feature
<222> 54..77
<223> /note=“hybridization site to IRES2 (hepatitis C virus)”
<220>
<221> misc_ feature
<222> 88..115
<223> /note=“hybridization site to CDS1 (hepatitis C virus)”
<220>
<221> misc_ feature
<222> 126..158
<223> /note=“hybridization site to IRES1 (hepatitis C virus)”
<220>
<221> misc_ feature
<222> 169..192
<223> /note=“hybridization site to IRES2 (hepatitis C virus)”
<220>
<221> misc_ feature
<222> 203..230
<223> /note=“hybridization site to CDS1 (hepatitis C virus)”
<220>
<221> misc_ feature
<222> 241..273
<223> /note=“hybridization site to IRES1 (hepatitis C virus)”
<220>
<221> misc_ feature
<222> 284..307
<223> /note=“hybridization site to IRES2 (hepatitis C virus)”
<220>
<221> misc_ feature
<222> 318..345
<223> /note=“hybridization site to IRES2 (hepatitis C virus)”
<400> 5

gcgccaagua uaguuccuca caggggagug auuuauggug gagaccccua aacgcuugug 60
gugcacgguc uacgggguac cgagaaguuu gggcgguggu ugguguuuuu uuuggcgcuu 120
gugggugguu uuuugcaggg gagugauuua ugguggaggc aagaguuugc uuauggugua 180
cggucuguga gaugacauca gguuugggcg gcgguuggug uuuuuuuugg augaggaacc 240
uaguucuuca uaggggagug auuuguggug gagacagcga guuguuugug gugcacgguu 300
uacgagaaga augaagaucu ggguggcggu ugguguuucu uuugguagca caugu 355

SEQ ID NO: 6

<211> 288
<212> RNA
<213> Artificial sequence
<220>
<223> Circular Artificial sequence to CDS2 (hepatitis C virus)
(circ_hcv_cds2)
<220>
<221> misc_ feature
<222> 4..14
<223> /note=“hybridization site to CDS2 (hepatitis C virus)”
<220>
<221> misc_ feature
<222> 28..38
<223> /note=“hybridization site to CDS2 (hepatitis C virus)”
<220>
<221> misc_ feature
<222> 52..62
<223> /note=“hybridization site to CDS2 (hepatitis C virus)”
<220>
<221> misc_ feature
<222> 76..86
<223> /note=“hybridization site to CDS2 (hepatitis C virus)”
<220>
<221> misc_ feature
<222> 100..110
<223> /note=“hybridization site to CDS2 (hepatitis C virus)”
<220>
<221> misc_ feature
<222> 124..134
<223> /note=“hybridization site to CDS2 (hepatitis C virus)”
<220>
<221> misc_ feature
<222> 148..158
<223> /note=“hybridization site to CDS2 (hepatitis C virus)”
<220>
<221> misc_ feature
<222> 172..182
<223> /note=“hybridization site to CDS2 (hepatitis C virus)”
<220>
<221> misc_ feature
<222> 196..206
<223> /note=“hybridization site to CDS2 (hepatitis C virus)”
<220>
<221> misc_ feature
<222> 220..230
<223> /note=“hybridization site to CDS2 (hepatitis C virus)”
<220>
<221> misc_ feature
<222> 244..254
<223> /note=“hybridization site to CDS2 (hepatitis C virus)”
<220>
<221> misc_ feature
<222> 266..278
<223> /note=“hybridization site to CDS2 (hepatitis C virus)”
<400> 6

cccccugggg cucugaugag gaaccucucu ggggucccca cagcgagucu ccuuggggcc 60
ccuagaauga aguucuuugg gguuucauag cacgguucuc cugggguuuu cauacgauug 120
ucucuugggg ucuugcgugu uuacccuuuu gggguccugc uaagggggcu uucuggggcc 180
uuucgaauaa gucuuuuugg ggccucguuu uaucacucuu cugggguucc cagccuuucc 240
cuucuugggg cuccuccgac caugccccuu gggguucuac ccuguaug 288

SEQ ID NO: 7

<211> 10722
<212> RNA
<213> Dengue virus
<220>
<221> misc_ feature
<222> 108..140
<223> /note=“target region of cHP”
<220>
<221> misc_ feature
<222> 10615..10646
<223> /note=“target region of 3UTR”
<400> 7

aguuguuagu cuacguggac cgacaaagac agauucuuug agggagcuaa gcucaacgua 60
guucuaacag uuuuuuaauu agagagcaga ucucugauga auaaccaacg gaaaaaggcg 120
aaaaacacgc cuuucaauau gcugaaacgc gagagaaacc gcgugucgac ugugcaacag 180
cugacaaaga gauucucacu uggaaugcug cagggacgag gaccauuaaa acuguucaug 240
gcccuggugg cguuccuucg uuuccuaaca aucccaccaa cagcagggau auugaagaga 300
uggggaacaa uuaaaaaauc aaaagcuauu aauguuuuga gaggguucag gaaagagauu 360
ggaaggaugc ugaacaucuu gaauaggaga cgcagaucug caggcaugau cauuaugcug 420
auuccaacag ugauggcguu ccauuuaacc acacguaacg gagaaccaca caugaucguc 480
agcagacaag agaaagggaa aagucuucug uuuaaaacag aggauggcgu gaacaugugu 540
acccucaugg ccauggaccu uggugaauug ugugaagaca caaucacgua caaguguccc 600
cuucucaggc agaaugagcc agaagacaua gacuguuggu gcaacucuac guccacgugg 660
guaacuuaug ggacguguac caccauggga gaacauagaa gagaaaaaag aucaguggca 720
cucguuccac augugggaau gggacuggag acacgaacug aaacauggau gucaucagaa 780
ggggccugga aacaugucca gagaauugaa acuuggaucu ugagacaucc aggcuucacc 840
augauggcag caauccuggc auacaccaua ggaacgacac auuuccaaag agcccugauu 900
uucaucuuac ugacagcugu cacuccuuca augacaaugc guugcauagg aaugucaaau 960
agagacuuug uggaaggggu uucaggagga agcuggguug acauagucuu agaacaugga 1020
agcuguguga cgacgauggc aaaaaacaaa ccaacauugg auuuugaacu gauaaaaaca 1080
gaagccaaac agccugccac ccuaaggaag uacuguauag aggcaaagcu aaccaacaca 1140
acaacagaau cucgcugccc aacacaaggg gaacccagcc uaaaugaaga gcaggacaaa 1200
agguucgucu gcaaacacuc caugguagac agaggauggg gaaauggaug uggacuauuu 1260
ggaaagggag gcauugugac cugugcuaug uucagaugca aaaagaacau ggaaggaaaa 1320
guugugcaac cagaaaacuu ggaauacacc auugugauaa caccucacuc aggggaagag 1380
caugcagucg gaaaugacac aggaaaacau ggcaaggaaa ucaaaauaac accacagagu 1440
uccaucacag aagcagaauu gacagguuau ggcacuguca caauggagug cucuccaaga 1500
acgggccucg acuucaauga gaugguguug cugcagaugg aaaauaaagc uuggcuggug 1560
cacaggcaau gguuccuaga ccugccguua ccaugguugc ccggagcgga cacacaaggg 1620
ucaaauugga uacagaaaga gacauugguc acuuucaaaa auccccaugc gaagaaacag 1680
gauguuguug uuuuaggauc ccaagaaggg gccaugcaca cagcacuuac aggggccaca 1740
gaaauccaaa ugucaucagg aaacuuacuc uucacaggac aucucaagug caggcugaga 1800
auggacaagc uacagcucaa aggaauguca uacucuaugu gcacaggaaa guuuaaaguu 1860
gugaaggaaa uagcagaaac acaacaugga acaauaguua ucagagugca auaugaaggg 1920
gacggcucuc caugcaagau cccuuuugag auaauggauu uggaaaaaag acaugucuua 1980
ggucgccuga uuacagucaa cccaauugug acagaaaaag auagcccagu caacauagaa 2040
gcagaaccuc cauucggaga cagcuacauc aucauaggag uagagccggg acaacugaag 2100
cucaacuggu uuaagaaagg aaguucuauc ggccaaaugu uugagacaac aaugaggggg 2160
gcgaagagaa uggccauuuu aggugacaca gccugggauu uuggauccuu gggaggagug 2220
uuuacaucua uaggaaaggc ucuccaccaa gucuuuggag caaucuaugg agcugccuuc 2280
agugggguuu cauggacuau gaaaauccuc auaggaguca uuaucacaug gauaggaaug 2340
aauucacgca gcaccucacu gucugugaca cuaguauugg ugggaauugu gacacuguau 2400
uugggaguca uggugcaggc cgauaguggu ugcguuguga gcuggaaaaa caaagaacug 2460
aaauguggca gugggauuuu caucacagac aacgugcaca cauggacaga acaauacaag 2520
uuccaaccag aauccccuuc aaaacuagcu ucagcuaucc agaaagccca ugaagagggc 2580
auuuguggaa uccgcucagu aacaagacug gagaaucuga uguggaaaca aauaacacca 2640
gaauugaauc acauucuauc agaaaaugag gugaaguuaa cuauuaugac aggagacauc 2700
aaaggaauca ugcaggcagg aaaacgaucu cugcggccuc agcccacuga gcugaaguau 2760
ucauggaaaa cauggggcaa agcaaaaaug cucucuacag agucucauaa ccagaccuuu 2820
cucauugaug gccccgaaac agcagaaugc cccaacacaa auagagcuug gaauucguug 2880
gaaguugaag acuauggcuu uggaguauuc accaccaaua uauggcuaaa auugaaagaa 2940
aaacaggaug uauucugcga cucaaaacuc augucagcgg ccauaaaaga caacagagcc 3000
guccaugccg auauggguua uuggauagaa agugcacuca augacacaug gaagauagag 3060
aaagccucuu ucauugaagu uaaaaacugc cacuggccaa aaucacacac ccucuggagc 3120
aauggagugc uagaaaguga gaugauaauu ccaaagaauc ucgcuggacc agugucucaa 3180
cacaacuaua gaccaggcua ccauacacaa auaacaggac cauggcaucu agguaagcuu 3240
gagauggacu uugauuucug ugauggaaca acagugguag ugacugagga cugcggaaau 3300
agaggacccu cuuugagaac aaccacugcc ucuggaaaac ucauaacaga auggugcugc 3360
cgaucuugca cauuaccacc gcuaagauac agaggugagg augggugcug guacgggaug 3420
gaaaucagac cauugaagga gaaagaagag aauuugguca acuccuuggu cacagcugga 3480
caugggcagg ucgacaacuu uucacuagga gucuugggaa uggcauuguu ccuggaggaa 3540
augcuuagga cccgaguagg aacgaaacau gcaauacuac uaguugcagu uucuuuugug 3600
acauugauca cagggaacau guccuuuaga gaccugggaa gagugauggu uaugguaggc 3660
gccacuauga cggaugacau agguaugggc gugacuuauc uugcccuacu agcagccuuc 3720
aaagucagac caacuuuugc agcuggacua cucuugagaa agcugaccuc caaggaauug 3780
augaugacua cuauaggaau uguacuccuc ucccagagca ccauaccaga gaccauucuu 3840
gaguugacug augcguuagc cuuaggcaug augguccuca aaauggugag aaauauggaa 3900
aaguaucaau uggcagugac uaucauggcu aucuugugcg ucccaaacgc agugauauua 3960
caaaacgcau ggaaagugag uugcacaaua uuggcagugg uguccguuuc cccacugcuc 4020
uuaacauccu cacagcaaaa aacagauugg auaccauuag cauugacgau caaaggucuc 4080
aauccaacag cuauuuuucu aacaacccuc ucaagaacca gcaagaaaag gagcuggcca 4140
uuaaaugagg cuaucauggc agucgggaug gugagcauuu uagccaguuc ucuccuaaaa 4200
aaugauauuc ccaugacagg accauuagug gcuggagggc uccucacugu gugcuacgug 4260
cucacuggac gaucggccga uuuggaacug gagagagcag ccgaugucaa augggaagac 4320
caggcagaga uaucaggaag caguccaauc cugucaauaa caauaucaga agaugguagc 4380
augucgauaa aaaaugaaga ggaagaacaa acacugacca uacucauuag aacaggauug 4440
cuggugaucu caggacuuuu uccuguauca auaccaauca cggcagcagc augguaccug 4500
ugggaaguga agaaacaacg ggccggagua uugugggaug uuccuucacc cccacccaug 4560
ggaaaggcug aacuggaaga uggagccuau agaauuaagc aaaaagggau ucuuggauau 4620
ucccagaucg gagccggagu uuacaaagaa ggaacauucc auacaaugug gcaugucaca 4680
cguggcgcug uucuaaugca uaaaggaaag aggauugaac caucaugggc ggacgucaag 4740
aaagaccuaa uaucauaugg aggaggcugg aaguuagaag gagaauggaa ggaaggagaa 4800
gaaguccagg uauuggcacu ggagccugga aaaaauccaa gagccgucca aacgaaaccu 4860
ggucuuuuca aaaccaacgc cggaacaaua ggugcuguau cucuggacuu uucuccugga 4920
acgucaggau cuccaauuau cgacaaaaaa ggaaaaguug ugggucuuua ugguaauggu 4980
guuguuacaa ggaguggagc auaugugagu gcuauagccc agacugaaaa aagcauugaa 5040
gacaacccag agaucgaaga ugacauuuuc cgaaagagaa gacugaccau cauggaccuc 5100
cacccaggag cgggaaagac gaagagauac cuuccggcca uagucagaga agcuauaaaa 5160
cgggguuuga gaacauuaau cuuggccccc acuagaguug uggcagcuga aauggaggaa 5220
gcccuuagag gacuuccaau aagauaccag accccagcca ucagagcuga gcacaccggg 5280
cgggagauug uggaccuaau gugucaugcc acauuuacca ugaggcugcu aucaccaguu 5340
agagugccaa acuacaaccu gauuaucaug gacgaagccc auuucacaga cccagcaagu 5400
auagcagcua gaggauacau cucaacucga guggagaugg gugaggcagc ugggauuuuu 5460
augacagcca cucccccggg aagcagagac ccauuuccuc agagcaaugc accaaucaua 5520
gaugaagaaa gagaaauccc ugaacguucg uggaauuccg gacaugaaug ggucacggau 5580
uuuaaaggga agacuguuug guucguucca aguauaaaag caggaaauga uauagcagcu 5640
ugccugagga aaaauggaaa gaaagugaua caacucagua ggaagaccuu ugauucugag 5700
uaugucaaga cuagaaccaa ugauugggac uucgugguua caacugacau uucagaaaug 5760
ggugccaauu ucaaggcuga gaggguuaua gaccccagac gcugcaugaa accagucaua 5820
cuaacagaug gugaagagcg ggugauucug gcaggaccua ugccagugac ccacucuagu 5880
gcagcacaaa gaagagggag aauaggaaga aauccaaaaa augagaauga ccaguacaua 5940
uacauggggg aaccucugga aaaugaugaa gacugugcac acuggaaaga agcuaaaaug 6000
cuccuagaua acaucaacac gccagaagga aucauuccua gcauguucga accagagcgu 6060
gaaaaggugg augccauuga uggcgaauac cgcuugagag gagaagcaag gaaaaccuuu 6120
guagacuuaa ugagaagagg agaccuacca gucugguugg ccuacagagu ggcagcugaa 6180
ggcaucaacu acgcagacag aagguggugu uuugauggag ucaagaacaa ccaaauccua 6240
gaagaaaacg uggaaguuga aaucuggaca aaagaagggg aaaggaagaa auugaaaccc 6300
agaugguugg augcuaggau cuauucugac ccacuggcgc uaaaagaauu uaaggaauuu 6360
gcagccggaa gaaagucucu gacccugaac cuaaucacag aaauggguag gcucccaacc 6420
uucaugacuc agaaggcaag agacgcacug gacaacuuag cagugcugca cacggcugag 6480
gcagguggaa gggcguacaa ccaugcucuc agugaacugc cggagacccu ggagacauug 6540
cuuuuacuga cacuucuggc uacagucacg ggagggaucu uuuuauucuu gaugagcgga 6600
aggggcauag ggaagaugac ccugggaaug ugcugcauaa ucacggcuag cauccuccua 6660
ugguacgcac aaauacagcc acacuggaua gcagcuucaa uaauacugga guuuuuucuc 6720
uaguuuugcu uauuccagaa ccugaaaaac agagaacacc ccaagacaac caacugaccu 6780
acguugucau agccauccuc acaguggugg ccgcaaccau ggcaaacgag auggguuucc 6840
uagaaaaaac gaagaaagau cucggauugg gaagcauugc aacccagcaa cccgagagca 6900
acauccugga cauagaucua cguccugcau cagcauggac gcuguaugcc guggccacaa 6960
cauuuguuac accaauguug agacauagca uugaaaauuc cucagugaau gugucccuaa 7020
cagcuauagc caaccaagcc acaguguuaa ugggucucgg gaaaggaugg ccauugucaa 7080
agauggacau cggaguuccc cuucucgcca uuggaugcua cucacaaguc aaccccauaa 7140
cucucacagc agcucuuuuc uuauugguag cacauuaugc caucauaggg ccaggacucc 7200
aagcaaaagc aaccagagaa gcucagaaaa gagcagcggc gggcaucaug aaaaacccaa 7260
cugucgaugg aauaacagug auugaccuag auccaauacc uuaugaucca aaguuugaaa 7320
agcaguuggg acaaguaaug cuccuagucc ucugcgugac ucaaguauug augaugagga 7380
cuacaugggc ucugugugag gcuuuaaccu uagcuaccgg gcccaucucc acauuguggg 7440
aaggaaaucc agggagguuu uggaacacua ccauugcggu gucaauggcu aacauuuuua 7500
gagggaguua cuuggccgga gcuggacuuc ucuuuucuau uaugaagaac acaaccaaca 7560
caagaagggg aacuggcaac auaggagaga cgcuuggaga gaaauggaaa agccgauuga 7620
acgcauuggg aaaaagugaa uuccagaucu acaagaaaag uggaauccag gaaguggaua 7680
gaaccuuagc aaaagaaggc auuaaaagag gagaaacgga ccaucacgcu gugucgcgag 7740
gcucagcaaa acugagaugg uucguugaga gaaacauggu cacaccagaa gggaaaguag 7800
uggaccucgg uuguggcaga ggaggcuggu cauacuauug uggaggacua aagaauguaa 7860
gagaagucaa aggccuaaca aaaggaggac caggacacga agaacccauc cccaugucaa 7920
cauaugggug gaaucuagug cgucuucaaa guggaguuga cguuuucuuc aucccgccag 7980
aaaaguguga cacauuauug ugugacauag gggagucauc accaaauccc acaguggaag 8040
caggacgaac acucagaguc cuuaacuuag uagaaaauug guugaacaac aacacucaau 8100
uuugcauaaa gguucucaac ccauauaugc ccucagucau agaaaaaaug gaagcacuac 8160
aaaggaaaua uggaggagcc uuagugagga auccacucuc acgaaacucc acacaugaga 8220
uguacugggu auccaaugcu uccgggaaca uagugucauc agugaacaug auuucaagga 8280
uguugaucaa cagauuuaca augagauaca agaaagccac uuacgagccg gauguugacc 8340
ucggaagcgg aacccguaac aucgggauug aaagugagau accaaaccua gauauaauug 8400
ggaaaagaau agaaaaaaua aagcaagagc augaaacauc auggcacuau gaccaagacc 8460
acccauacaa aacgugggca uaccauggua gcuaugaaac aaaacagacu ggaucagcau 8520
cauccauggu caacggagug gucaggcugc ugacaaaacc uugggacguc guccccaugg 8580
ugacacagau ggcaaugaca gacacgacuc cauuuggaca acagcgcguu uuuaaagaga 8640
aaguggacac gagaacccaa gaaccgaaag aaggcacgaa gaaacuaaug aaaauaacag 8700
cagaguggcu uuggaaagaa uuagggaaga aaaagacacc caggaugugc accagagaag 8760
aauucacaag aaaggugaga agcaaugcag ccuugggggc cauauucacu gaugagaaca 8820
aguggaaguc ggcacgugag gcuguugaag auaguagguu uugggagcug guugacaagg 8880
aaaggaaucu ccaucuugaa ggaaagugug aaacaugugu guacaacaug augggaaaaa 8940
gagagaagaa gcuaggggaa uucggcaagg caaaaggcag cagagccaua ugguacaugu 9000
ggcuuggagc acgcuucuua gaguuugaag cccuaggauu cuuaaaugaa gaucacuggu 9060
ucuccagaga gaacucccug aguggagugg aaggagaagg gcugcacaag cuagguuaca 9120
uucuaagaga cgugagcaag aaagagggag gagcaaugua ugccgaugac accgcaggau 9180
gggauacaag aaucacacua gaagaccuaa aaaaugaaga aaugguaaca aaccacaugg 9240
aaggagaaca caagaaacua gccgaggcca uuuucaaacu aacguaccaa aacaaggugg 9300
ugcgugugca aagaccaaca ccaagaggca caguaaugga caucauaucg agaagagacc 9360
aaagagguag uggacaaguu ggcaccuaug gacucaauac uuucaccaau auggaagccc 9420
aacuaaucag acagauggag ggagaaggag ucuuuaaaag cauucagcac cuaacaauca 9480
cagaagaaau cgcugugcaa aacugguuag caagaguggg gcgcgaaagg uuaucaagaa 9540
uggccaucag uggagaugau uguguuguga aaccuuuaga ugacagguuc gcaagcgcuu 9600
uaacagcucu aaaugacaug ggaaagauua ggaaagacau acaacaaugg gaaccuucaa 9660
gaggauggaa ugauuggaca caagugcccu ucuguucaca ccauuuccau gaguuaauca 9720
ugaaagacgg ucgcguacuc guuguuccau guagaaacca agaugaacug auuggcagag 9780
cccgaaucuc ccaaggagca ggguggucuu ugcgggagac ggccuguuug gggaagucuu 9840
acgcccaaau guggagcuug auguacuucc acagacgcga ccucaggcug gcggcaaaug 9900
cuauuugcuc ggcaguacca ucacauuggg uuccaacaag ucgaacaacc ugguccauac 9960
augcuaaaca ugaauggaug acaacggaag acaugcugac agucuggaac agggugugga 10020
uucaagaaaa cccauggaug gaagacaaaa cuccagugga aucaugggag gaaaucccau 10080
acuuggggaa aagagaagac caauggugcg gcucauugau uggguuaaca agcagggcca 10140
ccugggcaaa gaacauccaa gcagcaauaa aucaaguuag aucccuuaua ggcaaugaag 10200
aauacacaga uuacaugcca uccaugaaaa gauucagaag agaagaggaa gaagcaggag 10260
uucuguggua gaaagcaaaa cuaacaugaa acaaggcuag aagucagguc ggauuaagcc 10320
auaguacgga aaaaacuaug cuaccuguga gccccgucca aggacguuaa aagaagucag 10380
gccaucauaa augccauagc uugaguaaac uaugcagccu guagcuccac cugagaaggu 10440
guaaaaaauc cgggaggcca caaaccaugg aagcuguacg cauggcguag uggacuagcg 10500
guuagaggag accccucccu uacaaaucgc agcaacaaug ggggcccaag gcgagaugaa 10560
gcuguagucu cgcuggaagg acuagagguu agaggagacc cccccgaaac aaaaaacagc 10620
auauugacgc ugggaaagac cagagauccu gcugucuccu cagcaucauu ccaggcacag 10680
aacgccagaa aauggaaugg ugcuguugaa ucaacagguu cu 10722

SEQ ID NO: 8

<211> 293
<212> RNA
<213> Artificial sequence
<220>
<223> Circular Artificial sequence to 3UTR (Dengue virus)
(circ_dv_3utr)
<220>
<221> misc_ feature
<222> 7..38
<223> /note=“hybridization site to 3UTR (Dengue virus)”
<220>
<221> misc_ feature
<222> 48..79
<223> /note=“hybridization site to 3UTR (Dengue virus)”
<220>
<221> misc_ feature
<222> 89..120
<223> /note=“hybridization site to 3UTR (Dengue virus)”
<220>
<221> misc_ feature
<222> 130..161
<223> /note=“hybridization site to 3UTR (Dengue virus)”
<220>
<221> misc_ feature
<222> 171..202
<223> /note=“hybridization site to 3UTR (Dengue virus)”
<220>
<221> misc_ feature
<222> 212..243
<223> /note=“hybridization site to 3UTR (Dengue virus)”
<220>
<221> misc_ feature
<222> 253..284
<223> /note=“hybridization site to 3UTR (Dengue virus)”
<400> 8

gcgccgucuu uggucuuuuu uggcgucagu auguuguuuu uuaaccaucu uugguuuuuc 60
cuggcgucag ugugcuguua uaguaugguc ucugguuuuu uuuagcguua gugugcuguu 120
aaagcuuucu cucuggucuu uuccaguguc aauaugcugu uuagugugcu ucuuugguuu 180
uucuuggugu uggugugcug uuucagcaag uucucugguc uuuucuggcg uugauguguu 240
guucgcuggc gaucuuuggu cuuucucggu gucgauaugu uguuaguggc cca 293

SEQ ID NO: 9

<211> 300
<212> RNA
<213> Artificial sequence
<220>
<223> Circular Artificial sequence to ChP (Dengue virus)
(circ_dv_cHP_v1)
<220>
<221> misc_ feature
<222> 7..39
<223> /note=“hybridization site to ChP (Dengue virus)”
<220>
<221> misc_ feature
<222> 49..81
<223> /note=“hybridization site to ChP (Dengue virus)”
<220>
<221> misc_ feature
<222> 91..123
<223> /note=“hybridization site to ChP (Dengue virus)”
<220>
<221> misc_ feature
<222> 133..165
<223> /note=“hybridization site to ChP (Dengue virus)”
<220>
<221> misc_ feature
<222> 175..207
<223> /note=“hybridization site to ChP (Dengue virus)”
<220>
<221> misc_ feature
<222> 217..249
<223> /note=“hybridization site to ChP (Dengue virus)”
<220>
<221> misc_ feature
<222> 259..291
<223> /note=“hybridization site to ChP (Dengue virus)”
<400> 9

gcgccgaugu uggggggugu guuuuuuguc uuuuuucguu gggguguaau guugagaggc 60
guguuuuuug cuuuuuuuug ugcaccuuau auauugggag guguguuuuu cgcuuuuuuc 120
cgucuggcug gcauauuggg gggcguguuu uucguuuuuu uucguuacaa gcuaauauug 180
aagggugugu uuuucgccuu uuuccguguu aaagauaugu ugaaaggcgu guuuuucguu 240
uuuuuccgua aguuuuaaau auuggagggc guguuuuucg cuuuuuucug uagaccggaa 300

SEQ ID NO: 10

<211> 294
<212> RNA
<213> Artificial sequence
<220>
<223> Circular Artificial sequence to ChP2 (Dengue virus)
(circ_dv_cHP_v2)
<220>
<221> misc_ feature
<222> 7..39
<223> /note=“hybridization site to ChP2 (Dengue virus)”
<220>
<221> misc_ feature
<222> 55..87
<223> /note=“hybridization site to ChP2 (Dengue virus)”
<220>
<221> misc_ feature
<222> 103..135
<223> /note=“hybridization site to ChP2 (Dengue virus)”
<220>
<221> misc_ feature
<222> 151..183
<223> /note=“hybridization site to ChP2 (Dengue virus)”
<220>
<221> misc_ feature
<222> 199..231
<223> /note=“hybridization site to ChP2 (Dengue virus)”
<220>
<221> misc_ feature
<222> 247..279
<223> /note=“hybridization site to ChP2 (Dengue virus)”
<400> 10

gcgccgaugu ugagaggugu guuuuuugcu uuuuuucguu uauuacucca acagauauug 60
agaggcgugu uuuucgcuuu uuuucguaga agguaauaga aaauauuggg gggcguguuu 120
uucgucuuuu uucgugacuc uuacgaguuu auguugaaag guguguuuuu cgcuuuuuuu 180
uguagggaac agucgcaaau auuggggggu guguuuuucg ccuuuuuucg uggcaccgua 240
auccgcaugu ugaaaggugu guuuuucguu uuuuuccguc aacucguucu uaua 294

SEQ ID NO: 11

<211> 11840
<212> RNA
<213> Chikungunya virus
<220>
<221> misc_ feature
<222> 14..48
<223> /note=“target region of 5UTR”
<220>
<221> misc_ feature
<222> 11367..11399
<223> /note=“target region of RSE”
<400> 11

auggcugcgu gagacacacg uagccuacca guuucuuacu gcucuacucu gcaaagcaag 60
agauuaauaa cccaucaugg auccugugua cguggacaua gacgcugaca gcgccuuuuu 120
gaaggcccug caacgugcgu accccauguu ugagguggaa ccaaggcagg ucacaccgaa 180
ugaccaugcu aaugcuagag cguucucgca ucuagcuaua aaacuaauag agcaggaaau 240
ugaccccgac ucaaccaucc uggauaucgg cagugcgcca gcaaggagga ugaugucgga 300
caggaaguac cacugcgucu gcccgaugcg cagugcggaa gaucccgaga gacucgccaa 360
uuaugcgaga aagcuagcau cugccgcagg aaaaguccug gacagaaaca ucucuggaaa 420
gaucggggac uuacaagcag uaauggccgu gccagacacg gagacgccaa cauucugcuu 480
acacacagac gucucaugua gacagagagc agacgucgcu auauaccaag acgucuaugc 540
uguacacgca cccacgucgc uauaccacca ggcgauuaaa gggguccgag uggcguacug 600
gguuggguuc gacacaaccc cguucaugua caaugccaug gcgggugccu accccucaua 660
cucgacaaac ugggcagaug agcagguacu gaaggcuaag aacauaggau uauguucaac 720
agaccugacg gaagguagac gaggcaaguu gucuauuaug agagggaaaa agcuaaaacc 780
gugcgaccgu gugcuguucu caguaggguc aacgcucuac ccggaaagcc gcaagcuacu 840
uaagagcugg caccugccau cgguguucca uuuaaagggc aaacucagcu ucacaugccg 900
cugugauaca gugguuucgu gugagggcua cgucguuaag agaauaacga ugagcccagg 960
ccuuuaugga aaaaccacag gguaugcggu aacccaccac gcagacggau uccugaugug 1020
caagacuacc gacacgguug acggcgaaag arugucauuc ucggugugca cauacgugcc 1080
ggcgaccauu ugugaucaaa ugaccggcau ccuugcuaca gaagucacgc cggaggaugc 1140
acagaagcug uugguggggc ugaaccagag aauagugguu aacggcagaa cgcaacggaa 1200
uacgaacacc augaaaaauu aucugcuucc cguggucgcc caagccuuca guaagugggc 1260
aaaggagugc cggaaagaca uggaagauga aaaacuccug ggggucagag aaagaacacu 1320
gaccugcugc ugucuauggg cauucaagaa gcagaaaaca cacacggucu acaagaggcc 1380
ugauacccag ucaauucaga agguucaggc cgaguuugac agcuuugugg uaccgagucu 1440
guggucgucc ggguugucaa ucccuuugag gacuagaauc aaaugguugu uaagcaaggu 1500
gccaaaaacc gaccugaucc cauacagcgg agacgcccga gaagcccggg acgcagaaaa 1560
agaagcagag gaagaacgag aagcagaacu gacucgcgaa gcccuaccac cucuacaggc 1620
agcacaggaa gauguucagg ucgaaaucga cguggaacag cuugaggaca gagcgggcgc 1680
aggaauaaua gagacuccga gaggagcuau caaaguuacu gcccaaccaa cagaccacgu 1740
cgugggagag uaccugguac ucuccccgca gaccguacua cguagccaga agcucagucu 1800
gauucacgcu uuggcggagc aagugaagac gugcacgcac aacggacgag cagggaggua 1860
ugcggucgaa gcguacgacg gccgaguccu agugcccuca ggcuaugcaa ucucgccuga 1920
agacuuccag agucuaagcg aaagcgcaac gaugguguau aacgaaagag aguucguaaa 1980
cagaaagcua caccauauug cgaugcacgg accagcccug aacaccgacg aagagucgua 2040
ugagcuggug agggcagaga ggacagaaca cgaguacguc uacgacgugg aucagagaag 2100
augcuguaag aaggaagaag ccgcaggacu gguacuggug ggcgacuuga cuaauccgcc 2160
cuaccacgaa uucgcauaug aagggcuaaa aauccgcccu gccugcccau acaaaauugc 2220
agucauagga gucuucggag uaccgggauc uggcaaguca gcuauuauca agaaccuagu 2280
uaccaggcag gaccugguga cuagcggaaa gaaagaaaac ugccaagaaa ucaccaccga 2340
cgugaugaga cagagagguc uagagauauc ugcacguacg guugacucgc ugcucuugaa 2400
uggaugcaac agaccagucg acguguugua cguagacgag gcguuugcgu gccacucugg 2460
aacgcuacuu gcuuugaucg ccuuggugag accaaggcag aaaguuguac uuugugguga 2520
cccgaagcag ugcggcuucu ucaauaugau gcagaugaaa gucaacuaua aucacaacau 2580
cugcacccaa guguaccaca aaaguaucuc caggcggugu acacugccug ugaccgccau 2640
ugugucaucg uugcauuacg aaggcaaaau gcgcacuacg aaugaguaca acaagccgau 2700
uguaguggac acuacaggcu caacaaaacc ugacccugga gaccucgugu uaacgugcuu 2760
cagagggugg guuaaacaac ugcaaauuga cuaucgugga uacgagguca ugacagcagc 2820
cgcaucccaa ggguuaacca gaaaaggagu uuacgcaguu agacaaaaag uuaaugaaaa 2880
cccgcucuau gcaucaacgu cagagcacgu caacguacuc cuaacgcgua cggaagguaa 2940
acugguaugg aagacacuuu ccggcgaccc guggauaaag acgcugcaga acccaccgaa 3000
aggaaacuuc aaagcaacua uuaaggagug ggagguggag caugcaucaa uaauggcggg 3060
caucugcagu caccaaauga ccuucgauac auuccaaaau aaagccaacg uuuguugggc 3120
uaagagcuug gucccuaucc ucgaaacagc ggggauaaaa cuaaaugaua ggcagugguc 3180
ucagauaauu caagccuuca aagaagacaa agcauacuca ccugaaguag cccugaauga 3240
aauauguacg cgcauguaug ggguggaucu agacagcggg cuauuuucua aaccguuggu 3300
gucuguguau uacgcggaua accacuggga uaauaggccu ggagggaaaa uguucggauu 3360
uaaccccgag gcagcaucca uucuagaaag aaaguaucca uucacaaaag ggaaguggaa 3420
caucaacaag cagaucugcg ugacuaccag gaggauagaa gacuuuaacc cuaccaccaa 3480
caucauaccg gccaacagga gacuaccaca cucauuagug gccgaacacc gcccaguaaa 3540
aggggaaaga auggaauggc ugguuaacaa gauaaacggc caccacgugc uccuggucag 3600
uggcuauaac cuugcacugc cuacuaagag agucacuugg guagcgccgu uagguguccg 3660
cggagcggac uacacauaca accuagaguu gggucugcca gcaacgcuug guagguauga 3720
ccuagugguc auaaacaucc acacaccuuu ucgcauacac cauuaccaac agugcgucga 3780
ccacgcaaug aaacugcaaa ugcucggggg ugacucauug agacugcuca aaccgggcgg 3840
cucucuauug aucagagcau augguuacgc agauagaacc agugaacgag ucaucugcgu 3900
auugggacgc aaguuuagau cgucuagagc guugaaacca ccauguguca ccagcaacac 3960
ugagauguuu uuccuauuca gcaacuuuga caauggcaga aggaauuuca caacucaugu 4020
caugaacaau caacugaaug cagccuucgu aggacagguc acccgagcag gaugugcacc 4080
gucguaccgg guaaaacgca uggacaucgc gaagaacgau gaagagugcg uagucaacgc 4140
cgcuaacccu cgcggguuac cgggugrcgg uguuugcaag gcaguauaca aaaaauggcc 4200
ggaguccuuu aagaacagug caacaccagu gggaaccgca aaaacaguua ugugcgguac 4260
guauccagua auccacgcug uuggaccaaa cuucucuaau uauucggagu cugaagggga 4320
ccgggaauug gcagcugccu aucgagaagu cgcaaaggaa guaacuaggc ugggaguaaa 4380
uaguguagcu auaccucucc ucuccacagg uguauacuca ggagggaaag acaggcugac 4440
ccagucacug aaccaccucu uuacagccau ggacucgacg gaugcagacg uggucaucua 4500
cugccgcgac aaagaauggg agaagaaaau aucugaggcc auacagaugc ggacccaagu 4560
agagcugcug gaugagcaca ucuccauaga cugcgauauu guucgcgugc acccugacag 4620
cagcuuggca ggcagaaaag gauacagcac cacggaaggc gcacuguacu cauaucuaga 4680
agggacccgu uuucaucaga cggcugugga uauggcggag auacauacua uguggccaaa 4740
gcaaacagag gccaaugagc aagucugccu auaugcccug ggggaaagua uugaaucgau 4800
caggcagaaa ugcccggugg augaugcaga cgcaucaucu ccccccaaaa cugucccgug 4860
ccuuugccgu uacgcuauga cuccagaacg cgucacccgg cuucgcauga accacgucac 4920
aagcauaauu guguguucuu cguuuccccu cccaaaguac aaaauagaag gagugcaaaa 4980
agucaaaugc ucuaagguaa ugcuauuuga ccacaacgug ccaucgcgcg uaaguccaag 5040
ggaauauaka ucuucccagg agucugcaca ggaggcgagu acaaucacgu cacugacgca 5100
uagucaauuc gaccuaagcg uugauggcga gauacugccc gucccgucag accuggaugc 5160
ugacgcccca gcccuagaac cagcacuaga cgacggggcg acacacacgc ugccauccac 5220
aaccggaaac cuugcggccg ugucugauug gguaaugagc accguaccug ucgcgccgcc 5280
cagaagaagg cgagggagaa accugacugu gacaugugac gagagagaag ggaauauaac 5340
acccauggcu agcguccgau ucuuuagggc agagcugugu ccggucguac aagaaacagc 5400
ggagacgcgu gacacagcaa ugucucuuca ggcaccaccg aguaccgcca cggaaccgaa 5460
ucauccgccg aucuccuucg gagcaucaag cgagacguuc cccauuacau uuggggacuu 5520
caacgaagga gaaaucgaaa gcuugucuuc ugagcuacua acuuucggag acuucuuacc 5580
aggagaagug gaugacuuga cagacagcga cugguccacg ugcucagaca cggacgacga 5640
guuaugacua gacagggcag guggguauau auucucgucg gacaccgguc caggucauuu 5700
acaacagaag ucaguacgcc agucagugcu gccggugaac acccuggagg aaguccacga 5760
ggagaagugu uacccaccua agcuggauga agcaaaggag caacuauuac uuaagaaacu 5820
ccaggagagu gcauccaugg ccaacagaag cagguaucag ucgcgcaaag uagaaaacau 5880
gaaagcagca aucauccaga gacuaaagag aggcuguaga cuauacuuaa ugucagagac 5940
cccaaaaguc ccuacuuacc ggacuacaua uccggcgccu guguacucgc cuccgaucaa 6000
cguccgauug uccaaucccg aguccgcagu ggcagcaugc aaugaguucu uagcuagaaa 6060
cuauccaacu gucucaucau accaaauuac cgacgaguau gaugcauauc uagacauggu 6120
ggacgggucg gagaguugcc uggaccgagc gacauucaau ccgucaaaac ucaggagcua 6180
cccgaaacag cacgcuuacc acgcgcccuc caucagaagc gcuguaccgu ccccauucca 6240
gaacacacua cagaauguac uggcagcagc cacgaaaaga aacugcaacg ucacacagau 6300
gagggaauua cccacuuugg acucagcagu auucaacgug gaguguuuca aaaaauucgc 6360
augcaaccaa gaauacuggg aagaauuugc ugccagcccu auuaggauaa caacugagaa 6420
uuuagcaacc uauguuacua aacuaaaagg gccaaaagca gcagcgcuau ucgcaaaaac 6480
ccauaaucua cugccacuac aggaaguacc aauggauagg uucacaguag auaugaaaag 6540
ggacguaaag gugacuccug guacaaagca uacagaggaa agaccuaagg ugcagguuau 6600
acaggcggcu gaacccuugg cgacagcaua ccuauguggg auucacagag agcugguuag 6660
gaggcugaac gccguccucc uacccaaugu acauacacua uuugacaugu cugccgagga 6720
uuucgaugcc aucauagccg cacacuuuaa gccaggagac acuguuuugg aaacggacau 6780
agccuccuuu gauaagagcc aagaugauuc acuugcgcuu acugcuuuga ugcuguuaga 6840
ggauuuaggg guggaucacu cccugcugga cuugauagag gcugcuuucg gagagauuuc 6900
cagcugucac cuaccgacag guacgcgcuu caaguucggc gccaugauga aaucagguau 6960
guuccuaacu cuguucguca acacauuguu aaacaucacc aucgccagcc gagugcugga 7020
agaucgucug acaaaauccg cgugcgcggc cuucaucggc gacgacaaca uaauacaugg 7080
agucgucucc gaugaauuga uggcagccag augugccacu uggaugaaca uggaagugaa 7140
gaucauagau gcaguuguau ccuugaaagc cccuuacuuu uguggagggu uuauacugca 7200
cgauacugug acaggaacag cuugcagagu ggcagacccg cuaaaaaggc uuuuuaaacu 7260
gggcaaaccg cuagcggcag gugacgaaca agaugaagau agaagacgag cgcuggcuga 7320
cgaagugauc agauggcaac gaacagggcu aauugaugag cuggagaaag cgguauacuc 7380
uagguacgaa gugcagggua uaucaguugu gguaaugucc auggccaccu uugcaagcuc 7440
cagauccaac uucgagaagc ucagaggacc cgucauaacu uuguacggcg guccuaaaua 7500
gguacgcacu acagcuaccu auuuugcaga agccgacagc aaguaucuaa acacuaauca 7560
gcuacaaugg aguucauccc aacccaaacu uuuuacaaua ggagguacca gccucgaccc 7620
uggacuccgc gcccuacuau ccaagucauc aggcccagac cgcgcccuca gaggcaagcu 7680
gggcaacuug cccagcugau cucagcaguu aauaaacuga caaugcgcgc gguaccccaa 7740
cagaagccac gcaggaaucg gaagaauaag aagcaaaagc aaaaacaaca ggcgccacaa 7800
aacaacacaa aucaaaagaa gcagccaccu aaaaagaaac cggcucaaaa gaaaaagaag 7860
ccgggccgca gagagaggau gugcaugaaa aucgaaaaug auuguauuuu cgaagucaag 7920
cacgaaggua agguaacagg uuacgcgugc cugguggggg acaaaguaau gaaaccagca 7980
cacguaaagg ggaccaucga uaacgcggac cuggccaaac uggccuuuaa gcggucaucu 8040
aaguaugacc uugaaugcgc gcagauaccc gugcacauga aguccgacgc uucgaaguuc 8100
acccaugaga aaccggaggg guacuacaac uggcaccacg gagcaguaca guacucagga 8160
ggccgguuca ccaucccuac aggugcuggc aaaccagggg acagcggcag accgaucuuc 8220
gacaacaagg gacgcguggu ggccauaguc uuaggaggag cuaaugaagg agcccguaca 8280
gcccucucgg uggugaccug gaauaaagac auugucacua aaaucacccc cgagggggcc 8340
gaagagugga gucuugccau cccaguuaug ugccuguugg caaacaccac guuccccugc 8400
ucccagcccc cuugcacgcc cugcugcuac gaaaaggaac cggaggaaac ccuacgcaug 8460
cuugaggaca acgucaugag accuggguac uaucagcugc uacaagcauc cuuaacaugu 8520
ucuccccacc gccagcgacg cagcaccaag gacaacuuca augucuauaa agccacaaga 8580
ccauacuuag cucacugucc cgacugugga gaagggcacu cgugccauag ucccguagca 8640
cuagaacgca ucagaaauga agcgacagac gggacgcuga aaauccaggu cuccuugcaa 8700
aucggaauaa agacggauga cagccacgau uggaccaagc ugcguuauau ggacaaccac 8760
augccagcag acgcagagag ggcggggcua uuuguaagaa caucagcacc guguacgauu 8820
acuggaacaa ugggacacuu cauccuggcc cgauguccaa aaggggaaac ucugacggug 8880
ggauucacug acaguaggaa gauuagucac ucauguacgc acccauuuca ccacgacccu 8940
ccugugauag gucgggaaaa auuccauucc cgaccgcagc acgguaaaga gcuaccuugc 9000
agcacguacg ugcagagcac cgccgcaacu accgaggaga uagagguaca caugccccca 9060
gacaccccug aucgcacauu aaugucacaa caguccggca acguaaagau cacagucaau 9120
ggccagacgg ugcgguacaa guguaauugc gguggcucaa augaaggacu aacaacuaca 9180
gacaaaguga uuaauaacug caagguugau caaugucaug ccgcggucac caaucacaaa 9240
aaguggcagu auaacucccc ucuggucccg cguaaugcug aacuugggga ccgaaaagga 9300
aaaauucaca ucccguuucc gcuggcaaau guaacaugca gggugccuaa agcaaggaac 9360
cccaccguga cguacgggaa aaaccaaguc aucaugcuac uguauccuga ccacccaaca 9420
cuccuguccu accggaauau gggagaagaa ccaaacuauc aagaagagug ggugaugcau 9480
aagaaggaag ucgugcuaac cgugccgacu gaagggcucg aggucacgug gggcaacaac 9540
gagccguaua aguauuggcc gcaguuaucu acaaacggua cagcccaugg ccacccgcau 9600
gagauaauuc uguauuauua ugagcuguac cccacuauga cuguaguagu ugugucagug 9660
gccacguuca uacuccuguc gauggugggu auggcagcgg ggaugugcau gugugcacga 9720
cgcagaugca ucacaccgua ugaacugaca ccaggagcua ccgucccuuu ccugcuuagc 9780
cuaauaugcu gcaucagaac agcuaaagcg gccacauacc aagaggcugc gauauaccug 9840
uggaacgagc agcaaccuuu guuuuggcua caagcccuua uuccgcuggc agcccugauu 9900
guucuaugca acugucugag acucuuacca ugcugcugua aaacguuggc uuuuuuagcc 9960
guaaugagcg ucggugccca cacugugagc gcguacgaac acguaacagu gaucccgaac 10020
acggugggag uaccguauaa gacucuaguc aauagaccug gcuacagccc caugguauug 10080
gagauggaac uacugucagu cacuuuggag ccaacacuau cgcuugauua caucacgugc 10140
gaguacaaaa ccgucauccc gucuccguac gugaagugcu gcgguacagc agagugcaag 10200
gacaaaaacc uaccugacua cagcuguaag gucuucaccg gcgucuaccc auuuaugugg 10260
ggcggcgccu acugcuucug cgacgcugaa aacacgcagu ugagcgaagc acacguggag 10320
aaguccgaau caugcaaaac agaauuugca ucagcauaca gggcucauac cgcaucugca 10380
ucagcuaagc uccgcguccu uuaccaagga aauaacauca cuguaacugc cuaugcaaac 10440
ggcgaccaug ccgucacagu uaaggacgcc aaauucauug uggggccaau gucuucagcc 10500
uggacaccuu ucgacaacaa aauuguggug uacaaaggug acgucuauaa cauggacuac 10560
ccgcccuuug gcgcaggaag accaggacaa uuuggcgaua uccaaagucg cacaccugag 10620
aguaaagacg ucuaugcuaa uacacaacug guacugcaga gaccggcugu ggguacggua 10680
cacgugccau acucucaggc accaucuggc uuuaaguauu ggcuaaaaga acgcggggcg 10740
ucgcugcagc acacagcacc auuuggcugc caaauagcaa caaacccggu aagagcggug 10800
aacugcgccg uagggaacau gcccaucucc aucgacauac cggaagcggc cuucacuagg 10860
gucgucgacg cgcccucuuu aacggacaug ucgugcgagg uaccagccug cacccauucc 10920
ucagacuuug ggggcgucgc cauuauuaaa uaugcagcca gcaagaaagg caagugugcg 10980
gugcauucga ugacuaacgc cgucacuauu cgggaagcug agauagaagu ugaagggaau 11040
ucucagcugc aaaucucuuu cucgacggcc uuagccagcg ccgaauuccg cguacaaguc 11100
uguucuacac aaguacacug ugcagccgag ugccaccccc cgaaggacca cauagucaac 11160
uacccggcgu cacauaccac ccucgggguc caggacaucu ccgcuacggc gaugucaugg 11220
gugcagaaga ucacgggagg ugugggacug guuguugcug uugccgcacu gauucuaauc 11280
guggugcuau gcgugucguu cagcaggcac uaacuugaca auuaaguaug aagguauaug 11340
uguccccuaa gagacacacu guacauagca aauaaucuau agaucaaagg gcuacgcaac 11400
cccugaauag uaacaaaaua caaaaucacu aaaaauuaua aaaacagaaa aauacauaaa 11460
uagguauacg uguccccuaa gagacacauu guauguaggu gauaaguaua gaucaaaggg 11520
ccgaauaacc ccugaauagu aacaaaauau gaaaaucaau aaaaaucaua aaauagaaaa 11580
accauaaaca gaaguaguuc aaagggcuau aaaaccccug aauaguaaca aaacauaaaa 11640
uuaauaaaaa ucaaaugaau accauaauug gcaaacggaa gagauguagg uacuuaagcu 11700
uccuaaaagc agccgaacuc acuuugagaa guaggcauag cauaccgaac ucuuccacga 11760
uucuccgaac ccacagggac guaggagaug uuauuuuguu uuuaauauuu caaaaaaaaa 11820
aaaaaaaaaa aaaaaaaaaa 11840

SEQ ID NO: 12

<211> 294
<212> RNA
<213> Artificial sequence
<220>
<223> Circular Artificial sequence to 5UTR (Chikungunya virus)
(chikv_5utr1 )
<220>
<221> misc_ feature
<222> 7..41
<223> /note=“hybridization site to 5UTR (Chikungunya virus)”
<220>
<221> misc_ feature
<222> 55..89
<223> /note=“hybridization site to 5UTR (Chikungunya virus)”
<220>
<221> misc_ feature
<222> 103..137
<223> /note=“hybridization site to 5UTR (Chikungunya virus)”
<220>
<221> misc_ feature
<222> 151..185
<223> /note=“hybridization site to 5UTR (Chikungunya virus)”
<220>
<221> misc_ feature
<222> 199..233
<223> /note=“hybridization site to 5UTR (Chikungunya virus)”
<220>
<221> misc_ feature
<222> 247..281
<223> /note=“hybridization site to 5UTR (Chikungunya virus)”
<400> 12

gcgccgagua gagcagugag gaacuggugg guugcgugug uuuugucacu acguaguaga 60
gugguaggaa auuggugggc ugcgugugug gcuuccagug ccaguagagc ggugagagau 120
ugguagguua uguguguguu cucgcccgaa aguagagugg ugagggguug guagguuacg 180
uguguucaaa uaugacggag uagaguagug gggggcuggu ggguugugug uguucggaga 240
ccagacagua gaguaguaag agguuggugg gcuacgugug ugccacacca cccg 294

SEQ ID NO: 13

<211> 314
<212> RNA
<213> Artificial sequence
<220>
<223> Circular Artificial sequence to 5UTR (Chikungunya virus)
(chikv_5utr2)
<220>
<221> misc_ feature
<222> 7..41
<223> /note=“hybridization site to 5UTR (Chikungunya virus)”
<220>
<221> misc_ feature
<222> 51..85
<223> /note=“hybridization site to 5UTR (Chikungunya virus)”
<220>
<221> misc_ feature
<222> 95..129
<223> /note=“hybridization site to 5UTR (Chikungunya virus)”
<220>
<221> misc_ feature
<222> 139..173
<223> /note=“hybridization site to 5UTR (Chikungunya virus)”
<220>
<221> misc_ feature
<222> 183..217
<223> /note=“hybridization site to 5UTR (Chikungunya virus)”
<220>
<221> misc_ feature
<222> 227..261
<223> /note=“hybridization site to 5UTR (Chikungunya virus)”
<220>
<221> misc_ feature
<222> 271..305
<223> /note=“hybridization site to 5UTR (Chikungunya virus)”
<400> 13

gcgccgagua gagugguggg ggguugguag guugcgugug ucuuagcgcc aguagagcag 60
uaagggacug guaggcugcg ugugugguac uuacaguaga gcggugagag acuggugggc 120
uaugugugug ccauccuuag uagaguggua gggaguuggu gggcugugug uguggggccu 180
auaguagagu agugggagau ugguagguua cguguguagc ugcacgagua gagcaguggg 240
aaguuggugg guugugugug ucuccgacga aguagaguag uaagaaauug guggguuaug 300
uguguaccug aguc 314

SEQ ID NO: 14

<211> 288
<212> RNA
<213> Artificial sequence
<220>
<223> Circular Artificial sequence to RSE (Chikungunya virus)
(chikv_RSE1)
<220>
<221> misc_ feature
<222> 7..39
<223> /note=“hybridization site to RSE (Chikungunya virus)”
<220>
<221> misc_ feature
<222> 54..86
<223> /note=“hybridization site to RSE (Chikungunya virus)”
<220>
<221> misc_ feature
<222> 101..133
<223> /note=“hybridization site to RSE (Chikungunya virus)”
<220>
<221> misc_ feature
<222> 148..180
<223> /note=“hybridization site to RSE (Chikungunya virus)”
<220>
<221> misc_ feature
<222> 195..227
<223> /note=“hybridization site to RSE (Chikungunya virus)”
<220>
<221> misc_ feature
<222> 242..274
<223> /note=“hybridization site to RSE (Chikungunya virus)”
<400> 14

gcgccguugc guggcucuuu ggucuauaga uuauuuguuu gcggguugcc cucuugcgug 60
guucuuuggu cuguggauug uuugcuugcc cgcgagcagg uuguguaguc cuuugaucug 120
uagguuguuu guuguucgua acacuucuug cguagcucuu ugaucuauag guuauuugcu 180
cuagguucga accguugcgu ggucuuuuga ucuauggguu guuuguucau caaaaccuuu 240
cuuguguggc cuuuuggucu guagauuguu uguuuccaac gacguacu 288

SEQ ID NO: 15

<211> 288
<212> RNA
<213> Artificial Sequence
<220>
<223> Circular Artificial sequence to RSE2 (Chikungunya virus)
(chikvRSE)
<220>
<221> misc_ feature
<222> 7..39
<223> /note=“hybridization site to RSE2 (Chikungunya virus)”
<220>
<221> misc_ feature
<222> 54..86
<223> /note=“hybridization site to RSE2 (Chikungunya virus)”
<220>
<221> misc_ feature
<222> 101..133
<223> /note=“hybridization site to RSE2 (Chikungunya virus)”
<220>
<221> misc_ feature
<222> 148..180
<223> /note=“hybridization site to RSE2 (Chikungunya virus)”
<220>
<221> misc_ feature
<222> 195..227
<223> /note=“hybridization site to RSE2 (Chikungunya virus)”
<220>
<221> misc_ feature
<222> 242..274
<223> /note=“hybridization site to RSE2 (Chikungunya virus)”
<400> 15

gcgccguugc guggcucuuu ggucuauggg uuguuugcuu gcggguugcc cucuugugua 60
gcuuuuugau cuguggguua uuugcuugcc cgcgagcagg uugcguaguu cuuugauuug 120
uagguuguuu gcuguucgua acacuucuug cguaguuuuu ugaucuaugg auuguuugcu 180
cuagguucga accguugcgu agcucuuugg uuuauagauu auuugcucau caaaaccuuu 240
cuuguguggu ccuuugaucu auagguuauu ugcuuccaac gacguacu 288

SEQ ID NO: 16

<211> 620
<212> RNA
<213> Artificial sequence
<220>
<223> Broadspectrum circular RNA to hepatitis C virus and Dengue virus
(DENV1 cHP_ HCV CDS2_T)
<220>
<221> misc_ feature
<222> 16..48
<223> /note=“hybridization site to chp (Dengue virus)”
<220>
<221> misc_ feature
<222> 58..90
<223> /note=“hybridization site to chp (Dengue virus)”
<220>
<221> misc_ feature
<222> 100..132
<223> /note=“hybridization site to chp (Dengue virus)”
<220>
<221> misc_ feature
<222> 142..174
<223> /note=“hybridization site to chp (Dengue virus)”
<220>
<221> misc_ feature
<222> 184..216
<223> /note=“hybridization site to chp (Dengue virus)”
<220>
<221> misc_ feature
<222> 226..258
<223> /note=“hybridization site to chp (Dengue virus)”
<220>
<221> misc_ feature
<222> 268..300
<223> /note=“hybridization site to chp (Dengue virus)”
<220>
<221> misc_ feature
<222> 330..340
<223> /note=“hybridization site to CDS2 (hepatitis C virus)”
<220>
<221> misc_ feature
<222> 354..364
<223> /note=“hybridization site to CDS2 (hepatitis C virus)”
<220>
<221> misc_ feature
<222> 378..388
<223> /note=“hybridization site to CDS2 (hepatitis C virus)”
<220>
<221> misc_ feature
<222> 402..412
<223> /note=“hybridization site to CDS2 (hepatitis C virus)”
<220>
<221> misc_ feature
<222> 426..436
<223> /note=“hybridization site to CDS2 (hepatitis C virus)”
<220>
<221> misc_ feature
<222> 450..460
<223> /note=“hybridization site to CDS2 (hepatitis C virus)”
<220>
<221> misc_ feature
<222> 474..484
<223> /note=“hybridization site to CDS2 (hepatitis C virus)”
<220>
<221> misc_ feature
<222> 498..508
<223> /note=“hybridization site to CDS2 (hepatitis C virus)”
<220>
<221> misc_ feature
<222> 522..532
<223> /note=“hybridization site to CDS2 (hepatitis C virus)”
<220>
<221> misc_ feature
<222> 546..556
<223> /note=“hybridization site to CDS2 (hepatitis C virus)”
<220>
<221> misc_ feature
<222> 570..580
<223> /note=“hybridization site to CDS2 (hepatitis C virus)”
<220>
<221> misc_ feature
<222> 594..604
<223> /note=“hybridization site to CDS2 (hepatitis C virus)”
<400> 16

ggauccgcgg cgccgauguu ggggggugug uuuuuugucu uuuuucguug ggguguaaug 60
uugagaggcg uguuuuuugc uuuuuuuugu gcaccuuaua uauugggagg uguguuuuuc 120
gcuuuuuucc gucuggcugg cauauugggg ggcguguuuu ucguuuuuuu ucguuacaag 180
cuaauauuga aggguguguu uuucgccuuu uuccguguua aagauauguu gaaaggcgug 240
uuuuucguuu uuuuccguaa guuuuaaaua uuggagggcg uguuuuucgc uuuuuucugu 300
agaccggaac cgggaauuca cucgagcccc cuggggcucu gaugaggaac cucucugggg 360
uccccacagc gagucuccuu ggggccccua gaaugaaguu cuuugggguu ucauagcacg 420
guucuccugg gguuuucaua cgauugucuc uuggggucuu gcguguuuac ccuuuugggg 480
uccugcuaag ggggcuuucu ggggccuuuc gaauaagucu uuuuggggcc ucguuuuauc 540
acucuucugg gguucccagc cuuucccuuc uuggggcucc uccgaccaug ccccuugggg 600
uucuacccug uaugggaucc 620

SEQ ID NO: 17

<211> 298
<212> RNA
<213> Artificial sequence
<220>
<223> Broadspectrum circular RNA to hepatitis C virus and Dengue virus
(DENV1 cHP_ HCV CDS2_1)
<220>
<221> misc_ feature
<222> 7..22
<223> /note=“hybridization site to CDS2(2) (hepatitis C virus)”
<220>
<221> misc_ feature
<222> 35..67
<223> /note=“hybridization site to chp (Dengue virus)”
<220>
<221> misc_ feature
<222> 80..95
<223> /note=“hybridization site to CDS2(2) (hepatitis C virus)”
<220>
<221> misc_ feature
<222> 108..140
<223> /note=“hybridization site to chp (Dengue virus)”
<220>
<221> misc_ feature
<222> 153..168
<223> /note=“hybridization site to CDS2(2) (hepatitis C virus)”
<220>
<221> misc_ feature
<222> 181..213
<223> /note=“hybridization site to chp (Dengue virus)”
<220>
<221> misc_ feature
<222> 226..241
<223> /note=“hybridization site to CDS2(2) (hepatitis C virus)”
<220>
<221> misc_ feature
<222> 254..286
<223> /note=“hybridization site to chp (Dengue virus)”
<400> 17

gcgccgccug gcuugggguu uucccaggac ucuaauauug gaaggcgugu uuuuugucuu 60
uuuucguguc cugccuccuc uuagccuggg guuuuacgac cgcaccuaug uugagaggug 120
uguuuuuugc cuuuuuucgu uugauacccu cauuuaaucu ggggcucuag gugcagcaaa 180
auguuggagg gcguguuuuu cguuuuuuuc uguccugcac gaucucccga uuugggguuc 240
ucguugguga cguauauuga gggguguguu uuucgcuuuu uuucguauug gacgucgu 298

SEQ ID NO: 18

<211> 41
<212> RNA
<213> Artificial Sequence
<220>
<223> Artificial sequence for showing an squematic example of the mode
of action according to figure 4 wherein the sequence has no
particular value
<400> 18

aacgccaugc aagcgcgcag aucgagcugu gcgcuuuuuu u 41

SEQ ID NO: 19

<211> 299
<212> RNA
<213> Artificial sequence
<220>
<223> circ_wnv_slll_2
<400> 19

ugcaguuaga uaaacuuucc gguuugauuu ucucuucaaa agacaguucu ucgaacuucc 60
cggcuugguu uucuuuucaa aaauggggcc ccucaacuuc cuggucuggu uuucuccuua 120
aaaggcuaua cgaucaacuu ucuggccuga cuuucuucuu aaaaagcuau caucgcaacu 180
ucucggcuug acuuucucuu uaaaacccuc gguaaccaac uuuucggccu gauuuucuuc 240
ucaaaaguuu uggcgacuaa cuucuugguu uggcuuucuc cucaaaaaau auaaacagc 299

SEQ ID NO: 20

<211> 11029
<212> RNA
<213> West Nile virus
<220>
<223> Genome West Nile virus
<400> 20

aguaguucgc cugugugagc ugacaaacuu aguaguguuu gugaggauua acaacaauua 60
acacagugcg agcuguuucu uagcacgaag aucucgaugu cuaagaaacc aggagggccc 120
ggcaagagcc gggcugucaa uaugcuaaaa cgcggaaugc cccgcguguu guccuugauu 180
ggacugaaga gggcuauguu gagccugauc gacggcaagg ggccaauacg auuuguguug 240
gcucucuugg cguucuucag guucacagca auugcuccga cccgagcagu gcuggaucga 300
uggagaggug ugaacaaaca aacagcgaug aaacaccuuc ugaguuuuaa gaaggaacua 360
gggaccuuga ccagugcuau caaucggcgg agcucaaaac aaaagaaaag aggaggaaag 420
accggaauug cagucaugau uggccugauu gccagcguag gagcaguuac ccucucuaac 480
uuccaaggga aggugaugau gacgguaaau gcuacugacg ucacagaugu caucacgauu 540
ccaacagcug cuggaaagaa ccuaugcauu gucagagcaa uggauguggg auacaugugc 600
gaugauacua ucacuuauga augcccagug cugucggcug guaaugaucc agaagacauc 660
gacuguuggu gcacaaaguc agcagucuac gucagguaug gaagaugcac caagacacgc 720
cacucaagac gcagucggag gucacugaca gugcagacac acggagaaag cacucuagcg 780
aacaagaagg gggcuuggau ggacagcacc aaggccacaa gguauuuggu aaaaacagaa 840
ucauggaucu ugaggaaccc uggauaugcc cugguggcag ccgucauugg uuggaugcuu 900
gggagcaaca ccaugcagag aguuguguuu gucgugcuau ugcuuuuggu ggccccagcu 960
uacagcuuca acugccuugg aaugagcaac agagacuucu uggaaggagu gucuggagca 1020
acaugggugg auuugguucu cgaaggcgac agcugcguga cuaucauguc uaaggacaag 1080
ccuaccaucg augugaagau gaugaauaug gaggcggcca accuggcaga gguccgcagu 1140
uauugcuauu uggcuaccgu cagcgaucuc uccaccaaag cugcgugccc gaccauggga 1200
gaagcucaca augacaaacg ugcugaccca gcuuuugugu gcagacaagg agugguggac 1260
aggggcuggg gcaacggcug cggacuauuu ggcaaaggaa gcauugacac augcgccaaa 1320
uuugccugcu cuaccaaggc aauaggaaga accaucuuga aagagaauau caaguacgaa 1380
guggccauuu uuguccaugg accaacuacu guggagucgc acggaaacua cuccacacag 1440
guuggagcca cucaggcagg gagauucagc aucacuccug cggcgccuuc auacacacua 1500
aagcuuggag aauauggaga ggugacagug gacugugaac cacggucagg gauugacacc 1560
aaugcauacu acgugaugac uguuggaaca aagacguucu ugguccaucg ugagugguuc 1620
auggaccuca accucccuug gagcagugcu ggaaguacug uguggaggaa cagagagacg 1680
uuaauggagu uugaggaacc acacgccacg aagcagucug ugauagcauu gggcucacaa 1740
gagggagcuc ugcaucaagc uuuggcugga gccauuccug uggaauuuuc aagcaacacu 1800
gucaaguuga cgucggguca uuugaagugu agagugaaga uggaaaaauu gcaguugaag 1860
ggaacaaccu auggcgucug uucaaaggcu uucaaguuuc uugggacucc cgcagacaca 1920
ggucacggca cugugguguu ggaauugcag uacacuggca cggauggacc uugcaaaguu 1980
ccuaucucgu caguggcuuc auugaacgac cuaacgccag ugggcagauu ggucacuguc 2040
aacccuuuug uuucaguggc cacggccaac gcuaaggucc ugauugaauu ggaaccaccc 2100
uuuggagacu cauacauagu ggugggcaga ggagaacaac agaucaauca ccauuggcac 2160
aagucuggaa gcagcauugg caaagccuuu acaaccaccc ucaaaggagc gcagagacua 2220
gccgcucuag gagacacagc uugggacuuu ggaucaguug gagggguguu caccucaguu 2280
gggaaggcug uccaucaagu guucggagga gcauuccgcu cacuguucgg aggcaugucc 2340
uggauaacgc aaggauugcu gggggcucuc cuguugugga ugggcaucaa ugcucgugau 2400
agguccauag cucucacguu ucucgcaguu ggaggaguuc ugcucuuccu cuccgugaac 2460
gugcacgcug acacugggug ugccauagac aucagccggc aagagcugag auguggaagu 2520
ggaguguuca uacacaauga uguggaggcu uggauggacc gguacaagua uuacccugaa 2580
acgccacaag gccuagccaa gaucauucag aaagcucaua aggaaggagu gugcggucua 2640
cgaucaguuu ccagacugga gcaucaaaug ugggaagcag ugaaggacga gcugaacacu 2700
cuuuugaagg agaauggugu ggaccuuagu gucgugguug agaaacagga gggaauguac 2760
aagucagcac cuaaacgccu caccgccacc acggaaaaau uggaaauugg cuggaaggcc 2820
uggggaaaga guauuuuauu ugcaccagaa cucgccaaca acaccuuugu gguugauggu 2880
ccggagacca aggaaugucc gacucagaau cgcgcuugga auagcuuaga aguggaggau 2940
uuuggauuug gucucaccag cacucggaug uuccugaagg ucagagagag caacacaacu 3000
gaaugugacu cgaagaucau uggaacggcu gucaagaaca acuuggcgau ccacagugac 3060
cuguccuauu ggauugaaag caggcucaau gauacgugga agcuugaaag ggcaguucug 3120
ggugaaguca aaucauguac guggccugag acgcauaccu uguggggcga uggaauccuu 3180
gagagugacu ugauaauacc agucacacug gcgggaccac gaagcaauca caaucggaga 3240
ccuggguaca agacacaaaa ccagggccca ugggacgaag gccggguaga gauugacuuc 3300
gauuacugcc caggaacuac ggucacccug agugagagcu gcggacaccg uggaccugcc 3360
acucgcacca ccacagagag cggaaaguug auaacagauu ggugcugcag gagcugcacc 3420
uuaccaccac ugcgcuacca aacugacagc ggcuguuggu augguaugga gaucagacca 3480
cagagacaug augaaaagac ccucgugcag ucacaaguga augcuuauaa ugcugauaug 3540
auugacccuu uucaguuggg ccuucugguc guguucuugg ccacccagga gguccuucgc 3600
aagaggugga cagccaagau cagcaugcca gcuauacuga uugcucugcu aguccuggug 3660
uuugggggca uuacuuacac ugauguguua cgcuauguca ucuugguggg ggcagcuuuc 3720
gcagaaucua auucgggagg agacguggua cacuuggcgc ucauggcgac cuucaagaua 3780
caaccagugu uuaugguggc aucguuucuc aaagcgagau ggaccaacca ggagaacauu 3840
uuguugaugu uggcggcugu uuucuuucaa auggcuuauc acgaugcccg ccaaauucug 3900
cucugggaga ucccugaugu guugaauuca cuggcgguag cuuggaugau acugagagcc 3960
auaacauuca caacgacauc aaacgugguu guuccgcugc uagcccugcu aacacccggg 4020
cugagacgcu ugaaucugga uguguacagg auacugcugu ugauggucgg aauaggcagc 4080
uugaucaggg agaagaggag ugcagcugca aaaaagaaag gagcaagucu gcuaugcuug 4140
gcucuagccu caacaggacu uuucaacccc augauccuug cugcuggacu gauugcaugu 4200
gaucccaacc guaaacgcgg auggcccgca acugaaguga ugacagcugu cggccuaaug 4260
uuugccaucg ucggagggcu ggcagagcuu gacauugacu ccauggccau uccaaugacu 4320
aucgcggggc ucauguuugc ugcuuucgug auuucuggga aaucaacaga uauguggauu 4380
gagagaacgg cggacauuuc cugggaaagu gaugcagaaa uuacaggcuc gagcgaaaga 4440
guugaugugc ggcuugauga ugauggaaac uuccagcuca ugaaugaucc aggagcaccu 4500
uggaagauau ggaugcucag aauggucugu cucgcgauua gugcguacac ccccugggca 4560
aucuugcccu caguaguugg auuuuggaua acucuccaau acacaaagag aggaggcgug 4620
uugugggaca cucccucacc aaaggaguac aaaaaggggg acacgaccac cggcgucuac 4680
aggaucauga cucgugggcu gcucggcagu uaucaagcag gagcgggcgu gaugguugaa 4740
gguguuuucc acacccuuug gcauacaaca aaaggagccg cuuugaugag cggagagggc 4800
cgccuggacc cauacugggg cagugucaag gaggaucgac uuuguuacgg aggacccugg 4860
aaauugcagc acaaguggaa cgggcaggau gaggugcaga ugauuguggu ggaaccuggc 4920
aagaacguua agaacguccg gacgaaacca gggguguuca aaacaccuga aggagaaauc 4980
ggggccguga cuuuggacuu ccccacugga acaucaggcu caccaauagu ggacaaaaac 5040
ggugauguga uugggcuuua uggcaaugga gucauaaugc ccaacggcuc auacauaagc 5100
gcgauagugc agggugaaag gauggaugag ccaaucccag ccggauucga accugagaug 5160
cugaggaaaa aacagaucac uguacuggau cuccaucccg gcgccgguaa aacaaggagg 5220
auucugccac agaucaucaa agaggccaua aacagaagac ugagaacagc cgugcuagca 5280
ccaaccaggg uuguggcugc ugagauggcu gaagcacuga gaggacugcc cauccgguac 5340
cagacauccg cagugcccag agaacauaau ggaaaugaga uuguugaugu caugugucau 5400
gcuacccuca cccacaggcu gaugucuccu cacagggugc cgaacuacaa ccuguucgug 5460
auggaugagg cucauuucac cgacccagcu agcauugcag caagagguua cauuuccaca 5520
aaggucgagc uaggggaggc ggcggcaaua uucaugacag ccaccccacc aggcacuuca 5580
gauccauucc cagaguccaa uucaccaauu uccgacuuac agacugagau cccggaucga 5640
gcuuggaacu cuggauacga auggaucaca gaauacaccg ggaagacggu uugguuugug 5700
ccuaguguca agauggggaa ugagauugcc cuuugccuac aacgugcugg aaagaaagua 5760
guccaauuga acagaaaguc guacgagacg gaguacccaa aauguaagaa cgaugauugg 5820
gacuuuguua ucacaacaga cauaucugaa augggggcua acuucaaggc gagcagggug 5880
auugacagcc ggaagagugu gaaaccaacc aucauaacag aaggagaagg gagagugauc 5940
cugggagaac caucugcagu gacagcagcu agugccgccc agagacgugg acguaucggu 6000
agaaauccgu cgcaaguugg ugaugaguac uguuaugggg ggcacacgaa ugaagacgac 6060
ucgaacuucg cccauuggac ugaggcacga aucaugcugg acaacaucaa caugccaaac 6120
ggacugaucg cucaauucua ccaaccagag cgugagaagg uauauaccau ggauggggaa 6180
uaccggcuca gaggagaaga gagaaaaaac uuucuggaac uguugaggac ugcagaucug 6240
ccaguuuggc uggcuuacaa gguugcagcg gcuggagugu cauaccacga ccggaggugg 6300
ugcuuugaug guccuaggac aaacacaauu uuagaagaca acaacgaagu ggaagucauc 6360
acgaagcuug gugaaaggaa gauucugagg ccgcgcugga uugacgccag gguguacucg 6420
gaucaccagg cacuaaaggc guucaaggac uucgccucgg gaaaacguuc ucagauaggg 6480
cucauugagg uucugggaaa gaugccugag cacuucaugg ggaagacaug ggaagcacuu 6540
gacaccaugu acguuguggc cacugcagag aaaggaggaa gagcucacag aauggcccug 6600
gaggaacugc cagaugcucu ucagacaauu gccuugauug ccuuauugag ugugaugacc 6660
augggaguau ucuuccuccu caugcagcgg aagggcauug gaaagauagg uuugggaggc 6720
gcugucuugg gagucgcgac cuuuuucugu uggauggcug aaguuccagg aacgaagauc 6780
gccggaaugu ugcugcucuc ccuucucuug augauugugc uaauuccuga gccagagaag 6840
caacguucgc agacagacaa ccagcuagcc guguuccuga uuugugucau gacccuugug 6900
agcgcagugg cagccaacga gauggguugg cuagauaaga ccaagaguga cauaagcagu 6960
uuguuugggc aaagaauuga ggucaaggag aauuucagca ugggagaguu ccuucuggac 7020
uugaggccgg caacagccug gucacuguac gcugugacaa cagcgguccu cacuccacug 7080
cuaaagcauu ugaucacguc agauuacauc aacaccucau ugaccucaau aaacguucag 7140
gcaagugcac uauucacacu cgcgcgaggc uuccccuucg ucgauguugg agugucggcu 7200
cuccugcuag cagccggaug cuggggacaa gucacccuca ccguuacggu aacagcggca 7260
acacuccuuu uuugccacua ugccuacaug guucccgguu ggcaagcuga ggcaaugcgc 7320
ucagcccagc ggcggacagc ggccggaauc augaagaacg cuguagugga uggcaucgug 7380
gccacggacg ucccagaauu agagcgcacc acacccauca ugcagaagaa aguuggacag 7440
aucaugcuga ucuugguguc ucuagcugca guaguaguga acccgucugu gaagacagua 7500
cgagaagccg gaauuuugau cacggccgca gcggugacgc uuugggagaa uggagcaagc 7560
ucuguuugga acgcaacaac ugccaucgga cucugccaca ucaugcgugg ggguugguug 7620
ucaugucuau ccauaacaug gacacucaua aagaacaugg aaaaaccagg acuaaaaaga 7680
gguggggcaa aaggacgcac cuugggagag guuuggaaag aaagacucaa ccagaugaca 7740
aaagaagagu ucacuaggua ccgcaaagag gccaucaucg aagucgaucg cucagcggca 7800
aaacacgcca ggaaagaagg caaugucacu ggagggcauc cagucucuag gggcacagca 7860
aaacugagau ggcuggucga acggagguuu cucgaaccgg ucggaaaagu gauugaccuu 7920
ggauguggaa gaggcgguug guguuacuau auggcaaccc aaaaaagagu ccaagaaguc 7980
agaggguaca caaagggcgg ucccggacau gaagagcccc aacuagugca aaguuaugga 8040
uggaacauug ucaccaugaa gaguggagug gauguguucu acagaccuuc ugaguguugu 8100
gacacccucc uuugugacau cggagagucc ucgucaagug cugagguuga agagcauagg 8160
acgauucggg uccuugaaau gguugaggac uggcugcacc gagggccaag ggaauuuugc 8220
gugaaggugc ucugccccua caugccgaaa gucauagaga agauggagcu gcuccaacgc 8280
cgguaugggg ggggacuggu cagaaaccca cucucacgga auuccacgca cgagauguau 8340
ugggugaguc gagcuucagg caauguggua cauucaguga auaugaccag ccaggugcuc 8400
cuaggaagaa uggaaaaaag gaccuggaag ggaccccaau acgaggaaga uguaaacuug 8460
ggaaguggaa ccagggcggu gggaaaaccc cugcucaacu cagacaccag uaaaaucaag 8520
aacaggauug aacgacucag gcgugaguac aguucgacgu ggcaccacga ugagaaccac 8580
ccauauagaa ccuggaacua ucacggcagu uaugauguga agcccacagg cuccgccagu 8640
ucgcugguca auggaguggu caggcuccuc ucaaaaccau gggacaccau cacgaauguu 8700
accaccaugg ccaugacuga cacuacuccc uucgggcagc agcgaguguu caaagagaag 8760
guggacacga aagcuccuga accgccagaa ggagugaagu acgugcucaa cgagaccacc 8820
aacugguugu gggcguuuuu ggccagagaa aaacguccca gaaugugcuc ucgagaggaa 8880
uucauaagaa aggucaacag caaugcagcu uugggugcca uguuugaaga gcagaaucaa 8940
uggaggagcg ccagagaagc aguugaagau ccaaaauuuu gggagauggu ggaugaggag 9000
cgcgaggcac aucugcgggg ggaaugucac acuugcauuu acaacaugau gggaaagaga 9060
gagaaaaaac ccggagaguu cggaaaggcc aagggaagca gagccauuug guucaugugg 9120
cucggagcuc gcuuucugga guucgaggcu cuggguuuuc ucaaugaaga ccacuggcuu 9180
ggaagaaaga acucaggagg aggugucgag ggcuugggcc uccaaaaacu ggguuacauc 9240
cugcgugaag uuggcacccg gccugggggc aagaucuaug cugaugacac agcuggcugg 9300
gacacccgca ucacgagagc ugacuuggaa aaugaagcua aggugcuuga gcugcuugau 9360
ggggaacauc ggcgucuugc cagggccauc auugagcuca ccuaucguca caaaguugug 9420
aaagugaugc gcccggcugc ugauggaaga accgucaugg auguuaucuc cagagaagau 9480
cagaggggga guggacaagu ugucaccuac gcccuaaaca cuuucaccaa ccuggccguc 9540
cagcugguga ggaugaugga aggggaagga gugauuggcc cagaugaugu ggagaaacuc 9600
acaaaaggga aaggacccaa agucaggacc uggcuguuug agaaugggga agaaagacuc 9660
agccgcaugg cugucagugg agaugacugu gugguaaagc cccuggacga ucgcuuugcc 9720
accucgcucc acuuccucaa ugcuauguca aagguucgca aagacaucca agaguggaaa 9780
ccgucaacug gaugguauga uuggcagcag guuccauuuu gcucaaacca uuucacugaa 9840
uugaucauga aagauggaag aacacuggug guuccaugcc gaggacagga ugaauuggua 9900
ggcagagcuc gcauaucucc aggggccgga uggaacgucc gcgacacugc uugucuggcu 9960
aagucuuaug cccagaugug gcugcuucug uacuuccaca gaagagaccu gcggcucaug 10020
gccaacgcca uuugcuccgc ugucccugug aauugggucc cuaccggaag aaccacgugg 10080
uccauccaug caggaggaga guggaugaca acagaggaca uguuggaggu cuggaaccgu 10140
guuuggauag aggagaauga auggauggaa gacaaaaccc caguggagaa auggagugac 10200
gucccauauu caggaaaacg agaggacauc ugguguggca gccugauugg cacaagagcc 10260
cgagccacgu gggcagaaaa cauccaggug gcuaucaacc aagucagagc aaucaucgga 10320
gaugagaagu auguggauua caugaguuca cuaaagagau augaagacac aacuuugguu 10380
gaggacacag uacuguagau auuuaaucaa uuguaaauag acaauauaag uaugcauaaa 10440
aguguaguuu uauaguagua uuuaguggug uuaguguaaa uaguuaagaa aauuuugagg 10500
agaaagucag gccgggaagu ucccgccacc ggaaguugag uagacggugc ugccugcgac 10560
ucaaccccag gaggacuggg ugaacaaagc cgcgaaguga uccauguaag cccucagaac 10620
cguuucggaa ggaggacccc acauguugua acuucaaagc ccaaugucag accacgcuac 10680
ggcgugcuac ucugcggaga gugcagucug cgauagugcc ccaggaggac uggguuaaca 10740
aaggcaaacc aacgccccac gcggcccuag ccccgguaau gguguuaacc agggcgaaag 10800
gacuagaggu uagaggagac cccgcgguuu aaagugcacg gcccagccug gcugaagcug 10860
uaggucaggg gaaggacuag agguuagugg agaccccgug ccacaaaaca ccacaacaaa 10920
acagcauauu gacaccuggg auagacuagg agaucuucug cucugcacaa ccagccacac 10980
ggcacagugc gccgacaaug guggcuggug gugcgagaac acaggaucu 11029

SEQ ID NO: 21

<211> 270
<212> RNA
<213> Artificial Sequence
<220>
<223> dchp_wsll_A
<400> 21

ugcaguuaga uaauauuggg agguguguuu uuugccuuuu uccguagauc cucgcgaaac 60
uucuuggccu gguuuucuuu ucaaaagagu ccuuacguau auuggagggu guguuuuucg 120
cuuuuuuccg ucucggaaac gauaacuucc uggcuugauu uuuucuucaa aagcugcguu 180
aucuauauug agaggcgugu uuuucgucuu uuuccguucc aaccuggaga acuuuuuggc 240
cugauuuucu cuucaaaacc aggccguacc 270

SEQ ID NO: 22

<211> 270
<212> RNA
<213> Artificial Sequence
<220>
<223> dchp_wsll_B
<400> 22

ugcaguuaga uaauauugga aggcguguuu uuugcuuuuu uccguagauc cuaacggaac 60
uuccuggccu gauuuucuuc uuaaaagagu ccuuacguau auugaaaggu guguuuuucg 120
ccuuuuuccg ucucggaaac gauaacuuuc cggccugguu uuuuccuuaa aagcugcguu 180
aucuauauug aggggugugu uuuuugucuu uuuccguucc aaccuggaga acuuccuggu 240
uuggcuuuuu ucuugaaacc aggccguacc 270

SEQ ID NO: 23

<211> 270
<212> RNA
<213> Artificial Sequence
<220>
<223> dchp_wsll_C
<400> 23

ugcaguuaga uaauauuggg agguguguuu uuugccuuuu uccguagauc cucgcgaaac 60
uucuuggccu gguuuucuuu ucaaaagagu ccuuacguau auuggagggu guguuuuucg 120
cuuuuuuccg ucucggaaac gauaacuucc uggcuugauu uuuucuucaa aagcugcguu 180
aucuauauug agaggcgugu uuuucgucuu uuuccguucc aaccuggaga acuucccggc 240
cugacuuuuu ccuuaaaacc aggccguacc 270

SEQ ID NO: 24

<211> 299
<212> RNA
<213> Artificial Sequence
<220>
<223> circ_wnv_slll_1
<400> 24

ugcaguuaga uaaacuuucu gguuugguuu ucuccucaaa agacaguucu ucgaacuucc 60
cgguuugauu uucucuuuaa aaauggggcc ccucaacuuc uuggucugac uuucucuuca 120
aaaggcuaua cgaucaacuu cucggccuga uuuucuuuuc aaaaagcuau caucgcaacu 180
uccuggucug guuuucuucu caaaacccuc gguaaccaac uuuccggcuu gacuuucuuc 240
uuaaaacguu uugcgacaaa cuuuucggcu uggcuuucuc cuuaaaauau guaaacagc 299

SEQ ID NO: 25

<211> 33
<212> RNA
<213> Artificial Sequence
<220>
<223> Target sequence IRES Circ HCV1
<400> 25

cuccgccaug aaucacuccc cugugaggaa cua 33

SEQ ID NO: 26

<211> 24
<212> RNA
<213> Artificial Sequence
<220>
<223> Target sequence IRES Circ HCV2
<400> 26

ucucguagac cgugcaccau gagc 24

SEQ ID NO: 27

<211> 28
<212> RNA
<213> Artificial Sequence
<220>
<223> Target sequence cHP (CDS1) circ_hcv_cds1
<400> 27

ccaaaagaaa caccaaccgu cgcccaga 28

SEQ ID NO: 28

<211> 16
<212> RNA
<213> Artificial Sequence
<220>
<223> Target sequence CDS2 circ_hcv_cds2
<400> 28

ggggccccag g 16

SEQ ID NO: 29

<211> 32
<212> RNA
<213> Artificial Sequence
<220>
<223> Target sequence sHP (3′ UTR) circ_dv_3utr
<400> 29

aacagcauau ugacgcuggg aaagaccaga ga 32

SEQ ID NO: 30

<211> 33
<212> RNA
<213> Artificial Sequence
<220>
<223> Target sequence cHP½ circ_dv_cHP_v½
<400> 30

acggaaaaag gcgaaaaaca cgccuuucaa uau 33

SEQ ID NO: 31

<211> 34
<212> RNA
<213> Artificial Sequence
<220>
<223> Target sequence Chikv_re
<400> 31

gcugcuguaa aacguuggcu uuuuuagccg uaau 34

SEQ ID NO: 32

<211> 294
<212> RNA
<213> Artificial Sequence
<220>
<223> Broad Spectrum Circular Artificial sequence to DENV cHP and HCV
CDS (circ_dv_cHP_v1_circ_hcv_cds2_2)
<400> 32

gcgccgaugu ugaaaggugu guuuuucguu uuuuucuguc aaguguagga ucuucugggg 60
ucucgggccc cacgaccaau guuggggggc guguuuuucg cuuuuuucug ucaaaauaau 120
guugaccugg gguucuuuac ugaacucauu auguugggag gcguguuuuu ugucuuuuuu 180
uguuuuaucu acacccgucu gggguuuugu cggcgccacg acauguuggg ggguguguuu 240
uuugccuuuu uccgucucag ugcggcgccc uugggguucc cuuucgcauc acgu 294

SEQ ID NO: 33

<211> 35
<212> RNA
<213> Artificial Sequence
<220>
<223> Target sequence 5 UTR chikv5utr
<400> 33

acacacguag ccuaccaguu ucuuacugcu cuacu 35

SEQ ID NO: 34

<211> 29903
<212> RNA
<213> Coronaviridae
<220>
<223> NC_045512.2 Severe acute respiratory syndrome coronavirus 2
(SARS-CoV-2) isolate Wuhan-Hu-1, complete genome
<400> 34

auuaaagguu uauaccuucc cagguaacaa accaaccaac uuucgaucuc uuguagaucu 60
guucucuaaa cgaacuuuaa aaucugugug gcugucacuc ggcugcaugc uuagugcacu 120
cacgcaguau aauuaauaac uaauuacugu cguugacagg acacgaguaa cucgucuauc 180
uucugcaggc ugcuuacggu uucguccgug uugcagccga ucaucagcac aucuagguuu 240
cguccgggug ugaccgaaag guaagaugga gagccuuguc ccugguuuca acgagaaaac 300
acacguccaa cucaguuugc cuguuuuaca gguucgcgac gugcucguac guggcuuugg 360
agacuccgug gaggaggucu uaucagaggc acgucaacau cuuaaagaug gcacuugugg 420
cuuaguagaa guugaaaaag gcguuuugcc ucaacuugaa cagcccuaug uguucaucaa 480
acguucggau gcucgaacug caccucaugg ucauguuaug guugagcugg uagcagaacu 540
cgaaggcauu caguacgguc guagugguga gacacuuggu guccuugucc cucauguggg 600
cgaaauacca guggcuuacc gcaagguucu ucuucguaag aacgguaaua aaggagcugg 660
uggccauagu uacggcgccg aucuaaaguc auuugacuua ggcgacgagc uuggcacuga 720
uccuuaugaa gauuuucaag aaaacuggaa cacuaaacau agcaguggug uuacccguga 780
acucaugcgu gagcuuaacg gaggggcaua cacucgcuau gucgauaaca acuucugugg 840
cccugauggc uacccucuug agugcauuaa agaccuucua gcacgugcug guaaagcuuc 900
augcacuuug uccgaacaac uggacuuuau ugacacuaag agggguguau acugcugccg 960
ugaacaugag caugaaauug cuugguacac ggaacguucu gaaaagagcu augaauugca 1020
gacaccuuuu gaaauuaaau uggcaaagaa auuugacacc uucaaugggg aauguccaaa 1080
uuuuguauuu cccuuaaauu ccauaaucaa gacuauucaa ccaaggguug aaaagaaaaa 1140
gcuugauggc uuuaugggua gaauucgauc ugucuaucca guugcgucac caaaugaaug 1200
caaccaaaug ugccuuucaa cucucaugaa gugugaucau uguggugaaa cuucauggca 1260
gacgggcgau uuuguuaaag ccacuugcga auuuuguggc acugagaauu ugacuaaaga 1320
aggugccacu acuugugguu acuuacccca aaaugcuguu guuaaaauuu auuguccagc 1380
augucacaau ucagaaguag gaccugagca uagucuugcc gaauaccaua augaaucugg 1440
cuugaaaacc auucuucgua aggguggucg cacuauugcc uuuggaggcu guguguucuc 1500
uuauguuggu ugccauaaca agugugccua uuggguucca cgugcuagcg cuaacauagg 1560
uuguaaccau acagguguug uuggagaagg uuccgaaggu cuuaaugaca accuucuuga 1620
aauacuccaa aaagagaaag ucaacaucaa uauuguuggu gacuuuaaac uuaaugaaga 1680
gaucgccauu auuuuggcau cuuuuucugc uuccacaagu gcuuuugugg aaacugugaa 1740
agguuuggau uauaaagcau ucaaacaaau uguugaaucc ugugguaauu uuaaaguuac 1800
aaaaggaaaa gcuaaaaaag gugccuggaa uauuggugaa cagaaaucaa uacugagucc 1860
ucuuuaugca uuugcaucag aggcugcucg uguuguacga ucaauuuucu cccgcacucu 1920
ugaaacugcu caaaauucug ugcguguuuu acagaaggcc gcuauaacaa uacuagaugg 1980
aauuucacag uauucacuga gacucauuga ugcuaugaug uucacaucug auuuggcuac 2040
uaacaaucua guuguaaugg ccuacauuac aggugguguu guucaguuga cuucgcagug 2100
gcuaacuaac aucuuuggca cuguuuauga aaaacucaaa cccguccuug auuggcuuga 2160
agagaaguuu aaggaaggug uagaguuucu uagagacggu ugggaaauug uuaaauuuau 2220
cucaaccugu gcuugugaaa uugucggugg acaaauuguc accugugcaa aggaaauuaa 2280
ggagaguguu cagacauucu uuaagcuugu aaauaaauuu uuggcuuugu gugcugacuc 2340
uaucauuauu gguggagcua aacuuaaagc cuugaauuua ggugaaacau uugucacgca 2400
cucaaaggga uuguacagaa aguguguuaa auccagagaa gaaacuggcc uacucaugcc 2460
ucuaaaagcc ccaaaagaaa uuaucuucuu agagggagaa acacuuccca cagaaguguu 2520
aacagaggaa guugucuuga aaacugguga uuuacaacca uuagaacaac cuacuaguga 2580
agcuguugaa gcuccauugg uugguacacc aguuuguauu aacgggcuua uguugcucga 2640
aaucaaagac acagaaaagu acugugcccu ugcaccuaau augaugguaa caaacaauac 2700
cuucacacuc aaaggcggug caccaacaaa gguuacuuuu ggugaugaca cugugauaga 2760
agugcaaggu uacaagagug ugaauaucac uuuugaacuu gaugaaagga uugauaaagu 2820
acuuaaugag aagugcucug ccuauacagu ugaacucggu acagaaguaa augaguucgc 2880
cuguguugug gcagaugcug ucauaaaaac uuugcaacca guaucugaau uacuuacacc 2940
acugggcauu gauuuagaug aguggaguau ggcuacauac uacuuauuug augagucugg 3000
ugaguuuaaa uuggcuucac auauguauug uucuuucuac ccuccagaug aggaugaaga 3060
agaaggugau ugugaagaag aagaguuuga gccaucaacu caauaugagu augguacuga 3120
agaugauuac caagguaaac cuuuggaauu uggugccacu ucugcugcuc uucaaccuga 3180
agaagagcaa gaagaagauu gguuagauga ugauagucaa caaacuguug gucaacaaga 3240
cggcagugag gacaaucaga caacuacuau ucaaacaauu guugagguuc aaccucaauu 3300
agagauggaa cuuacaccag uuguucagac uauugaagug aauaguuuua gugguuauuu 3360
aaaacuuacu gacaauguau acauuaaaaa ugcagacauu guggaagaag cuaaaaaggu 3420
aaaaccaaca gugguuguua augcagccaa uguuuaccuu aaacauggag gagguguugc 3480
aggagccuua aauaaggcua cuaacaaugc caugcaaguu gaaucugaug auuacauagc 3540
uacuaaugga ccacuuaaag ugggugguag uuguguuuua agcggacaca aucuugcuaa 3600
acacugucuu cauguugucg gcccaaaugu uaacaaaggu gaagacauuc aacuucuuaa 3660
gagugcuuau gaaaauuuua aucagcacga aguucuacuu gcaccauuau uaucagcugg 3720
uauuuuuggu gcugacccua uacauucuuu aagaguuugu guagauacug uucgcacaaa 3780
ugucuacuua gcugucuuug auaaaaaucu cuaugacaaa cuuguuucaa gcuuuuugga 3840
aaugaagagu gaaaagcaag uugaacaaaa gaucgcugag auuccuaaag aggaaguuaa 3900
gccauuuaua acugaaagua aaccuucagu ugaacagaga aaacaagaug auaagaaaau 3960
caaagcuugu guugaagaag uuacaacaac ucuggaagaa acuaaguucc ucacagaaaa 4020
cuuguuacuu uauauugaca uuaauggcaa ucuucaucca gauucugcca cucuuguuag 4080
ugacauugac aucacuuucu uaaagaaaga ugcuccauau auagugggug auguuguuca 4140
agaggguguu uuaacugcug ugguuauacc uacuaaaaag gcugguggca cuacugaaau 4200
gcuagcgaaa gcuuugagaa aagugccaac agacaauuau auaaccacuu acccggguca 4260
ggguuuaaau gguuacacug uagaggaggc aaagacagug cuuaaaaagu guaaaagugc 4320
cuuuuacauu cuaccaucua uuaucucuaa ugagaagcaa gaaauucuug gaacuguuuc 4380
uuggaauuug cgagaaaugc uugcacaugc agaagaaaca cgcaaauuaa ugccugucug 4440
uguggaaacu aaagccauag uuucaacuau acagcguaaa uauaagggua uuaaaauaca 4500
agagggugug guugauuaug gugcuagauu uuacuuuuac accaguaaaa caacuguagc 4560
gucacuuauc aacacacuua acgaucuaaa ugaaacucuu guuacaaugc cacuuggcua 4620
uguaacacau ggcuuaaauu uggaagaagc ugcucgguau augagaucuc ucaaagugcc 4680
agcuacaguu ucuguuucuu caccugaugc uguuacagcg uauaaugguu aucuuacuuc 4740
uucuucuaaa acaccugaag aacauuuuau ugaaaccauc ucacuugcug guuccuauaa 4800
agauuggucc uauucuggac aaucuacaca acuagguaua gaauuucuua agagagguga 4860
uaaaagugua uauuacacua guaauccuac cacauuccac cuagauggug aaguuaucac 4920
cuuugacaau cuuaagacac uucuuucuuu gagagaagug aggacuauua agguguuuac 4980
aacaguagac aacauuaacc uccacacgca aguuguggac augucaauga cauauggaca 5040
acaguuuggu ccaacuuauu uggauggagc ugauguuacu aaaauaaaac cucauaauuc 5100
acaugaaggu aaaacauuuu auguuuuacc uaaugaugac acucuacgug uugaggcuuu 5160
ugaguacuac cacacaacug auccuaguuu ucuggguagg uacaugucag cauuaaauca 5220
cacuaaaaag uggaaauacc cacaaguuaa ugguuuaacu ucuauuaaau gggcagauaa 5280
caacuguuau cuugccacug cauuguuaac acuccaacaa auagaguuga aguuuaaucc 5340
accugcucua caagaugcuu auuacagagc aagggcuggu gaagcugcua acuuuugugc 5400
acuuaucuua gccuacugua auaagacagu aggugaguua ggugauguua gagaaacaau 5460
gaguuacuug uuucaacaug ccaauuuaga uucuugcaaa agagucuuga acguggugug 5520
uaaaacuugu ggacaacagc agacaacccu uaagggugua gaagcuguua uguacauggg 5580
cacacuuucu uaugaacaau uuaagaaagg uguucagaua ccuuguacgu gugguaaaca 5640
agcuacaaaa uaucuaguac aacaggaguc accuuuuguu augaugucag caccaccugc 5700
ucaguaugaa cuuaagcaug guacauuuac uugugcuagu gaguacacug guaauuacca 5760
guguggucac uauaaacaua uaacuucuaa agaaacuuug uauugcauag acggugcuuu 5820
acuuacaaag uccucagaau acaaaggucc uauuacggau guuuucuaca aagaaaacag 5880
uuacacaaca accauaaaac caguuacuua uaaauuggau gguguuguuu guacagaaau 5940
ugacccuaag uuggacaauu auuauaagaa agacaauucu uauuucacag agcaaccaau 6000
ugaucuugua ccaaaccaac cauauccaaa cgcaagcuuc gauaauuuua aguuuguaug 6060
ugauaauauc aaauuugcug augauuuaaa ccaguuaacu gguuauaaga aaccugcuuc 6120
aagagagcuu aaaguuacau uuuucccuga cuuaaauggu gauguggugg cuauugauua 6180
uaaacacuac acacccucuu uuaagaaagg agcuaaauug uuacauaaac cuauuguuug 6240
gcauguuaac aaugcaacua auaaagccac guauaaacca aauaccuggu guauacguug 6300
ucuuuggagc acaaaaccag uugaaacauc aaauucguuu gauguacuga agucagagga 6360
cgcgcaggga auggauaauc uugccugcga agaucuaaaa ccagucucug aagaaguagu 6420
ggaaaauccu accauacaga aagacguucu ugaguguaau gugaaaacua ccgaaguugu 6480
aggagacauu auacuuaaac cagcaaauaa uaguuuaaaa auuacagaag agguuggcca 6540
cacagaucua auggcugcuu auguagacaa uucuagucuu acuauuaaga aaccuaauga 6600
auuaucuaga guauuagguu ugaaaacccu ugcuacucau gguuuagcug cuguuaauag 6660
ugucccuugg gauacuauag cuaauuaugc uaagccuuuu cuuaacaaag uuguuaguac 6720
aacuacuaac auaguuacac gguguuuaaa ccguguuugu acuaauuaua ugccuuauuu 6780
cuuuacuuua uugcuacaau uguguacuuu uacuagaagu acaaauucua gaauuaaagc 6840
aucuaugccg acuacuauag caaagaauac uguuaagagu gucgguaaau uuugucuaga 6900
ggcuucauuu aauuauuuga agucaccuaa uuuuucuaaa cugauaaaua uuauaauuug 6960
guuuuuacua uuaaguguuu gccuagguuc uuuaaucuac ucaaccgcug cuuuaggugu 7020
uuuaaugucu aauuuaggca ugccuucuua cuguacuggu uacagagaag gcuauuugaa 7080
cucuacuaau gucacuauug caaccuacug uacugguucu auaccuugua guguuugucu 7140
uagugguuua gauucuuuag acaccuaucc uucuuuagaa acuauacaaa uuaccauuuc 7200
aucuuuuaaa ugggauuuaa cugcuuuugg cuuaguugca gagugguuuu uggcauauau 7260
ucuuuucacu agguuuuucu auguacuugg auuggcugca aucaugcaau uguuuuucag 7320
cuauuuugca guacauuuua uuaguaauuc uuggcuuaug ugguuaauaa uuaaucuugu 7380
acaaauggcc ccgauuucag cuaugguuag aauguacauc uucuuugcau cauuuuauua 7440
uguauggaaa aguuaugugc auguuguaga cgguuguaau ucaucaacuu guaugaugug 7500
uuacaaacgu aauagagcaa caagagucga auguacaacu auuguuaaug guguuagaag 7560
guccuuuuau gucuaugcua auggagguaa aggcuuuugc aaacuacaca auuggaauug 7620
uguuaauugu gauacauucu gugcugguag uacauuuauu agugaugaag uugcgagaga 7680
cuugucacua caguuuaaaa gaccaauaaa uccuacugac cagucuucuu acaucguuga 7740
uaguguuaca gugaagaaug guuccaucca ucuuuacuuu gauaaagcug gucaaaagac 7800
uuaugaaaga cauucucucu cucauuuugu uaacuuagac aaccugagag cuaauaacac 7860
uaaagguuca uugccuauua auguuauagu uuuugauggu aaaucaaaau gugaagaauc 7920
aucugcaaaa ucagcgucug uuuacuacag ucagcuuaug ugucaaccua uacuguuacu 7980
agaucaggca uuagugucug auguugguga uagugcggaa guugcaguua aaauguuuga 8040
ugcuuacguu aauacguuuu caucaacuuu uaacguacca auggaaaaac ucaaaacacu 8100
aguugcaacu gcagaagcug aacuugcaaa gaaugugucc uuagacaaug ucuuaucuac 8160
uuuuauuuca gcagcucggc aaggguuugu ugauucagau guagaaacua aagauguugu 8220
ugaaugucuu aaauugucac aucaaucuga cauagaaguu acuggcgaua guuguaauaa 8280
cuauaugcuc accuauaaca aaguugaaaa caugacaccc cgugaccuug gugcuuguau 8340
ugacuguagu gcgcgucaua uuaaugcgca gguagcaaaa agucacaaca uugcuuugau 8400
auggaacguu aaagauuuca ugucauuguc ugaacaacua cgaaaacaaa uacguagugc 8460
ugcuaaaaag aauaacuuac cuuuuaaguu gacaugugca acuacuagac aaguuguuaa 8520
uguuguaaca acaaagauag cacuuaaggg ugguaaaauu guuaauaauu gguugaagca 8580
guuaauuaaa guuacacuug uguuccuuuu uguugcugcu auuuucuauu uaauaacacc 8640
uguucauguc augucuaaac auacugacuu uucaagugaa aucauaggau acaaggcuau 8700
ugaugguggu gucacucgug acauagcauc uacagauacu uguuuugcua acaaacaugc 8760
ugauuuugac acaugguuua gccagcgugg ugguaguuau acuaaugaca aagcuugccc 8820
auugauugcu gcagucauaa caagagaagu ggguuuuguc gugccugguu ugccuggcac 8880
gauauuacgc acaacuaaug gugacuuuuu gcauuucuua ccuagaguuu uuagugcagu 8940
ugguaacauc uguuacacac caucaaaacu uauagaguac acugacuuug caacaucagc 9000
uuguguuuug gcugcugaau guacaauuuu uaaagaugcu ucugguaagc caguaccaua 9060
uuguuaugau accaauguac uagaagguuc uguugcuuau gaaaguuuac gcccugacac 9120
acguuaugug cucauggaug gcucuauuau ucaauuuccu aacaccuacc uugaagguuc 9180
uguuagagug guaacaacuu uugauucuga guacuguagg cacggcacuu gugaaagauc 9240
agaagcuggu guuuguguau cuacuagugg uagaugggua cuuaacaaug auuauuacag 9300
aucuuuacca ggaguuuucu gugguguaga ugcuguaaau uuacuuacua auauguuuac 9360
accacuaauu caaccuauug gugcuuugga cauaucagca ucuauaguag cuggugguau 9420
uguagcuauc guaguaacau gccuugccua cuauuuuaug agguuuagaa gagcuuuugg 9480
ugaauacagu cauguaguug ccuuuaauac uuuacuauuc cuuaugucau ucacuguacu 9540
cuguuuaaca ccaguuuacu cauucuuacc ugguguuuau ucuguuauuu acuuguacuu 9600
gacauuuuau cuuacuaaug auguuucuuu uuuagcacau auucagugga ugguuauguu 9660
cacaccuuua guaccuuucu ggauaacaau ugcuuauauc auuuguauuu ccacaaagca 9720
uuucuauugg uucuuuagua auuaccuaaa gagacgugua gucuuuaaug guguuuccuu 9780
uaguacuuuu gaagaagcug cgcugugcac cuuuuuguua aauaaagaaa uguaucuaaa 9840
guugcguagu gaugugcuau uaccucuuac gcaauauaau agauacuuag cucuuuauaa 9900
uaaguacaag uauuuuagug gagcaaugga uacaacuagc uacagagaag cugcuuguug 9960
ucaucucgca aaggcucuca augacuucag uaacucaggu ucugauguuc uuuaccaacc 10020
accacaaacc ucuaucaccu cagcuguuuu gcagaguggu uuuagaaaaa uggcauuccc 10080
aucugguaaa guugaggguu guaugguaca aguaacuugu gguacaacua cacuuaacgg 10140
ucuuuggcuu gaugacguag uuuacugucc aagacaugug aucugcaccu cugaagacau 10200
gcuuaacccu aauuaugaag auuuacucau ucguaagucu aaucauaauu ucuugguaca 10260
ggcugguaau guucaacuca ggguuauugg acauucuaug caaaauugug uacuuaagcu 10320
uaagguugau acagccaauc cuaagacacc uaaguauaag uuuguucgca uucaaccagg 10380
acagacuuuu ucaguguuag cuuguuacaa ugguucacca ucugguguuu accaaugugc 10440
uaugaggccc aauuucacua uuaaggguuc auuccuuaau gguucaugug guaguguugg 10500
uuuuaacaua gauuaugacu gugucucuuu uuguuacaug caccauaugg aauuaccaac 10560
uggaguucau gcuggcacag acuuagaagg uaacuuuuau ggaccuuuug uugacaggca 10620
aacagcacaa gcagcuggua cggacacaac uauuacaguu aauguuuuag cuugguugua 10680
cgcugcuguu auaaauggag acaggugguu ucucaaucga uuuaccacaa cucuuaauga 10740
cuuuaaccuu guggcuauga aguacaauua ugaaccucua acacaagacc auguugacau 10800
acuaggaccu cuuucugcuc aaacuggaau ugccguuuua gauaugugug cuucauuaaa 10860
agaauuacug caaaauggua ugaauggacg uaccauauug gguagugcuu uauuagaaga 10920
ugaauuuaca ccuuuugaug uuguuagaca augcucaggu guuacuuucc aaagugcagu 10980
gaaaagaaca aucaagggua cacaccacug guuguuacuc acaauuuuga cuucacuuuu 11040
aguuuuaguc cagaguacuc aauggucuuu guucuuuuuu uuguaugaaa augccuuuuu 11100
accuuuugcu auggguauua uugcuauguc ugcuuuugca augauguuug ucaaacauaa 11160
gcaugcauuu cucuguuugu uuuuguuacc uucucuugcc acuguagcuu auuuuaauau 11220
ggucuauaug ccugcuaguu gggugaugcg uauuaugaca ugguuggaua ugguugauac 11280
uaguuugucu gguuuuaagc uaaaagacug uguuauguau gcaucagcug uaguguuacu 11340
aauccuuaug acagcaagaa cuguguauga ugauggugcu aggagagugu ggacacuuau 11400
gaaugucuug acacucguuu auaaaguuua uuaugguaau gcuuuagauc aagccauuuc 11460
caugugggcu cuuauaaucu cuguuacuuc uaacuacuca gguguaguua caacugucau 11520
guuuuuggcc agagguauug uuuuuaugug uguugaguau ugcccuauuu ucuucauaac 11580
ugguaauaca cuucagugua uaaugcuagu uuauuguuuc uuaggcuauu uuuguacuug 11640
uuacuuuggc cucuuuuguu uacucaaccg cuacuuuaga cugacucuug guguuuauga 11700
uuacuuaguu ucuacacagg aguuuagaua uaugaauuca cagggacuac ucccacccaa 11760
gaauagcaua gaugccuuca aacucaacau uaaauuguug gguguuggug gcaaaccuug 11820
uaucaaagua gccacuguac agucuaaaau gucagaugua aagugcacau caguagucuu 11880
acucucaguu uugcaacaac ucagaguaga aucaucaucu aaauuguggg cucaaugugu 11940
ccaguuacac aaugacauuc ucuuagcuaa agauacuacu gaagccuuug aaaaaauggu 12000
uucacuacuu ucuguuuugc uuuccaugca gggugcugua gacauaaaca agcuuuguga 12060
agaaaugcug gacaacaggg caaccuuaca agcuauagcc ucagaguuua guucccuucc 12120
aucauaugca gcuuuugcua cugcucaaga agcuuaugag caggcuguug cuaaugguga 12180
uucugaaguu guucuuaaaa aguugaagaa gucuuugaau guggcuaaau cugaauuuga 12240
ccgugaugca gccaugcaac guaaguugga aaagauggcu gaucaagcua ugacccaaau 12300
guauaaacag gcuagaucug aggacaagag ggcaaaaguu acuagugcua ugcagacaau 12360
gcuuuucacu augcuuagaa aguuggauaa ugaugcacuc aacaacauua ucaacaaugc 12420
aagagauggu uguguucccu ugaacauaau accucuuaca acagcagcca aacuaauggu 12480
ugucauacca gacuauaaca cauauaaaaa uacgugugau gguacaacau uuacuuaugc 12540
aucagcauug ugggaaaucc aacagguugu agaugcagau aguaaaauug uucaacuuag 12600
ugaaauuagu auggacaauu caccuaauuu agcauggccu cuuauuguaa cagcuuuaag 12660
ggccaauucu gcugucaaau uacagaauaa ugagcuuagu ccuguugcac uacgacagau 12720
gucuugugcu gccgguacua cacaaacugc uugcacugau gacaaugcgu uagcuuacua 12780
caacacaaca aagggaggua gguuuguacu ugcacuguua uccgauuuac aggauuugaa 12840
augggcuaga uucccuaaga gugauggaac ugguacuauc uauacagaac uggaaccacc 12900
uuguagguuu guuacagaca caccuaaagg uccuaaagug aaguauuuau acuuuauuaa 12960
aggauuaaac aaccuaaaua gagguauggu acuugguagu uuagcugcca caguacgucu 13020
acaagcuggu aaugcaacag aagugccugc caauucaacu guauuaucuu ucugugcuuu 13080
ugcuguagau gcugcuaaag cuuacaaaga uuaucuagcu agugggggac aaccaaucac 13140
uaauuguguu aagauguugu guacacacac ugguacuggu caggcaauaa caguuacacc 13200
ggaagccaau auggaucaag aauccuuugg uggugcaucg uguugucugu acugccguug 13260
ccacauagau cauccaaauc cuaaaggauu uugugacuua aaagguaagu auguacaaau 13320
accuacaacu ugugcuaaug acccuguggg uuuuacacuu aaaaacacag ucuguaccgu 13380
cugcgguaug uggaaagguu auggcuguag uugugaucaa cuccgcgaac ccaugcuuca 13440
gucagcugau gcacaaucgu uuuuaaacgg guuugcggug uaagugcagc ccgucuuaca 13500
ccgugcggca caggcacuag uacugauguc guauacaggg cuuuugacau cuacaaugau 13560
aaaguagcug guuuugcuaa auuccuaaaa acuaauuguu gucgcuucca agaaaaggac 13620
gaagaugaca auuuaauuga uucuuacuuu guaguuaaga gacacacuuu cucuaacuac 13680
caacaugaag aaacaauuua uaauuuacuu aaggauuguc cagcuguugc uaaacaugac 13740
uucuuuaagu uuagaauaga cggugacaug guaccacaua uaucacguca acgucuuacu 13800
aaauacacaa uggcagaccu cgucuaugcu uuaaggcauu uugaugaagg uaauugugac 13860
acauuaaaag aaauacuugu cacauacaau uguugugaug augauuauuu caauaaaaag 13920
gacugguaug auuuuguaga aaacccagau auauuacgcg uauacgccaa cuuaggugaa 13980
cguguacgcc aagcuuuguu aaaaacagua caauucugug augccaugcg aaaugcuggu 14040
auuguuggug uacugacauu agauaaucaa gaucucaaug guaacuggua ugauuucggu 14100
gauuucauac aaaccacgcc agguagugga guuccuguug uagauucuua uuauucauug 14160
uuaaugccua uauuaaccuu gaccagggcu uuaacugcag agucacaugu ugacacugac 14220
uuaacaaagc cuuacauuaa gugggauuug uuaaaauaug acuucacgga agagagguua 14280
aaacucuuug accguuauuu uaaauauugg gaucagacau accacccaaa uuguguuaac 14340
uguuuggaug acagaugcau ucugcauugu gcaaacuuua auguuuuauu cucuacagug 14400
uucccaccua caaguuuugg accacuagug agaaaaauau uuguugaugg uguuccauuu 14460
guaguuucaa cuggauacca cuucagagag cuagguguug uacauaauca ggauguaaac 14520
uuacauagcu cuagacuuag uuuuaaggaa uuacuugugu augcugcuga cccugcuaug 14580
cacgcugcuu cugguaaucu auuacuagau aaacgcacua cgugcuuuuc aguagcugca 14640
cuuacuaaca auguugcuuu ucaaacuguc aaacccggua auuuuaacaa agacuucuau 14700
gacuuugcug ugucuaaggg uuucuuuaag gaaggaaguu cuguugaauu aaaacacuuc 14760
uucuuugcuc aggaugguaa ugcugcuauc agcgauuaug acuacuaucg uuauaaucua 14820
ccaacaaugu gugauaucag acaacuacua uuuguaguug aaguuguuga uaaguacuuu 14880
gauuguuacg augguggcug uauuaaugcu aaccaaguca ucgucaacaa ccuagacaaa 14940
ucagcugguu uuccauuuaa uaaauggggu aaggcuagac uuuauuauga uucaaugagu 15000
uaugaggauc aagaugcacu uuucgcauau acaaaacgua augucauccc uacuauaacu 15060
caaaugaauc uuaaguaugc cauuagugca aagaauagag cucgcaccgu agcugguguc 15120
ucuaucugua guacuaugac caauagacag uuucaucaaa aauuauugaa aucaauagcc 15180
gccacuagag gagcuacugu aguaauugga acaagcaaau ucuauggugg uuggcacaac 15240
auguuaaaaa cuguuuauag ugauguagaa aacccucacc uuauggguug ggauuauccu 15300
aaaugugaua gagccaugcc uaacaugcuu agaauuaugg ccucacuugu ucuugcucgc 15360
aaacauacaa cguguuguag cuugucacac cguuucuaua gauuagcuaa ugagugugcu 15420
caaguauuga gugaaauggu cauguguggc gguucacuau auguuaaacc agguggaacc 15480
ucaucaggag augccacaac ugcuuaugcu aauaguguuu uuaacauuug ucaagcuguc 15540
acggccaaug uuaaugcacu uuuaucuacu gaugguaaca aaauugccga uaaguauguc 15600
cgcaauuuac aacacagacu uuaugagugu cucuauagaa auagagaugu ugacacagac 15660
uuugugaaug aguuuuacgc auauuugcgu aaacauuucu caaugaugau acucucugac 15720
gaugcuguug uguguuucaa uagcacuuau gcaucucaag gucuaguggc uagcauaaag 15780
aacuuuaagu caguucuuua uuaucaaaac aauguuuuua ugucugaagc aaaauguugg 15840
acugagacug accuuacuaa aggaccucau gaauuuugcu cucaacauac aaugcuaguu 15900
aaacagggug augauuaugu guaccuuccu uacccagauc caucaagaau ccuaggggcc 15960
ggcuguuuug uagaugauau cguaaaaaca gaugguacac uuaugauuga acgguucgug 16020
ucuuuagcua uagaugcuua cccacuuacu aaacauccua aucaggagua ugcugauguc 16080
uuucauuugu acuuacaaua cauaagaaag cuacaugaug aguuaacagg acacauguua 16140
gacauguauu cuguuaugcu uacuaaugau aacacuucaa gguauuggga accugaguuu 16200
uaugaggcua uguacacacc gcauacaguc uuacaggcug uuggggcuug uguucuuugc 16260
aauucacaga cuucauuaag auguggugcu ugcauacgua gaccauucuu auguuguaaa 16320
ugcuguuacg accaugucau aucaacauca cauaaauuag ucuugucugu uaauccguau 16380
guuugcaaug cuccagguug ugaugucaca gaugugacuc aacuuuacuu aggagguaug 16440
agcuauuauu guaaaucaca uaaaccaccc auuaguuuuc cauugugugc uaauggacaa 16500
guuuuugguu uauauaaaaa uacauguguu gguagcgaua auguuacuga cuuuaaugca 16560
auugcaacau gugacuggac aaaugcuggu gauuacauuu uagcuaacac cuguacugaa 16620
agacucaagc uuuuugcagc agaaacgcuc aaagcuacug aggagacauu uaaacugucu 16680
uaugguauug cuacuguacg ugaagugcug ucugacagag aauuacaucu uucaugggaa 16740
guugguaaac cuagaccacc acuuaaccga aauuaugucu uuacugguua ucguguaacu 16800
aaaaacagua aaguacaaau aggagaguac accuuugaaa aaggugacua uggugaugcu 16860
guuguuuacc gagguacaac aacuuacaaa uuaaauguug gugauuauuu ugugcugaca 16920
ucacauacag uaaugccauu aagugcaccu acacuagugc cacaagagca cuauguuaga 16980
auuacuggcu uauacccaac acucaauauc ucagaugagu uuucuagcaa uguugcaaau 17040
uaucaaaagg uugguaugca aaaguauucu acacuccagg gaccaccugg uacugguaag 17100
agucauuuug cuauuggccu agcucucuac uacccuucug cucgcauagu guauacagcu 17160
ugcucucaug ccgcuguuga ugcacuaugu gagaaggcau uaaaauauuu gccuauagau 17220
aaauguagua gaauuauacc ugcacgugcu cguguagagu guuuugauaa auucaaagug 17280
aauucaacau uagaacagua ugucuuuugu acuguaaaug cauugccuga gacgacagca 17340
gauauaguug ucuuugauga aauuucaaug gccacaaauu augauuugag uguugucaau 17400
gccagauuac gugcuaagca cuauguguac auuggcgacc cugcucaauu accugcacca 17460
cgcacauugc uaacuaaggg cacacuagaa ccagaauauu ucaauucagu guguagacuu 17520
augaaaacua uagguccaga cauguuccuc ggaacuuguc ggcguugucc ugcugaaauu 17580
guugacacug ugagugcuuu gguuuaugau aauaagcuua aagcacauaa agacaaauca 17640
gcucaaugcu uuaaaauguu uuauaagggu guuaucacgc augauguuuc aucugcaauu 17700
aacaggccac aaauaggcgu gguaagagaa uuccuuacac guaacccugc uuggagaaaa 17760
gcugucuuua uuucaccuua uaauucacag aaugcuguag ccucaaagau uuugggacua 17820
ccaacucaaa cuguugauuc aucacagggc ucagaauaug acuaugucau auucacucaa 17880
accacugaaa cagcucacuc uuguaaugua aacagauuua auguugcuau uaccagagca 17940
aaaguaggca uacuuugcau aaugucugau agagaccuuu augacaaguu gcaauuuaca 18000
agucuugaaa uuccacguag gaauguggca acuuuacaag cugaaaaugu aacaggacuc 18060
uuuaaagauu guaguaaggu aaucacuggg uuacauccua cacaggcacc uacacaccuc 18120
aguguugaca cuaaauucaa aacugaaggu uuauguguug acauaccugg cauaccuaag 18180
gacaugaccu auagaagacu caucucuaug auggguuuua aaaugaauua ucaaguuaau 18240
gguuacccua acauguuuau cacccgcgaa gaagcuauaa gacauguacg ugcauggauu 18300
ggcuucgaug ucgaggggug ucaugcuacu agagaagcug uugguaccaa uuuaccuuua 18360
cagcuagguu uuucuacagg uguuaaccua guugcuguac cuacagguua uguugauaca 18420
ccuaauaaua cagauuuuuc cagaguuagu gcuaaaccac cgccuggaga ucaauuuaaa 18480
caccucauac cacuuaugua caaaggacuu ccuuggaaug uagugcguau aaagauugua 18540
caaauguuaa gugacacacu uaaaaaucuc ucugacagag ucguauuugu cuuaugggca 18600
cauggcuuug aguugacauc uaugaaguau uuugugaaaa uaggaccuga gcgcaccugu 18660
ugucuaugug auagacgugc cacaugcuuu uccacugcuu cagacacuua ugccuguugg 18720
caucauucua uuggauuuga uuacgucuau aauccguuua ugauugaugu ucaacaaugg 18780
gguuuuacag guaaccuaca aagcaaccau gaucuguauu gucaagucca ugguaaugca 18840
cauguagcua guugugaugc aaucaugacu aggugucuag cuguccacga gugcuuuguu 18900
aagcguguug acuggacuau ugaauauccu auaauuggug augaacugaa gauuaaugcg 18960
gcuuguagaa agguucaaca caugguuguu aaagcugcau uauuagcaga caaauuccca 19020
guucuucacg acauugguaa cccuaaagcu auuaagugug uaccucaagc ugauguagaa 19080
uggaaguucu augaugcaca gccuuguagu gacaaagcuu auaaaauaga agaauuauuc 19140
uauucuuaug ccacacauuc ugacaaauuc acagauggug uaugccuauu uuggaauugc 19200
aaugucgaua gauauccugc uaauuccauu guuuguagau uugacacuag agugcuaucu 19260
aaccuuaacu ugccugguug ugaugguggc aguuuguaug uaaauaaaca ugcauuccac 19320
acaccagcuu uugauaaaag ugcuuuuguu aauuuaaaac aauuaccauu uuucuauuac 19380
ucugacaguc caugugaguc ucauggaaaa caaguagugu cagauauaga uuauguacca 19440
cuaaagucug cuacguguau aacacguugc aauuuaggug gugcugucug uagacaucau 19500
gcuaaugagu acagauugua ucucgaugcu uauaacauga ugaucucagc uggcuuuagc 19560
uuguggguuu acaaacaauu ugauacuuau aaccucugga acacuuuuac aagacuucag 19620
aguuuagaaa auguggcuuu uaauguugua aauaagggac acuuugaugg acaacagggu 19680
gaaguaccag uuucuaucau uaauaacacu guuuacacaa aaguugaugg uguugaugua 19740
gaauuguuug aaaauaaaac aacauuaccu guuaauguag cauuugagcu uugggcuaag 19800
cgcaacauua aaccaguacc agaggugaaa auacucaaua auuugggugu ggacauugcu 19860
gcuaauacug ugaucuggga cuacaaaaga gaugcuccag cacauauauc uacuauuggu 19920
guuuguucua ugacugacau agccaagaaa ccaacugaaa cgauuugugc accacucacu 19980
gucuuuuuug augguagagu ugauggucaa guagacuuau uuagaaaugc ccguaauggu 20040
guucuuauua cagaagguag uguuaaaggu uuacaaccau cuguaggucc caaacaagcu 20100
agucuuaaug gagucacauu aauuggagaa gccguaaaaa cacaguucaa uuauuauaag 20160
aaaguugaug guguugucca acaauuaccu gaaacuuacu uuacucagag uagaaauuua 20220
caagaauuua aacccaggag ucaaauggaa auugauuucu uagaauuagc uauggaugaa 20280
uucauugaac gguauaaauu agaaggcuau gccuucgaac auaucguuua uggagauuuu 20340
agucauaguc aguuaggugg uuuacaucua cugauuggac uagcuaaacg uuuuaaggaa 20400
ucaccuuuug aauuagaaga uuuuauuccu auggacagua caguuaaaaa cuauuucaua 20460
acagaugcgc aaacagguuc aucuaagugu guguguucug uuauugauuu auuacuugau 20520
gauuuuguug aaauaauaaa aucccaagau uuaucuguag uuucuaaggu ugucaaagug 20580
acuauugacu auacagaaau uucauuuaug cuuuggugua aagauggcca uguagaaaca 20640
uuuuacccaa aauuacaauc uagucaagcg uggcaaccgg guguugcuau gccuaaucuu 20700
uacaaaaugc aaagaaugcu auuagaaaag ugugaccuuc aaaauuaugg ugauagugca 20760
acauuaccua aaggcauaau gaugaauguc gcaaaauaua cucaacugug ucaauauuua 20820
aacacauuaa cauuagcugu acccuauaau augagaguua uacauuuugg ugcugguucu 20880
gauaaaggag uugcaccagg uacagcuguu uuaagacagu gguugccuac ggguacgcug 20940
cuugucgauu cagaucuuaa ugacuuuguc ucugaugcag auucaacuuu gauuggugau 21000
ugugcaacug uacauacagc uaauaaaugg gaucucauua uuagugauau guacgacccu 21060
aagacuaaaa auguuacaaa agaaaaugac ucuaaagagg guuuuuucac uuacauuugu 21120
ggguuuauac aacaaaagcu agcucuugga gguuccgugg cuauaaagau aacagaacau 21180
ucuuggaaug cugaucuuua uaagcucaug ggacacuucg caugguggac agccuuuguu 21240
acuaauguga augcgucauc aucugaagca uuuuuaauug gauguaauua ucuuggcaaa 21300
ccacgcgaac aaauagaugg uuaugucaug caugcaaauu acauauuuug gaggaauaca 21360
aauccaauuc aguugucuuc cuauucuuua uuugacauga guaaauuucc ccuuaaauua 21420
agggguacug cuguuauguc uuuaaaagaa ggucaaauca augauaugau uuuaucucuu 21480
cuuaguaaag guagacuuau aauuagagaa aacaacagag uuguuauuuc uagugauguu 21540
cuuguuaaca acuaaacgaa caauguuugu uuuucuuguu uuauugccac uagucucuag 21600
ucaguguguu aaucuuacaa ccagaacuca auuacccccu gcauacacua auucuuucac 21660
acgugguguu uauuacccug acaaaguuuu cagauccuca guuuuacauu caacucagga 21720
cuuguucuua ccuuucuuuu ccaauguuac uugguuccau gcuauacaug ucucugggac 21780
caaugguacu aagagguuug auaacccugu ccuaccauuu aaugauggug uuuauuuugc 21840
uuccacugag aagucuaaca uaauaagagg cuggauuuuu gguacuacuu uagauucgaa 21900
gacccagucc cuacuuauug uuaauaacgc uacuaauguu guuauuaaag ucugugaauu 21960
ucaauuuugu aaugauccau uuuugggugu uuauuaccac aaaaacaaca aaaguuggau 22020
ggaaagugag uucagaguuu auucuagugc gaauaauugc acuuuugaau augucucuca 22080
gccuuuucuu auggaccuug aaggaaaaca ggguaauuuc aaaaaucuua gggaauuugu 22140
guuuaagaau auugaugguu auuuuaaaau auauucuaag cacacgccua uuaauuuagu 22200
gcgugaucuc ccucaggguu uuucggcuuu agaaccauug guagauuugc caauagguau 22260
uaacaucacu agguuucaaa cuuuacuugc uuuacauaga aguuauuuga cuccugguga 22320
uucuucuuca gguuggacag cuggugcugc agcuuauuau guggguuauc uucaaccuag 22380
gacuuuucua uuaaaauaua augaaaaugg aaccauuaca gaugcuguag acugugcacu 22440
ugacccucuc ucagaaacaa aguguacguu gaaauccuuc acuguagaaa aaggaaucua 22500
ucaaacuucu aacuuuagag uccaaccaac agaaucuauu guuagauuuc cuaauauuac 22560
aaacuugugc ccuuuuggug aaguuuuuaa cgccaccaga uuugcaucug uuuaugcuug 22620
gaacaggaag agaaucagca acuguguugc ugauuauucu guccuauaua auuccgcauc 22680
auuuuccacu uuuaaguguu auggaguguc uccuacuaaa uuaaaugauc ucugcuuuac 22740
uaaugucuau gcagauucau uuguaauuag aggugaugaa gucagacaaa ucgcuccagg 22800
gcaaacugga aagauugcug auuauaauua uaaauuacca gaugauuuua caggcugcgu 22860
uauagcuugg aauucuaaca aucuugauuc uaagguuggu gguaauuaua auuaccugua 22920
uagauuguuu aggaagucua aucucaaacc uuuugagaga gauauuucaa cugaaaucua 22980
ucaggccggu agcacaccuu guaauggugu ugaagguuuu aauuguuacu uuccuuuaca 23040
aucauauggu uuccaaccca cuaauggugu ugguuaccaa ccauacagag uaguaguacu 23100
uucuuuugaa cuucuacaug caccagcaac uguuugugga ccuaaaaagu cuacuaauuu 23160
gguuaaaaac aaauguguca auuucaacuu caaugguuua acaggcacag guguucuuac 23220
ugagucuaac aaaaaguuuc ugccuuucca acaauuuggc agagacauug cugacacuac 23280
ugaugcuguc cgugauccac agacacuuga gauucuugac auuacaccau guucuuuugg 23340
uggugucagu guuauaacac caggaacaaa uacuucuaac cagguugcug uucuuuauca 23400
ggauguuaac ugcacagaag ucccuguugc uauucaugca gaucaacuua cuccuacuug 23460
gcguguuuau ucuacagguu cuaauguuuu ucaaacacgu gcaggcuguu uaauaggggc 23520
ugaacauguc aacaacucau augaguguga cauacccauu ggugcaggua uaugcgcuag 23580
uuaucagacu cagacuaauu cuccucggcg ggcacguagu guagcuaguc aauccaucau 23640
ugccuacacu augucacuug gugcagaaaa uucaguugcu uacucuaaua acucuauugc 23700
cauacccaca aauuuuacua uuaguguuac cacagaaauu cuaccagugu cuaugaccaa 23760
gacaucagua gauuguacaa uguacauuug uggugauuca acugaaugca gcaaucuuuu 23820
guugcaauau ggcaguuuuu guacacaauu aaaccgugcu uuaacuggaa uagcuguuga 23880
acaagacaaa aacacccaag aaguuuuugc acaagucaaa caaauuuaca aaacaccacc 23940
aauuaaagau uuuggugguu uuaauuuuuc acaaauauua ccagauccau caaaaccaag 24000
caagagguca uuuauugaag aucuacuuuu caacaaagug acacuugcag augcuggcuu 24060
caucaaacaa uauggugauu gccuugguga uauugcugcu agagaccuca uuugugcaca 24120
aaaguuuaac ggccuuacug uuuugccacc uuugcucaca gaugaaauga uugcucaaua 24180
cacuucugca cuguuagcgg guacaaucac uucugguugg accuuuggug caggugcugc 24240
auuacaaaua ccauuugcua ugcaaauggc uuauagguuu aaugguauug gaguuacaca 24300
gaauguucuc uaugagaacc aaaaauugau ugccaaccaa uuuaauagug cuauuggcaa 24360
aauucaagac ucacuuucuu ccacagcaag ugcacuugga aaacuucaag auguggucaa 24420
ccaaaaugca caagcuuuaa acacgcuugu uaaacaacuu agcuccaauu uuggugcaau 24480
uucaaguguu uuaaaugaua uccuuucacg ucuugacaaa guugaggcug aagugcaaau 24540
ugauagguug aucacaggca gacuucaaag uuugcagaca uaugugacuc aacaauuaau 24600
uagagcugca gaaaucagag cuucugcuaa ucuugcugcu acuaaaaugu cagagugugu 24660
acuuggacaa ucaaaaagag uugauuuuug uggaaagggc uaucaucuua uguccuuccc 24720
ucagucagca ccucauggug uagucuucuu gcaugugacu uaugucccug cacaagaaaa 24780
gaacuucaca acugcuccug ccauuuguca ugauggaaaa gcacacuuuc cucgugaagg 24840
ugucuuuguu ucaaauggca cacacugguu uguaacacaa aggaauuuuu augaaccaca 24900
aaucauuacu acagacaaca cauuuguguc ugguaacugu gauguuguaa uaggaauugu 24960
caacaacaca guuuaugauc cuuugcaacc ugaauuagac ucauucaagg aggaguuaga 25020
uaaauauuuu aagaaucaua caucaccaga uguugauuua ggugacaucu cuggcauuaa 25080
ugcuucaguu guaaacauuc aaaaagaaau ugaccgccuc aaugagguug ccaagaauuu 25140
aaaugaaucu cucaucgauc uccaagaacu uggaaaguau gagcaguaua uaaaauggcc 25200
augguacauu uggcuagguu uuauagcugg cuugauugcc auaguaaugg ugacaauuau 25260
gcuuugcugu augaccaguu gcuguaguug ucucaagggc uguuguucuu guggauccug 25320
cugcaaauuu gaugaagacg acucugagcc agugcucaaa ggagucaaau uacauuacac 25380
auaaacgaac uuauggauuu guuuaugaga aucuucacaa uuggaacugu aacuuugaag 25440
caaggugaaa ucaaggaugc uacuccuuca gauuuuguuc gcgcuacugc aacgauaccg 25500
auacaagccu cacucccuuu cggauggcuu auuguuggcg uugcacuucu ugcuguuuuu 25560
cagagcgcuu ccaaaaucau aacccucaaa aagagauggc aacuagcacu cuccaagggu 25620
guucacuuug uuugcaacuu gcuguuguug uuuguaacag uuuacucaca ccuuuugcuc 25680
guugcugcug gccuugaagc cccuuuucuc uaucuuuaug cuuuagucua cuucuugcag 25740
aguauaaacu uuguaagaau aauaaugagg cuuuggcuuu gcuggaaaug ccguuccaaa 25800
aacccauuac uuuaugaugc caacuauuuu cuuugcuggc auacuaauug uuacgacuau 25860
uguauaccuu acaauagugu aacuucuuca auugucauua cuucagguga uggcacaaca 25920
aguccuauuu cugaacauga cuaccagauu ggugguuaua cugaaaaaug ggaaucugga 25980
guaaaagacu guguuguauu acacaguuac uucacuucag acuauuacca gcuguacuca 26040
acucaauuga guacagacac ugguguugaa cauguuaccu ucuucaucua caauaaaauu 26100
guugaugagc cugaagaaca uguccaaauu cacacaaucg acgguucauc cggaguuguu 26160
aauccaguaa uggaaccaau uuaugaugaa ccgacgacga cuacuagcgu gccuuuguaa 26220
gcacaagcug augaguacga acuuauguac ucauucguuu cggaagagac agguacguua 26280
auaguuaaua gcguacuucu uuuucuugcu uucgugguau ucuugcuagu uacacuagcc 26340
auccuuacug cgcuucgauu gugugcguac ugcugcaaua uuguuaacgu gagucuugua 26400
aaaccuucuu uuuacguuua cucucguguu aaaaaucuga auucuucuag aguuccugau 26460
cuucuggucu aaacgaacua aauauuauau uaguuuuucu guuuggaacu uuaauuuuag 26520
ccauggcaga uuccaacggu acuauuaccg uugaagagcu uaaaaagcuc cuugaacaau 26580
ggaaccuagu aauagguuuc cuauuccuua cauggauuug ucuucuacaa uuugccuaug 26640
ccaacaggaa uagguuuuug uauauaauua aguuaauuuu ccucuggcug uuauggccag 26700
uaacuuuagc uuguuuugug cuugcugcug uuuacagaau aaauuggauc accgguggaa 26760
uugcuaucgc aauggcuugu cuuguaggcu ugauguggcu cagcuacuuc auugcuucuu 26820
ucagacuguu ugcgcguacg cguuccaugu ggucauucaa uccagaaacu aacauucuuc 26880
ucaacgugcc acuccauggc acuauucuga ccagaccgcu ucuagaaagu gaacucguaa 26940
ucggagcugu gauccuucgu ggacaucuuc guauugcugg acaccaucua ggacgcugug 27000
acaucaagga ccugccuaaa gaaaucacug uugcuacauc acgaacgcuu ucuuauuaca 27060
aauugggagc uucgcagcgu guagcaggug acucagguuu ugcugcauac agucgcuaca 27120
ggauuggcaa cuauaaauua aacacagacc auuccaguag cagugacaau auugcuuugc 27180
uuguacagua agugacaaca gauguuucau cucguugacu uucagguuac uauagcagag 27240
auauuacuaa uuauuaugag gacuuuuaaa guuuccauuu ggaaucuuga uuacaucaua 27300
aaccucauaa uuaaaaauuu aucuaaguca cuaacugaga auaaauauuc ucaauuagau 27360
gaagagcaac caauggagau ugauuaaacg aacaugaaaa uuauucuuuu cuuggcacug 27420
auaacacucg cuacuuguga gcuuuaucac uaccaagagu guguuagagg uacaacagua 27480
cuuuuaaaag aaccuugcuc uucuggaaca uacgagggca auucaccauu ucauccucua 27540
gcugauaaca aauuugcacu gacuugcuuu agcacucaau uugcuuuugc uuguccugac 27600
ggcguaaaac acgucuauca guuacgugcc agaucaguuu caccuaaacu guucaucaga 27660
caagaggaag uucaagaacu uuacucucca auuuuucuua uuguugcggc aauaguguuu 27720
auaacacuuu gcuucacacu caaaagaaag acagaaugau ugaacuuuca uuaauugacu 27780
ucuauuugug cuuuuuagcc uuucugcuau uccuuguuuu aauuaugcuu auuaucuuuu 27840
gguucucacu ugaacugcaa gaucauaaug aaacuuguca cgccuaaacg aacaugaaau 27900
uucuuguuuu cuuaggaauc aucacaacug uagcugcauu ucaccaagaa uguaguuuac 27960
agucauguac ucaacaucaa ccauauguag uugaugaccc guguccuauu cacuucuauu 28020
cuaaauggua uauuagagua ggagcuagaa aaucagcacc uuuaauugaa uugugcgugg 28080
augaggcugg uucuaaauca cccauucagu acaucgauau cgguaauuau acaguuuccu 28140
guuuaccuuu uacaauuaau ugccaggaac cuaaauuggg uagucuugua gugcguuguu 28200
cguucuauga agacuuuuua gaguaucaug acguucgugu uguuuuagau uucaucuaaa 28260
cgaacaaacu aaaaugucug auaauggacc ccaaaaucag cgaaaugcac cccgcauuac 28320
guuuggugga cccucagauu caacuggcag uaaccagaau ggagaacgca guggggcgcg 28380
aucaaaacaa cgucggcccc aagguuuacc caauaauacu gcgucuuggu ucaccgcucu 28440
cacucaacau ggcaaggaag accuuaaauu cccucgagga caaggcguuc caauuaacac 28500
caauagcagu ccagaugacc aaauuggcua cuaccgaaga gcuaccagac gaauucgugg 28560
uggugacggu aaaaugaaag aucucagucc aagaugguau uucuacuacc uaggaacugg 28620
gccagaagcu ggacuucccu auggugcuaa caaagacggc aucauauggg uugcaacuga 28680
gggagccuug aauacaccaa aagaucacau uggcacccgc aauccugcua acaaugcugc 28740
aaucgugcua caacuuccuc aaggaacaac auugccaaaa ggcuucuacg cagaagggag 28800
cagaggcggc agucaagccu cuucucguuc cucaucacgu agucgcaaca guucaagaaa 28860
uucaacucca ggcagcagua ggggaacuuc uccugcuaga auggcuggca auggcgguga 28920
ugcugcucuu gcuuugcugc ugcuugacag auugaaccag cuugagagca aaaugucugg 28980
uaaaggccaa caacaacaag gccaaacugu cacuaagaaa ucugcugcug aggcuucuaa 29040
gaagccucgg caaaaacgua cugccacuaa agcauacaau guaacacaag cuuucggcag 2910
acguggucca gaacaaaccc aaggaaauuu uggggaccag gaacuaauca gacaaggaac 29160
ugauuacaaa cauuggccgc aaauugcaca auuugccccc agcgcuucag cguucuucgg 29220
aaugucgcgc auuggcaugg aagucacacc uucgggaacg ugguugaccu acacaggugc 29280
caucaaauug gaugacaaag auccaaauuu caaagaucaa gucauuuugc ugaauaagca 29340
uauugacgca uacaaaacau ucccaccaac agagccuaaa aaggacaaaa agaagaaggc 29400
ugaugaaacu caagccuuac cgcagagaca gaagaaacag caaacuguga cucuucuucc 29460
ugcugcagau uuggaugauu ucuccaaaca auugcaacaa uccaugagca gugcugacuc 29520
aacucaggcc uaaacucaug cagaccacac aaggcagaug ggcuauauaa acguuuucgc 29580
uuuuccguuu acgauauaua gucuacucuu gugcagaaug aauucucgua acuacauagc 29640
acaaguagau guaguuaacu uuaaucucac auagcaaucu uuaaucagug uguaacauua 29700
gggaggacuu gaaagagcca ccacauuuuc accgaggcca cgcggaguac gaucgagugu 29760
acagugaaca augcuaggga gagcugccua uauggaagag cccuaaugug uaaaauuaau 29820
uuuaguagug cuauccccau gugauuuuaa uagcuucuua ggagaaugac aaaaaaaaaa 29880
aaaaaaaaaa aaaaaaaaaa aaa 29903

SEQ ID NO: 35

<211> 33
<212> RNA
<213> Artificial Sequence
<220>
<223> Target sequence RSE Circ chikvRSE
<400> 35

agcaaauaau cuauagauca aagggcuacg caa 33

SEQ ID NO: 36

<211> 294
<212> RNA
<213> Artificial Sequence
<220>
<223> Artificial circular ARN 1; target disruption structure comprised
in SARS-CoV-2 3′UTR replication site
<400> 36

ugcaguuaga uacugcuugu guuauguggu ugugaggguu uauuuugaga uccugcuggu 60
uacuugugcu guguaguuau gagaguuugu uuuggauacc uuuagccugc uuguguugug 120
ugguugcgag gauuuauucu gcucggaaac gauuuguuug uguuauguag uuguggggau 180
ucauucuggc ugcuuccucu uuauuugugu uguguaguua cgggaguucg uuuuguccca 240
cauagaguug cuugugcuau gugguuacgg ggauuuauuu ugccagggcc aaac 294

SEQ ID NO: 37

<211> 29
<212> RNA
<213> Artificial Sequence
<220>
<223> Target sequence SL III 3′ UTR circ_wnv_slll_½
<400> 37

uuuugaggag aaagucaggc cgggaaguu 29

SEQ ID NO: 38

<211> 294
<212> RNA
<213> Artificial Sequence
<220>
<223> Artificial circular ARN 2; target disruption structure comprised
in SARS-CoV-2 3′UTR replication site
<400> 38

ugcaguuaga uacugcuugu guuauguggu ugugaggguu uauuuugaga uccugcuggu 60
uacuugugcu guguaguuau gagaguuugu uuuggauacc uuuagccugc uuguguugug 120
ugguugcgag gauuuauucu gcucggaaac gauuuguuug uguuauguag uuguggggau 180
ucauucuggc ugcuuccucu uuguuugugu uguguaguua cggggguucg uuuuguccca 240
cauagaguua cuugugcuau guaguuauga ggauucauuu ugccagggcc aaac 294

SEQ ID NO: 39

<211> 294
<212> RNA
<213> Artificial Sequence
<220>
<223> Circ Chikv_re
<400> 39

gcgccgauua uggcuaaaag agucaacguu uuguaguagc ugcggguugc ccucauuacg 60
guuaagaggg uuaauguuuu auagcagcug cccgcgagca ggauuauggu uggaaagguu 120
aacguuuugc agcagcguuc guaacacuuc auuauggcua aggaagccaa cguuuugugg 180
uagccuaggu ucgaaccgau uauggcuaga ggggucagcg uuuuauggua gccaucaaaa 240
ccuuucauua cggcugagag agccaauguu uuacaguagc uccaacgacg uacu 294

SEQ ID NO: 40

<211> 294
<212> RNA
<213> Artificial Sequence
<220>
<223> Artificial circular RNA 3; target disruption structure comprised
in SARS-CoV-2 3′UTR replication site
<400> 40

ugcaguuaga uacugcuugu guuauguggu ugugaggguu uauuuugaga uccugcuggu 60
uguuugugcu auguaguuac gaggguucau uuuggauacc uuuagccugc uuguguugug 120
ugguugcgag gauuuauucu gcucggaaac gauuuguuug uguuauguag uuguggggau 180
ucauucuggc ugcuuccucu cugcuugugc uguguaguua ugagaguuug uuuuguccca 240
cauagaguua uuuguguugu guaguugugg gaguuuguuu ugccagggcc aaac 294

SEQ ID NO: 41

<211> 294
<212> RNA
<213> Artificial Sequence
<220>
<223> Artificial circular RNA 4; target disruption structure comprised
in SARS-CoV-2 3′UTR replication site
<400> 41

ugcaguuaga uauuguuugu gcuauguggu uguggggauu cauucugaug ucguauuguu 60
ugcuuguguu gugugguuac gggaauuugu uuugaacucu uaguuguugu uugugcugug 120
uaguuguggg gguuuguucu gauguucuga ucuuuguuug uguuauguag uuacgaggau 180
uuauucuggu gcaacaaguu cuauuugugu uaugugguug cggggguucg uuuugcccgg 240
ccuucgcuua cuuguguugu guaguuguga ggauucguuu uggaugcauc gaac 294

SEQ ID NO: 42

<211> 294
<212> RNA
<213> Artificial Sequence
<220>
<223> Artificial circular RNA 5; target disruption structure comprised
in SARS-CoV-2 3′UTR replication site
<400> 42

ugcaguuaga uacuauuugu gcuauguagu ugcggggguu uguuuugugg cuuucuuacc 60
uguuuguguu augugguugu gaggauuugu uuuggccgac ucguuccugc uugugcugug 120
uaguuguggg aguucauuuu guucagaguu agccuacuug uguuauguag uuaugagagu 180
ucguuuugga gagacgcaca cuguuugugu ugugugguua uggggauucg uuuugugggu 240
ccggcuccug uuugugcugu gugguuauga gaauuuguuc ugaagauuua uucc 294

SEQ ID NO: 43

<211> 292
<212> RNA
<213> Artificial Sequence
<220>
<223> Artificial circular RNA 1; target disruption structure comprised
in SARS-CoV-2 5′UTR (SIII)
<400> 43

ugcaguuaga uaagaucugc aggggguuga ggguugguug auccaggcug cuagaucuau 60
aagggguugg gaguugguuc uuuagcuauc ccagauuugu gagggguugg ggguugguua 120
ggcuuacagu uaagaucuau gggagguugg agguugguua ccgccgaguu auagauuuau 180
aggggaucgg aaguugguua aagcuagggc gaagaucugc gggggguugg aaguugguua 240
uacaacgaau uaagauuuau ggggggucgg ggguugguuu aguaguugga ug 292

SEQ ID NO: 44

<211> 292
<212> RNA
<213> Artificial Sequence
<220>
<223> Artificial circular RNA 2; target disruption structure comprised
in SARS-CoV-2 5′UTR (SIII)
<400> 44

ugcaguuaga uaggaucuau aagggguugg aaguugguua uguggucgcg cgaggucugu 60
aagagauuga aaguugguug ucacugacgu aaagauuuac gagagaucga ggguugguug 120
aaugucgguc ugggguuugu gggggguugg agguugguua uugguauaac ggaggucuau 180
gagagguuga ggguugguuc gcgauagaca ccgggucugc aggaggucgg aaguugguua 240
guugaacaga cuggguuuau ggggggucga gaguugguua uuuguuccga gg 292

SEQ ID NO: 45

<211> 292
<212> RNA
<213> Artificial Sequence
<220>
<223> Artificial circular RNA 3; target disruption structure comprised
in SARS-CoV-2 5′UTR (SIII)
<400> 45

ugcaguuaga uaagguuugc gagggauugg aaguugguug aaauguggaa ggggauuugc 60
gagagguuga aaguugguuu uuauuaaagg uaggaucuac aagggauuga agguugguua 120
cccaguagcu caggguuuac aggaggucgg agguugguuu uucuggauag aaaggucuau 180
aagagguuga ggguugguuu ggagauugua acggauuuau gagagaucga gaguugguua 240
aaaacaagga acggauuuac aagggguugg ggguugguua uacuuagcgg ug 292

SEQ ID NO: 46

<211> 282
<212> RNA
<213> Artificial Sequence
<220>
<223> Artificial circular RNA 1; target hybridization region:
SARS-CoV-2 Target A
<400> 46

ugcaguuaga uauggugcug ugucgucguc uauucuaagu uugggagauc cugcuggugg 60
uauuauguua cugucuguuc uaaauuugag gauaccuuua gcuggugucg uguuauuguc 120
uguuuuggac uuaggcucgg aaacgauugg uacuguguca cugucuguuu uaggcuugaa 180
gcugcuuccu cuugguguua ugucaucguc uauuuugaac uuggguccca cauagagugg 240
uaccaugucg uugucuguuc uggauuuaaa ccagggccaa ac 282

SEQ ID NO: 47

<211> 282
<212> RNA
<213> Artificial Sequence
<220>
<223> Artificial circular RNA 2; target hybridization region:
SARS-CoV-2 Target A
<400> 47

ugcaguuaga uaugguacug ugucacuguc uguucugaac uugagcguuc acgucucugg 60
ugcuauguug ucgucuauuc uaaacuuaag aacuagguau aguggugucg uguuaucguc 120
uauuuugggc uugggggcuc cccuuggugg uaccauguca ccgucuauuc ugaauuuaaa 180
ucucccauag ugugguaucg uguugccguc uauuuuagac uuggaccacc gucuguuugg 240
uacuauguug cugucuguuu ugaacuugag aucagauuaa gg 282

SEQ ID NO: 48

<211> 282
<212> RNA
<213> Artificial Sequence
<220>
<223> Artificial circular RNA 3; target hybridization region:
SARS-CoV-2 Target A
<400> 48

ugcaguuaga uaugguaccg ugucgucguc uauuuuggau uuaaaagauc cugcuggugg 60
uauuaugucg uuguuuguuc ugaguuuaaa gauaccuuua gcugguacca uguuaccguc 120
uguucuaagu uuaaacucgg aaacgauugg uauuguguug ucguuuguuc uagauuuaaa 180
gcugcuuccu cuugguaucg ugucgccguc uauucugagc uuaaauccca cauagagugg 240
uacuauguug uugucuauuu ugaguuuaaa ccagggccaa ac 282

SEQ ID NO: 49

<211> 282
<212> RNA
<213> Artificial Sequence
<220>
<223> Artificial circular RNA 4; target hybridization region:
SARS-CoV-2 Target A
<400> 49

ugcaguuaga uaugguaccg ugucgccguu uguucugagc uuaaaguucu ugcgcucugg 60
uaucauguug cuguuuauuc uaggcuuaaa ucggccgucu ggugguauug ugucguuguc 120
uguucugagu uuaaaucucu uucccggugg uacuguguug ucgucuauuc uggguuuaaa 180
acucgacccc ggugguacca ugucaccguc uguuuuagac uuaaaguuac aagucgcugg 240
uauuauguug cugucuauuc uagauuuaaa auucccccag cc 282

SEQ ID NO: 50

<211> 299
<212> RNA
<213> Artificial Sequence
<220>
<223> Artificial circular RNA 1; target hybridization region:
SARS-CoV-2 Target C
<400> 50

ugcaguuaga uauagaggcc uugguggcag guuucuuggu gaagugagua uaguagaggu 60
cuuaguagca gguuuuuuag ugauguaacc uauguagaag ccucagcagc agauuucuug 120
gugggggguc aaggguaggg gucuuggugg uagauuuuuu gguggcacuu gaggaguagg 180
aguuucggcg guggauuucu uagugggcua ggaaauuugg gagcuuuggu gguagguuuu 240
uuggugaggu gggaggguug ggggcuucgg uagcggauuu uuuaguggaa cugaaagcg 299

SEQ ID NO: 51

<211> 292
<212> RNA
<213> Artificial Sequence
<220>
<223> Artificial circular RNA 2; target hybridization region:
SARS-CoV-2 Target C
<400> 51

ugcaguuaga uauaggaguu ucggcggcgg guuuuuuggu gaaaguugaa uuuagaaguu 60
ucgguggcgg guuucuuagu gagaaauuca gauagaaguu uuggcagugg auuucuuggu 120
gaggaauggg uuuggaagcc uugguagugg auuuuuuagu ggccacccuu gguagaagcu 180
uuggugguag auuuuuuggu ggguaacuua auugggaguu uugguggcag guuuuuuagu 240
gauaggagac cguagggguu ucagcaguag auuucuuagu gggagacaga cu 292

SEQ ID NO: 52

<211> 292
<212> RNA
<213> Artificial Sequence
<220>
<223> Artificial circular RNA 3; target hybridization region:
SARS-CoV-2 Target C
<400> 52

ugcaguuaga uauaggaguc ucggcagcgg auuuuuuagu ggacaugacu uuuagaggcc 60
uugguagcgg auuucuuagu ggucuacagc gaugggaguu uuggcggugg guuuuuuggu 120
gaccucuaau guuggaggcu ucagcagcag guuuuuuagu gauaagagua gguaggggcc 180
uuaguagcag auuucuuggu gggauucgaa uuuagagguu uuaguaguag guuucuuagu 240
gaagggcuuu auuggggguu uuagcggcag auuuuuuggu ggauucgauc ug 292

SEQ ID NO: 53

<211> 292
<212> RNA
<213> Artificial Sequence
<220>
<223> Artificial circular RNA 4; target hybridization region:
SARS-CoV-2 Target C
<400> 53

ugcaguuaga uauggaaguu uuggcagugg guuucuuagu ggugggacuu gauagaaguc 60
uuagugguag guuucuuggu ggggcggagu guuggaggcu uugguagcgg guuuuuuagu 120
ggggcuaucu cgugggaguc uugguaguag auuucuuggu gaaggaugcg gguggaaguc 180
uuagcggcgg auuuuuuggu ggccaguuag uauggggguu uugguagcag guuuuuuggu 240
gauaguagac ucuggaggcu ucgguagcag auuuuuuagu gaguuauaaa gg 292

SEQ ID NO: 54

<211> 270
<212> RNA
<213> Artificial Sequence
<220>
<223> Artificial circular RNA 1; target hybridization region:
SARS-CoV-2 Target D
<400> 54

ugcaguuaga uaaaaccgua agcagccugu agaagguaga cgaguccaga cguacaaacc 60
guaggcagcu ugcagaagau agacgaagaa cgaguacuaa accgugggca guuugcgggg 120
gauagacgaa gcuuggagac uaaaccguaa guagucugug ggggauggac gaauuucugg 180
caauaaaccg uagguaguuu gcagagggua gacgagcucg uagcuagaaa ccgugaguag 240
ccuguagagg auggacgacu gagaccucaa 270

SEQ ID NO: 55

<211> 270
<212> RNA
<213> Artificial Sequence
<220>
<223> Artificial circular RNA 2; target hybridization region:
SARS-CoV-2 Target D
<400> 55

ugcaguuaga uaaaaccgua ggcagccugu aggagguaga cgaguuuucu cacggaaacc 60
guggguagcu uguaggagau agacgaguug aggaccagaa accguaagug guuugcagga 120
gauggacgag uuugggcagu uaaaccguga gcagucugca ggggguagac gaauguucgg 180
agcaaaaccg ugaguaguuu guagaaggug gacgagagga gaguaggaaa ccguaagcag 240
ucuguagggg auggacgauu uauaaucagu 270

SEQ ID NO: 56

<211> 270
<212> RNA
<213> Artificial Sequence
<220>
<223> Artificial circular RNA 3; target hybridization region:
SARS-CoV-2 Target D
<400> 56

ugcaguuaga uaaagccgug agcggcuugu ggaagauaga ugaaaggauu ggaaaagacc 60
guaagcagcu uguagaagau gggugacaag uaauggaaaa auuguaagug gucugcggaa 120
ggugggugga uaaggaacuu gagaucguag guaguuugua gaggguaggu gggacuaugg 180
ggaugaacug ugggcggucu gcaggaggug gacgagagag guacauaaga ccguaaguag 240
uuuguggggg augggcgaug uauugcacac 270

SEQ ID NO: 57

<211> 270
<212> RNA
<213> Artificial Sequence
<220>
<223> Artificial circular RNA 4; target hybridization region:
SARS-CoV-2 Target D
<400> 57

ugcaguuaga uaaagccgug agcggcuugu ggaagauaga ugaaaggauu ggaaaagacc 60
guaagcagcu uguagaagau gggugacaag uaauggaaaa auuguaagug gucugcggaa 120
ggugggugga uaaggaacuu gagaucguag guaguuugua gaggguaggu gggacuaugg 180
ggaugaacug ugggcggucu gcaggaggug gacgagagag guacauaaag ccguaaguag 240
cuuguagggg auagauggug uauugcacac 270

SEQ ID NO: 58

<211> 27
<212> RNA
<213> Artificial Sequence
<220>
<223> SARS-CoV-2 target hybridization region - comprised in
5′UTR SLII
<400> 58

aaccaacuuu cgaucucuug uagaucu 27

SEQ ID NO: 59

<211> 33
<212> RNA
<213> Artificial Sequence
<220>
<223> SARS-CoV-2 target hybridization region - Target A
<400> 59

uuuaaguuua gaauagacgg ugacauggua cca 33

SEQ ID NO: 60

<211> 30
<212> RNA
<213> Artificial Sequence
<220>
<223> SARS-CoV-2 target hybridization region - Target C
<400> 60

ucacuaagaa aucugcugcu gaggcuucua 30

SEQ ID NO: 61

<211> 31
<212> RNA
<213> Artificial Sequence
<220>
<223> SARS-CoV-2 target hybridization region - Target D
<400> 61

ucgucuaucu ucugcaggcu gcuuacgguu u 31

SEQ ID NO: 62

<211> 35
<212> RNA
<213> Artificial Sequence
<220>
<223> SARS-CoV-2 target hybridization region - comprised in
3UTR Replication Site
<400> 62

cagaaugaau ucucguaacu acauagcaca aguag 35

SEQ ID NO: 63

<211> 289
<212> RNA
<213> Artificial Sequence
<220>
<223> Artificial circular RNA against WNV and DENV (circWD1-in vitro)
<400> 63

ugcaguuaga uaugcaguua gauaauauug ggaggugugu uuuuugccuu uuuccguaga 60
uccucgcgaa acuucuuggc cugguuuucu uuucaaaaga guccuuacgu auauuggagg 120
guguguuuuu cgcuuuuuuc cgucucggaa acgauaacuu ccuggcuuga uuuuuucuuc 180
aaaagcugcg uuaucuauau ugagaggcgu guuuuucguc uuuuuccguu ccaaccugga 240
gaacuuuuug gccugauuuu cucuucaaaa ccaggccgua ccgacaguc 289

SEQ ID NO: 64

<211> 307
<212> RNA
<213> Artificial Sequence
<220>
<223> Artificial circular RNA against HCV (circHCV-in vitro)
<400> 64

ugcaguuaga uacccccugg ggcucugaug aggaaccucu cugggguccc cacagcgagu 60
cuccuugggg ccccuagaau gaaguucuuu gggguuucau agcacgguuc uccugggguu 120
uucauacgau ugucucuugg ggucuugcgu guuuacccuu uugggguccu gcuaaggggg 180
cuuucugggg ccuuucgaau aagucuuuuu ggggccucgu uuuaucacuc uucugggguu 240
cccagccuuu cccuucuugg ggcuccuccg accaugcccc uugggguucu acccuguaug 300
gacaguc 307

>SEQ ID 65

CCCGGTTCCGCACTACTTGTGCTGTGTAGTTACGAGGATTCGTTCTGACC
GTGTCTTCGCTATTTGTGCTATGTAGTTATGGGAGTTTATTCTGCTGCTC
GTGGACCTACTTGTGTTATGTAGTTGTGGGAGTTCGTTCTGGGGCAGTTC
GCTCTATTTGTGCTGTGTGGTTGCGGGGGTTCATTTTGATCCCGATAAGC
CTACTTGTGTTGTGTAGTTATGAGGGTTTGTTTTGACCATACGACAGCTA
CTTGTGCTATGTGGTTGTGAGGATTCATTCTGGACAGTC

>SEQ ID 66

CCCGGTTCCGCAAGATTTGTAGGAGGTCGAAAGTTGGTTTACAACGCTAC
TGGGGTCTACAGGAGGTTGAAGGTTGGTTGTCATAATGCAGTGGATCTAC
AGGGGGTCGAGAGTTGGTTGCCGCCATTCACAGGGTCTGCAGGGGGTCGG
AAGTTGGTTAGCAGATGTTTGAAGGTCTACGAGAGATTGGGGGTTGGTTA
GGCTCGGAAGAAAGATTTATAGGGGGTTGGAGGTTGGTTCCAGTCAGTTT
ACGGATCTACAAGGGGTTGGGAGTTGGTTGACAGTC

>SEQ ID 67

CCCGGTTCCGCAAGATTTGTAGGAGGTCGAAAGTTGGTTTACAACGCTAC
TGGGGTCTACAGGAGGTTGAAGGTTGGTTGTCATAATGCAGTGGATCTAC
AGGGGGTCGAGAGTTGGTTGCCGCCATTCACAGGGTCTGCGAGAGGTCGG
AAGTTGGTTAGCAGATGTTTGAAGGTCTACGAGAGATTGGGGGTTGGTTA
GGCTCGGAAGAAAGATTTATAGGGGGTTGGAGGTTGGTTCCAGTCAGTTT
ACGGATCTACAAGGGGTTGGGAGTTGGTTGACAGTC

>SEQ ID 68

CCCGGTTCCGCATGGTATTGTGTTACCGTCTATTTTAAGCTTAAGGCCCC
CAAACTAGTTGGTGCCGTGTTGCCGTCTGTTCTAAGCTTAAGCTCGATCC
CGTGTTTGGTGCTATGTCACCGTCTATTCTAGACTTAGGCCGTCACTATC
ATTTGGTGTTGTGTTATTGTCTATTTTGGACTTGAACATGATTACACAAC
TGGTATCGTGTTGTCGTCTGTTTTAGACTTAGGTCGCTTTTCTTTCCTGG
TACCATGTCGCCGTCTGTTTTGGGCTTGAGGACAGTC

>SEQ ID 69

CCCGGTTCCGCATGGTATTATGTTGCCGTCTATTCTGGACTTAAACATTC
CGTCATAGCTGGTGTCGTGTTATCGTCTATTCTAAACTTGAGGTCACAGT
CGAACCTGGTGCTATGTCGTTGTCTGTTCTGGGTTTAAGCTACTTTTTCA
TGATGGTACTGTGTTACTGTCTGTTCTAGACTTGGGCGAGCAGAGATCTT
TGGTACTATGTCACCGTCTATTTTAAGCTTGGGATTAAAGCACCTGGTGG
TACCGTGTCGCTGTCTATTTTGGGCTTAGAGACAGTC

>SEQ ID 70

CCCGGTTCCGCATGGTATTATGTTATTGTTTGTTCTGGGCTTAAAGAAGT
ACCATTAGGTGGTATCGTGTTGTTGTTTATTCTAGATTTAAAGACCTGGG
GGCATCTGGTACTGTGTTGTCGTCTATTTTAGACTTAAAGTGGGCGATCG
AGTTGGTACCATGTCATCGTTTATTTTAGGCTTAAACACAGATTCGCCTC
TGGTACTATGTCGCCGTTTATTTTGGGTTTAAATATGCGTGGAGCAATGG
TATCATGTTACCGTCTATTTTGAACTTAAAGACAGTC

>SEQ ID 71

CCCGGTTCCGCATAGGGGCCTTGGCAGTAGGTTTTTTAGTGGAGTAGCCA
TTTAGGGGTTTCAGTGGTAGATTTCTTAGTGGAACCATATTGTAGGAGTC
TCGGTAGTGGATTTCTTGGTGAATCGCTTGGCTGGAAGCCTTAGTAGCAG
GTTTTTTGGTGGCAGCTAAGCATAGAGGCCTCGGTAGTAGATTTTTTAGT
GATCGTAGAGGATAGGGGCTTTGGCAGCAGATTTTTTGGTGGGAGATTGC
CTTAGGAGCCTCGGCAGCAGGTTTCTTGGTGAGACAGTC

>SEQ ID 72

CCCGGTTCCGCATAGGAGCCTTAGCGGTAGGTTTCTTAGTGAACTATTAT
TTTAGAAGCTTTAGTAGCGGATTTTTTGGTGGTTTTTATAACTGGAGGTT
TCAGTGGCAGGTTTTTTAGTGGAGGAGTGAGCTGGAAGTCTCGGTAGCAG
GTTTTTTGGTGATGGCATTGACTGGAAGTCTTGGCGGTAGATTTCTTGGT
GAGTCTCACCGATGGAAGTTTCAGCGGCGGGTTTCTTGGTGGTGAATGAA
CTTAGGGGCTTCAGCGGCGGATTTCTTAGTGGGACAGTC

SEQ ID NO.:73

5′-FAM-ACCCCGCATTACGTTTGGTGGACC-BHQ1-3′

SEQ ID NO.:74

5′-GACCCCAAAATCAGCGAAAT-3′

SEQ ID NO.:75

5′-TCTGGTTACTGCCAGTTGAATCTG-3

### Target 1: IRES 1 (Domain II)

SEQ ID NO.: 76,

CUCCGCCAUGAAUCACUCCCCUGUGAGGAACUACUGUCUUCACGCAGAAA
GCGCCUAGCCAUGGCGUUAGUAUGAGUGUCGUACAGCCUCCAGGCCC

### Target 2: IRES 2 (Domain IV)

SEQ ID NO. : 77

UCUCGUAGACCGUGCACCAUGAGCACAAAUCCU

### Target 3: CDS1 (cHP)

SEQ ID NO. : 78;

CCAAAAGAAACACCAACCGUCGCCCAGAAGACGUUAAGUUCCCGGGCGGC
GG

### Target 4: CDS2 (SL427)

SEQ ID NO.: 79;

GAUCGUUGGCGGAGUAUACUUGUUGCCGCGCAGGGGCCCCAGGUUGGGUG
UGCGCACGACAAGGAAAACUUCGGAGCGGUC

### CHIKV

### Target 1: 5′UTR

SEQ ID NO.: 80,

AUGGCUGCGUGAGACACACGUAGCCUACCAGUUUCUUACUGCUCUACUCU
GCAAAGCAAGAGAUUAAUAA

### Target 2: Repetitive Sequence Element:

SEQ ID NO.: 81,

AGCAAAUAAUCUAUAGAUCAAAGGGCUACGCAACCCCUGAA

### Target 3: Recoding Element:

SEQ ID NO.: 82,

UGUCUGAGACUCUUACCAUGCUGCUGUAAAACGUUGGCUUUUUUAGCCGU AAUGAGCGUCGGUGCCCAC

### WNV

### Target 1: SLII

SEQ ID NO.: 83,

UUUUGAGGAGAAAGUCAGGCCGGGAAGUUCCCGCCACCGGAAGUUGAGUA
GACGGUGCUGCCUGCGA

### SARS-CoV-2

### Target 1: Replication Site Fully Conserved in Pangolin and RatG13

SEQ ID NO.: 84,

CAGAAUGAAUUCUCGUAACUACAUAGCACAAGUAGAUGUAGUUAACUU

### Target 3: Target A

SEQ ID NO.: 85,

CUUUAAGUUUAGAAUAGACGGUGACAUGGUACCACAUAUAUCACGUCAAC
GUCUUA

### Target 4: Target C

SEQ ID NO.: 86

UCACUAAGAAAUCUGCUGCUGAGGCUUCUAAGAAGCCCUCGGCAAA

### Target 5: Target D

SEQ ID NO.: 87,

UCGUCUAUCUUCUGCAGGCUGCUUACGGUUUCGUCCGUGUUGCAGCGAU

>SEQ ID 88

CCCGGTTCCGCACTACTTGTGTTATGTAGTTACGAGGATTCGTTTTGCTC
TTTTCTCCCTTATTTGTGTTGTGTAGTTATGGGAGTTTGTTCTGGCGTGG
TACGTCTTACTTGTGTTGTGTGGTTATGAGAGTTCATTCTGTATCACCAC
TAGCTGCTTGTGCTGTGTAGTTATGAGGATTTATTCTGGAATACCGTTCC
CTGTTTGTGCTGTGTGGTTATGGGAGTTCGTTCTGTGAGGTGTTCCACTG
CTTGTGCTATGTGGTTACGAGAATTTGTTTTGGACAGTC

>SEQ ID 89

CCCGGTTCCGCACTACTTGTGCTGTGTAGTTACGAGGATTCGTTCTGACC
GTGTCTTCGCTATTTGTGCTATGTAGTTATGGGAGTTTATTCTGCAACCA
GTGGACCTACTTGTGTTATGTAGTTGTGGGAGTTCGTTCTGGGGCAGTTC
GCTCTATTTGTGCTGTGTGGTTGCGGGGGTTCATTTTGATCCCGATAAGC
CTACTTGTGTTGTGTAGTTATGAGGGTTTGTTTTGACCATACGACAGCTA
CTTGTGCTATGTGGTTGTGAGGATTCATTCTGGACAGTC

### EXAMPLES

### Example 1

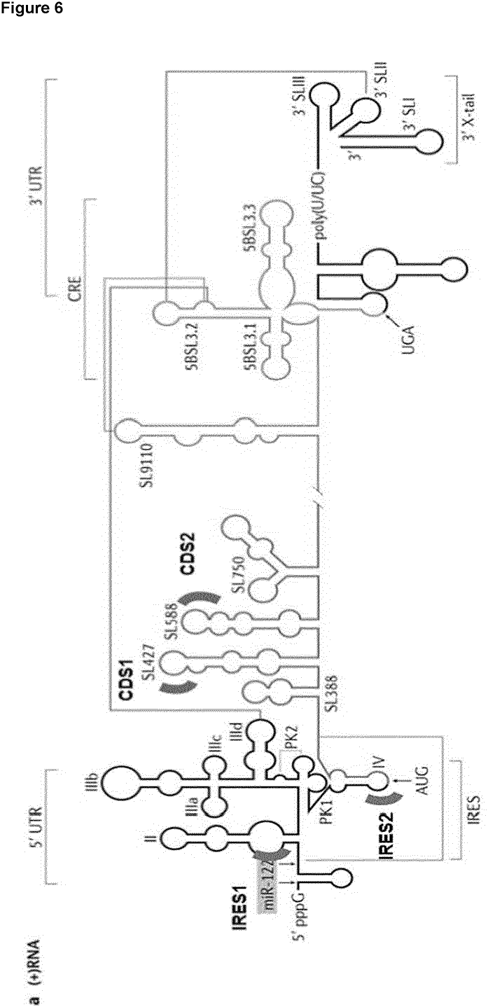

We have designed and tested four circRNAs (circ_hcv_ires1 (SEQ ID NO.: 2), circ_hcv_cds1 (SEQ ID NO.: 4), circ_hcv_cds2 (SEQ ID NO.: 6) y circ_hcv_combo1 (SEQ ID NO.: 5) against two different regions of HCV genome (see SEQ ID NO.: 1) as previously disclosed in the section Production of circRNAs. Circ_hcv_ires1 contains 7 hybridization sites of length 33 nucleotides that target a sequence in the IRES element. Circ_hcv_cds1 contains 8 hybridization sites of length 28 nucleotides that target a sequence in the coding region. Circ_hcv_cds2 contains 12 hybridization sites that target a sequence in the coding region. Circ_hcv_combo1 contains 3 hybridization sites per target region (IRES1 (SEQ ID NO.: 25), IRES2 (SEQ ID NO.: 26) and cHP (CDS1) (SEQ ID NO.: 27)). The target regions in the HCV genome are depicted in FIG. 6 and SEQ ID NO.: 1. Both IRES1 (domain IV) and IRES2 (domain V) target disruption structures were considered SRVVLC in C Romero-López et al. 2007 (Cell Mol Life Sci . 2007 Nov;64(22):2994-3006. doi: 10.1007/s00018-007-7345-y) https://pubmed.ncbi.nlm.nih.gov/17938858/. Both CDS1 (cHP) and CDS2 (SL427) target disruption structures were considered SRVVLC in Pirakitikulr et al. 2016 (Molecular Cell 62, 111-120 Apr. 7, 2016 ^{a}2016 Elsevier Inc. http://dx.doi.org/10.1016/j.molcel.2016.01.024).

### Cell Cultures and Transfection

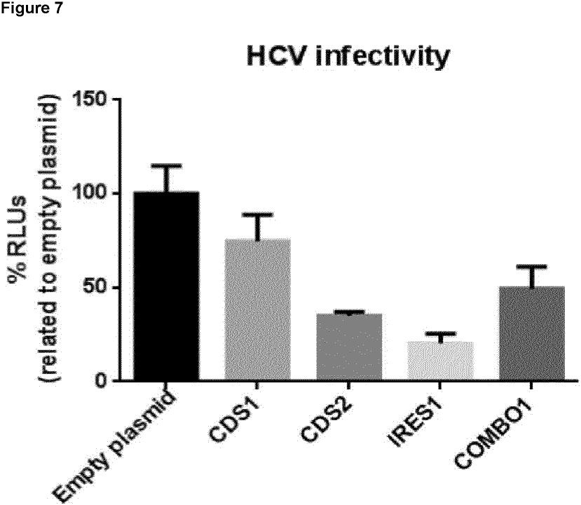

We transfected each circRNA in Huh7 cells and 24h post-transfection we infected the cells with an HCV derivative that expresses luciferase. After 48 hours post-infection, we measured the luciferase levels and compared the values to a control (mock transfected). We carried out five biological replicates for each condition. All circRNAs tested inhibit HCV infection by a significant amount. As shown in FIG. 7 all circRNAs designed are capable of lowering the infection with different efficiencies that range between 30% and 70% with respect to the control.

### Example 2

We have designed and tested three circRNAs (circ_dv_3utr (SEQ ID NO.: 8), circ_dv_cHP_v1 (SEQ ID NO.: 9) and circ_dv_cHP_v2 (SEQ ID NO.: 10)) against two different regions of DENV (Dengue) genome (SEQ ID NO.: 7) as previously disclosed in the section Production of circRNAs. Plasmid to generate DENV (pFK-DVs-R2A) carrying the Renilla luciferase reporter gene has been previously described (Scaturro, P. et al. Characterization of the mode of action of a potent dengue virus capsid inhibitor. J. Virol. 88, 11540-55 (2014)). All three circRNAs contain 7 hybridization regions that target the corresponding regions in the DENV genome (See FIG. 8 and SEQ ID NO.: 7). The target disruption structure corresponding to circ_dv_3utr (sHP) was considered SRVVLC in Huber et al. 2019 (Nature Communications volume 10, Article number: 1408 (2019)), and the target disruption structure cHP was considered SRVVLC in Fernández-Sanlés et al. 2017 (Front Microbiol. 2017; 8: 546,doi:10.3389/fmicb.2017.00546).

### Cell Cultures and Transfection

The human embryonic kidney cell line HEK293 was maintained in Dulbecco’s modified Eagle’s medium (DMEM, Invitrogen, Carlsbad, CA) supplemented with 10% heat inactivated fetal bovine serum (FBS) and 10% non-essential amino acids. Cells were grown in an incubator with 5% CO2 at 37° C. 1 10⁵ HEK293 cells/well were seeded in 24-well plates the day before transfection. 2 micrograms of each plasmid containing the cirRNAs or the empty plasmid were transfected using Lipofectamine 2000 (Invitrogen) following manufacturer’s instructions. After overnight incubation, DMEM medium was removed and cells were washed with 1X PBS. Cells were inoculated with Dengue Virus for 4 h at 37° C. Finally, the virus containing media was replaced with fresh media. Forty-eight hours post infection, Luciferase activity was assayed. Cells were washed with 1X PBS, lysed in 150 µl of Renilla lysis buffer and frozen. Upon thawing, lysates were resuspended by pipetting. 4 µl of the lysates were mixed with 20 µl of Renila Luciferase Assay Buffer and 1/200 of substrate from the Renilla Luciferase assay system (Promega) and measured immediately in a luminometer for 2 s. Mean relative light units (RLU) were plotted as percentage relative to control infections (cells transfected with the circoVIR plasmid).

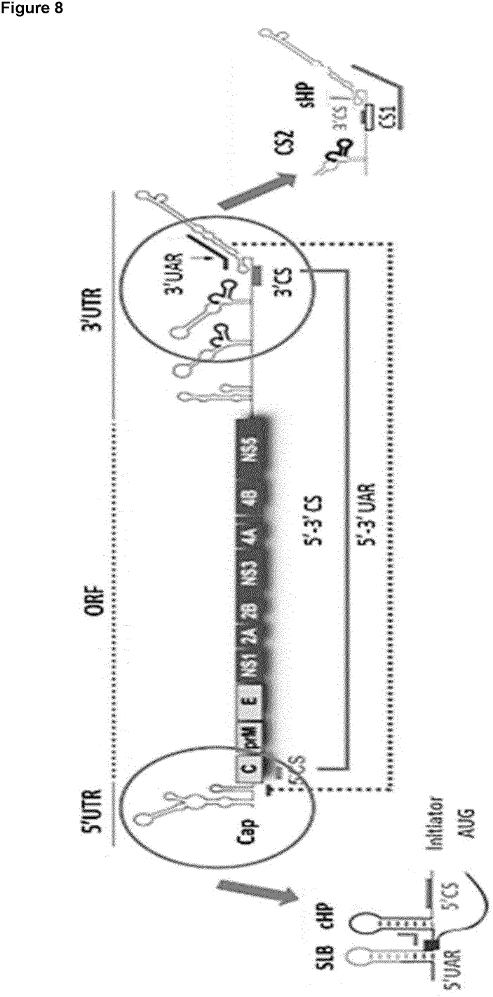

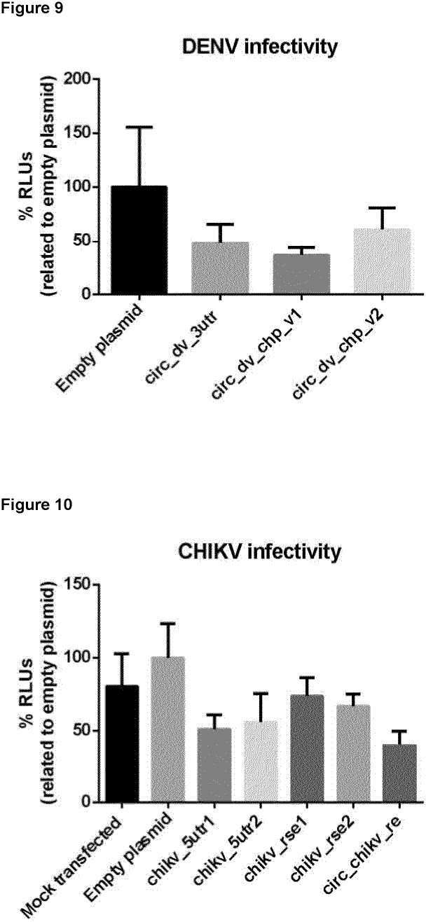

Results are shown in FIG. 9 and depict the significant inhibition of DENV infection with respect to the control (empty plasmid).

### Example 3

We have designed and tested five circRNAs (chikv_5utr1 (SEQ ID NO.: 12), chikv_5utr2 (SEQ ID NO.: 13), chikv_RSE1 (SEQ ID NO.: 14), chikv_RSE2 (SEQ ID NO.: 15) and chikv_RE (SEQ ID NO.: 39) against three different regions of CHIKV (Chikungunya) genome (SEQ ID NO.: 11) as previously disclosed in the section Production of circRNAs. The plasmid to generate CHIKV carrying the reporter gene of Gaussian luciferase (kindly provided by Dr. Merits, University of Tartu, Estonia) is based directly on the viral sequence isolated from a human patient from La Reunion (isolate LR2006_OPY1 (DQ443544). All five circRNAs contain 6 hybridization regions that target the corresponding regions in the CHIKV genome (SEQ ID NO.: 11). Both the target disruption structure en the 5′ UTR of CHIKV and the target disruption structure “Repetitive Sequence Structure” were considered SRVVLC in Hossain Khan et al., 2002 (Journal of General Virology (2002), 83, 3075-3084). The Recoding Element, third target disruption structure for CHIKV, was considered SRVVLC in A. Kendra et al., 2018 (J. Biol. Chem. (2018) 293(45) 17536 -17545).

### Cell Cultures and Transfection

The human embryonic kidney cell line HEK293 was maintained in Dulbecco’s modified Eagle’s medium (DMEM, Invitrogen, Carlsbad, CA) supplemented with 10% heat inactivated fetal bovine serum (FBS) and 10% non-essential amino acids. Cells were grown in an incubator with 5% CO2 at 37° C. 1 10⁵ HEK293 cells/well were seeded in 24-well plates the day before transfection. 2 micrograms of each plasmid containing the cirRNAs or the empty plasmid were transfected using Lipofectamine 2000 (Invitrogen) following manufacturer’s instructions. After overnight incubation, DMEM medium was removed and cells were washed with 1X PBS. Cells were inoculated with Chikungunya Virus for 1 h at 37° C. Finally, the virus containing media was replaced with fresh media. Sixteen hours post infection, Luciferase activity was assayed. The supernatant of the cells was collected and inactivated with UV for 10 minutes. 4 µl of the supernatant were mixed with 20 µl of Renila Luciferase Assay Buffer and 1/200 of substrate from the Renilla Luciferase assay system (Promega) and measured immediately in a luminometer for 2 s. Mean relative light units (RLU) were plotted as percentage relative to control infections (cells transfected with the circoVIR plasmid).

Results are shown in FIG. 10 and depict the significant inhibition of CHIKV infection with respect to the control (empty plasmid).

### Example 4

We have designed and tested three circRNA (DENV1 cHP_ HCV CDS2_1 (SEQ ID NO.: 17), DENV cHP_ HCV CDS2_2 (circ_dv_cHP_v1_circ_hcv_cds2_2, SEQ ID NO.: 32), and (DENV1 cHP_ HCV CDS2_T (SEQ ID NO.: 16) against one region of DENV (Dengue) genome (SEQ ID NO.: 7) and one region of HCV genome (SEQ ID NO.: 1) as previously disclosed in the section Production of circRNAs. Plasmid to generate HCV (pFK-Luc-Jc1) carrying the Firefly luciferase reporter gene has been previously described (Wakita T et al., Production of infectious hepatitis C virus in tissue culture from a cloned viral genome, Nat Med, 2005;11:791-796). Plasmid to generate DENV (pFK-DVs-R2A) carrying the Renilla luciferase reporter gene has been previously described (Scaturro, P. et al., Characterization of the mode of action of a potent dengue virus capsid inhibitor, J. Virol. 88, 11540-55 (2014)).

The first 2 circRNAs contain 6 hybridization regions that target the corresponding regions in the DENV genome and the HCV genome, and the last one contains 7 against DENV and 12 against HCV.

### Cell Cultures and Transfection

The human embryonic kidney cell line HEK293 was maintained in Dulbecco’s modified Eagle’s medium (DMEM, Invitrogen, Carlsbad, CA) supplemented with 10% heat inactivated fetal bovine serum (FBS) and 10% non-essential amino acids. Cells were grown in an incubator with 5% CO2 at 37° C. 1 10⁵ HEK293 cells/well were seeded in 24-well plates the day before transfection. 2 micrograms of each plasmid containing the cirRNAs or the empty plasmid were transfected using Lipofectamine 2000 (Invitrogen) following manufacturer’s instructions. After overnight incubation, DMEM medium was removed and cells were washed with 1X PBS. Cells were inoculated with Dengue Virus for 4 h at 37° C. Finally, the virus containing media was replaced with fresh media. Forty-eight hours post infection, Luciferase activity was assayed. Cells were washed with 1X PBS, lysed in 150 µl of Renilla lysis buffer and frozen. Upon thawing, lysates were resuspended by pipetting. 4 µl of the lysates were mixed with 20 µl of Renila Luciferase Assay Buffer and 1/200 of substrate from the Renilla Luciferase assay system (Promega) and measured immediately in a luminometer for 2 s. Mean relative light units (RLU) were plotted as percentage relative to control infections (cells transfected with the circoVIR plasmid).

The human hepatocarcinoma cell line Huh7/Scr was maintained in Dulbecco’s modified Eagle’s medium (DMEM, Invitrogen, Carlsbad, CA) supplemented with 10% heat inactivated fetal bovine serum (FBS) and 10% non-essential amino acids. Cells were grown in an incubator with 5% CO2 at 37° C. 2.5 10⁴ Huh7/Scr cells/well were seeded in 24-well plates the day before transfection. 2 micrograms of each plasmid containing the cirRNAs or the empty plasmid were transfected using Lipofectamine 2000 (Invitrogen) following manufacturer’s instructions.

After overnight incubation, DMEM medium was removed and cells were washed with 1X PBS. Cells were inoculated with the different viruses for 4 h at 37° C. Finally, the virus containing media was replaced with fresh media. Forty-eight hours post infection, Luciferase activity was assayed. Cells were washed with 1X PBS, lysed in 150 µl of Passive lysis buffer and frozen. Upon thawing, lysates were resuspended by pipetting. 50 µl of the lysates were mixed with 25 µl of Luciferase Assay Reagent (Promega) and incubated 5 minutes at room temperature. Afterwards the luciferase activity was measured in a luminometer for 2 s. Mean relative light units (RLU) were plotted as percentage relative to control infections (cells transfected with the circoVIR plasmid).

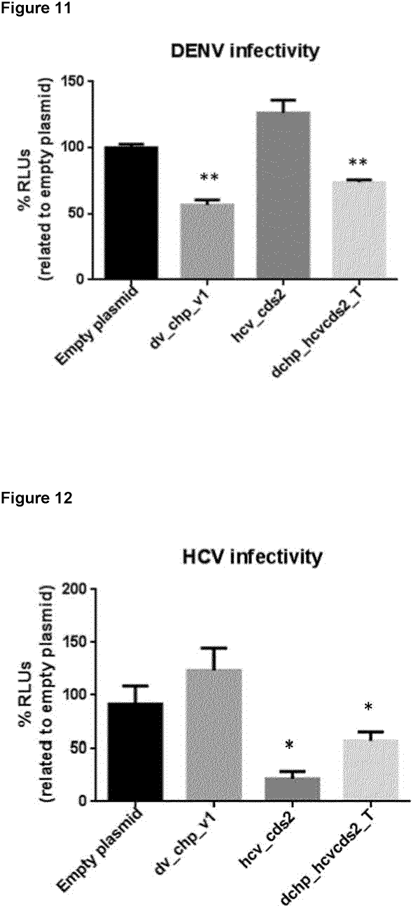

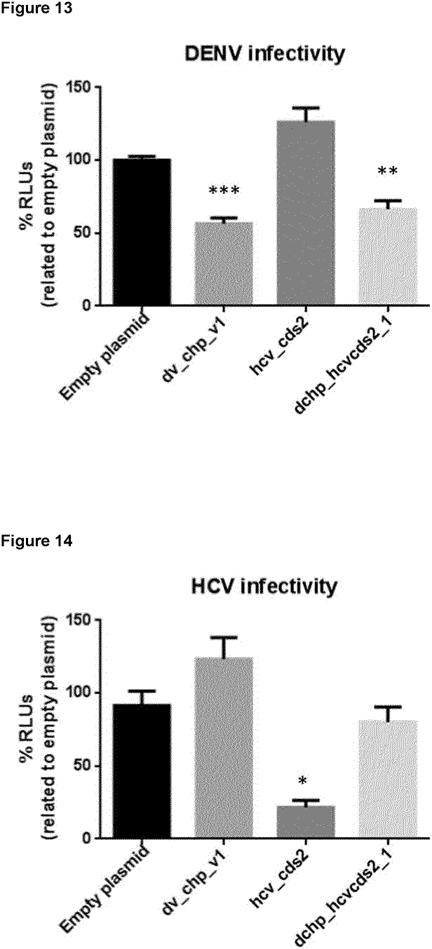

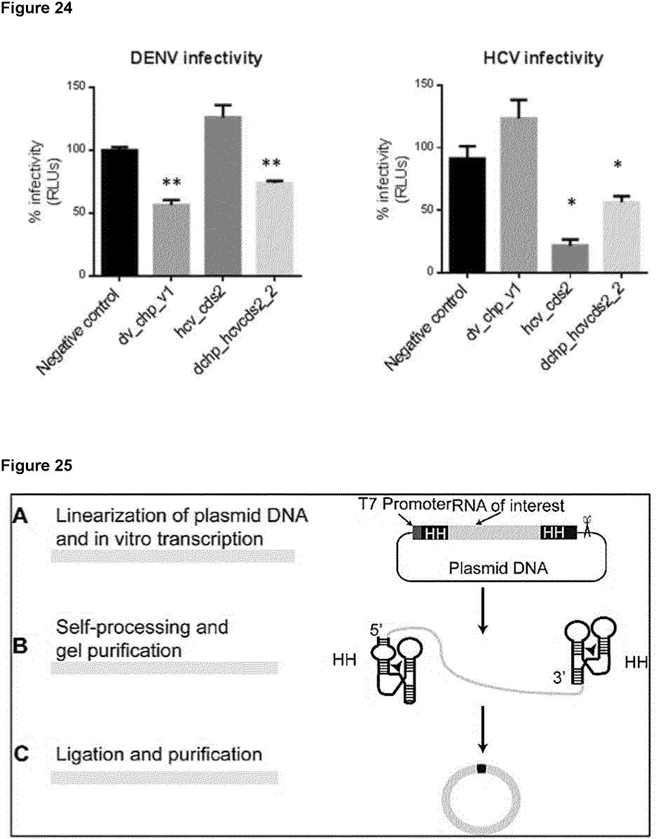

Results for each circRNA against both DENV and HCV along with the corresponding positive and negative controls from examples 1 and 2 are shown in FIGS. 11, 12, 1314 and 24. Inhibition is shown for all circRNAs in both viral infections, demonstrating the broadspectrum capabilities of the design circRNAs.

### Example 5

We have designed and tested two circRNAs (circ_wnv_slll_1 (SEQ ID NO.: 24), circ_wnv_slll_2 (SEQ ID NO.: 19)) against one region of WNV (West Nile Virus) genome (SEQ ID NO.: 20) as previously disclosed in the section Production of circRNAs. Plasmid to generate WNV carrying the Nanoluc luciferase reporter gene was kindly provided by Dr. Merits, University of Tartu, Estonia. Both circRNAs contain 7 hybridization regions that target the corresponding region in the WNV genome. The target disruption structure SLII was considered SRVVLC in Fernández-Sanlés et al., 2017 (Front Microbiol. 2017; 8: 546 doi: 10.3389/fmicb.2017.00546).

### Cell Cultures and Transfection

The human embryonic kidney cell line HEK293 was maintained in Dulbecco’s modified Eagle’s medium (DMEM, Invitrogen, Carlsbad, CA) supplemented with 10% heat inactivated fetal bovine serum (FBS) and 10% non-essential amino acids. Cells were grown in an incubator with 5% CO2 at 37° C. 1 10⁵ HEK293 cells/well were seeded in 24-well plates the day before transfection. 2 micrograms of each plasmid containing the circRNAs or the empty plasmid were transfected using Lipofectamine 2000 (Invitrogen) following manufacturer’s instructions. After overnight incubation, DMEM medium was removed and cells were washed with 1X PBS. Cells were inoculated with WNV for 4 h at 37° C. Finally, the virus containing media was replaced with fresh media. Forty-eight hours post infection, Luciferase activity was assayed. Cells were washed with 1X PBS, lysed in 150 µl of Renilla lysis buffer and frozen. Upon thawing, lysates were resuspended by pipetting. 4 µl of the lysates were mixed with 20 µl of Renila Luciferase Assay Buffer and 1/200 of substrate from the Renilla Luciferase assay system (Promega) and measured immediately in a luminometer for 2 s. Mean relative light units (RLU) were plotted as percentage relative to control infections (cells transfected with the circoVIR plasmid).

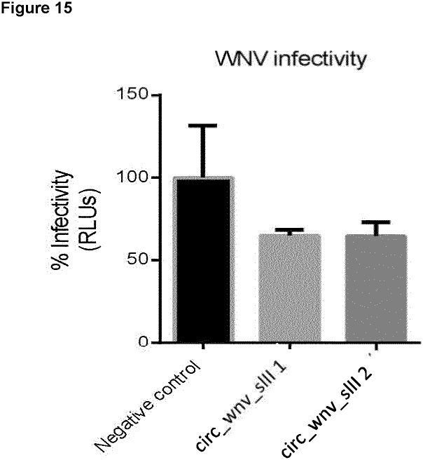

Results are shown in FIG. 15 and depict the significant inhibition of WNV infection with respect to the control (empty plasmid).

### Example 6

We have designed and tested three circRNA (dchp_wslll_A (SEQ ID NO.: 21), dchp_wslll_B (SEQ ID NO.: 22) and dchp_wslll_C (SEQ ID NO.: 23) against one region of DENV (Dengue) genome (SEQ ID NO: 7) and one region of WNV genome (SEQ ID NO.: 20) as previously disclosed in the section Production of circRNAs. Plasmid to generate DENV (pFK-DVs-R2A) carrying the Renilla luciferase reporter gene has been previously described (Scaturro, P. et al., Characterization of the mode of action of a potent dengue virus capsid inhibitor, J. Virol. 88, 11540-55 (2014)). Plasmid to generate WNV carrying the Nanoluc luciferase reporter gene was kindly provided by Dr. Merits, University of Tartu, Estonia. The circRNAs contain 6 hybridization regions that target the corresponding regions in the DENV genome and the WNV genome.

### Cell Cultures and Transfection

The human embryonic kidney cell line HEK293 was maintained in Dulbecco’s modified Eagle’s medium (DMEM, Invitrogen, Carlsbad, CA) supplemented with 10% heat inactivated fetal bovine serum (FBS) and 10% non-essential amino acids. Cells were grown in an incubator with 5% CO2 at 37° C. 1x10⁵ HEK293 cells/well and were seeded in 24-well plates the day before transfection. 2 micrograms of each plasmid containing the circRNAs or the empty plasmid were transfected using Lipofectamine 2000 (Invitrogen) following manufacturer’s instructions. After overnight incubation, DMEM medium was removed and cells were washed with 1X PBS. Cells were inoculated with Dengue Virus or West Nile Virus for 4h at 37° C. Finally, the virus containing media was replaced with fresh media. Forty-eight hours post infection, Luciferase activity was assayed. Cells were washed with 1X PBS, lysed in 150 µl of Renilla lysis buffer and frozen. Upon thawing, lysates were resuspended by pipetting. 4 µl of the lysates were mixed with 20 µl of Renila Luciferase Assay Buffer and 1/200 of substrate from the Renilla Luciferase assay system (Promega) and measured immediately in a luminometer for 2 s. Mean relative light units (RLU) were plotted as percentage relative to control infections (cells transfected with the circoVIR plasmid).

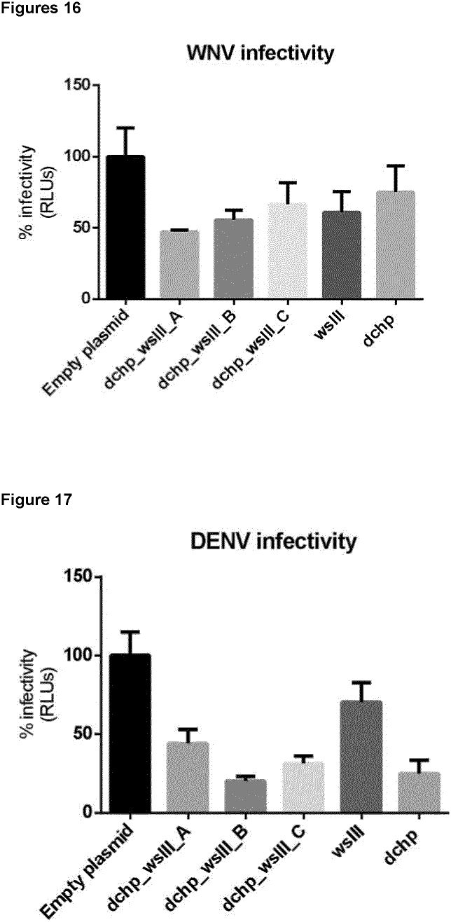

Results for each circRNA against both DENV and WNV along with the corresponding positive and negative controls from examples 2 and 5 are shown in FIGS. 16 and 17. Inhibition is shown for the circRNAs in both viral infections, demonstrating the broadspectrum capabilities of the designed circRNAs.

### Example 7

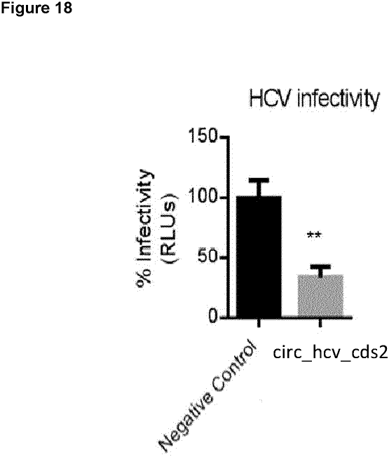

We tested whether circ_hcv_cds2 (SEQ ID NO.: 6) may inhibit infections already established. For this, Huh7/Scr cells were infected with HCVJc1-luc and 48 hours later transfected with circ_hcv_cds2 (SEQ ID NO.: 6). Luciferase values were measured 24 hours post-transfection. Importantly, circ_hcv_cds2 inhibited infectivity with similar efficiency as when cells were expressing the circ_hcv_cds2 before infection (FIG. 18).

### Example 8

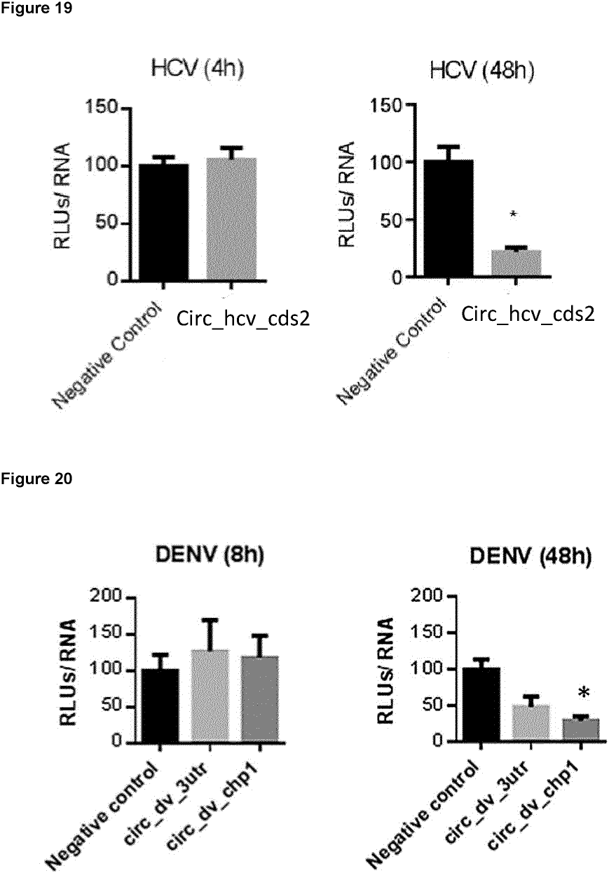

Next, we examine whether the circ_hcv_cds2 (SEQ ID NO.: 6) is indeed inhibiting the function described for the target sequence, viral RNA replication. For this, we used HCV RNA replicons that harbour a luciferase reporter gene but not the viral structural genes required for encapsidation. Thus, these replicons allow efficient translation and replication of the viral RNA genome but not virion production. Huh7/Scr cells were transfected with circ_hcv_cds2 (SEQ ID NO.: 6) or the corresponding empty plasmid and the next day, transfected with the HCV replicon. Luciferase values were measured at 4 hours and 48 hours post-HCV replicon transfection. These times were selected because already established kinetics prove that luciferase production derived at 4 hours is solely from HCV RNA translation and at 48 hours from both translation and replication. Indeed, a decrease in viral infectivity was observed at 48 but not at 4 hours post-transfection (FIG. 19) indicating that circ_hcv_cds2 impairs viral RNA replication.

### Example 9

Circ_dv_3utr (SEQ ID NO.: 8) and circ_dv_cHP_v1 (SEQ ID NO.: 9), designed to target structures within the DENV RNA genome directing RNA replication, were tested to see if they inhibit DENV RNA replication following the same procedure as in Example 8. The results show that circ_dv_3utr and circ_dv_cHP_v1 inhibit luciferase expression levels at 48 hours when the RNA genome is translated and replicated but not at 8 hours when is solely translated (FIG. 20).

### Example 10

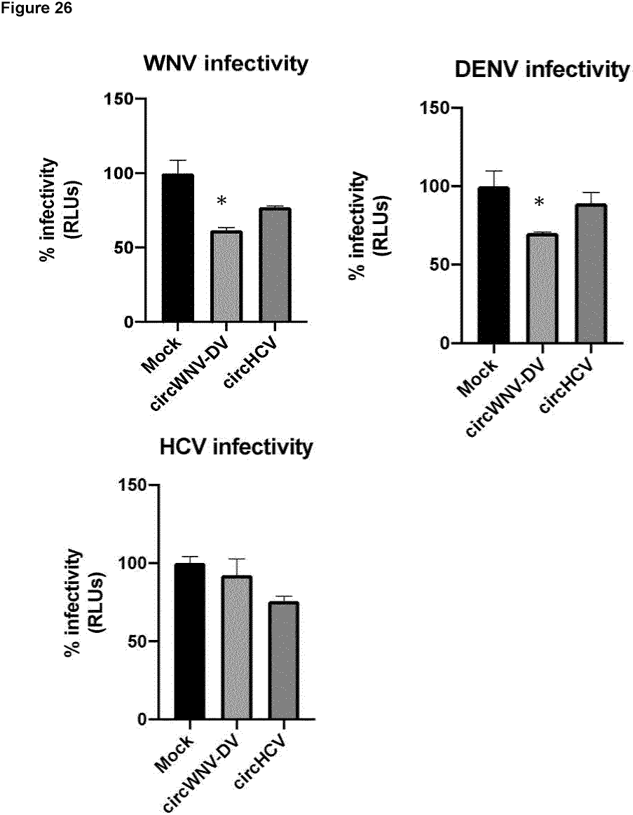

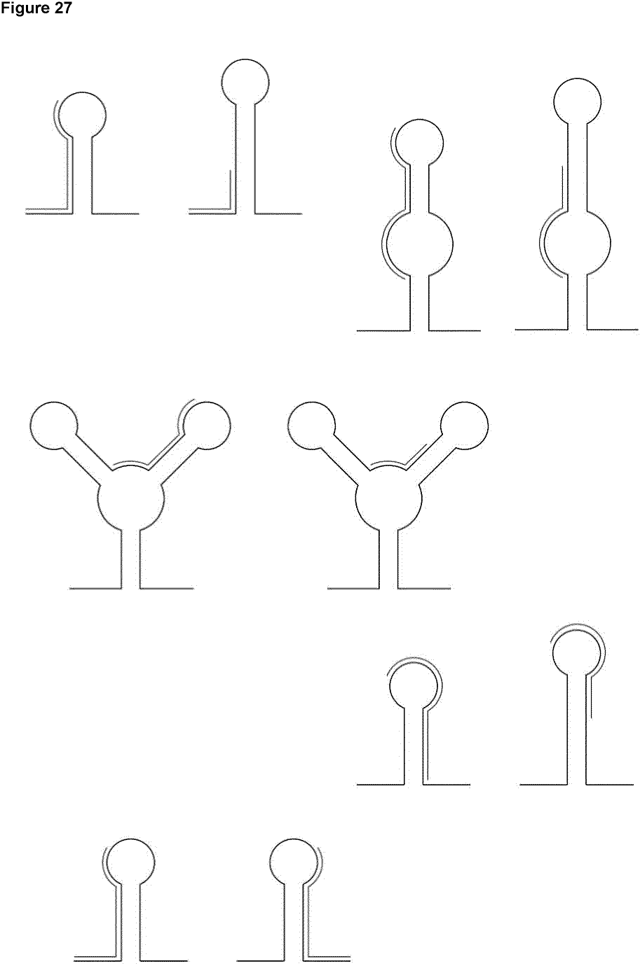

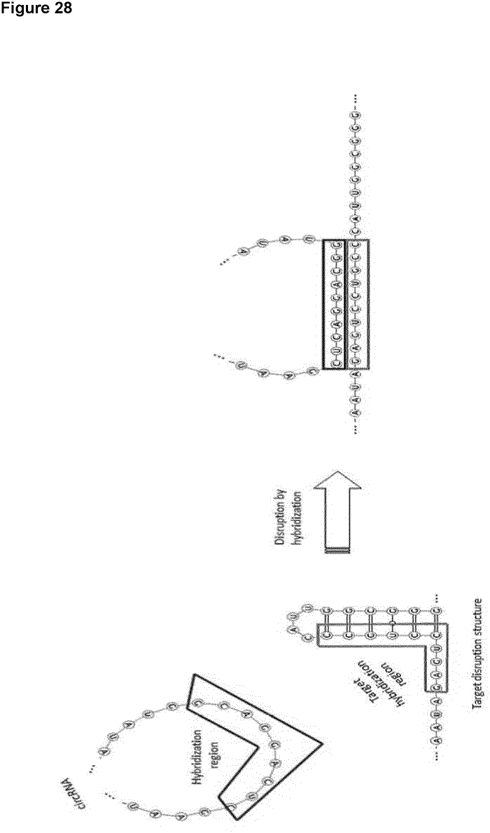

The human embryonic kidney cell line HEK293 and the Hepatocarcinoma cell line Huh7 were maintained in Dulbecco’s modified Eagle’s medium (DMEM, Invitrogen, Carlsbad, CA) supplemented with 10% heat inactivated fetal bovine serum (FBS) and 10% non-essential amino acids. Cells were grown in an incubator with 5% CO2 at 37° C. 1x10⁵ HEK293 cells/well or 4x10⁴ Huh7/cells and were seeded in 24-well plates the day before transfection. 100 nanograms of circular RNAs against WNV-DENV (circWD1-in vitro SEQ ID NO.: 63) or circular RNA against HCV (circHCV-in vitro SEQ ID NO.: 64) were transfected using Lipofectamine 2000 (Invitrogen) following manufacturer’s instructions. After overnight incubation, DMEM medium was removed and cells were washed with 1X PBS. HEK293 cells were inoculated with Dengue Virus or West Nile Virus for 4 h at 37° C. In parallel, Huh7 cells were inoculated with Hepatitis C virus for 4h at 37° C. Finally, the virus containing media was replaced with fresh media. Forty-eight hours post infection, Luciferase activity was assayed. Cells were washed with 1X PBS, lysed in 150 µl of Renilla lysis buffer (HEK293) or 150 µl of Passive lysis buffer and frozen. Upon thawing, lysates were resuspended by pipetting. In the case of DENV and WNV, 4 µl of the lysates were mixed with 20 µl of Renila Luciferase Assay Buffer and 1/200 of substrate from the Renilla Luciferase assay system (Promega) and measured immediately in a luminometer for 2 s. In the case of HCV, 50 µl of the lysates were mixed with 25 µl of Luciferase Assay Reagent (LARII) for 5 minutes and measured immediately in a luminometer for 2 s. Mean relative light units (RLU) were plotted as percentage relative to control infections (cells transfected with the circoVIR plasmid) in FIG. 26.

### Example 11

We have designed and tested twelve circRNA (circ_SARS_tA_4 (SEQ ID NO.: 49), circ_SARS_tA_5 (SEQ ID NO.: 68), circ_SARS_tA_6 (SEQ ID NO.: 69), circ_SARS_tA_7 (SEQ ID NO.: 70), circ_SARS_tC_3 (SEQ ID NO.: 52), circ_SARS_tC_5 (SEQ ID NO.: 71), circ_SARS_tC_6 (SEQ ID NO.: 72), circ_SARS_3utr_6 (SEQ ID NO.:63), circ_SARS_3utr_7 (SEQ ID NO.: 89), circ_SARS_3utr_8 (SEQ ID NO.:65), circ_SARS_5utr_4 (SEQ ID NO.: 66), circ_SARS_5utr_5 (SEQ ID NO.: 67) against four regions of SARS-CoV-2 genome (SEQ ID NO: 58; SEQ ID NO: 59; SEQ ID NO: 60; SEQ ID NO: 62) as previously disclosed in the section Production of circRNAs. SARS-CoV-2 strain hCoV-19/Spain/VH000001133/2020 (EPI_ISL_418860) was kindly provided by Miguel Chillon, Universitat Autonoma de Barcelona. The designed circRNAs contain between 6 and 7 hybridization regions that target the corresponding regions in the SARS-CoV-2 genome. The target disruption structure Replication Site was considered SRVVLC in J. Goebel et al., 2004 (American Society for Microbiology. Journal of VirologyVolume 78, Issue 14, Pages 7846-7851https://doi.org/10.1128/JVI.78.14.7846-7851.2004). The target disruption structure SL-2 was considered SRVVLC in Chen et al., 2010 (Virology 401 (2010) 29-41 doi: 10.1016/j.virol.2010.02.007). The rest of the target disruption structures (A,C and D) were considered to be viral conserved structures, following the RNAz approach mentioned above, in Rangan et al., 2020 (doi: https://doi.org/10.1101/2020.03.27.012906).

### Example 12. Cell Cultures and Transfection

The green monkey kidney epithelial cell line VERO E6 was maintained in Dulbecco’s modified Eagle’s medium (DMEM, Invitrogen, Carlsbad, CA) supplemented with 10% heat inactivated fetal bovine serum (FBS) and 10% non-essential amino acids. Cells were grown in an incubator with 5% CO2 at 37° C. 6x104 VERO E6 cells per well were seeded in 24-well plates the day before transfection. 2 micrograms of each plasmid containing the circRNAs or the empty plasmid were transfected using Lipofectamine 2000 (Invitrogen) following manufacturer’s instructions. After overnight incubation, DMEM medium was removed and cells were washed with 1X PBS. Cells were inoculated with SARS-CoV-2 for 1 h at 37° C. Finally, the virus containing media was replaced with fresh media. Forty-eight hours post infection, supernatant was collected and inactivated and viral RNA was extracted using Quick-RNA Viral Kit (Zymo Research, Irvine, USA). SARS-CoV-2 RNA levels were measured by qPCR using qScript XLT One-Step RT-qPCR ToughMix, ROX (Quanta Biosciences, Berverly, USA), using the specific probe 2019-nCoV_N1-P, 5′-FAM-ACCCCGCATTACGTTTGGTGGACC-BHQ1-3′ (SEQ ID NO.:73); as well as primers 2019-nCoV_N1-F, 5′-GACCCCAAAATCAGCGAAAT-3′ (SEQ ID NO.: 74); and 2019-nCoV_N1-R, 5′-TCTGGTTACTGCCAGTTGAATCTG-3′ (SEQ ID NO.: 75) (Biomers, Ulm, Germany). Mean relative RNA levels were plotted as percentage relative to control infections (cells transfected with the circoVIR plasmid).

Results for each circRNA against SARS-CoV-2 are shown in FIG. 33. Inhibition is shown for the circRNAs in SARS-CoV-2 viral infection, demonstrating the antiviral effect of the designed circRNAs.

### Example 13

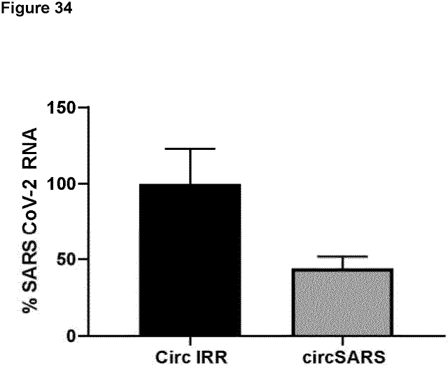

The green monkey kidney epithelial cell line VERO E6 was maintained in Dulbecco’s modified Eagle’s medium (DMEM, Invitrogen, Carlsbad, CA) supplemented with 10% heat inactivated fetal bovine serum (FBS) and 10% non-essential amino acids. Cells were grown in an incubator with 5% CO2 at 37° C. 6x104 VERO E6 cells per well were seeded in 24-well plates the day before transfection. 100 nanograms of circular RNAs against SARS-CoV-2 (circSARS-in vitro SEQ ID NO.: 70) or circular RNA against WNV-DENV (circWD1-in vitro SEQ ID NO.: 21) were transfected using Lipofectamine 2000 (Invitrogen) following manufacturer’s instructions. After overnight incubation, DMEM medium was removed and cells were washed with 1X PBS. Cells were inoculated with SARS-CoV-2 for 1 h at 37° C. Finally, the virus containing media was replaced with fresh media. Forty-eight hours post infection, supernatant was collected and inactivated and viral RNA was extracted using Quick-RNA Viral Kit (Zymo Research, Irvine, USA). SARS-CoV-2 RNA levels were measured by qPCR using qScript XLT One-Step RT-qPCR ToughMix, ROX (Quanta Biosciences, Berverly, USA), using the specific probe 2019-nCoV_N1-P, 5′-FAM-ACCCCGCATTACGTTTGGTGGACC-BHQ1-3′ (SEQ ID NO.:73); as well as primers 2019-nCoV_N1-F, 5′-GACCCCAAAATCAGCGAAAT-3′ (SEQ ID NO.: 74); and 2019-nCoV_N1-R, 5′-TCTGGTTACTGCCAGTTGAATCTG-3′ (SEQ ID NO.: 75) (Biomers, Ulm, Germany). Mean relative RNA levels were plotted as percentage relative to control infections (cells transfected with an irrelevant circRNA) in FIG. 34.

### Example 14

In this example we present some of the input files for RNAiFold to both check whether a target disruption structure is indeed possible to disrupt by hybridization (from which immediately follows the design of the circRNA with the hybridization region/s to disrupt it).

For HCV target disruption structure cHP and corresponding target hybridization region (SEQ ID NO 27, highlighted in bold):

#RNAscdstr((((((((((((((((((((((((((((&)))))))))))
))))))))))))))))),,,,,,,,,,,,,,,,,,,,,,,,#RNAseqco
nNNNNNNNNNNNNNNNNNNNNNNNNNNNN&CCAAAAGAAACACCAACCGU
CGCCCAGAAGACGUUAAGUUCCCGGGCGGCGG

The input “((((((((((((((((((((((((((((&)))))))))))))))))))))))))))),,,,,,,,,,,,,,,,,,,,,,,,” means that the software should look for a nucleotide sequence that is k nucleotides in length, wherein the nucleotides located at positions corresponding to a open bracket “(” should be base paired to the nucleotides located at positions corresponding to a closed bracket “)” in the Minimum Free Energy structure. The comas “,” mean that the nucleotides in the corresponding positions can be base paired or not in the Minimum Free Energy Structure. The & symbol separates both strands. Note that the base paired nucleotides on the second strand (target disruption structure) correspond to the target hybridization region.

Further, the input:

“NNNNNNNNNNNNNNNNNNNNNNNNNNNN&CCAAAAGAAACACCAACCGU”
CGCCCAGAAGACGUUAAGUUCCCGGGCGGCGG "

means fixes the nucleotides that are available for selection, where N means any nucleotide (A,C,G or U). In this case, the nucleotides corresponding to the target disruption structure are fixed and all the nucleotides corresponding to the hybridization region are unfixed (N- any nucleotide). The results are shown herein below:

UCUGGGCGACGGUUGGUGUUUCUUUUGG&CCAAAAGAAACACCAACCGUC
GCCCAGAAGACGUUAAGUUCCCGGGCGGCGG

UUUGGGCGACGGUUGGUGUUUCUUUUGG&CCAAAAGAAACACCAACCGUC
GCCCAGAAGACGUUAAGUUCCCGGGCGGCGG

UCUGGGUGACGGUUGGUGUUUCUUUUGG&CCAAAAGAAACACCAACCGUC
GCCCAGAAGACGUUAAGUUCCCGGGCGGCGG

UUUGGGUGACGGUUGGUGUUUCUUUUGG&CCAAAAGAAACACCAACCGUC
GCCCAGAAGACGUUAAGUUCCCGGGCGGCGG

UCUGGGCGGCGGUUGGUGUUUCUUUUGG&CCAAAAGAAACACCAACCGUC
GCCCAGAAGACGUUAAGUUCCCGGGCGGCGG

UUUGGGCGGCGGUUGGUGUUUCUUUUGG&CCAAAAGAAACACCAACCGUC
GCCCAGAAGACGUUAAGUUCCCGGGCGGCGG

UCUGGGUGGCGGUUGGUGUUUCUUUUGG&CCAAAAGAAACACCAACCGUC
GCCCAGAAGACGUUAAGUUCCCGGGCGGCGG

UUUGGGUGGCGGUUGGUGUUUCUUUUGG&CCAAAAGAAACACCAACCGUC
GCCCAGAAGACGUUAAGUUCCCGGGCGGCGG

UCUGGGCGAUGGUUGGUGUUUCUUUUGG&CCAAAAGAAACACCAACCGUC
GCCCAGAAGACGUUAAGUUCCCGGGCGGCGG

UUUGGGCGAUGGUUGGUGUUUCUUUUGG&CCAAAAGAAACACCAACCGUC
GCCCAGAAGACGUUAAGUUCCCGGGCGGCGG

For CHIKV target disruption structure RSE and corresponding target hybridization region (SEQ ID NO 35):

#RNAscdstr(((((((((((((((((((((((((((((((((&))))))
))))))))))))))))))))))))))).#RNAseqconNNNNNNNNNNNN NNNNNNNNNNNNNNNNNNNNN&AGCAAAUAAUCUAUAGAUCAAAGGGCUA CGCAACCCCUGAA

The results are shown herein below:

UUGCGUGGCCCUUUGGUCUGUAGGUUGUUUGCU&AGCAAAUAAUCUAUAG
AUCAAAGGGCUACGCAACCCCUGAA

UUGUGUGGCCCUUUGGUCUGUAGGUUGUUUGCU&AGCAAAUAAUCUAUAG
AUCAAAGGGCUACGCAACCCCUGAA

UUGCGUAGCCCUUUGGUCUGUAGGUUGUUUGCU&AGCAAAUAAUCUAUAG
AUCAAAGGGCUACGCAACCCCUGAA

UUGUGUAGCCCUUUGGUCUGUAGGUUGUUUGCU&AGCAAAUAAUCUAUAG
AUCAAAGGGCUACGCAACCCCUGAA

UUGCGUGGUCCUUUGGUCUGUAGGUUGUUUGCU&AGCAAAUAAUCUAUAG
AUCAAAGGGCUACGCAACCCCUGAA

UUGUGUGGUCCUUUGGUCUGUAGGUUGUUUGCU&AGCAAAUAAUCUAUAG
AUCAAAGGGCUACGCAACCCCUGAA

UUGCGUAGUCCUUUGGUCUGUAGGUUGUUUGCU&AGCAAAUAAUCUAUAG
AUCAAAGGGCUACGCAACCCCUGAA

UUGUGUAGUCCUUUGGUCUGUAGGUUGUUUGCU&AGCAAAUAAUCUAUAG
AUCAAAGGGCUACGCAACCCCUGAA

UUGCGUAGCUCUUUGGUCUGUAGGUUGUUUGCU&AGCAAAUAAUCUAUAG
AUCAAAGGGCUACGCAACCCCUGAA

UUGUGUAGCUCUUUGGUCUGUAGGUUGUUUGCU&AGCAAAUAAUCUAUAG
AUCAAAGGGCUACGCAACCCCUGAA

For WNV target disruption structure SLII and corresponding target hybridization region (SEQ ID NO 37):

#RNAscdstr((((((((((((((((((((((((((((&))))))))))) ))))))))))))))))),,,,,,,,,,,,,,,,,,,,,,,,#RNAseqco n NNNNNNNNNNNNNNNNNNNNNNNNNNNNNNNNN&UUUUGAGGAGAAAGU CAGGCCGGGAAGUUCCCGCCACCGGAAGUUGAGUAGACGGUGCUGCCUGC GA

The results are shown herein below:

GAUUUUCUGGCCUGGCUUUUUCCUCAAGA&UUUUGAGGAGAAAGUCAGGC CGGGAAGUUCCCGCCACCGGAAGUUGAGUAGACGGUGCUGCCUGCGA

AAUUUUCUGGCCUGGCUUUUUCCUCAAGA&UUUUGAGGAGAAAGUCAGGC CGGGAAGUUCCCGCCACCGGAAGUUGAGUAGACGGUGCUGCCUGCGA

GAUUUUUUGGCCUGGCUUUUUCCUCAAGA&UUUUGAGGAGAAAGUCAGGC CGGGAAGUUCCCGCCACCGGAAGUUGAGUAGACGGUGCUGCCUGCGA

AAUUUUUUGGCCUGGCUUUUUCCUCAAGA&UUUUGAGGAGAAAGUCAGGC CGGGAAGUUCCCGCCACCGGAAGUUGAGUAGACGGUGCUGCCUGCGA

GAUUUUCUGGCUUGGCUUUUUCCUCAAGA&UUUUGAGGAGAAAGUCAGGC CGGGAAGUUCCCGCCACCGGAAGUUGAGUAGACGGUGCUGCCUGCGA

AAUUUUCUGGCUUGGCUUUUUCCUCAAGA&UUUUGAGGAGAAAGUCAGGC CGGGAAGUUCCCGCCACCGGAAGUUGAGUAGACGGUGCUGCCUGCGA

GAUUUUUUGGCUUGGCUUUUUCCUCAAGA&UUUUGAGGAGAAAGUCAGGC CGGGAAGUUCCCGCCACCGGAAGUUGAGUAGACGGUGCUGCCUGCGA

AAUUUUUUGGCUUGGCUUUUUCCUCAAGA&UUUUGAGGAGAAAGUCAGGC CGGGAAGUUCCCGCCACCGGAAGUUGAGUAGACGGUGCUGCCUGCGA

GACUUUCUGGCCUGGCUUUUUCCUCAAGA&UUUUGAGGAGAAAGUCAGGC CGGGAAGUUCCCGCCACCGGAAGUUGAGUAGACGGUGCUGCCUGCGA

AACUUUCUGGCCUGGCUUUUUCCUCAAGA&UUUUGAGGAGAAAGUCAGGC CGGGAAGUUCCCGCCACCGGAAGUUGAGUAGACGGUGCUGCCUGCGA

For SARS-Cov2 target disruption structure Replication Site and corresponding target hybridization region (SEQ ID NO 62):

#RNAscdstr(((((((((((((((((((((((((((((((((((&)))) ))))))))))))))))))))))))))))))).............#RNAse qconNNNNNNNNNNNNNNNNNNNNNNNNNNNNNNNNN&CAGAAUGAAUUC UCGUAACUACAUAGCACAAGUAGAUGUAGUUAACUU

The results are shown herein below:

CUACUUGUGCUAUGUAGUUACGGGAGUUCGUUCUG&CAGAAUGAAUUCUC GUAACUACAUAGCACAAGUAGAUGUAGUUAACUU

UUACUUGUGCUAUGUAGUUACGGGAGUUCGUUCUG&CAGAAUGAAUUCUC GUAACUACAUAGCACAAGUAGAUGUAGUUAACUU

CUGCUUGUGCUAUGUAGUUACGGGAGUUCGUUCUG&CAGAAUGAAUUCUC GUAACUACAUAGCACAAGUAGAUGUAGUUAACUU

UUGCUUGUGCUAUGUAGUUACGGGAGUUCGUUCUG&CAGAAUGAAUUCUC GUAACUACAUAGCACAAGUAGAUGUAGUUAACUU

CUAUUUGUGCUAUGUAGUUACGGGAGUUCGUUCUG&CAGAAUGAAUUCUC GUAACUACAUAGCACAAGUAGAUGUAGUUAACUU

UUAUUUGUGCUAUGUAGUUACGGGAGUUCGUUCUG&CAGAAUGAAUUCUC GUAACUACAUAGCACAAGUAGAUGUAGUUAACUU

CUGUUUGUGCUAUGUAGUUACGGGAGUUCGUUCUG&CAGAAUGAAUUCUC GUAACUACAUAGCACAAGUAGAUGUAGUUAACUU

UUGUUUGUGCUAUGUAGUUACGGGAGUUCGUUCUG&CAGAAUGAAUUCUC GUAACUACAUAGCACAAGUAGAUGUAGUUAACUU

CUGCUUGUGUUAUGUAGUUACGGGAGUUCGUUCUG&CAGAAUGAAUUCUC GUAACUACAUAGCACAAGUAGAUGUAGUUAACUU

UUGCUUGUGUUAUGUAGUUACGGGAGUUCGUUCUG&CAGAAUGAAUUCUC GUAACUACAUAGCACAAGUAGAUGUAGUUAACUU

In all cases, including those not depicted above, RNAiFold return multiple solutions, i.e., sequences corresponding to potential hybridization regions that shall disrupt by hybridization the corresponding target disruption structures.

Lasltly, we present herein the well-established dot bracket notation used in the present invention to obtain the target hybridization region (highlighted herein in bold) for each virus:

### HCV

### Target 1: IRES 1 (Domain II)

SEQ ID NO.: 76, FIG. 1a in Romero et al. 2007 (Cell Mol Life Sci. 2007 Nov;64(22):2994-3006. doi: 10.1007/s00018-007-7345-y.):

CUCCGCCAUGAAUCACUCCCCUGUGAGGAACUACUGUCUUCACGCAGAAA
GCGCCUAGCCAUGGCGUUAGUAUGAGUGUCGUACAGCCUCCAGGCCC...
................((((.((((.....(((((..(((.((...((((
((.......))))))....)).)))..).))))))))))))...

### Target 2: IRES 2 (Domain IV)

SEQ ID NO. : 77, FIG. 1a in Romero et al. (Cell Mol Life Sci. 2007 Nov;64(22):2994-3006. doi: 10.1007/s00018-007-7345-y.):

UCUCGUAGACCGUGCACCAUGAGCACAAAUCCU...........((((..
.....)))).......

### Target 3: CDS1 (cHP)

SEQ ID NO. : 78; FIG. S1 B in Pirakitikulr et al.2016 (Pirakitikulr et al., 2016, Molecular Cell 62, 111-120 Apr. 7, 2016 Elsevier Inc):

CCAAAAGAAACACCAACCGUCGCCCAGAAGACGUUAAGUUCCCGGGC
GGCGG ................(((((((((.(..(((.....)))..
))))))))))

### Target 4: CDS2 (SL427)

SEQ ID NO.: 79; FIG. 2a in Pirakitikulr et al.2016 (Pirakitikulr et al., 2016, Molecular Cell 62, 111-120 Apr. 7, 2016 Elsevier Inc).

GAUCGUUGGCGGAGUAUACUUGUUGCCGCGCAGGGGCCCCAGGUUGGGUG
UGCGCACGACAAGGAAAACUUCGGAGCGGUC(((((((..((((((...(
((((((..(((((...((((......)))).))))).)))))))....))
)))).)))))))

### DNV

### Target 1 : sHP

SEQ ID NO.: 29; FIG. 1c in Huber et al., 2019 (Huber et al. Nature Communications volume 10, Article number: 1408, 2019)

AACAGCAUAUUGACGCUGGGAAAGACCAGAGA...............(((
(......))))...

### Target 2: cHP

SEQ ID NO.: 30; FIG. 2B in Sanlés et al. 2017 (Sanlés et al. 2017. Front Microbiol.; 8: 546.):

ACGGAAAAAGGCGAAAAACACGCCUUUCAAUAU......(((((.(....
....))))))......

### CHIKV

### Target 1: 5′UTR

SEQ ID NO.: 80, FIG. 3A in Khan et al. 2002 (Khan et al. Journal Of General Virology Volume 83, Issue 12):

AUGGCUGCGUGAGACACACGUAGCCUACCAGUUUCUUACUGCUCUACUCU
GCAAAGCAAGAGAUUAAUAA..(((((((((.....)))))))))....(
(((((((.((((........))..)).)))))))).....

### Target 2: Repetitive Sequence Element

SEQ ID NO.: 81, FIG. 4B in Khan et al. 2002 (Khan et al. Journal Of General Virology Volume 83, Issue 12):

AGCAAAUAAUCUAUAGAUCAAAGGGCUACGCAACCCCUGAA.........
........(((..(((........))).))).

### Target 3: Recoding Element

SEQ ID NO.: 82, FIG. 4D in Kendra et al. 2018 (Kendra et al. 2018 Protein Synthesis And Degradation| Volume 293, Issue 45, P17536-17545, Nov. 09, 2018):

UGUCUGAGACUCUUACCAUGCUGCUGUAAAACGUUGGCUUUUUUAGCCGU AAUGAGCGUCGGUGCCCAC.((((((..(((((((..(((....)))...
....((((.....)))))))).)))..)))).))....

### WNV

### Target 1: SLII

SEQ ID NO.: 83, FIG. 3 in Sanlés et al. 2017 (Sanlés et al. 2017. Front Microbiol.; 8: 546.):

UUUUGAGGAGAAAGUCAGGCCGGGAAGUUCCCGCCACCGGAAGUUGAGUA
GACGGUGCUGCCUGCGA.............(.((((((((((...)))))
.(((((.............)))))..))))))..

### SARS-CoV-2

### Target 1: Replication Site Fully Conserved in Pangolin and RatG13

SEQ ID NO.: 84, FIG. 1 in Goebel et al. 2004 (Goebel et al.. Journal Of Virology, July 2004, p. 7846-7851):

CAGAAUGAAUUCUCGUAACUACAUAGCACAAGUAGAUGUAGUUAACUU..
.............(((((((((.((....))..)))))))))....

### Target 2: SL-2

SEQ ID NO.: 58, FIG. 3b in Cheng Chen et al. 2010 (Cheng chen et al. Virology, Volume 401, Issue 1, 25 May 2010, Pages 29-41)

AACCAACUUUCGAUCUCUUGUAGAUCU ...........(((((.....)
)))).

### Target 3: Target A

SEQ ID NO.: 85, FIG. 2 in Rangan et al. 2020 (Rangan et al. bioRxiv preprint doi: https://doi.org/10.1101/2020.03.27.012906):

CUUUAAGUUUAGAAUAGACGGUGACAUGGUACCACAUAUAUCACGUCAAC
GUCUUA...............(((((.((((.(((((.......))))).
)))).)))))..

### Target 4: Target C

SEQ ID NO.: 86, FIG. 2 in Rangan et al. 2020 (Rangan et al. bioRxiv preprint doi: https://doi.org/10.1101/2020.03.27.012906):

UCACUAAGAAAUCUGCUGCUGAGGCUUCUAAGAAGCCCUCGGCAAA....
...........((((((((((...)))).))))))))..

### Target 5: Target D

SEQ ID NO.: 87, FIG. 2 in Rangan et al. 2020 (Rangan et al. bioRxiv preprint doi: https://doi.org/10.1101/2020.03.27.012906):

UCGUCUAUCUUCUGCAGGCUGCUUACGGUUUCGUCCGUGUUGCAGCGAU.
................(((((.((((......)))))..)))))...

### Further Items of the Invention

The present invention also comprises the following items:

1. An artificial circular RNA between 200 and 600 nucleotides having 6 to 20 hybridization regions of sizes between 10 and 50 nucleotides against one or more structured regions of one or more RNA viral genomes, wherein such hybridization regions have sequences that are different among each other, wherein the one or more structured regions are structured regions vital for the viral life cycle (SRVVLC) preceded by a single stranded region, and wherein the artificial circular RNA is capable of disrupting the structure of the one or more SRVVLC, rendering the virus less infective.

2. The artificial circular RNA of item 1, wherein the hybridization regions are:
a) separated by non-hybridization regions of sizes up to 20 nucleotides; or
b) are not separated by non-hybridization regions; or
c) are overlapping.

3. The artificial circular RNA according to any of the preceding items, wherein the target of the hybridization regions is a SRVVLC of the IRES element of the viral genome.

4. The artificial circular RNA according to any of the items 1 o 2, wherein the target of the hybridization regions is a SRVVLC of the 5′UTR of the viral genome.

5. The artificial circular RNA according to any of the items 1 or 2, wherein the target of the hybridization regions is a SRVVLC of the CDS of the viral genome.

6. The artificial circular RNA according to items 1 or 2, wherein the target of the hybridization regions is a SRVVLC of the 3′UTR of the viral genome.

7. The artificial circular RNA, according to any of items 1 to 6, wherein the target of the hybridization regions is a combination of two or more of the IRES element, the region of the 5′UTR, the region of the CDS or the region of the 3′UTR of the viral genome.

8. The artificial circular RNA according to item 3 or 7, wherein said viral genome is selected from HCV, HAV, Poliovirus, Coxsackie B virus and rhinovirus (common cold).

9. The artificial circular RNA according to any of items 4 to 7, wherein said viral genome is selected from HCV, Dengue, Zika, Chikungunya, West Nile and Yellow Fever virus.

10. The artificial circular RNA according to any of items 1 to 9, wherein said circular RNA has a broad spectrum activity against two or more RNA viral genome.

11. A composition comprising the artificial circular RNA according to any of the previous items.

12. A kit comprising the artificial circular RNA according to any of items 1 to 10 or the composition according to item 11, and instructions for using said circular RNA or composition.

13. The artificial circular RNA according to any of items 1 to 10, or the composition according to item 11 or the kit according to items 12, wherein said circular RNA comprises the sequence selected from SEQ ID NO: 2, 4, 5, 6 (for HCV); 8, 9, 10 (for Dengue virus); 12, 13, 14, 15, 39 (for Chikungunya virus); 16 and 17 (Broadspectrum activity for both HCV and Dengue Virus); 24 and 19 (for West Nile Virus); 21, 22 and 23 (Broad spectrum activity for both Dengue and West Nile Viruses); 32 (Broad spectrum activity for both HCV and Dengue Virus), whrerin the one or more targets of the 6 to 20 hybridization regions is comprised in SEQ ID NO.: 1, SEQ ID NO.: 7, SEQ ID NO.: 11 and/or SEQ ID NO.: 20.

14. The artificial circular RNA according to any of items 1 to 10 or 13, or the composition according to item 11 or 13 or the kit according to item 12 or 13 for use in preventing and/or treating a viral infection.

15. The artificial circular RNA or the composition or the kit for use according to item 14, wherein said viral infection is caused by HCV, HAV, Poliovirus, Coxsackie B virus, rhinovirus (common cold), Dengue, Zika, Chikungunya, West Nile or Yellow Fever virus.

1. An artificial circular RNA suitable for disrupting by hybridization one or more target disruption structures of one or more RNA fragments,
(a) wherein the artificial circular RNA comprises between 150 and 800 nucleotides, preferably between 200 and 600 nucleotides;
(b) wherein the artificial circular RNA comprises two or more hybridization regions which:
(i) completely hybridize with at least one target hybridization region comprised in the one or more target disruption structures of the one or more RNA fragments; and
(ii) have a total of between 7 and 100 nucleotides, preferably between 10 and 50 nucleotides;
(c) wherein the one or more target disruption structures:
(i) comprises at least a hairpin loop preceded or followed by a region of unpaired nucleotides; and
(ii) comprises at least one target hybridization region which comprises a single-stranded region of at least 2 nucleotides, preferably 3 nucleotides or more preceded or followed by a double-stranded region of at least 5 nucleotides, preferably 10 nucleotides or more, wherein the at least one target hybridization region completely hybridizes with each of the two or more hybridization regions of the artificial circular RNA; and
(d) wherein the two or more hybridization regions comprised in the artificial circular RNA are further characterized because, when hybridizing with the target hybridization region, the energy of hybridization, as measured by RNAcofold, between the hybridization region and the at least one target hybridization region is more negative than the energy of the target disruption region, thereby disrupting the target disruption structure.

2. The artificial circular RNA of claim 1 wherein the artificial circular RNA comprises between 6 and 20 hybridization regions.

3. The artificial circular RNA of claim 2 wherein at least two, and preferably all, of the hybridization regions are capable of completely hybridizing with the same target hybridization region.

4. The artificial circular RNA of any one of claims 2-3 wherein at least two, and preferably all, of the hybridization regions have different nucleotide sequences.

5. The artificial circular RNA of any one of claims 2-4, wherein the 2 or more hybridization regions are:
a) separated by non-hybridization regions of sizes up to 20 nucleotides; or
b) are not separated by non-hybridization regions; or
c) are overlapping.

6. The artificial circular RNA of any one of the preceding claims, wherein the one or more RNA fragments is selected from mRNA, tRNA, rRNA, non-coding RNA and viral genomic RNA.

7. The artificial circular RNA of claim 6, wherein the one or more RNA fragments is viral genomic RNA.

8. The artificial circular RNA of claim 7, wherein the one or more RNA fragments is positive-sense single-stranded viral genomic RNA.

9. The artificial circular RNA of any of claims 6 to 7 wherein the viral genomic RNA is selected from Influenza virus, HAV, Poliovirus, Coxsackie B virus, Coronavirus and Rhinovirus (common cold).

10. The artificial circular RNA of any of claims 6 to 8, wherein the viral genomic RNA is selected from Hepatitis C virus, Dengue, Zika, Chikungunya, West Nile and Yellow Fever virus.

11. The artificial circular RNA of any of claims 6 to 8, wherein the viral genomic RNA is from Severe acute respiratory syndrome coronavirus 2 (SARS-CoV-2).

12. The artificial circular RNA of any of claims 7 to 11, wherein the at least one or more target disruption structures are selected from the group consisting of:
(a) Internal Ribosome Entry (IRES) Domain IV and Domain V, capsid-coding region hairpin element (cHP) or SL427 from Hepatitis C Virus,
(b) short Stem Loop (sHP) or capsid-coding region hairpin element (cHP) from Dengue virus,
(c) 5′ untranslated region (5′UTR), Repetitive Sequence Element (RSE) or Recoding Element from Chikungunya,
(d) Stem loop III (SLIII) from West Nile, and/or
(e) SL-2, Replication site, Target A, Target C, Target D from Coronavirus, preferably from Severe acute respiratory syndrome coronavirus 2 (SARS-CoV-2).

13. The artificial circular RNA according to any of claims 7 to 12, wherein the at least one or more target disruption structures comprise the target disruption structures selected from the list consisting of SEQ ID NOs.:76, 77, 78 and/or 79 from Hepatitis C virus, and wherein the target hybridization regions, for each of these target disruption structures, are respectively selected from SEQ ID NOs: 25, 26, 27 and 28.

14. The artificial circular RNA according to any of claims 7 to 12, wherein the at least one or more target disruption structures comprise the target disruption structures selected from the list consisting of SEQ ID NOs.:29 and/or 30 from Dengue virus, and wherein the target hybridization regions, for each of these target disruption structures, are respectively selected from SEQ ID NOs.: 29 and 30.

15. The artificial circular RNA according to any of claims 7 to 12, wherein the at least one or more target disruption structures comprise the target disruption structures selected from the list consisting of SEQ ID NOs.:80, 81 and/or 82 from Chikungunya, and wherein the target hybridization regions, for each of these target disruption structures, are respectively selected from SEQ ID NOs.: 33, 35, and 31 .

16. The artificial circular RNA according to any of claims 7 to 12, wherein the at least one or more target disruption structures comprise the target disruption structure of SEQ ID NO.: 83 from West Nile and wherein the target hybridization regions is SEQ ID NO.: 37.

17. The artificial circular RNA according any of claims 7 to 12, wherein the at least one or more target disruption structures comprise the target disruption structures selected from the list consisting of SEQ ID NOs.: 84, 58, 85, 86, and/or 87 from Severe acute respiratory syndrome coronavirus 2 (SARS-CoV-2), and wherein the target hybridization regions, for each of these target disruption structures, are respectively selected from SEQ ID NOs.: 62, 58, 59, 60, and 61.

18. The artificial circular RNA according any of claims 7 to 12, wherein the at least one or more target disruption structures comprise the target disruption structures selected from the list consisting of SEQ ID NOs.: 30 and/or 79 from Dengue Virus and Hepatitis C Virus, and wherein the target hybridization regions, for each of these target disruption structures, are elected from SEQ ID NOs.: 30 and 28.

19. The artificial circular RNA according any of claims 7 to 12, wherein the at least one or more target disruption structures comprise the target disruption structures selected from the list consisting of SEQ ID NOs.: 30 and/or 83 from Dengue Virus and West Nile Virus, and wherein the target hybridization regions, for each of these target disruption structures, are respectively selected from SEQ ID NOs.: 30 and 37.

20. A composition comprising the artificial circular RNA as defined in any of the previous claims.

21. A kit comprising the artificial circular RNA as defined in any of claims 1 to 19 or comprising the composition as defined in claim 20, and instructions for using the artificial circular RNA or composition.

22. The artificial circular RNA of any of claims 1 to 19, or the composition of claim 20, or the kit of claim 21, wherein the sequence of the artificial RNA comprises, or preferably, consists of, the following nucleotides defined in: SEQ ID NO: 2, 3, 4, 5, 6 (for Hepatitis C virus); 8, 9, 10 (for Dengue virus); 12, 13, 14, 15, 39 (for Chikungunya virus); 16 and 17 (broad spectrum activity for both Hepatitis C virus and Dengue Virus); 24 and 19 (for West Nile Virus); 21, 22 and 23 (broad spectrum activity for both Dengue and West Nile Viruses); 32 (broad spectrum activity for both Hepatitis C virus and Dengue Virus); 36, 38, 40, 41, 42, 43, 44, 45, 46, 47, 48, 49, 50, 51, 52, 53, 54, 55, 56, 57, 65, 66, 67, 68, 69, 70, 71, 72 (for Severe acute respiratory syndrome coronavirus 2).

23. The artificial circular RNA of any of claims 1 to 19 or the composition of claim 20, or the kit of claim 21, wherein the one or more target hybridization region of the artificial circular RNA which completely hybridize with the two or more hybridization regions is comprised in the artificial RNA defined in SEQ ID NO.: 1, SEQ ID NO.: 7, SEQ ID NO.: 11, SEQ ID NO.: 34 and/or SEQ ID NO.: 20.

24. The artificial circular RNA of any one of claims 1 to 19 and 22 and 23, or the composition of any of claims 20 to 23, or the kit of any one of claims 21 to 23 for use as a medicament.

25. The artificial circular RNA of any one of claims 1 to 19 and 22 to 24, or the composition of any of claims 20 to 24, or the kit of any one of claims 21 to 24 for use in a method of preventing and/or treating a viral infection.

26. The artificial circular RNA of any one of claims 1 to 19 and 22 to 25, or the composition of any of claims 20 to 25, or the kit of any one of claims 21 to 26 for use according to claim 25, wherein the viral infection is caused by Hepatitis C virus, Hepatitis A virus, Poliovirus, Influenza virus, Coxsackie B virus, rhinovirus (common cold), Dengue, Zika, Chikungunya, West Nile, Yellow Fever virus or coronavirus, such as SARS and/or MERS, preferably SARS-CoV-2.

27. A method of screening for artificial circular RNA comprising two or more hybridization regions capable of disrupting by hybridization one or more target disruption structures of one or more RNA fragments, wherein the target disruption structures are defined as:
iii. a first region with at least a hairpin loop preceded or followed by a second region of unpaired nucleotides; and
iv. as comprising at least one target hybridization region which comprises a single-stranded region of at least 2 nucleotides, preferably 3 nucleotides or more preceded or followed by a double-stranded region of at least 5 nucleotides, preferably 10 nucleotides or more, and
wherein the method comprises the steps of:
d) identifying the two or more hybridization regions of the artificial circular RNA as those regions that have a total of between 7 and 100 nucleotides in length, preferably between 10 and 50 nucleotides that, when hybridizing with the at least one target hybridization region, the energy of the hybridization between the two or more hybridization regions and the at least one target hybridization region is more negative than the energy of the target disruption structure, thereby disrupting the one or more target disruption structure; wherein the the two or more hybridization regions comprised in the artificial circular RNA, are identified by RNA inverse folding tools, such as NUPACK, RNAifold, or MoiRNAiFold;
e) designing an artificial circular RNA comprising the two or more hybridization regions capable of disrupting the one or more target disruption structures as identified in step a), wherein said artificial circular RNA is between 150 and 800 nucleotides in length, preferably between 200 and 600 nucleotides; and
f) optionally selecting the artificial circular RNA capable of disrupting by hybridization the one or more target disruption structures as designed in step b), and optionally packaging it into a product.
